(19) United States
(12) Patent Application Publication
(10) Pub. No.: US 2026/0248910 A1
(43) Pub. Date: Aug. 27, 2026

(54) SOYBEAN ALLERGY ANTIGEN

(71) Applicant: HOYU CO., LTD., Aichi (JP)

(72) Inventors: Kayoko Matsunaga, Aichi (JP); Akiko Yagami, Aichi (JP); Naoshi Shimojo, Aichi (JP); Masashi Nakamura, Aichi (JP); Nayu Sato, Aichi (JP)

(73) Assignee: HOYU CO., LTD., Aichi (JP)

(21) Appl. No.: 19/329,452

(22) Filed: Sep. 15, 2025

Related U.S. Application Data
(63) Continuation of application No. 18/462,987, filed on Sep. 7, 2023, now Pat. No. 12,440,558.
Application No. 18/462,987 is a division of application No. 16/081,032, filed on Mar. 3, 2017, now abandoned.

(30) Foreign Application Priority Data
Mar. 3, 2016 (JP) 2016-041547

(51) Int. Cl. A61K 39/35; A23L 11/30; A61K 38/00; A61K 49/00; A61P 37/08; C07K 4/10; C07K 14/415; C07K 14/47; G01N 33/53

(52) U.S. Cl. CPC A61K 39/35; A23L 11/30; A61K 49/00; A61K 49/0006; A61P 37/08; C07K 4/10; C07K 14/415; C07K 14/47; G01N 33/53; A61K 38/00; G01N 2800/24

(57) ABSTRACT

The present invention provides novel antigens of an allergy to soybean, methods and kits for diagnosing an allergy to soybean, pharmaceutical compositions comprising such an antigen, soybeans or processed products of soybean in which such an antigen is eliminated, and a tester for determining the presence or absence of a soybean antigen in an object of interest.

### CROSS-REFERENCE TO RELATED APPLICATIONS

This application is a continuation application of U.S. patent application Ser. No. 18/462,987, filed on Sep. 7, 2023, which is a divisional application of U.S. patent application Ser. No. 16/081,032, filed on Aug. 30, 2018, which is a U.S. national stage filing, under 35 U.S.C. § 371(c), of International Application No. PCT/JP2017/008593, filed on Mar. 3, 2017, which claims priority to Japanese Patent Application No. 2016-041547, filed on Mar. 3, 2016. The entire contents of each of the aforementioned applications are incorporated herein by reference.

### REFERENCE TO SEQUENCE LISTING

The instant application contains a Sequence Listing XML file which has been submitted electronically in XML format and is hereby incorporated by reference in its entirety. Said XML file, created on Aug. 30, 2023, is named Seq_Listing_129249_00204.XML and is 702,767 bytes in size.

### TECHNICAL FIELD

The present invention relates to a novel antigen of an allergy to soybean. The present invention also relates to a kit, a composition, and a method for diagnosing allergy to soybean. The present invention also relates to a pharmaceutical composition comprising such an antigen and soybean or processed products of soybean in which such an antigen is eliminated or reduced. The present invention further relates to a tester for determining the presence or absence of a soybean antigen in an object of interest.

### BACKGROUND ART

In serum and tissues of allergic patients, IgE antibodies specific to particular antigens are produced. Physiological consequences caused by interaction between such IgE antibodies and such particular antigens elicit allergic reactions.

In the process of production of conventional allergy testing agents, antigen reagents are commonly prepared simply by grinding a candidate allergenic food, material or the like (Patent Literature 1). As seen above, conventional antigen reagents do not necessarily contain only a particular antigenic protein inducing an allergic reaction (allergen component), and rather contain different types of protein components. Thus, conventional antigen reagents contain varied amounts of allergen components. For this reason, the only case where conventional allergy tests have permitted detection of a positive allergic reaction is when in a conventional antigen reagent containing many types of proteins, a particular protein acting as an allergen component is present in an amount exceeding a threshold that allows determination of a positive reaction for binding to an IgE antibody. However, no determination of a positive reaction was possible and diagnosis efficiency was not sufficiently high when using a conventional allergy testing agent in patients possessing an IgE antibody binding to an allergen component present in small amounts in an allergen such as food.

The severity and symptoms of an allergic reaction do not necessarily correlate with the content of an allergen component. Even when a patient's IgE antibody reacts with an allergen component present in trace amounts in a candidate allergic food, material or the like, the allergic reaction may develop allergic symptoms or may affect the severity of those symptoms.

An attempt to increase the diagnostic efficiency is being made by examining IgE antibodies to components composing a crude antigen to distinguish sensitization that directly contributes to a diagnosis from sensitization based on cross-antigenicity by a pan-allergen or the like. 8 soybean allergens are currently registered to the World Health Organization/International Union of Immunological Societies (WHO/IUIS).

However, while it is necessary to exhaustively identify allergen components in candidate allergic foods and materials in order to enhance the reliability of allergy tests, the patient detection rate by the measurement of such allergenic components is far insufficient. Identification of novel allergens in soybean is very important not only for increasing the precision of diagnosis, but also for determining targets of therapeutic agents and low allergenic foods.

Meanwhile, in the field of protein separation and purification, various efforts have conventionally been made to develop methods for separating and purifying a protein or nucleic acid of interest from cell extracts or the like. Such methods may well be exemplified by dialysis based on salt concentration, and centrifugal separation.

Other efforts have been made to develop many purification methods based on electric charges of protein or nucleic acid residues or on the difference in molecular weight. Electric charge-based purification methods can be exemplified by column chromatography using ion exchange resins, and isoelectric focusing. Purifications based on molecular weight difference can be exemplified by centrifugal separation, molecular-sieve column chromatography, and SDS-PAGE (sodium dodecyl sulfate-polyacrylamide gel electrophoresis).

In recent years, a method for separating and purifying many different proteins from a small amount of sample has been used, which is more specifically a two-dimensional electrophoresis consisting of isoelectric focusing in the first dimension, followed by SDS-PAGE in the second dimension. The present applicant has conventionally developed some 2D electrophoresis methods with high separation ability (Patent Literature 2-5).

### CITATION LIST

### Patent Literature

PTL1: Japanese Patent Application Publication No. JP 2002-286716
PTL2: Japanese Patent Application Publication No. JP 2011-33544
PTL3: Japanese Patent Application Publication No. JP 2011-33546
PTL4: Japanese Patent Application Publication No. JP 2011-33547
PTL5: Japanese Patent Application Publication No. JP 2011-33548

### SUMMARY OF INVENTION

### Technical Problem

The present invention provides novel antigens of an allergy to soybean. The present invention also provides methods and kits for diagnosing allergy to soybean. The present invention also provides pharmaceutical compositions comprising such an antigen and soybean or processed products of soybean in which such an antigen is eliminated or reduced. The present invention further provides testers for determining the presence or absence of a soybean antigen in an object of interest.

### Solution to Problem

In order to solve the aforementioned problems, the present inventors had made intensive studies to identify causative antigens of an allergy to soybean. As a result, the inventors succeeded in identifying novel antigens to which an IgE antibody in the serum of a soybean-allergic patient specifically binds. The present invention has been completed based on this finding.

Thus, in one embodiment, the present invention can be as defined below.
[1] A kit for diagnosing an allergy to a soybean, the kit comprising, as an antigen, at least one of proteins defined below in any of the following (1) to (8):
   (1) (1A) Bowman-Birk type proteinase inhibitor D-II or a variant thereof, which is defined below in any of (1A-a) to (1A-e):
(1A-a) a protein comprising an amino acid sequence with deletion, substitution, insertion or addition of one or several amino acids in SEQ ID NO: 2;
(1A-b) a protein comprising an amino acid sequence having at least 70% identity to the amino acid sequence of SEQ ID NO: 2;
(1A-c) a protein comprising an amino acid sequence encoded by a nucleotide sequence with deletion, substitution, insertion or addition of one or several nucleotides in SEQ ID NO: 1;
(1A-d) a protein comprising an amino acid sequence encoded by a nucleotide sequence having at least 70% identity to the nucleotide sequence of SEQ ID NO: 1; or
(1A-e) a protein comprising an amino acid sequence encoded by a nucleic acid that hybridizes under stringent conditions with a nucleic acid having a nucleotide sequence complementary to the nucleotide sequence of SEQ ID NO: 1; or
(1B) a protein comprising at least one amino acid sequence selected from the group consisting of SEQ ID NOs: 17-20;
   (2) (2A) Ribulose bisphosphate carboxylase large chain or a variant thereof, which is defined below in any of (2A-a) to (2A-e):
(2A-a) a protein comprising an amino acid sequence with deletion, substitution, insertion or addition of one or several amino acids in SEQ ID NO: 4;
(2A-b) a protein comprising an amino acid sequence having at least 70% identity to the amino acid sequence of SEQ ID NO: 4;
(2A-c) a protein comprising an amino acid sequence encoded by a nucleotide sequence with deletion, substitution, insertion or addition of one or several nucleotides in SEQ ID NO: 3;
(2A-d) a protein comprising an amino acid sequence encoded by a nucleotide sequence having at least 70% identity to the nucleotide sequence of SEQ ID NO: 3; or
(2A-e) a protein comprising an amino acid sequence encoded by a nucleic acid that hybridizes under stringent conditions with a nucleic acid having a nucleotide sequence complementary to the nucleotide sequence of SEQ ID NO: 3; or
(2B) a protein comprising at least one amino acid sequence selected from the group consisting of SEQ ID NOs: 21-28;
   (3) (3A) Gamma-glutamyl hydrolase or a variant thereof, which is defined below in any of (3A-a) to (3A-e):
(3A-a) a protein comprising an amino acid sequence with deletion, substitution, insertion or addition of one or several amino acids in SEQ ID NO: 6;
(3A-b) a protein comprising an amino acid sequence having at least 70% identity to the amino acid sequence of SEQ ID NO: 6;
(3A-c) a protein comprising an amino acid sequence encoded by a nucleotide sequence with deletion, substitution, insertion or addition of one or several nucleotides in SEQ ID NO: 5;
(3A-d) a protein comprising an amino acid sequence encoded by a nucleotide sequence having at least 70% identity to the nucleotide sequence of SEQ ID NO: 5; or
(3A-e) a protein comprising an amino acid sequence encoded by a nucleic acid that hybridizes under stringent conditions with a nucleic acid having a nucleotide sequence complementary to the nucleotide sequence of SEQ ID NO: 5; or
(3B) a protein comprising an amino acid sequence of SEQ ID NO: 29 or 30;
   (4) (4A) Guanine nucleotide-binding protein subunit beta-like protein or a variant thereof, which is defined below in any of (4A-a) to (4A-e):
(4A-a) a protein comprising an amino acid sequence with deletion, substitution, insertion or addition of one or several amino acids in SEQ ID NO: 8;
(4A-b) a protein comprising an amino acid sequence having at least 70% identity to the amino acid sequence of SEQ ID NO: 8;
(4A-c) a protein comprising an amino acid sequence encoded by a nucleotide sequence with deletion, substitution, insertion or addition of one or several nucleotides in SEQ ID NO: 7;
(4A-d) a protein comprising an amino acid sequence encoded by a nucleotide sequence having at least 70% identity to the nucleotide sequence of SEQ ID NO: 7; or
(4A-e) a protein comprising an amino acid sequence encoded by a nucleic acid that hybridizes under stringent conditions with a nucleic acid having a nucleotide sequence complementary to the nucleotide sequence of SEQ ID NO: 7; or
(4B) a protein comprising an amino acid sequence of SEQ ID NO: 31 or 32;
   (5) (5A) Protein SLE3 or a variant thereof, which is defined below in any of (5A-a) to (5A-e):
(5A-a) a protein comprising an amino acid sequence with deletion, substitution, insertion or addition of one or several amino acids in SEQ ID NO: 10;
(5A-b) a protein comprising an amino acid sequence having at least 70% identity to the amino acid sequence of SEQ ID NO: 10;
(5A-c) a protein comprising an amino acid sequence encoded by a nucleotide sequence with deletion, substitution, insertion or addition of one or several nucleotides in SEQ ID NO: 9;
(5A-d) a protein comprising an amino acid sequence encoded by a nucleotide sequence having at least 70% identity to the nucleotide sequence of SEQ ID NO: 9; or
(5A-e) a protein comprising an amino acid sequence encoded by a nucleic acid that hybridizes under stringent conditions with a nucleic acid having a nucleotide sequence complementary to the nucleotide sequence of SEQ ID NO: 9; or
(5B) a protein comprising an amino acid sequence of SEQ ID NO: 33 or 34;
   (6) (6A) Seed biotin-containing protein SBP65 or a variant thereof, which is defined below in any of (6A-a) to (6A-e):
(6A-a) a protein comprising an amino acid sequence with deletion, substitution, insertion or addition of one or several amino acids in SEQ ID NO: 12;
(6A-b) a protein comprising an amino acid sequence having at least 70% identity to the amino acid sequence of SEQ ID NO: 12;
(6A-c) a protein comprising an amino acid sequence encoded by a nucleotide sequence with deletion, substitution, insertion or addition of one or several nucleotides in SEQ ID NO: 11;
(6A-d) a protein comprising an amino acid sequence encoded by a nucleotide sequence having at least 70% identity to the nucleotide sequence of SEQ ID NO: 11; or
(6A-e) a protein comprising an amino acid sequence encoded by a nucleic acid that hybridizes under stringent conditions with a nucleic acid having a nucleotide sequence complementary to the nucleotide sequence of SEQ ID NO: 11; or
(6B) a protein comprising at least one amino acid sequence selected from the group consisting of SEQ ID NOs: 35-53;
   (7) (7A) Ferritin-2 or a variant thereof, which is defined below in any of (7A-a) to (7A-e):
(7A-a) a protein comprising an amino acid sequence with deletion, substitution, insertion or addition of one or several amino acids in SEQ ID NO: 14;
(7A-b) a protein comprising an amino acid sequence having at least 70% identity to the amino acid sequence of SEQ ID NO: 14;
(7A-c) a protein comprising an amino acid sequence encoded by a nucleotide sequence with deletion, substitution, insertion or addition of one or several nucleotides in SEQ ID NO: 13;
(7A-d) a protein comprising an amino acid sequence encoded by a nucleotide sequence having at least 70% identity to the nucleotide sequence of SEQ ID NO: 13; or
(7A-e) a protein comprising an amino acid sequence encoded by a nucleic acid that hybridizes under stringent conditions with a nucleic acid having a nucleotide sequence complementary to the nucleotide sequence of SEQ ID NO: 13; or
(7B) a protein comprising at least one amino acid sequence selected from the group consisting of SEQ ID NOs: 54-61;
   (8) (8A) Cell division cycle protein 48 homolog or a variant thereof, which is defined below in any of (8A-a) to (8A-e):
(8A-a) a protein comprising an amino acid sequence with deletion, substitution, insertion or addition of one or several amino acids in SEQ ID NO: 16;
(8A-b) a protein comprising an amino acid sequence having at least 70% identity to the amino acid sequence of SEQ ID NO: 16;
(8A-c) a protein comprising an amino acid sequence encoded by a nucleotide sequence with deletion, substitution, insertion or addition of one or several nucleotides in SEQ ID NO: 15;
(8A-d) a protein comprising an amino acid sequence encoded by a nucleotide sequence having at least 70% identity to the nucleotide sequence of SEQ ID NO: 15; or
(8A-e) a protein comprising an amino acid sequence encoded by a nucleic acid that hybridizes under stringent conditions with a nucleic acid having a nucleotide sequence complementary to the nucleotide sequence of SEQ ID NO: 15; or
(8B) a protein comprising at least one amino acid sequence selected from the group consisting of SEQ ID NOs: 62-68.
[2] A composition for diagnosing an allergy to a soybean, comprising, as an antigen, at least one of proteins as defined above in any of (1) to (8) of [1].
[3] A method for providing an indicator for diagnosing an allergy to a soybean in a subject, the method comprising the steps of:
   (i) contacting a sample obtained from the subject with an antigen, wherein the sample is a solution comprising an Ig antibody;
   (ii) detecting binding between the IgE antibody present in the sample from the subject and the antigen; and
   (iii) when the binding between the IgE antibody in the subject and the antigen is detected, an indicator of the fact that the subject is allergic to a soybean is provided;
   wherein the antigen is at least one of proteins as defined above in any of (1) to (8) of [1].
[4] A pharmaceutical composition comprising at least one of proteins as defined above in any of (1) to (8) of [1].
[5] The pharmaceutical composition as set forth in [4], wherein the pharmaceutical composition is intended for the treatment of an allergy to a soybean.
[6] A soybean or processed product of soybean, in which an antigen is eliminated or reduced, wherein antigen is at least one of proteins as defined above in any of (1) to (8) of [1].
[7] A tester for determining the presence or absence of a soybean antigen in an object of interest, comprising an antibody that binds to at least one of proteins as defined above in any of (1) to (8) of [1].
[8] A soybean-derived antigen which is at least one of proteins as defined above in any of (1) to (8) of [1] and is causative of an allergy to soybean.
[9] A kit for diagnosing an allergy to a soybean, the kit comprising, as an antigen, at least one of proteins defined below in any one of (1α) to (55α):
   (1α) (1αA1) endoplasmin homolog isoform X2 or a variant thereof, which is defined below in any of (1αA1-a) to (1αA1-e):
(1αA1-a) a protein comprising an amino acid sequence with deletion, substitution, insertion or addition of one or several amino acids in SEQ ID NO: 70;
(1αA1-b) a protein comprising an amino acid sequence having at least 70% identity to the amino acid sequence of SEQ ID NO: 70;
(1αA1-c) a protein comprising an amino acid sequence encoded by a nucleotide sequence with deletion, substitution, insertion or addition of one or several nucleotides in SEQ ID NO: 69;
(1αA1-d) a protein comprising an amino acid sequence encoded by a nucleotide sequence having at least 70% identity to the nucleotide sequence of SEQ ID NO: 69; or
(1αA1-e) a protein comprising an amino acid sequence encoded by a nucleic acid that hybridizes under stringent conditions with a nucleic acid having a nucleotide sequence complementary to the nucleotide sequence of SEQ ID NO: 69;
(1αB1) a protein comprising at least one amino acid sequence selected from the group consisting of SEQ ID NOs: 71-87,
(1αA2) endoplasmin homolog isoform X2 or a variant thereof, which is defined below in any of (1αA2-a) to (1αA2-e):
(1αA2-a) a protein comprising an amino acid sequence with deletion, substitution, insertion or addition of one or several amino acids in SEQ ID NO: 89;
(1αA2-b) a protein comprising an amino acid sequence having at least 70% identity to the amino acid sequence of SEQ ID NO: 89;
(1αA2-c) a protein comprising an amino acid sequence encoded by a nucleotide sequence with deletion, substitution, insertion or addition of one or several nucleotides in SEQ ID NO: 88;
(1αA2-d) a protein comprising an amino acid sequence encoded by a nucleotide sequence having at least 70% identity to the nucleotide sequence of SEQ ID NO: 88; or
(1αA2-e) a protein comprising an amino acid sequence encoded by a nucleic acid that hybridizes under stringent conditions with a nucleic acid having a nucleotide sequence complementary to the nucleotide sequence of SEQ ID NO: 88; or
(1αB2) a protein comprising at least one amino acid sequence selected from the group consisting of SEQ ID NOs: 90-104;
   (2α) (2αA1) heat shock cognate protein 80 or a variant thereof, which is defined below in any of (2αA1-a) to (2αA1-e):
(2αA1-a) a protein comprising an amino acid sequence with deletion, substitution, insertion or addition of one or several amino acids in SEQ ID NO: 106;
(2αA1-b) a protein comprising an amino acid sequence having at least 70% identity to the amino acid sequence of SEQ ID NO: 106;
(2αA1-c) a protein comprising an amino acid sequence encoded by a nucleotide sequence with deletion, substitution, insertion or addition of one or several nucleotides in SEQ ID NO: 105;
(2αA1-d) a protein comprising an amino acid sequence encoded by a nucleotide sequence having at least 70% identity to the nucleotide sequence of SEQ ID NO: 105; or
(2αA1-e) a protein comprising an amino acid sequence encoded by a nucleic acid that hybridizes under stringent conditions with a nucleic acid having a nucleotide sequence complementary to the nucleotide sequence of SEQ ID NO: 105; or
(2αB1) a protein comprising at least one amino acid sequence selected from the group consisting of SEQ ID NOs: 107-120;
(2αA2) heat shock protein 90-1 or a variant thereof, which is defined below in any of (2αA2-a) to (2αA2-e):
(2αA2-a) a protein comprising an amino acid sequence with deletion, substitution, insertion or addition of one or several amino acids in SEQ ID NO: 122;
(2αA2-b) a protein comprising an amino acid sequence having at least 70% identity to the amino acid sequence of SEQ ID NO: 122;
(2αA2-c) a protein comprising an amino acid sequence encoded by a nucleotide sequence with deletion, substitution, insertion or addition of one or several nucleotides in SEQ ID NO: 121;
(2αA2-d) a protein comprising an amino acid sequence encoded by a nucleotide sequence having at least 70% identity to the nucleotide sequence of SEQ ID NO: 121; or
(2αA2-e) a protein comprising an amino acid sequence encoded by a nucleic acid that hybridizes under stringent conditions with a nucleic acid having a nucleotide sequence complementary to the nucleotide sequence of SEQ ID NO: 121; or
(2αB2) a protein comprising at least one amino acid sequence selected from the group consisting of SEQ ID NOs: 123-137;
(2αA3) hypothetical protein GLYMA_02G302500 or a variant thereof, which is defined below in any of (2αA3-a) to (2αA3-e):
(2αA3-a) a protein comprising an amino acid sequence with deletion, substitution, insertion or addition of one or several amino acids in SEQ ID NO: 139;
(2αA3-b) a protein comprising an amino acid sequence having at least 70% identity to the amino acid sequence of SEQ ID NO: 139;
(2αA3-c) a protein comprising an amino acid sequence encoded by a nucleotide sequence with deletion, substitution, insertion or addition of one or several nucleotides in SEQ ID NO: 138;
(2αA3-d) a protein comprising an amino acid sequence encoded by a nucleotide sequence having at least 70% identity to the nucleotide sequence of SEQ ID NO: 138; or
(2αA3-e) a protein comprising an amino acid sequence encoded by a nucleic acid that hybridizes under stringent conditions with a nucleic acid having a nucleotide sequence complementary to the nucleotide sequence of SEQ ID NO: 138; or
(2αB3) a protein comprising at least one amino acid sequence selected from the group consisting of SEQ ID NOs: 140-153;
   (4α) (4αA) embryo-specific urease isoform X1 or a variant thereof, which is defined below in any of (4αA-a) to (4αA-e):
(4αA-a) a protein comprising an amino acid sequence with deletion, substitution, insertion or addition of one or several amino acids in SEQ ID NO: 155;
(4αA-b) a protein comprising an amino acid sequence having at least 70% identity to the amino acid sequence of SEQ ID NO: 155;
(4αA-c) a protein comprising an amino acid sequence encoded by a nucleotide sequence with deletion, substitution, insertion or addition of one or several nucleotides in SEQ ID NO: 154;
(4αA-d) a protein comprising an amino acid sequence encoded by a nucleotide sequence having at least 70% identity to the nucleotide sequence of SEQ ID NO: 154; or
(4αA-e) a protein comprising an amino acid sequence encoded by a nucleic acid that hybridizes under stringent conditions with a nucleic acid having a nucleotide sequence complementary to the nucleotide sequence of SEQ ID NO: 154; or
(4αB) a protein comprising at least one amino acid sequence selected from the group consisting of SEQ ID NOs: 156 and 157;
   (5α) (5αA) alpha-1,4 glucan phosphorylase L isozyme, chloroplastic/amyloplastic or a variant thereof, which is defined below in any of (5αA-a) to (5αA-e):
(5αA-a) a protein comprising an amino acid sequence with deletion, substitution, insertion or addition of one or several amino acids in SEQ ID NO: 159;
(5αA-b) a protein comprising an amino acid sequence having at least 70% identity to the amino acid sequence of SEQ ID NO: 159;
(5αA-c) a protein comprising an amino acid sequence encoded by a nucleotide sequence with deletion, substitution, insertion or addition of one or several nucleotides in SEQ ID NO: 158;
(5αA-d) a protein comprising an amino acid sequence encoded by a nucleotide sequence having at least 70% identity to the nucleotide sequence of SEQ ID NO: 158; or
(5αA-e) a protein comprising an amino acid sequence encoded by a nucleic acid that hybridizes under stringent conditions with a nucleic acid having a nucleotide sequence complementary to the nucleotide sequence of SEQ ID NO: 158; or
(5αB) a protein comprising at least one amino acid sequence selected from the group consisting of SEQ ID NOs: 160 and 161;
   (6α) (6αA1) aconitate hydratase 1 or a variant thereof, which is defined below in any of (6αA1-a) to (6αA1-e):
(6αA1-a) a protein comprising an amino acid sequence with deletion, substitution, insertion or addition of one or several amino acids in SEQ ID NO: 163;
(6αA1-b) a protein comprising an amino acid sequence having at least 70% identity to the amino acid sequence of SEQ ID NO: 163;
(6αA1-c) a protein comprising an amino acid sequence encoded by a nucleotide sequence with deletion, substitution, insertion or addition of one or several nucleotides in SEQ ID NO: 162;
(6αA1-d) a protein comprising an amino acid sequence encoded by a nucleotide sequence having at least 70% identity to the nucleotide sequence of SEQ ID NO: 162; or
(6αA1-e) a protein comprising an amino acid sequence encoded by a nucleic acid that hybridizes under stringent conditions with a nucleic acid having a nucleotide sequence complementary to the nucleotide sequence of SEQ ID NO: 162;
(6αB1) a protein comprising at least one amino acid sequence selected from the group consisting of SEQ ID NOs: 164-167; or
(6αA2) aconitate hydratase 1 or a variant thereof, which is defined below in any of (6αA2-a) to (6αA2-e):
(6αA2-a) a protein comprising an amino acid sequence with deletion, substitution, insertion or addition of one or several amino acids in SEQ ID NO: 169;
(6αA2-b) a protein comprising an amino acid sequence having at least 70% identity to the amino acid sequence of SEQ ID NO: 169;
(6αA2-c) a protein comprising an amino acid sequence encoded by a nucleotide sequence with deletion, substitution, insertion or addition of one or several nucleotides in SEQ ID NO: 168;
(6αA2-d) a protein comprising an amino acid sequence encoded by a nucleotide sequence having at least 70% identity to the nucleotide sequence of SEQ ID NO: 168; or
(6αA2-e) a protein comprising an amino acid sequence encoded by a nucleic acid that hybridizes under stringent conditions with a nucleic acid having a nucleotide sequence complementary to the nucleotide sequence of SEQ ID NO: 168; or
(6αB2) a protein comprising at least one amino acid sequence selected from the group consisting of SEQ ID NOs: 170-172;
   (7α) (7αA) methionine synthase or a variant thereof, which is defined below in any of (7αA-a) to (7αA-e):
(7αA-a) a protein comprising an amino acid sequence with deletion, substitution, insertion or addition of one or several amino acids in SEQ ID NO: 174;
(7αA-b) a protein comprising an amino acid sequence having at least 70% identity to the amino acid sequence of SEQ ID NO: 174;
(7αA-c) a protein comprising an amino acid sequence encoded by a nucleotide sequence with deletion, substitution, insertion or addition of one or several nucleotides in SEQ ID NO: 173;
(7αA-d) a protein comprising an amino acid sequence encoded by a nucleotide sequence having at least 70% identity to the nucleotide sequence of SEQ ID NO: 173; or
(7αA-e) a protein comprising an amino acid sequence encoded by a nucleic acid that hybridizes under stringent conditions with a nucleic acid having a nucleotide sequence complementary to the nucleotide sequence of SEQ ID NO: 173; or
(7αB) a protein comprising at least one amino acid sequence selected from the group consisting of SEQ ID NOs: 175-187;
   (8α) (8αA) transketolase, chloroplastic or a variant thereof, which is defined below in any of (8αA-a) to (8αA-e):
(8αA-a) a protein comprising an amino acid sequence with deletion, substitution, insertion or addition of one or several amino acids in SEQ ID NO: 189;
(8αA-b) a protein comprising an amino acid sequence having at least 70% identity to the amino acid sequence of SEQ ID NO: 189;
(8αA-c) a protein comprising an amino acid sequence encoded by a nucleotide sequence with deletion, substitution, insertion or addition of one or several nucleotides in SEQ ID NO: 188;
(8αA-d) a protein comprising an amino acid sequence encoded by a nucleotide sequence having at least 70% identity to the nucleotide sequence of SEQ ID NO: 188; or
(8αA-e) a protein comprising an amino acid sequence encoded by a nucleic acid that hybridizes under stringent conditions with a nucleic acid having a nucleotide sequence complementary to the nucleotide sequence of SEQ ID NO: 188; or
(8αB) a protein comprising at least one amino acid sequence selected from the group consisting of SEQ ID NOs: 190-199;
   (9α) (9αA) lysine-tRNA ligase, cytoplasmic-like isoform X2 or a variant thereof, which is defined below in any of (9αA-a) to (9αA-e):
(9αA-a) a protein comprising an amino acid sequence with deletion, substitution, insertion or addition of one or several amino acids in SEQ ID NO: 201;
(9αA-b) a protein comprising an amino acid sequence having at least 70% identity to the amino acid sequence of SEQ ID NO: 201;
(9αA-c) a protein comprising an amino acid sequence encoded by a nucleotide sequence with deletion, substitution, insertion or addition of one or several nucleotides in SEQ ID NO: 200;
(9αA-d) a protein comprising an amino acid sequence encoded by a nucleotide sequence having at least 70% identity to the nucleotide sequence of SEQ ID NO: 200; or
(9αA-e) a protein comprising an amino acid sequence encoded by a nucleic acid that hybridizes under stringent conditions with a nucleic acid having a nucleotide sequence complementary to the nucleotide sequence of SEQ ID NO: 200; or (9αB) a protein comprising an amino acid sequence of SEQ ID NO: 202;
   (10α) (10αA1) chaperonin CPN60-2, mitochondrial or a variant thereof, which is defined below in any of (10αA1-a) to (10αA1-e):
(10αA1-a) a protein comprising an amino acid sequence with deletion, substitution, insertion or addition of one or several amino acids in SEQ ID NO: 204;
(10αA1-b) a protein comprising an amino acid sequence having at least 70% identity to the amino acid sequence of SEQ ID NO: 204;
(10αA1-c) a protein comprising an amino acid sequence encoded by a nucleotide sequence with deletion, substitution, insertion or addition of one or several nucleotides in SEQ ID NO: 203;
(10αA1-d) a protein comprising an amino acid sequence encoded by a nucleotide sequence having at least 70% identity to the nucleotide sequence of SEQ ID NO: 203; or
(10αA1-e) a protein comprising an amino acid sequence encoded by a nucleic acid that hybridizes under stringent conditions with a nucleic acid having a nucleotide sequence complementary to the nucleotide sequence of SEQ ID NO: 203;
(10αB1) a protein comprising at least one amino acid sequence selected from the group consisting of SEQ ID NOs: 205-218;
(10αA2) chaperonin CPN60-2, mitochondrial-like isoform X1 or a variant thereof, which is defined below in any of (10αA2-a) to (10αA2-e):
(10αA2-a) a protein comprising an amino acid sequence with deletion, substitution, insertion or addition of one or several amino acids in SEQ ID NO: 220;
(10αA2-b) a protein comprising an amino acid sequence having at least 70% identity to the amino acid sequence of SEQ ID NO: 220;
(10αA2-c) a protein comprising an amino acid sequence encoded by a nucleotide sequence with deletion, substitution, insertion or addition of one or several nucleotides in SEQ ID NO: 219;
(10αA2-d) a protein comprising an amino acid sequence encoded by a nucleotide sequence having at least 70% identity to the nucleotide sequence of SEQ ID NO: 219; or
(10αA2-e) a protein comprising an amino acid sequence encoded by a nucleic acid that hybridizes under stringent conditions with a nucleic acid having a nucleotide sequence complementary to the nucleotide sequence of SEQ ID NO: 219; or
(10αB2) a protein comprising at least one amino acid sequence selected from the group consisting of SEQ ID NOs: 221-231;
   (11α) (11αA) polyadenylate-binding protein 8 isoform X3 or a variant thereof, which is defined below in any of (11αA-a) to (11αA-e):
(11αA-a) a protein comprising an amino acid sequence with deletion, substitution, insertion or addition of one or several amino acids in SEQ ID NO: 233;
(11αA-b) a protein comprising an amino acid sequence having at least 70% identity to the amino acid sequence of SEQ ID NO: 233;
(11αA-c) a protein comprising an amino acid sequence encoded by a nucleotide sequence with deletion, substitution, insertion or addition of one or several nucleotides in SEQ ID NO: 232;
(11αA-d) a protein comprising an amino acid sequence encoded by a nucleotide sequence having at least 70% identity to the nucleotide sequence of SEQ ID NO: 232; or
(11αA-e) a protein comprising an amino acid sequence encoded by a nucleic acid that hybridizes under stringent conditions with a nucleic acid having a nucleotide sequence complementary to the nucleotide sequence of SEQ ID NO: 232; or
(11αB) a protein comprising at least one amino acid sequence selected from the group consisting of SEQ ID NOs: 234-237;
   (12α) (12αA) pyrophosphate-fructose 6-phosphate 1-phosphotransferase subunit alpha-like or a variant thereof, which is defined below in any of (12αA-a) to (12αA-e):
(12αA-a) a protein comprising an amino acid sequence with deletion, substitution, insertion or addition of one or several amino acids in SEQ ID NO: 239;
(12αA-b) a protein comprising an amino acid sequence having at least 70% identity to the amino acid sequence of SEQ ID NO: 239;
(12αA-c) a protein comprising an amino acid sequence encoded by a nucleotide sequence with deletion, substitution, insertion or addition of one or several nucleotides in SEQ ID NO: 238;
(12αA-d) a protein comprising an amino acid sequence encoded by a nucleotide sequence having at least 70% identity to the nucleotide sequence of SEQ ID NO: 238; or
(12αA-e) a protein comprising an amino acid sequence encoded by a nucleic acid that hybridizes under stringent conditions with a nucleic acid having a nucleotide sequence complementary to the nucleotide sequence of SEQ ID NO: 238; or
(12αB) a protein comprising an amino acid sequence of SEQ ID NO: 240;
   (13α) (13αA1) protein disulfide isomerase-like protein precursor or a variant thereof, which is defined below in any of (13αA1-a) to (13αA1-e):
(13αA1-a) a protein comprising an amino acid sequence with deletion, substitution, insertion or addition of one or several amino acids in SEQ ID NO: 242;
(13αA1-b) a protein comprising an amino acid sequence having at least 70% identity to the amino acid sequence of SEQ ID NO: 242;
(13αA1-c) a protein comprising an amino acid sequence encoded by a nucleotide sequence with deletion, substitution, insertion or addition of one or several nucleotides in SEQ ID NO: 241;
(13αA1-d) a protein comprising an amino acid sequence encoded by a nucleotide sequence having at least 70% identity to the nucleotide sequence of SEQ ID NO: 241; or
(13αA1-e) a protein comprising an amino acid sequence encoded by a nucleic acid that hybridizes under stringent conditions with a nucleic acid having a nucleotide sequence complementary to the nucleotide sequence of SEQ ID NO: 241;
(13αB1) a protein comprising at least one amino acid sequence selected from the group consisting of SEQ ID NOs: 243-252;
(13αA2) protein disulfide-isomerase or a variant thereof, which is defined below in any of (13αA2-a) to (13αA2-e):
(13αA2-a) a protein comprising an amino acid sequence with deletion, substitution, insertion or addition of one or several amino acids in SEQ ID NO: 254;
(13αA2-b) a protein comprising an amino acid sequence having at least 70% identity to the amino acid sequence of SEQ ID NO: 254;
(13αA2-c) a protein comprising an amino acid sequence encoded by a nucleotide sequence with deletion, substitution, insertion or addition of one or several nucleotides in SEQ ID NO: 253;
(13αA2-d) a protein comprising an amino acid sequence encoded by a nucleotide sequence having at least 70% identity to the nucleotide sequence of SEQ ID NO: 253; or
(13αA2-e) a protein comprising an amino acid sequence encoded by a nucleic acid that hybridizes under stringent conditions with a nucleic acid having a nucleotide sequence complementary to the nucleotide sequence of SEQ ID NO: 253; or
(13αB2) a protein comprising at least one amino acid sequence selected from the group consisting of SEQ ID NOs: 255-278;
   (14α) (14αA1) V-type proton ATPase subunit B 2 or a variant thereof, which is defined below in any of (14αA1-a) to (14αA1-e):
(14αA1-a) a protein comprising an amino acid sequence with deletion, substitution, insertion or addition of one or several amino acids in SEQ ID NO: 280;
(14αA1-b) a protein comprising an amino acid sequence having at least 70% identity to the amino acid sequence of SEQ ID NO: 280;
(14αA1-c) a protein comprising an amino acid sequence encoded by a nucleotide sequence with deletion, substitution, insertion or addition of one or several nucleotides in SEQ ID NO: 279;
(14αA1-d) a protein comprising an amino acid sequence encoded by a nucleotide sequence having at least 70% identity to the nucleotide sequence of SEQ ID NO: 279; or
(14αA1-e) a protein comprising an amino acid sequence encoded by a nucleic acid that hybridizes under stringent conditions with a nucleic acid having a nucleotide sequence complementary to the nucleotide sequence of SEQ ID NO: 279;
(14αB1) a protein comprising at least one amino acid sequence selected from the group consisting of SEQ ID NOs: 281-284;
(14αA2) V-type proton ATPase subunit B 2 or a variant thereof, which is defined below in any of (14αA2-a) to (14αA2-e):
(14αA2-a) a protein comprising an amino acid sequence with deletion, substitution, insertion or addition of one or several amino acids in SEQ ID NO: 286;
(14αA2-b) a protein comprising an amino acid sequence having at least 70% identity to the amino acid sequence of SEQ ID NO: 286;
(14αA2-c) a protein comprising an amino acid sequence encoded by a nucleotide sequence with deletion, substitution, insertion or addition of one or several nucleotides in SEQ ID NO: 285;
(14αA2-d) a protein comprising an amino acid sequence encoded by a nucleotide sequence having at least 70% identity to the nucleotide sequence of SEQ ID NO: 285; or
(14αA2-e) a protein comprising an amino acid sequence encoded by a nucleic acid that hybridizes under stringent conditions with a nucleic acid having a nucleotide sequence complementary to the nucleotide sequence of SEQ ID NO: 285; or
(14αB2) a protein comprising at least one amino acid sequence selected from the group consisting of SEQ ID NOs: 287-289;
   (15, 16α) (15, 16αA1) UTP-glucose-1-phosphate uridylyltransferase or a variant thereof, which is defined below in any of (15, 16αA1-a) to (15, 16αA1-e):
(15, 16αA1-a) a protein comprising an amino acid sequence with deletion, substitution, insertion or addition of one or several amino acids in SEQ ID NO: 291;
(15, 16αA1-b) a protein comprising an amino acid sequence having at least 70% identity to the amino acid sequence of SEQ ID NO: 291;
(15, 16αA1-c) a protein comprising an amino acid sequence encoded by a nucleotide sequence with deletion, substitution, insertion or addition of one or several nucleotides in SEQ ID NO: 290;
(15, 16αA1-d) a protein comprising an amino acid sequence encoded by a nucleotide sequence having at least 70% identity to the nucleotide sequence of SEQ ID NO: 290; or
(15, 16αA1-e) a protein comprising an amino acid sequence encoded by a nucleic acid that hybridizes under stringent conditions with a nucleic acid having a nucleotide sequence complementary to the nucleotide sequence of SEQ ID NO: 290;
(15, 16αB1) a protein comprising at least one amino acid sequence selected from the group consisting of SEQ ID NOs: 292-304;
(15, 16αA2) hypothetical protein GLYMA_02G241100 or a variant thereof, which is defined below in any of (15, 16αA2-a) to (15, 16αA2-e):
(15, 16αA2-a) a protein comprising an amino acid sequence with deletion, substitution, insertion or addition of one or several amino acids in SEQ ID NO: 306;
(15, 16αA2-b) a protein comprising an amino acid sequence having at least 70% identity to the amino acid sequence of SEQ ID NO: 306;
(15, 16αA2-c) a protein comprising an amino acid sequence encoded by a nucleotide sequence with deletion, substitution, insertion or addition of one or several nucleotides in SEQ ID NO: 305;
(15, 16αA2-d) a protein comprising an amino acid sequence encoded by a nucleotide sequence having at least 70% identity to the nucleotide sequence of SEQ ID NO: 305; or
(15, 16αA2-e) a protein comprising an amino acid sequence encoded by a nucleic acid that hybridizes under stringent conditions with a nucleic acid having a nucleotide sequence complementary to the nucleotide sequence of SEQ ID NO: 305; or
(15, 16αB2) a protein comprising at least one amino acid sequence selected from the group consisting of SEQ ID NOs: 307-318;
   (17α) (17αA1) guanosine nucleotide diphosphate dissociation inhibitor 2 or a variant thereof, which is defined below in any of (17αA1-a) to (17αA1-e):
(17αA1-a) a protein comprising an amino acid sequence with deletion, substitution, insertion or addition of one or several amino acids in SEQ ID NO: 320;
(17αA1-b) a protein comprising an amino acid sequence having at least 70% identity to the amino acid sequence of SEQ ID NO: 320;
(17αA1-c) a protein comprising an amino acid sequence encoded by a nucleotide sequence with deletion, substitution, insertion or addition of one or several nucleotides in SEQ ID NO: 319;
(17αA1-d) a protein comprising an amino acid sequence encoded by a nucleotide sequence having at least 70% identity to the nucleotide sequence of SEQ ID NO: 319; or
(17αA1-e) a protein comprising an amino acid sequence encoded by a nucleic acid that hybridizes under stringent conditions with a nucleic acid having a nucleotide sequence complementary to the nucleotide sequence of SEQ ID NO: 319;
(17αB1) a protein comprising at least one amino acid sequence selected from the group consisting of SEQ ID NOs: 321-325;
(17αA2) rab GDP dissociation inhibitor alpha-like or a variant thereof, which is defined below in any of (17αA2-a) to (17αA2-e):
(17αA2-a) a protein comprising an amino acid sequence with deletion, substitution, insertion or addition of one or several amino acids in SEQ ID NO: 327;
(17αA2-b) a protein comprising an amino acid sequence having at least 70% identity to the amino acid sequence of SEQ ID NO: 327;
(17αA2-c) a protein comprising an amino acid sequence encoded by a nucleotide sequence with deletion, substitution, insertion or addition of one or several nucleotides in SEQ ID NO: 326;
(17αA2-d) a protein comprising an amino acid sequence encoded by a nucleotide sequence having at least 70% identity to the nucleotide sequence of SEQ ID NO: 326; or
(17αA2-e) a protein comprising an amino acid sequence encoded by a nucleic acid that hybridizes under stringent conditions with a nucleic acid having a nucleotide sequence complementary to the nucleotide sequence of SEQ ID NO: 326; or
(17αB2) a protein comprising at least one amino acid sequence selected from the group consisting of SEQ ID NOs: 328-332;
   (18α) (18αA) hypothetical protein GLYMA_10G028300 or a variant thereof, which is defined below in any of (18αA-a) to (18αA-e):
(18αA-a) a protein comprising an amino acid sequence with deletion, substitution, insertion or addition of one or several amino acids in SEQ ID NO: 334;
(18αA-b) a protein comprising an amino acid sequence having at least 70% identity to the amino acid sequence of SEQ ID NO: 334;
(18αA-c) a protein comprising an amino acid sequence encoded by a nucleotide sequence with deletion, substitution, insertion or addition of one or several nucleotides in SEQ ID NO: 333;
(18αA-d) a protein comprising an amino acid sequence encoded by a nucleotide sequence having at least 70% identity to the nucleotide sequence of SEQ ID NO: 333; or
(18αA-e) a protein comprising an amino acid sequence encoded by a nucleic acid that hybridizes under stringent conditions with a nucleic acid having a nucleotide sequence complementary to the nucleotide sequence of SEQ ID NO: 333; or
(18αB) a protein comprising at least one amino acid sequence selected from the group consisting of SEQ ID NOs: 335-339;
   (19α) (19αA) sucrose binding protein homolog S-64 or a variant thereof, which is defined below in any of (19αA-a) to (19αA-e):
(19αA-a) a protein comprising an amino acid sequence with deletion, substitution, insertion or addition of one or several amino acids in SEQ ID NO: 341;
(19αA-b) a protein comprising an amino acid sequence having at least 70% identity to the amino acid sequence of SEQ ID NO: 341;
(19αA-c) a protein comprising an amino acid sequence encoded by a nucleotide sequence with deletion, substitution, insertion or addition of one or several nucleotides in SEQ ID NO: 340;
(19αA-d) a protein comprising an amino acid sequence encoded by a nucleotide sequence having at least 70% identity to the nucleotide sequence of SEQ ID NO: 340; or
(19αA-e) a protein comprising an amino acid sequence encoded by a nucleic acid that hybridizes under stringent conditions with a nucleic acid having a nucleotide sequence complementary to the nucleotide sequence of SEQ ID NO: 340; or
(19αB) a protein comprising at least one amino acid sequence selected from the group consisting of SEQ ID NOs: 342-346;
   (25α) (25αA1) succinyl-CoA ligase [ADP-forming]subunit beta, mitochondrial or a variant thereof, which is defined below in any of (25αA1-a) to (25αA1-e):
(25αA1-a) a protein comprising an amino acid sequence with deletion, substitution, insertion or addition of one or several amino acids in SEQ ID NO: 348;
(25αA1-b) a protein comprising an amino acid sequence having at least 70% identity to the amino acid sequence of SEQ ID NO: 348;
(25αA1-c) a protein comprising an amino acid sequence encoded by a nucleotide sequence with deletion, substitution, insertion or addition of one or several nucleotides in SEQ ID NO: 347;
(25αA1-d) a protein comprising an amino acid sequence encoded by a nucleotide sequence having at least 70% identity to the nucleotide sequence of SEQ ID NO: 347; or
(25αA1-e) a protein comprising an amino acid sequence encoded by a nucleic acid that hybridizes under stringent conditions with a nucleic acid having a nucleotide sequence complementary to the nucleotide sequence of SEQ ID NO: 347;
(25αB1) a protein comprising at least one amino acid sequence selected from the group consisting of SEQ ID NOs: 349-356;
(25αA2) succinyl-CoA ligase [ADP-forming]subunit beta, mitochondrial or a variant thereof, which is defined below in any of (25αA2-a) to (25αA2-e):
(25αA2-a) a protein comprising an amino acid sequence with deletion, substitution, insertion or addition of one or several amino acids in SEQ ID NO: 358;
(25αA2-b) a protein comprising an amino acid sequence having at least 70% identity to the amino acid sequence of SEQ ID NO: 358;
(25αA2-c) a protein comprising an amino acid sequence encoded by a nucleotide sequence with deletion, substitution, insertion or addition of one or several nucleotides in SEQ ID NO: 357;
(25αA2-d) a protein comprising an amino acid sequence encoded by a nucleotide sequence having at least 70% identity to the nucleotide sequence of SEQ ID NO: 357; or
(25αA2-e) a protein comprising an amino acid sequence encoded by a nucleic acid that hybridizes under stringent conditions with a nucleic acid having a nucleotide sequence complementary to the nucleotide sequence of SEQ ID NO: 357; or (25αB2) a protein comprising at least one amino acid sequence selected from the group consisting of SEQ ID NOs: 359-365;
   (26α) (26αA1) phosphoglycerate kinase, cytosolic or a variant thereof, which is defined below in any of (26αA1-a) to (26αA1-e):
(26αA1-a) a protein comprising an amino acid sequence with deletion, substitution, insertion or addition of one or several amino acids in SEQ ID NO: 367;
(26αA1-b) a protein comprising an amino acid sequence having at least 70% identity to the amino acid sequence of SEQ ID NO: 367;
(26αA1-c) a protein comprising an amino acid sequence encoded by a nucleotide sequence with deletion, substitution, insertion or addition of one or several nucleotides in SEQ ID NO: 366;
(26αA1-d) a protein comprising an amino acid sequence encoded by a nucleotide sequence having at least 70% identity to the nucleotide sequence of SEQ ID NO: 366; or
(26αA1-e) a protein comprising an amino acid sequence encoded by a nucleic acid that hybridizes under stringent conditions with a nucleic acid having a nucleotide sequence complementary to the nucleotide sequence of SEQ ID NO: 366;
(26αB1) a protein comprising at least one amino acid sequence selected from the group consisting of SEQ ID NOs: 368-382;
(26αA2) phosphoglycerate kinase, cytosolic or a variant thereof, which is defined below in any of (26αA2-a) to (26αA2-e):
(26αA2-a) a protein comprising an amino acid sequence with deletion, substitution, insertion or addition of one or several amino acids in SEQ ID NO: 384;
(26αA2-b) a protein comprising an amino acid sequence having at least 70% identity to the amino acid sequence of SEQ ID NO: 384;
(26αA2-c) a protein comprising an amino acid sequence encoded by a nucleotide sequence with deletion, substitution, insertion or addition of one or several nucleotides in SEQ ID NO: 383;
(26αA2-d) a protein comprising an amino acid sequence encoded by a nucleotide sequence having at least 70% identity to the nucleotide sequence of SEQ ID NO: 383; or
(26αA2-e) a protein comprising an amino acid sequence encoded by a nucleic acid that hybridizes under stringent conditions with a nucleic acid having a nucleotide sequence complementary to the nucleotide sequence of SEQ ID NO: 383;
(26αB2) a protein comprising at least one amino acid sequence selected from the group consisting of SEQ ID NOs: 385-392;
(26αA3) phosphoglycerate kinase, cytosolic or a variant thereof, which is defined below in any of (26αA3-a) to (26αA3-e):
(26αA3-a) a protein comprising an amino acid sequence with deletion, substitution, insertion or addition of one or several amino acids in SEQ ID NO: 394;
(26αA3-b) a protein comprising an amino acid sequence having at least 70% identity to the amino acid sequence of SEQ ID NO: 394;
(26αA3-c) a protein comprising an amino acid sequence encoded by a nucleotide sequence with deletion, substitution, insertion or addition of one or several nucleotides in SEQ ID NO: 393;
(26αA3-d) a protein comprising an amino acid sequence encoded by a nucleotide sequence having at least 70% identity to the nucleotide sequence of SEQ ID NO: 393; or
(26αA3-e) a protein comprising an amino acid sequence encoded by a nucleic acid that hybridizes under stringent conditions with a nucleic acid having a nucleotide sequence complementary to the nucleotide sequence of SEQ ID NO: 393; or
(26αB3) a protein comprising at least one amino acid sequence selected from the group consisting of SEQ ID NOs: 395-409;
   (27α) (27αA1) alcohol dehydrogenase 1 or a variant thereof, which is defined below in any of (27αA1-a) to (27αA1-e):
(27αA1-a) a protein comprising an amino acid sequence with deletion, substitution, insertion or addition of one or several amino acids in SEQ ID NO: 411;
(27αA1-b) a protein comprising an amino acid sequence having at least 70% identity to the amino acid sequence of SEQ ID NO: 411;
(27αA1-c) a protein comprising an amino acid sequence encoded by a nucleotide sequence with deletion, substitution, insertion or addition of one or several nucleotides in SEQ ID NO: 410;
(27αA1-d) a protein comprising an amino acid sequence encoded by a nucleotide sequence having at least 70% identity to the nucleotide sequence of SEQ ID NO: 410; or
(27αA1-e) a protein comprising an amino acid sequence encoded by a nucleic acid that hybridizes under stringent conditions with a nucleic acid having a nucleotide sequence complementary to the nucleotide sequence of SEQ ID NO: 410;
(27αB1) a protein comprising at least one amino acid sequence selected from the group consisting of SEQ ID NOs: 412-416;
(27αA2) alcohol dehydrogenase 1 or a variant thereof, which is defined below in any of (27αA2-a) to (27αA2-e):
(27αA2-a) a protein comprising an amino acid sequence with deletion, substitution, insertion or addition of one or several amino acids in SEQ ID NO: 418;
(27αA2-b) a protein comprising an amino acid sequence having at least 70% identity to the amino acid sequence of SEQ ID NO: 418;
(27αA2-c) a protein comprising an amino acid sequence encoded by a nucleotide sequence with deletion, substitution, insertion or addition of one or several nucleotides in SEQ ID NO: 417;
(27αA2-d) a protein comprising an amino acid sequence encoded by a nucleotide sequence having at least 70% identity to the nucleotide sequence of SEQ ID NO: 417; or
(27αA2-e) a protein comprising an amino acid sequence encoded by a nucleic acid that hybridizes under stringent conditions with a nucleic acid having a nucleotide sequence complementary to the nucleotide sequence of SEQ ID NO: 417;
(27αB2) a protein comprising at least one amino acid sequence selected from the group consisting of SEQ ID NOs: 419 and 420;
(27αA3) alcohol dehydrogenase 1-like or a variant thereof, which is defined below in any of (27αA3-a) to (27αA3-e):
(27αA3-a) a protein comprising an amino acid sequence with deletion, substitution, insertion or addition of one or several amino acids in SEQ ID NO: 422;
(27αA3-b) a protein comprising an amino acid sequence having at least 70% identity to the amino acid sequence of SEQ ID NO: 422;
(27αA3-c) a protein comprising an amino acid sequence encoded by a nucleotide sequence with deletion, substitution, insertion or addition of one or several nucleotides in SEQ ID NO: 421;
(27αA3-d) a protein comprising an amino acid sequence encoded by a nucleotide sequence having at least 70% identity to the nucleotide sequence of SEQ ID NO: 421; or
(27αA3-e) a protein comprising an amino acid sequence encoded by a nucleic acid that hybridizes under stringent conditions with a nucleic acid having a nucleotide sequence complementary to the nucleotide sequence of SEQ ID NO: 421; or
(27αB3) a protein comprising at least one amino acid sequence selected from the group consisting of SEQ ID NOs: 423-427;
   (28α) (28αA) 35 kDa seed maturation protein isoform X1 or a variant thereof, which is defined below in any of (28αA-a) to (28αA-e):
(28αA-a) a protein comprising an amino acid sequence with deletion, substitution, insertion or addition of one or several amino acids in SEQ ID NO: 429;
(28αA-b) a protein comprising an amino acid sequence having at least 70% identity to the amino acid sequence of SEQ ID NO: 429;
(28αA-c) a protein comprising an amino acid sequence encoded by a nucleotide sequence with deletion, substitution, insertion or addition of one or several nucleotides in SEQ ID NO: 428;
(28αA-d) a protein comprising an amino acid sequence encoded by a nucleotide sequence having at least 70% identity to the nucleotide sequence of SEQ ID NO: 428; or
(28αA-e) a protein comprising an amino acid sequence encoded by a nucleic acid that hybridizes under stringent conditions with a nucleic acid having a nucleotide sequence complementary to the nucleotide sequence of SEQ ID NO: 428; or
(28αB) a protein comprising at least one amino acid sequence selected from the group consisting of SEQ ID NOs: 430-433;
(29-33α) (29-33αA1) glyceraldehyde-3-phosphate dehydrogenase isoform X1 or a variant thereof, which is defined below in any of (29-33αA1-a) to (29-33αA1-e):
(29-33αA1-a) a protein comprising an amino acid sequence with deletion, substitution, insertion or addition of one or several amino acids in SEQ ID NO: 435;
(29-33αA1-b) a protein comprising an amino acid sequence having at least 70% identity to the amino acid sequence of SEQ ID NO: 435;
(29-33αA1-c) a protein comprising an amino acid sequence encoded by a nucleotide sequence with deletion, substitution, insertion or addition of one or several nucleotides in SEQ ID NO: 434;
(29-33αA1-d) a protein comprising an amino acid sequence encoded by a nucleotide sequence having at least 70% identity to the nucleotide sequence of SEQ ID NO: 434; or
(29-33αA1-e) a protein comprising an amino acid sequence encoded by a nucleic acid that hybridizes under stringent conditions with a nucleic acid having a nucleotide sequence complementary to the nucleotide sequence of SEQ ID NO: 434;
(29-33αB1) a protein comprising at least one amino acid sequence selected from the group consisting of SEQ ID NOs: 436-439;
(29-33αA2) glyceraldehyde-3-phosphate dehydrogenase, cytosolic or a variant thereof, which is defined below in any of (29-33αA2-a) to (29-33αA2-e):
(29-33αA2-a) a protein comprising an amino acid sequence with deletion, substitution, insertion or addition of one or several amino acids in SEQ ID NO: 441;
(29-33αA2-b) a protein comprising an amino acid sequence having at least 70% identity to the amino acid sequence of SEQ ID NO: 441;
(29-33αA2-c) a protein comprising an amino acid sequence encoded by a nucleotide sequence with deletion, substitution, insertion or addition of one or several nucleotides in SEQ ID NO: 440;
(29-33αA2-d) a protein comprising an amino acid sequence encoded by a nucleotide sequence having at least 70% identity to the nucleotide sequence of SEQ ID NO: 440; or
(29-33αA2-e) a protein comprising an amino acid sequence encoded by a nucleic acid that hybridizes under stringent conditions with a nucleic acid having a nucleotide sequence complementary to the nucleotide sequence of SEQ ID NO: 440;
(29-33αB2) a protein comprising at least one amino acid sequence selected from the group consisting of SEQ ID NOs: 442-445;
(29-33αA3) uncharacterized protein LOC100782924 or a variant thereof, which is defined below in any of (29-33αA3-a) to (29-33αA3-e):
(29-33αA3-a) a protein comprising an amino acid sequence with deletion, substitution, insertion or addition of one or several amino acids in SEQ ID NO: 447;
(29-33αA3-b) a protein comprising an amino acid sequence having at least 70% identity to the amino acid sequence of SEQ ID NO: 447;
(29-33αA3-c) a protein comprising an amino acid sequence encoded by a nucleotide sequence with deletion, substitution, insertion or addition of one or several nucleotides in SEQ ID NO: 446;
(29-33αA3-d) a protein comprising an amino acid sequence encoded by a nucleotide sequence having at least 70% identity to the nucleotide sequence of SEQ ID NO: 446; or
(29-33αA3-e) a protein comprising an amino acid sequence encoded by a nucleic acid that hybridizes under stringent conditions with a nucleic acid having a nucleotide sequence complementary to the nucleotide sequence of SEQ ID NO: 446; or
(29-33αB3) a protein comprising at least one amino acid sequence selected from the group consisting of SEQ ID NOs: 448-451,
   (34α) (34αA1) bifunctional UDP-glucose 4-epimerase and UDP-xylose 4-epimerase 1-like or a variant thereof, which is defined below in any of (34αA1-a) to (34αA1-e):
(34αA1-a) a protein comprising an amino acid sequence with deletion, substitution, insertion or addition of one or several amino acids in SEQ ID NO: 453;
(34αA1-b) a protein comprising an amino acid sequence having at least 70% identity to the amino acid sequence of SEQ ID NO: 453;
(34αA1-c) a protein comprising an amino acid sequence encoded by a nucleotide sequence with deletion, substitution, insertion or addition of one or several nucleotides in SEQ ID NO: 452;
(34αA1-d) a protein comprising an amino acid sequence encoded by a nucleotide sequence having at least 70% identity to the nucleotide sequence of SEQ ID NO: 452; or
(34αA1-e) a protein comprising an amino acid sequence encoded by a nucleic acid that hybridizes under stringent conditions with a nucleic acid having a nucleotide sequence complementary to the nucleotide sequence of SEQ ID NO: 452;
(34αB1) a protein comprising an amino acid sequence of SEQ ID NO: 454;
(34αA2) uncharacterized protein LOC100803483 or a variant thereof, which is defined below in any of (34αA2-a) to (34αA2-e):
(34αA2-a) a protein comprising an amino acid sequence with deletion, substitution, insertion or addition of one or several amino acids in SEQ ID NO: 456;
(34αA2-b) a protein comprising an amino acid sequence having at least 70% identity to the amino acid sequence of SEQ ID NO: 456;
(34αA2-c) a protein comprising an amino acid sequence encoded by a nucleotide sequence with deletion, substitution, insertion or addition of one or several nucleotides in SEQ ID NO: 455;
(34αA2-d) a protein comprising an amino acid sequence encoded by a nucleotide sequence having at least 70% identity to the nucleotide sequence of SEQ ID NO: 455; or
(34αA2-e) a protein comprising an amino acid sequence encoded by a nucleic acid that hybridizes under stringent conditions with a nucleic acid having a nucleotide sequence complementary to the nucleotide sequence of SEQ ID NO: 455; or
(34αB2) a protein comprising at least one amino acid sequence selected from the group consisting of SEQ ID NOs: 457 and 458;
   (35α) (35αA1) elongation factor 1-alpha or a variant thereof, which is defined below in any of (35αA1-a) to (35αA1-e):
(35αA1-a) a protein comprising an amino acid sequence with deletion, substitution, insertion or addition of one or several amino acids in SEQ ID NO: 460;
(35αA1-b) a protein comprising an amino acid sequence having at least 70% identity to the amino acid sequence of SEQ ID NO: 460;
(35αA1-c) a protein comprising an amino acid sequence encoded by a nucleotide sequence with deletion, substitution, insertion or addition of one or several nucleotides in SEQ ID NO: 459;
(35αA1-d) a protein comprising an amino acid sequence encoded by a nucleotide sequence having at least 70% identity to the nucleotide sequence of SEQ ID NO: 459; or
(35αA1-e) a protein comprising an amino acid sequence encoded by a nucleic acid that hybridizes under stringent conditions with a nucleic acid having a nucleotide sequence complementary to the nucleotide sequence of SEQ ID NO: 459; (35αB1) a protein comprising at least one amino acid sequence selected from the group consisting of SEQ ID NOs: 461-466;
(35αA2) elongation factor 1-alpha or a variant thereof, which is defined below in any of (35αA2-a) to (35αA2-e):
(35αA2-a) a protein comprising an amino acid sequence with deletion, substitution, insertion or addition of one or several amino acids in SEQ ID NO: 468;
(35αA2-b) a protein comprising an amino acid sequence having at least 70% identity to the amino acid sequence of SEQ ID NO: 468;
(35αA2-c) a protein comprising an amino acid sequence encoded by a nucleotide sequence with deletion, substitution, insertion or addition of one or several nucleotides in SEQ ID NO: 467;
(35αA2-d) a protein comprising an amino acid sequence encoded by a nucleotide sequence having at least 70% identity to the nucleotide sequence of SEQ ID NO: 467; or
(35αA2-e) a protein comprising an amino acid sequence encoded by a nucleic acid that hybridizes under stringent conditions with a nucleic acid having a nucleotide sequence complementary to the nucleotide sequence of SEQ ID NO: 467; (35αB2) a protein comprising at least one amino acid sequence selected from the group consisting of SEQ ID NOs: 469-471;
(35αA3) elongation factor-1A isoform X1 or a variant thereof, which is defined below in any of (35αA3-a) to (35αA3-e):
(35αA3-a) a protein comprising an amino acid sequence with deletion, substitution, insertion or addition of one or several amino acids in SEQ ID NO: 473;
(35αA3-b) a protein comprising an amino acid sequence having at least 70% identity to the amino acid sequence of SEQ ID NO: 473;
(35αA3-c) a protein comprising an amino acid sequence encoded by a nucleotide sequence with deletion, substitution, insertion or addition of one or several nucleotides in SEQ ID NO: 472;
(35αA3-d) a protein comprising an amino acid sequence encoded by a nucleotide sequence having at least 70% identity to the nucleotide sequence of SEQ ID NO: 472; or
(35αA3-e) a protein comprising an amino acid sequence encoded by a nucleic acid that hybridizes under stringent conditions with a nucleic acid having a nucleotide sequence complementary to the nucleotide sequence of SEQ ID NO: 472; or
(35αB3) a protein comprising at least one amino acid sequence selected from the group consisting of SEQ ID NOs: 474-477;
   (36, 37α) (36, 37αA1) seed maturation protein PM34 or a variant thereof, which is defined below in any of (36, 37αA1-a) to (36, 37αA1-e):
(36, 37αA1-a) a protein comprising an amino acid sequence with deletion, substitution, insertion or addition of one or several amino acids in SEQ ID NO: 479;
(36, 37αA1-b) a protein comprising an amino acid sequence having at least 70% identity to the amino acid sequence of SEQ ID NO: 479;
(36, 37αA1-c) a protein comprising an amino acid sequence encoded by a nucleotide sequence with deletion, substitution, insertion or addition of one or several nucleotides in SEQ ID NO: 478;
(36, 37αA1-d) a protein comprising an amino acid sequence encoded by a nucleotide sequence having at least 70% identity to the nucleotide sequence of SEQ ID NO: 478; or
(36, 37αA1-e) a protein comprising an amino acid sequence encoded by a nucleic acid that hybridizes under stringent conditions with a nucleic acid having a nucleotide sequence complementary to the nucleotide sequence of SEQ ID NO: 478;
(36, 37αB1) a protein comprising at least one amino acid sequence selected from the group consisting of SEQ ID NOs: 480-483;
(36, 37αA2) uncharacterized protein LOC100809384 or a variant thereof, which is defined below in any of (36, 37αA2-a) to (36, 37αA2-e):
(36, 37αA2-a) a protein comprising an amino acid sequence with deletion, substitution, insertion or addition of one or several amino acids in SEQ ID NO: 485;
(36, 37αA2-b) a protein comprising an amino acid sequence having at least 70% identity to the amino acid sequence of SEQ ID NO: 485;
(36, 37αA2-c) a protein comprising an amino acid sequence encoded by a nucleotide sequence with deletion, substitution, insertion or addition of one or several nucleotides in SEQ ID NO: 484;
(36, 37αA2-d) a protein comprising an amino acid sequence encoded by a nucleotide sequence having at least 70% identity to the nucleotide sequence of SEQ ID NO: 484; or
(36, 37αA2-e) a protein comprising an amino acid sequence encoded by a nucleic acid that hybridizes under stringent conditions with a nucleic acid having a nucleotide sequence complementary to the nucleotide sequence of SEQ ID NO: 484;
(36, 37αB2) a protein comprising at least one amino acid sequence selected from the group consisting of SEQ ID NOs: 486 and 487;
(36, 37αA3) hypothetical protein GLYMA_10G155300 or a variant thereof, which is defined below in any of (36, 37αA3-a) to (36, 37αA3-e):
(36, 37αA3-a) a protein comprising an amino acid sequence with deletion, substitution, insertion or addition of one or several amino acids in SEQ ID NO: 489;
(36, 37αA3-b) a protein comprising an amino acid sequence having at least 70% identity to the amino acid sequence of SEQ ID NO: 489;
(36, 37αA3-c) a protein comprising an amino acid sequence encoded by a nucleotide sequence with deletion, substitution, insertion or addition of one or several nucleotides in SEQ ID NO: 488;
(36, 37αA3-d) a protein comprising an amino acid sequence encoded by a nucleotide sequence having at least 70% identity to the nucleotide sequence of SEQ ID NO: 488; or
(36, 37αA3-e) a protein comprising an amino acid sequence encoded by a nucleic acid that hybridizes under stringent conditions with a nucleic acid having a nucleotide sequence complementary to the nucleotide sequence of SEQ ID NO: 488; or
(36, 37αB3) a protein comprising at least one amino acid sequence selected from the group consisting of SEQ ID NOs: 490-493;
   (38α) (38αA) seed biotin-containing protein SBP65-like isoform X1 or a variant thereof, which is defined below in any of (38αA-a) to (38αA-e):
(38αA-a) a protein comprising an amino acid sequence with deletion, substitution, insertion or addition of one or several amino acids in SEQ ID NO: 495;
(38αA-b) a protein comprising an amino acid sequence having at least 70% identity to the amino acid sequence of SEQ ID NO: 495;
(38αA-c) a protein comprising an amino acid sequence encoded by a nucleotide sequence with deletion, substitution, insertion or addition of one or several nucleotides in SEQ ID NO: 494;
(38αA-d) a protein comprising an amino acid sequence encoded by a nucleotide sequence having at least 70% identity to the nucleotide sequence of SEQ ID NO: 494; or
(38αA-e) a protein comprising an amino acid sequence encoded by a nucleic acid that hybridizes under stringent conditions with a nucleic acid having a nucleotide sequence complementary to the nucleotide sequence of SEQ ID NO: 494; or (38αB) a protein comprising at least one amino acid sequence selected from the group consisting of SEQ ID NOs: 496-513;
   (39α) (39αA) ATP synthase subunit 0, mitochondrial-like or a variant thereof, which is defined below in any of (39αA-a) to (39αA-e):
(39αA-a) a protein comprising an amino acid sequence with deletion, substitution, insertion or addition of one or several amino acids in SEQ ID NO: 515;
(39αA-b) a protein comprising an amino acid sequence having at least 70% identity to the amino acid sequence of SEQ ID NO: 515;
(39αA-c) a protein comprising an amino acid sequence encoded by a nucleotide sequence with deletion, substitution, insertion or addition of one or several nucleotides in SEQ ID NO: 514;
(39αA-d) a protein comprising an amino acid sequence encoded by a nucleotide sequence having at least 70% identity to the nucleotide sequence of SEQ ID NO: 514; or
(39αA-e) a protein comprising an amino acid sequence encoded by a nucleic acid that hybridizes under stringent conditions with a nucleic acid having a nucleotide sequence complementary to the nucleotide sequence of SEQ ID NO: 514; or
(39αB) a protein comprising an amino acid sequence of SEQ ID NO: 516;
   (40, 56α) (40, 56αA) P24 oleosin isoform A or a variant thereof, which is defined below in any of (40, 56αA-a) to (40, 56αA-e):
(40, 56αA-a) a protein comprising an amino acid sequence with deletion, substitution, insertion or addition of one or several amino acids in SEQ ID NO: 518;
(40, 56αA-b) a protein comprising an amino acid sequence having at least 70% identity to the amino acid sequence of SEQ ID NO: 518;
(40, 56αA-c) a protein comprising an amino acid sequence encoded by a nucleotide sequence with deletion, substitution, insertion or addition of one or several nucleotides in SEQ ID NO: 517;
(40, 56αA-d) a protein comprising an amino acid sequence encoded by a nucleotide sequence having at least 70% identity to the nucleotide sequence of SEQ ID NO: 517; or
(40, 56αA-e) a protein comprising an amino acid sequence encoded by a nucleic acid that hybridizes under stringent conditions with a nucleic acid having a nucleotide sequence complementary to the nucleotide sequence of SEQ ID NO: 517; or
(40, 56αB) a protein comprising at least one amino acid sequence selected from the group consisting of SEQ ID NOs: 519 and 520;
   (41α) (41αA) uncharacterized protein At3g49720-like isoform X2 or a variant thereof, which is defined below in any of (41αA-a) to (41αA-e):
   (41αA-a) a protein comprising an amino acid sequence with deletion, substitution, insertion or addition of one or several amino acids in SEQ ID NO: 522;
(41αA-b) a protein comprising an amino acid sequence having at least 70% identity to the amino acid sequence of SEQ ID NO: 522;
(41αA-c) a protein comprising an amino acid sequence encoded by a nucleotide sequence with deletion, substitution, insertion or addition of one or several nucleotides in SEQ ID NO: 521;
(41αA-d) a protein comprising an amino acid sequence encoded by a nucleotide sequence having at least 70% identity to the nucleotide sequence of SEQ ID NO: 521; or
(41αA-e) a protein comprising an amino acid sequence encoded by a nucleic acid that hybridizes under stringent conditions with a nucleic acid having a nucleotide sequence complementary to the nucleotide sequence of SEQ ID NO: 521; or
(41αB) a protein comprising an amino acid sequence of SEQ ID NO: 523;
   (42, 43α) (42, 43αA1) superoxide dismutase [Mn], mitochondrial or a variant thereof, which is defined below in any of (42, 43αA1-a) to (42, 43αA1-e):
(42, 43αA1-a) a protein comprising an amino acid sequence with deletion, substitution, insertion or addition of one or several amino acids in SEQ ID NO: 525;
(42, 43αA1-b) a protein comprising an amino acid sequence having at least 70% identity to the amino acid sequence of SEQ ID NO: 525;
(42, 43αA1-c) a protein comprising an amino acid sequence encoded by a nucleotide sequence with deletion, substitution, insertion or addition of one or several nucleotides in SEQ ID NO: 524;
(42, 43αA1-d) a protein comprising an amino acid sequence encoded by a nucleotide sequence having at least 70% identity to the nucleotide sequence of SEQ ID NO: 524; or
(42, 43αA1-e) a protein comprising an amino acid sequence encoded by a nucleic acid that hybridizes under stringent conditions with a nucleic acid having a nucleotide sequence complementary to the nucleotide sequence of SEQ ID NO: 524;
(42, 43αB1) a protein comprising at least one amino acid sequence selected from the group consisting of SEQ ID NOs: 526-531;
(42, 43αA2) hypothetical protein GLYMA_04G221300 or a variant thereof, which is defined below in any of (42, 43αA2-a) to (42, 43αA2-e):
(42, 43αA2-a) a protein comprising an amino acid sequence with deletion, substitution, insertion or addition of one or several amino acids in SEQ ID NO: 533;
(42, 43αA2-b) a protein comprising an amino acid sequence having at least 70% identity to the amino acid sequence of SEQ ID NO: 533;
(42, 43αA2-c) a protein comprising an amino acid sequence encoded by a nucleotide sequence with deletion, substitution, insertion or addition of one or several nucleotides in SEQ ID NO: 532;
(42, 43αA2-d) a protein comprising an amino acid sequence encoded by a nucleotide sequence having at least 70% identity to the nucleotide sequence of SEQ ID NO: 532; or
(42, 43αA2-e) a protein comprising an amino acid sequence encoded by a nucleic acid that hybridizes under stringent conditions with a nucleic acid having a nucleotide sequence complementary to the nucleotide sequence of SEQ ID NO: 532; or
(42, 43αB2) a protein comprising at least one amino acid sequence selected from the group consisting of SEQ ID NOs: 534-539;
   (45α) (45αA1) uncharacterized protein LOC100499771 or a variant thereof, which is defined below in any of (45αA1-a) to (45αA1-e):
(45αA1-a) a protein comprising an amino acid sequence with deletion, substitution, insertion or addition of one or several amino acids in SEQ ID NO: 541;
(45αA1-b) a protein comprising an amino acid sequence having at least 70% identity to the amino acid sequence of SEQ ID NO: 541;
(45αA1-c) a protein comprising an amino acid sequence encoded by a nucleotide sequence with deletion, substitution, insertion or addition of one or several nucleotides in SEQ ID NO: 540;
(45αA1-d) a protein comprising an amino acid sequence encoded by a nucleotide sequence having at least 70% identity to the nucleotide sequence of SEQ ID NO: 540; or
(45αA1-e) a protein comprising an amino acid sequence encoded by a nucleic acid that hybridizes under stringent conditions with a nucleic acid having a nucleotide sequence complementary to the nucleotide sequence of SEQ ID NO: 540;
(45αB1) a protein comprising at least one amino acid sequence selected from the group consisting of SEQ ID NOs: 542-545;
(45αA2) hypothetical protein GLYMA_09G192800 or a variant thereof, which is defined below in any of (45αA2-a) to (45αA2-e):
(45αA2-a) a protein comprising an amino acid sequence with deletion, substitution, insertion or addition of one or several amino acids in SEQ ID NO: 547;
(45αA2-b) a protein comprising an amino acid sequence having at least 70% identity to the amino acid sequence of SEQ ID NO: 547;
(45αA2-c) a protein comprising an amino acid sequence encoded by a nucleotide sequence with deletion, substitution, insertion or addition of one or several nucleotides in SEQ ID NO: 546;
(45αA2-d) a protein comprising an amino acid sequence encoded by a nucleotide sequence having at least 70% identity to the nucleotide sequence of SEQ ID NO: 546; or
(45αA2-e) a protein comprising an amino acid sequence encoded by a nucleic acid that hybridizes under stringent conditions with a nucleic acid having a nucleotide sequence complementary to the nucleotide sequence of SEQ ID NO: 546; or
(45αB2) a protein comprising at least one amino acid sequence selected from the group consisting of SEQ ID NOs: 548-551;
   (46α) (46αA) seed maturation protein PM22 or a variant thereof, which is defined below in any of (46αA-a) to (46αA-e):
(46αA-a) a protein comprising an amino acid sequence with deletion, substitution, insertion or addition of one or several amino acids in SEQ ID NO: 553;
(46αA-b) a protein comprising an amino acid sequence having at least 70% identity to the amino acid sequence of SEQ ID NO: 553;
(46αA-c) a protein comprising an amino acid sequence encoded by a nucleotide sequence with deletion, substitution, insertion or addition of one or several nucleotides in SEQ ID NO: 552;
(46αA-d) a protein comprising an amino acid sequence encoded by a nucleotide sequence having at least 70% identity to the nucleotide sequence of SEQ ID NO: 552; or
(46αA-e) a protein comprising an amino acid sequence encoded by a nucleic acid that hybridizes under stringent conditions with a nucleic acid having a nucleotide sequence complementary to the nucleotide sequence of SEQ ID NO: 552; or
(46αB) a protein comprising an amino acid sequence of SEQ ID NO: 554;
   (47α) (47αA1) eukaryotic translation initiation factor 5A-2 or a variant thereof, which is defined below in any of (47αA1-a) to (47αA1-e):
(47αA1-a) a protein comprising an amino acid sequence with deletion, substitution, insertion or addition of one or several amino acids in SEQ ID NO: 556;
(47αA1-b) a protein comprising an amino acid sequence having at least 70% identity to the amino acid sequence of SEQ ID NO: 556;
(47αA1-c) a protein comprising an amino acid sequence encoded by a nucleotide sequence with deletion, substitution, insertion or addition of one or several nucleotides in SEQ ID NO: 555;
(47αA1-d) a protein comprising an amino acid sequence encoded by a nucleotide sequence having at least 70% identity to the nucleotide sequence of SEQ ID NO: 555; or
(47αA1-e) a protein comprising an amino acid sequence encoded by a nucleic acid that hybridizes under stringent conditions with a nucleic acid having a nucleotide sequence complementary to the nucleotide sequence of SEQ ID NO: 555;
(47αB1) a protein comprising at least one amino acid sequence selected from the group consisting of SEQ ID NOs: 557 and 558;
(47αA2) uncharacterized protein LOC100305510 or a variant thereof, which is defined below in any of (47αA2-a) to (47αA2-e):
(47αA2-a) a protein comprising an amino acid sequence with deletion, substitution, insertion or addition of one or several amino acids in SEQ ID NO: 560;
(47αA2-b) a protein comprising an amino acid sequence having at least 70% identity to the amino acid sequence of SEQ ID NO: 560;
(47αA2-c) a protein comprising an amino acid sequence encoded by a nucleotide sequence with deletion, substitution, insertion or addition of one or several nucleotides in SEQ ID NO: 559;
(47αA2-d) a protein comprising an amino acid sequence encoded by a nucleotide sequence having at least 70% identity to the nucleotide sequence of SEQ ID NO: 559; or
(47αA2-e) a protein comprising an amino acid sequence encoded by a nucleic acid that hybridizes under stringent conditions with a nucleic acid having a nucleotide sequence complementary to the nucleotide sequence of SEQ ID NO: 559; or
(47αB2) a protein comprising at least one amino acid sequence selected from the group consisting of SEQ ID NOs: 561-563;
   (48α) (48αA) uncharacterized protein LOC100306570 or a variant thereof, which is defined below in any of (48αA-a) to (48αA-e):
(48αA-a) a protein comprising an amino acid sequence with deletion, substitution, insertion or addition of one or several amino acids in SEQ ID NO: 565;
(48αA-b) a protein comprising an amino acid sequence having at least 70% identity to the amino acid sequence of SEQ ID NO: 565;
(48αA-c) a protein comprising an amino acid sequence encoded by a nucleotide sequence with deletion, substitution, insertion or addition of one or several nucleotides in SEQ ID NO: 564;
(48αA-d) a protein comprising an amino acid sequence encoded by a nucleotide sequence having at least 70% identity to the nucleotide sequence of SEQ ID NO: 564; or
(48αA-e) a protein comprising an amino acid sequence encoded by a nucleic acid that hybridizes under stringent conditions with a nucleic acid having a nucleotide sequence complementary to the nucleotide sequence of SEQ ID NO: 564; or
(48αB) a protein comprising at least one amino acid sequence selected from the group consisting of SEQ ID NOs: 566-570;
   (50α) (50αA) uncharacterized protein LOC100813859 or a variant thereof, which is defined below in any of (50αA-a) to (50αA-e):
(50αA-a) a protein comprising an amino acid sequence with deletion, substitution, insertion or addition of one or several amino acids in SEQ ID NO: 572;
(50αA-b) a protein comprising an amino acid sequence having at least 70% identity to the amino acid sequence of SEQ ID NO: 572;
(50αA-c) a protein comprising an amino acid sequence encoded by a nucleotide sequence with deletion, substitution, insertion or addition of one or several nucleotides in SEQ ID NO: 571;
(50αA-d) a protein comprising an amino acid sequence encoded by a nucleotide sequence having at least 70% identity to the nucleotide sequence of SEQ ID NO: 571; or
(50αA-e) a protein comprising an amino acid sequence encoded by a nucleic acid that hybridizes under stringent conditions with a nucleic acid having a nucleotide sequence complementary to the nucleotide sequence of SEQ ID NO: 571; or
   (50αB) a protein comprising at least one amino acid sequence selected from the group consisting of SEQ ID NOs: 573-576;
   (51α) (51αA) 14-3-3-like protein or a variant thereof, which is defined below in any of (51αA-a) to (51αA-e):
(51αA-a) a protein comprising an amino acid sequence with deletion, substitution, insertion or addition of one or several amino acids in SEQ ID NO: 578;
(51αA-b) a protein comprising an amino acid sequence having at least 70% identity to the amino acid sequence of SEQ ID NO: 578;
(51αA-c) a protein comprising an amino acid sequence encoded by a nucleotide sequence with deletion, substitution, insertion or addition of one or several nucleotides in SEQ ID NO: 577;
(51αA-d) a protein comprising an amino acid sequence encoded by a nucleotide sequence having at least 70% identity to the nucleotide sequence of SEQ ID NO: 577; or
(51αA-e) a protein comprising an amino acid sequence encoded by a nucleic acid that hybridizes under stringent conditions with a nucleic acid having a nucleotide sequence complementary to the nucleotide sequence of SEQ ID NO: 577; or
(51αB) a protein comprising at least one amino acid sequence selected from the group consisting of SEQ ID NOs: 579-582;
   (52α) (52αA) probable fructokinase-4 or a variant thereof, which is defined below in any of (52αA-a) to (52αA-e):
(52αA-a) a protein comprising an amino acid sequence with deletion, substitution, insertion or addition of one or several amino acids in SEQ ID NO: 584;
(52αA-b) a protein comprising an amino acid sequence having at least 70% identity to the amino acid sequence of SEQ ID NO: 584;
(52αA-c) a protein comprising an amino acid sequence encoded by a nucleotide sequence with deletion, substitution, insertion or addition of one or several nucleotides in SEQ ID NO: 583;
(52αA-d) a protein comprising an amino acid sequence encoded by a nucleotide sequence having at least 70% identity to the nucleotide sequence of SEQ ID NO: 583; or
(52αA-e) a protein comprising an amino acid sequence encoded by a nucleic acid that hybridizes under stringent conditions with a nucleic acid having a nucleotide sequence complementary to the nucleotide sequence of SEQ ID NO: 583; or
(52αB) a protein comprising at least one amino acid sequence selected from the group consisting of SEQ ID NOs: 585-588;
   (53α) (53αA1) triosephosphate isomerase isoform X1 or a variant thereof, which is defined below in any of (53αA1-a) to (53αA1-e):
(53αA1-a) a protein comprising an amino acid sequence with deletion, substitution, insertion or addition of one or several amino acids in SEQ ID NO: 590;
(53αA1-b) a protein comprising an amino acid sequence having at least 70% identity to the amino acid sequence of SEQ ID NO: 590;
(53αA1-c) a protein comprising an amino acid sequence encoded by a nucleotide sequence with deletion, substitution, insertion or addition of one or several nucleotides in SEQ ID NO: 589;
(53αA1-d) a protein comprising an amino acid sequence encoded by a nucleotide sequence having at least 70% identity to the nucleotide sequence of SEQ ID NO: 589; or
(53αA1-e) a protein comprising an amino acid sequence encoded by a nucleic acid that hybridizes under stringent conditions with a nucleic acid having a nucleotide sequence complementary to the nucleotide sequence of SEQ ID NO: 589;
(53αB1) a protein comprising at least one amino acid sequence selected from the group consisting of SEQ ID NOs: 591-597;
(53αA2) triosephosphate isomerase, cytosolic or a variant thereof, which is defined below in any of (53αA2-a) to (53αA2-e):
(53αA2-a) a protein comprising an amino acid sequence with deletion, substitution, insertion or addition of one or several amino acids in SEQ ID NO: 599;
(53αA2-b) a protein comprising an amino acid sequence having at least 70% identity to the amino acid sequence of SEQ ID NO: 599;
(53αA2-c) a protein comprising an amino acid sequence encoded by a nucleotide sequence with deletion, substitution, insertion or addition of one or several nucleotides in SEQ ID NO: 598;
(53αA2-d) a protein comprising an amino acid sequence encoded by a nucleotide sequence having at least 70% identity to the nucleotide sequence of SEQ ID NO: 598; or
(53αA2-e) a protein comprising an amino acid sequence encoded by a nucleic acid that hybridizes under stringent conditions with a nucleic acid having a nucleotide sequence complementary to the nucleotide sequence of SEQ ID NO: 598; or
(53αB2) a protein comprising at least one amino acid sequence selected from the group consisting of SEQ ID NOs: 600-604;
   (54α) (54αA1) superoxide dismutase [Fe], chloroplastic isoform X1 or a variant thereof, which is defined below in any of (54αA1-a) to (54αA1-e):
(54αA1-a) a protein comprising an amino acid sequence with deletion, substitution, insertion or addition of one or several amino acids in SEQ ID NO: 606;
(54αA1-b) a protein comprising an amino acid sequence having at least 70% identity to the amino acid sequence of SEQ ID NO: 606;
(53αA1-c) a protein comprising an amino acid sequence encoded by a nucleotide sequence with deletion, substitution, insertion or addition of one or several nucleotides in SEQ ID NO: 605;
(54αA1-d) a protein comprising an amino acid sequence encoded by a nucleotide sequence having at least 70% identity to the nucleotide sequence of SEQ ID NO: 605; or
(54αA1-e) a protein comprising an amino acid sequence encoded by a nucleic acid that hybridizes under stringent conditions with a nucleic acid having a nucleotide sequence complementary to the nucleotide sequence of SEQ ID NO: 605; (54αB1) a protein comprising at least one amino acid sequence selected from the group consisting of SEQ ID NOs: 607-611;
(54αA2) iron-superoxide dismutase or a variant thereof, which is defined below in any of (54αA2-a) to (54αA2-e):
(54αA2-a) a protein comprising an amino acid sequence with deletion, substitution, insertion or addition of one or several amino acids in SEQ ID NO: 613;
(54αA2-b) a protein comprising an amino acid sequence having at least 70% identity to the amino acid sequence of SEQ ID NO: 613;
(54αA2-c) a protein comprising an amino acid sequence encoded by a nucleotide sequence with deletion, substitution, insertion or addition of one or several nucleotides in SEQ ID NO: 612;
(54αA2-d) a protein comprising an amino acid sequence encoded by a nucleotide sequence having at least 70% identity to the nucleotide sequence of SEQ ID NO: 612; or
(54αA2-e) a protein comprising an amino acid sequence encoded by a nucleic acid that hybridizes under stringent conditions with a nucleic acid having a nucleotide sequence complementary to the nucleotide sequence of SEQ ID NO: 612; or
(54αB2) a protein comprising at least one amino acid sequence selected from the group consisting of SEQ ID NOs: 614-616; or
   (55α) (55αA1) 51 kDa seed maturation protein precursor or a variant thereof, which is defined below in any of (55αA1-a) to (55αA1-e):
(55αA1-a) a protein comprising an amino acid sequence with deletion, substitution, insertion or addition of one or several amino acids in SEQ ID NO: 618;
(55αA1-b) a protein comprising an amino acid sequence having at least 70% identity to the amino acid sequence of SEQ ID NO: 618;
(55αA1-c) a protein comprising an amino acid sequence encoded by a nucleotide sequence with deletion, substitution, insertion or addition of one or several nucleotides in SEQ ID NO: 617;
(55αA1-d) a protein comprising an amino acid sequence encoded by a nucleotide sequence having at least 70% identity to the nucleotide sequence of SEQ ID NO: 617; or
(55αA1-e) a protein comprising an amino acid sequence encoded by a nucleic acid that hybridizes under stringent conditions with a nucleic acid having a nucleotide sequence complementary to the nucleotide sequence of SEQ ID NO: 617;
(55αB1) a protein comprising at least one amino acid sequence selected from the group consisting of SEQ ID NOs: 619-635;
(55αA2) Lea protein precursor or a variant thereof, which is defined below in any of (55αA2-a) to (55αA2-e):
(55αA2-a) a protein comprising an amino acid sequence with deletion, substitution, insertion or addition of one or several amino acids in SEQ ID NO: 637;
(55αA2-b) a protein comprising an amino acid sequence having at least 70% identity to the amino acid sequence of SEQ ID NO: 637;
(55αA2-c) a protein comprising an amino acid sequence encoded by a nucleotide sequence with deletion, substitution, insertion or addition of one or several nucleotides in SEQ ID NO: 636;
(55αA2-d) a protein comprising an amino acid sequence encoded by a nucleotide sequence having at least 70% identity to the nucleotide sequence of SEQ ID NO: 636; or
(55αA2-e) a protein comprising an amino acid sequence encoded by a nucleic acid that hybridizes under stringent conditions with a nucleic acid having a nucleotide sequence complementary to the nucleotide sequence of SEQ ID NO: 636; or
(55αB2) a protein comprising at least one amino acid sequence selected from the group consisting of SEQ ID NOs: 638-649.
[10] A composition for diagnosing an allergy to a soybean, comprising, as an antigen, at least one of proteins as defined above in any of (1α) to (55α) of [9].
[11] A method for providing an indicator for diagnosing an allergy to a soybean in a subject, the method comprising the steps of:
   (i) contacting a sample obtained from the subject with an antigen, wherein the sample is a solution comprising an Ig antibody;
   (ii) detecting binding between the IgE antibody present in the sample from the subject and the antigen; and
   (iii) when the binding between the IgE antibody in the subject and the antigen is detected, an indicator of the fact that the subject is allergic to a soybean is provided;
   wherein the antigen is at least one of proteins as defined above in any of (1α) to (55α) of [9].
[12] A pharmaceutical composition comprising at least one of proteins as defined above in any of (1α) to (55α) of [9].
[13] The pharmaceutical composition as set forth in [12], wherein the pharmaceutical composition is intended for the treatment of an allergy to a soybean.
[14] A soybean or processed products of soybean in which an antigen is eliminated or reduced, wherein the antigen is at least one of proteins as defined above in any of (1α) to (55α) of [9].
[15] A tester for determining the presence or absence of a soybean antigen in an object of interest, comprising an antibody that binds to at least one of proteins as defined above in any of (1α) to (55α) of [9].
[16] A soybean-derived antigen which is at least one of proteins as defined above in any of (1α) to (55α) of [9] and is causative of an allergy to soybean.

### Advantageous Effects of Invention

The present invention can provide novel antigens of an allergy to soybean. Since the novel antigens (allergen components) that trigger a soybean allergy were identified according to this invention, this invention can provide highly sensitive methods and kits for diagnosing an allergy to soybean, pharmaceutical compositions comprising such an antigen, soybean or processed products of soybean in which such an antigen is eliminated or reduced.

### BRIEF DESCRIPTION OF DRAWINGS

FIG. 1 is a photograph of a gel showing a protein electrophoretic pattern in two-dimensional electrophoresis of proteins contained in soybean. The bands at the left of the photograph are bands of molecular weight markers.

FIG. 2 is a photograph of an immunoblot of a two-dimensional electrophoretic pattern of proteins contained in soybean stained with serum of a healthy subject.

FIG. 3A is a photograph of an immunoblot of a two-dimensional electrophoretic pattern of proteins contained in soybean stained with serum of Soybean-allergic patient 1. The arrows indicate spots where the serum of the soybean-allergic patient specifically reacted.

FIG. 3B is a photograph of an immunoblot of a two-dimensional electrophoretic pattern of proteins contained in soybean stained with serum of Soybean-allergic patient 2. The arrows indicate spots where the serum of the soybean-allergic patient specifically reacted.

FIG. 3C is a photograph of an immunoblot of a two-dimensional electrophoretic pattern of proteins contained in soybean stained with serum of Soybean-allergic patient 3. The arrows indicate spots where the serum of the soybean-allergic patient specifically reacted.

FIG. 3D is a photograph of an immunoblot of a two-dimensional electrophoretic pattern of proteins contained in soybean stained with serum of Soybean-allergic patient 4. The arrows indicate spots where the serum of the soybean-allergic patient specifically reacted.

FIG. 4 is a photograph of an immunoblot of a two-dimensional electrophoretic pattern of proteins contained in soybean stained with serum of a healthy subject.

FIG. 5A is a photograph of an immunoblot of a two-dimensional electrophoretic pattern of proteins contained in soybean stained with serum of Soybean-allergic patient 1α. The spots indicated with a white line or enclosed in a square are spot where the serum of the soybean-allergic patient specifically reacted. The numbers (n) indicated are the numbers of the spots and correspond to the spots indicated with “nα” herein.

FIG. 5B is a photograph of an immunoblot of a two-dimensional electrophoretic pattern of proteins contained in soybean stained with serum of Soybean-allergic patient 2α. The spots indicated with a white line or enclosed in a square are spot where the serum of the soybean-allergic patient specifically reacted. The numbers (n) indicated are the numbers of the spots and correspond to the spots indicated with “nα” herein.

FIG. 5C is a photograph of an immunoblot of a two-dimensional electrophoretic pattern of proteins contained in soybean stained with serum of Soybean-allergic patient 3α. The spots indicated with a white line or enclosed in a square are spot where the serum of the soybean-allergic patient specifically reacted. The numbers (n) indicated are the numbers of the spots and correspond to the spots indicated with “nα” herein.

FIG. 5D is a photograph of an immunoblot of a two-dimensional electrophoretic pattern of proteins contained in soybean stained with serum of Soybean-allergic patient 4α. The spots indicated with a while line are spots where the serum of the soybean-allergic patient specifically reacted. The numbers (n) indicated are the numbers of the spots and correspond to the spots indicated with “nα” herein.

FIG. 5E is a photograph of an immunoblot of a two-dimensional electrophoretic pattern of proteins contained in soybean stained with serum of Soybean-allergic patient 5α. The spots indicated with a while line are spots where the serum of the soybean-allergic patient specifically reacted. The numbers (n) indicated are the numbers of the spots and correspond to the spots indicated with “nα” herein.

FIG. 5F is a photograph of an immunoblot of a two-dimensional electrophoretic pattern of proteins contained in soybean stained with serum of Soybean-allergic patient 6α. The spots indicated with a white line or enclosed in a square are spot where the serum of the soybean-allergic patient specifically reacted. The numbers (n) indicated are the numbers of the spots and correspond to the spots indicated with “nα” herein.

FIG. 5G is a photograph of an immunoblot of a two-dimensional electrophoretic pattern of proteins contained in soybean stained with serum of Soybean-allergic patient 7α. The spots enclosed in a square are spots where the serum of the soybean-allergic patient specifically reacted. The numbers (n) indicated are the numbers of the spots and correspond to the spots indicated with “nα” herein.

FIG. 5H is a photograph of an immunoblot of a two-dimensional electrophoretic pattern of proteins contained in soybean stained with serum of Soybean-allergic patient 8α. The spots indicated with a white line or enclosed in a square are spot where the serum of the soybean-allergic patient specifically reacted. The numbers (n) indicated are the numbers of the spots and correspond to the spots indicated with “nα” herein.

FIG. 5I is a photograph of an immunoblot of a two-dimensional electrophoretic pattern of proteins contained in soybean stained with serum of Soybean-allergic patient 9α. The spots indicated with a white line or enclosed in a square are spot where the serum of the soybean-allergic patient specifically reacted. The numbers (n) indicated are the numbers of the spots and correspond to the spots indicated with “nα” herein.

### DESCRIPTION OF EMBODIMENTS

The present invention will be described in detail below, but the present invention is not limited to them.

Unless otherwise defined herein, all scientific and technical terms used in relation to the present invention shall have meanings commonly understood by those skilled in the art.

Amino acid sequences herein are represented by one-letter amino acid symbols well known to those skilled in the art and the left end corresponds to the amino terminal and the right end corresponds to the carboxy terminal.

As referred to herein, the “allergy” refers to the state in which, when a certain antigen enters the body of a living individual sensitized to said antigen, the living individual shows a hypersensitive reaction detrimental to him/her. In blood and tissues of individuals with many food-allergic diseases, IgE antibodies specific to antigens are produced. IgE antibodies bind to mast cells or basophils. When an antigen specific to such an IgE antibody enters again the body of a patient with an allergic disease, said antigen combines with the IgE antibody bound to mast cells or basophils, and the IgE antibody crosslinks said antigen on the cell surface, resulting in physiological effects of IgE antibody-antigen interaction. Examples of such physiological effects include release of histamine, serotonin, heparin, eosinophil chemotactic factors, leucotrienes, or the like. These released substances provoke an allergic reaction resulting from the combination of an IgE antibody with particular antigens. Such allergic reactions caused by particular antigens occur through the aforementioned pathway.

As referred to herein, the “allergy to soybean” refers to the state in which an individual has an allergic reaction caused by proteins, etc. present in soybean which act as an antigen. The allergy to soybean can produce an allergic reaction upon contact with, or consumption of, an antigen present in soybean. In general, allergic reactions caused by consumption of foods are particularly referred to as “food allergies”. The allergy to soybean may be a food allergy.

As referred to herein, the “antigen” refers to a substance that provokes an allergic reaction, and is also referred to as an “allergen component”. The antigen is preferably a protein.

As referred to herein, the “protein” refers to a molecule having a structure in which naturally occurring amino acids are joined together by peptide bond. The number of amino acids present in a protein is not particularly limited, but proteins having about 2 to 50 amino acids joined together by peptide bond are in some cases called “peptides”. In the case where amino acids can form different enantiomers, the amino acids are understood to form an L-enantiomer, unless otherwise indicated. The amino acid sequences of proteins or peptides as used herein are represented by one-letter symbols of amino acids in accordance with standard usage and the notational convention commonly used in the art. The leftward direction represents the amino-terminal direction, and the rightward direction represents the carboxy-terminal direction.

### Identification of Antigens

Proteins contained in soybean were analyzed by the aforementioned technique to identify causative antigens of an allergy to soybean. To be specific, soybean proteins were subjected to two-dimensional electrophoresis under the conditions described below.

The electrophoresis in the first dimension was isoelectric focusing, which was performed using isoelectric focusing gels with a gel-strip length of 5 to 10 cm and a gel pH range of 3 to 10. The pH gradient of the gels in the direction of electrophoresis was as follows: with the total gel-strip length being taken as 1, the gel-strip length up to pH 5 was “a=0.15 to 0.3”, the gel-strip length from pH 5 to 7 was “b=0.4 to 0.7”, and the gel-strip length above pH 7 was “c=0.15 to 0.3”. More specifically, the isoelectric focusing was performed using the IPG gels, Immobiline Drystrip (pH3-10NL), produced by GE Healthcare Bio-Sciences Corporation (hereinafter abbreviated as “GE”). The electrophoresis system used was IPGphor produced by GE. The maximum current of the electrophoresis system was limited to 75 μA per gel strip. The voltage program adopted to perform the first-dimensional isoelectric focusing was as follows: (1) a constant voltage step was performed at a constant voltage of 300 V until the volt-hours reached 750 Vhr (the current variation width during electrophoresis for 30 minutes before the end of this step was 5 μA); (2) the voltage was increased gradually to 1000 V for 300 Vhr; (3) the voltage was further increased gradually to 5000 V for 4500 Vhr; and then (4) the voltage was held at a constant voltage of 5000 V until the total Vhr reached 12000.

The electrophoresis in the second dimension was SDS-PAGE, which was performed using polyacrylamide gels whose gel concentration at the distal end in the direction of electrophoresis was set to 3 to 6% and whose gel concentration at the proximal end was set to a higher value than that at the distal end. More specifically, the SDS-PAGE was performed using NuPAGE 4-12% Bris-Tris Gels (IPG well, Mini, 1 mm) produced by Life Technologies. The electrophoresis system used was XCell SureLock Mini-Cell produced by Life Technologies. The electrophoresis was run at a constant voltage of 200 V for about 45 minutes using an electrophoresis buffer composed of 50 mM MOPS, 50 mM Tris base, 0.1% (w/v) SDS and 1 mM EDTA.

As a result, the following spots in a two-dimensional electrophoresis gel run under the conditions described above for proteins in soybean have been revealed to exhibit specific binding to IgE antibodies from soybean-allergic patients.
Spot 1: Molecular weight 1 to 30 kDa, pl 3.0 to 7.0
Spot 2: Molecular weight 30 to 75 kDa, pl 4.0 to 8.5
Spot 3: Molecular weight 20 to 50 kDa, pl 7.0 to 12.0
Spot 4: Molecular weight 20 to 50 kDa, pl 6.0 to 10.0
Spot 5: Molecular weight 2 to 30 kDa, pl 4.0 to 9.0
Spot 6: Molecular weight 50 to 100 kDa, pl 5.0 to 10.0
Spot 7: Molecular weight 5 to 37 kDa, pl 3.0 to 7.0
Spot 8: Molecular weight 75 to 200 kDa, pl 4.0 to 6.0
Spot 1α: Molecular weight 70 to 120 kDa, pl 2.5 to 6.5
Spot 2α: Molecular weight 50 to 120 kDa, pl 2.5 to 6.5
Spot 4α: Molecular weight 70 to 120 kDa, pl 3.5 to 7.5
Spot 5α: Molecular weight 70 to 170 kDa, pl 3.5 to 7.5
Spot 6α: Molecular weight 70 to 120 kDa, pl 3.5 to 7.5
Spot 7α: Molecular weight 50 to 120 kDa, pl 3.5 to 7.5
Spot 8α: Molecular weight 50 to 120 kDa, pl 3.5 to 8.5
Spot 9α: Molecular weight 50 to 120 kDa, pl 3.5 to 8.5
Spot 10α: Molecular weight 30 to 90 kDa, pl 3.5 to 7.5
Spot 11α: Molecular weight 30 to 90 kDa, pl 3.5 to 8.5
Spot 12α: Molecular weight 30 to 90 kDa, pl 3.5 to 8.5
Spot 13α: Molecular weight 30 to 90 kDa, pl 3.5 to 7.5
Spot 14α: Molecular weight 30 to 90 kDa, pl 2.5 to 6.5
Spot 15α, 16α: Molecular weight 20 to 90 kDa, pl 3.5 to 7.5
Spot 17α: Molecular weight 20 to 90 kDa, pl 3.5 to 7.5
Spot 18α: Molecular weight 30 to 90 kDa, pl 3.5 to 8.5
Spot 29α: Molecular weight 30 to 90 kDa, pl 3.5 to 8.5
Spot 25α: Molecular weight 20 to 90 kDa, pl 3.5 to 7.5
Spot 26α: Molecular weight 20 to 90 kDa, pl 3.5 to 7.5
Spot 27α: Molecular weight 20 to 90 kDa, pl 3.5 to 7.5
Spot 28α, Molecular weight 10 to 70 kDa, pl 3.5 to 7.5
Spot 29α, 30α, 31α, 32, 33α Molecular weight 10 to 70 kDa, pl 3.5 to 8.5
Spot 34α: Molecular weight 10 to 70 kDa, pl 5.5 to 10.5
Spot 35α: Molecular weight 20 to 90 kDa, pl 5.5 to 10.5
Spot 36α, 37α: Molecular weight 10 to 70 kDa, pl 3.5 to 8.5
Spot 38α: Molecular weight 70 to 170 kDa, pl 5.5 to 10.5
Spot 39α: Molecular weight 5 to 60 kDa, pl 5.5 to 10.5
Spot 40α, 56α: Molecular weight 5 to 60 kDa, pl 5.5 to 10.5
Spot 41α: Molecular weight 5 to 60 kDa, pl 5.5 to 10.5
Spot 42α, 43: Molecular weight to 60 kDa, pl 5.5 to 10.5
Spot 45α: Molecular weight 2 to 50 kDa, pl 3.5 to 7.5
Spot 46α: Molecular weight 2 to 50 kDa, pl 3.5 to 7.5
Spot 47α: Molecular weight 2 to 50 kDa, pl 3.5 to 7.5
Spot 48α: Molecular weight 2 to 50 kDa, pl 3.5 to 8.5
Spot 51α: Molecular weight 2 to 50 kDa, pl 2.5 to 6.5
Spot 51α: Molecular weight S to 60 kDa, pl 2.5 to 6.5
Spot 53α: Molecular weight 10 to 70 kDa, pl 3.5 to 7.5
Spot 53α: Molecular weight 5 to 60 kDa, pl 3.5 to 8.5
Spot 55α: Molecular weight S to 60 kDa, pl 3.5 to 8.5
Spot 55α: Molecular weight 30 to 90 kDa, pl 5.5 to 10.5

### Antigen

### (1) Antigen in Spot 1

As the result of sequence identification of the antigen in spot 1 by mass spectroscopy, the following amino acid sequences were detected.

(SEQ ID NO: 17)

DQSSSYDDDEYSK

(SEQ ID NO: 18)

KSDQSSSYDDDEYSK

(SEQ ID NO: 19)

SDQSSSYDDDEYSK

(SEQ ID NO: 20)

SYDDDEYSK

Also, the mass spectroscopic data obtained for spot 1 on a mass spectrometer was analyzed by comparing the data against the Uniprot protein data, and as a result, the antigen in question was identified as Bowman-Birk type proteinase inhibitor D-II (amino acid sequence: SEQ ID NO: 2, encoding nucleotide sequence: SEQ ID NO: 1).

Accordingly, in the present invention, the antigen in spot 1 can be any of (1A-a) to (1A-e) and (1B) as defined below:
(1A-a) a protein comprising an amino acid sequence with deletion, substitution, insertion or addition of one or several amino acids in SEQ ID NO: 2;
(1A-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 2;
(1A-c) a protein comprising an amino acid sequence encoded by a nucleotide sequence with deletion, substitution, insertion or addition of one or several nucleotides in SEQ ID NO: 1;
(1A-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 1;
(1A-e) a protein comprising an amino acid sequence encoded by a nucleic acid that hybridizes under stringent conditions with a nucleic acid having a nucleotide sequence complementary to the nucleotide sequence of SEQ ID NO: 1;
(1B) a protein comprising at least one amino acid sequence selected from the group consisting of SEQ ID NOs: 17-20, preferably a protein comprising at least 2 or 3 or all sequences of the amino acid sequences; more preferably a protein comprising at least one amino acid sequence selected from the group consisting of SEQ ID NOs: 17-19, preferably a protein comprising at least 2 or all sequences of the amino acid sequences. As referred to above, the amino acid sequence of any of SEQ ID NOs: 17-20 may be an amino acid sequence derived therefrom by deletion, substitution, insertion or addition of one or several amino acids.

The proteins of (1A-a) to (1A-e) and (1B) as defined above also include those proteins whose amino acid residues are modified by phosphorylation, sugar chain modification, aminoacylation, ring-opening, deamination or the like.

The proteins of (1A-a) to (1A-e) and (1B) as defined above can be proteins that are found in a protein spot with a molecular weight of around 1 to 30 kDa, preferably around 2 to 25 kDa, more preferably around 5 to 20 kDa and an isoelectric point of 3.0 to 7.0, preferably 4.0 to 6.0 on gels used in the two-dimensional electrophoresis performed under the conditions described above in the subsection titled “Identification of antigens”.

Preferably, a protein that is an antigen in spot 1 has activity equivalent to that of the protein having the amino acid sequence of SEQ ID NO: 2 (Bowman-Birk type proteinase inhibitor D-II) or causative of an allergy to soybean.

### (2) Antigen in Spot 2

As the result of sequence identification of the antigen in spot 2 by mass spectroscopy, the following amino acid sequences were detected.

(SEQ ID NO: 21)

ALRALRLEDLRIPTAYIK

(SEQ ID NO: 22)

ALRLSGGDHVHAGTVVGK

(SEQ ID NO: 23)

LEGEREITLGFVDLLRDDFVEK

(SEQ ID NO: 24)

LRLEDLRIPTAYIK

(SEQ ID NO: 25)

LTYYTPDYETK

(SEQ ID NO: 26)

TDGLTSLDRYK

(SEQ ID NO: 27)

TFQGPPHGIQVERD

(SEQ ID NO: 28)

TFQGPPHGIQVERDK

Also, the mass spectroscopic data obtained for spot 2 on a mass spectrometer was analyzed by comparing the data against the Uniprot protein data, and as a result, the antigen in question was identified as Ribulose bisphosphate carboxylase large chain (amino acid sequence: SEQ ID NO: 4, encoding nucleotide sequence: SEQ ID NO: 3).

Accordingly, in the present invention, the antigen in spot 2 can be any of (2A-a) to (2A-e) and (2B) as defined below:
(2A-a) a protein comprising an amino acid sequence with deletion, substitution, insertion or addition of one or several amino acids in SEQ ID NO: 4;
(2A-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 4;
(2A-c) a protein comprising an amino acid sequence encoded by a nucleotide sequence with deletion, substitution, insertion or addition of one or several nucleotides in SEQ ID NO: 3;
(2A-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 3;
(2A-e) a protein comprising an amino acid sequence encoded by a nucleic acid that hybridizes under stringent conditions with a nucleic acid having a nucleotide sequence complementary to the nucleotide sequence of SEQ ID NO: 3;
(2B) a protein comprising at least one amino acid sequence selected from the group consisting of SEQ ID NOs: 21-28, preferably a protein comprising at least 2, 3, 4, 5, 6 or 7 or all sequences of the amino acid sequences. As referred to above, the amino acid sequence of any of SEQ ID NOs: 21-28 may be an amino acid sequence derived therefrom by deletion, substitution, insertion or addition of one or several amino acids.

The proteins of (2A-a) to (2A-e) and (2B) as defined above also include those proteins whose amino acid residues are modified by phosphorylation, sugar chain modification, aminoacylation, ring-opening, deamination or the like.

The proteins of (2A-a) to (2A-e) and (2B) as defined above can be proteins that are found in a protein spot with a molecular weight of around 30 to 75 kDa, preferably around 37 to 70 kDa, more preferably around 40 to 60 kDa and an isoelectric point of 4.0 to 8.5, preferably 5.5 to 7.5 on gels used in the two-dimensional electrophoresis performed under the conditions described above in the subsection titled “Identification of antigens”.

Preferably, a protein that is an antigen in spot 2 has activity equivalent to that of the protein having the amino acid sequence of SEQ ID NO: 4 (Ribulose bisphosphate carboxylase large chain) or causative of an allergy to soybean.

### (3) Antigen in Spot 3

As the result of sequence identification of the antigen in spot 3 by mass spectroscopy, the following amino acid sequences were detected.

(SEQ ID NO: 29)

APHTEDAIRVTQSTANFFISEARK

(SEQ ID NO: 30)

FVESGGARVIPLIYNESPENLNK

Also, the mass spectroscopic data obtained for spot 3 on a mass spectrometer was analyzed by comparing the data against the Uniprot protein data, and as a result, the antigen in question was identified as Gamma-glutamyl hydrolase (amino acid sequence: SEQ ID NO: 6, encoding nucleotide sequence: SEQ ID NO: 5).

Accordingly, in the present invention, the antigen in spot 3 can be any of (3A-a) to (3A-e) and (3B) as defined below:
(3A-a) a protein comprising an amino acid sequence with deletion, substitution, insertion or addition of one or several amino acids in SEQ ID NO: 6;
(3A-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 6;
(3A-c) a protein comprising an amino acid sequence encoded by a nucleotide sequence with deletion, substitution, insertion or addition of one or several nucleotides in SEQ ID NO: 5;
(3A-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 5.
(3A-e) a protein comprising an amino acid sequence encoded by a nucleic acid that hybridizes under stringent conditions with a nucleic acid having a nucleotide sequence complementary to the nucleotide sequence of SEQ ID NO: 5;
(3B) a protein comprising an amino acid sequence of SEQ ID NO: 29 or 30, preferably a protein comprising the amino acid sequences of SEQ ID NO: 29 and 30. As referred to above, the amino acid sequence(s) of SEQ ID NOs: 29 and/or 30 may be an amino acid sequence(s) derived therefrom by deletion, substitution, insertion or addition of one or several amino acids.

The proteins of (3A-a) to (3A-e) and (3B) as defined above also include those proteins whose amino acid residues are modified by phosphorylation, sugar chain modification, aminoacylation, ring-opening, deamination or the like.

The proteins of (3A-a) to (3A-e) and (3B) as defined above can be proteins that are found in a protein spot with a molecular weight of around 20 to 50 kDa, preferably around 25 to 40 kDa, more preferably around 25 to 37 kDa and an isoelectric point of 7.0 to 12.0, preferably 8.0 to 10.0 on gels used in the two-dimensional electrophoresis performed under the conditions described above in the subsection titled “Identification of antigens”.

Preferably, a protein that is an antigen in spot 3 has activity equivalent to that of the protein having the amino acid sequence of SEQ ID NO: 6 (Gamma-glutamyl hydrolase) or causative of an allergy to soybean.

### (4) Antigen in Spot 4

As the result of sequence identification of the antigen in spot 4 by mass spectroscopy, the following amino acid sequences were detected.

(SEQ ID NO: 31)
IWDLESK
(SEQ ID NO: 32)
SIVEDLK

Also, the mass spectroscopic data obtained for spot 4 on a mass spectrometer was analyzed by comparing the data against the Uniprot protein data, and as a result, the antigen in question was identified as Guanine nucleotide-binding protein subunit beta-like protein (amino acid sequence: SEQ ID NO: 8, encoding nucleotide sequence: SEQ ID NO: 7).

Accordingly, in the present invention, the antigen in spot 4 can be any of (4-a) to (4-e) and (4B) as defined below:
(4-a) a protein comprising an amino acid sequence with deletion, substitution, insertion or addition of one or several amino acids in SEQ ID NO: 8;
(4-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 8;
(4-c) a protein comprising an amino acid sequence encoded by a nucleotide sequence with deletion, substitution, insertion or addition of one or several nucleotides in SEQ ID NO: 7;
(4A-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 7;
(4A-e) a protein comprising an amino acid sequence encoded by a nucleic acid that hybridizes under stringent conditions with a nucleic acid having a nucleotide sequence complementary to the nucleotide sequence of SEQ ID NO: 7;
(4B) a protein comprising an amino acid sequence of SEQ ID NO: 31 or 32, preferably a protein comprising the amino acid sequences of SEQ ID NO: 31 and 32. As referred to above, the amino acid sequence(s) of SEQ ID NOs: 31 and/or 32 may be an amino acid sequence(s) derived therefrom by deletion, substitution, insertion or addition of one or several amino acids.

The proteins of (4A-a) to (4A-e) and (4B) as defined above also include those proteins whose amino acid residues are modified by phosphorylation, sugar chain modification, aminoacylation, ring-opening, deamination or the like.

The proteins of (4A-a) to (4A-e) and (4B) as defined above can be proteins that are found in a protein spot with a molecular weight of around 20 to 50 kDa, preferably around 25 to 40 kDa, more preferably around 25 to 37 kDa and an isoelectric point of 6.0 to 10.0, preferably 7.0 to 9.0 on gels used in the two-dimensional electrophoresis performed under the conditions described above in the subsection titled “Identification of antigens”.

Preferably, a protein that is an antigen in spot 4 has activity equivalent to that of the protein having the amino acid sequence of SEQ ID NO: 8 (Guanine nucleotide-binding protein subunit beta-like protein) or causative of an allergy to soybean.

### (5) Antigen in Spot 5

As the result of sequence identification of the antigen in spot 5 by mass spectroscopy, the following amino acid sequences were detected.

(SEQ ID NO: 33)
QELDERARQGETVVPGGTGGK
(SEQ ID NO: 34)
SGEERAQEEAIDIDESK

Also, the mass spectroscopic data obtained for spot 5 on a mass spectrometer was analyzed by comparing the data against the Uniprot protein data, and as a result, the antigen in question was identified as Protein SLE3 (amino acid sequence: SEQ ID NO: 10, encoding nucleotide sequence: SEQ ID NO: 9).

Accordingly, in the present invention, the antigen in spot 5 can be any of (5A-a) to (5A-e) and (5B) as defined below:
(5A-a) a protein comprising an amino acid sequence with deletion, substitution, insertion or addition of one or several amino acids in SEQ ID NO: 10;
(5A-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 10;
(5A-c) a protein comprising an amino acid sequence encoded by a nucleotide sequence with deletion, substitution, insertion or addition of one or several nucleotides in SEQ ID NO: 9;
(5A-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 9;
(5A-e) a protein comprising an amino acid sequence encoded by a nucleic acid that hybridizes under stringent conditions with a nucleic acid having a nucleotide sequence complementary to the nucleotide sequence of SEQ ID NO: 9;
(5B) a protein comprising an amino acid sequence of SEQ ID NO: 33 or 34, preferably a protein comprising the amino acid sequences of SEQ ID NO: 33 and 34. As referred to above, the amino acid sequence(s) of SEQ ID NOs: 33 and/or 34 may be an amino acid sequence(s) derived therefrom by deletion, substitution, insertion or addition of one or several amino acids.

The proteins of (5A-a) to (5A-e) and (5B) as defined above also include those proteins whose amino acid residues are modified by phosphorylation, sugar chain modification, aminoacylation, ring-opening, deamination or the like.

The proteins of (5A-a) to (5A-e) and (5B) as defined above can be proteins that are found in a protein spot with a molecular weight of around 2 to 30 kDa, preferably around 3 to 25 kDa, more preferably around 5 to 20 kDa and an isoelectric point of 4.0 to 9.0, preferably 5.0 to 7.0 on gels used in the two-dimensional electrophoresis performed under the conditions described above in the subsection titled “Identification of antigens”.

Preferably, a protein that is an antigen in spot 5 has activity equivalent to that of the protein having the amino acid sequence of SEQ ID NO: 10 (Protein SLE3) or causative of an allergy to soybean.

### (6) Antigen in Spot 6

As the result of sequence identification of the antigen in spot 6 by mass spectroscopy, the following amino acid sequences were detected.

(SEQ ID NO: 35)
AAAHVVEGAAGYAGHK
(SEQ ID NO: 36)
AKDYTLQAAEK
(SEQ ID NO: 37)
ARETHELGAHFESLADK
(SEQ ID NO: 38)
ATAVGWAAAHFSAEK
(SEQ ID NO: 39)
DTPQGSIEALQAGERVK
(SEQ ID NO: 40)
DYTLQAAEK
(SEQ ID NO: 41)
EEAQRELEAK
(SEQ ID NO: 42)
EGTGSIVFTAIGETVSSAGEK
(SEQ ID NO: 43)
ERGRESGGQVVAEK
(SEQ ID NO: 44)
GLAASAGETAK
(SEQ ID NO: 45)
GQQGYAVTK
(SEQ ID NO: 46)
KPSQPQEAEERPSEGIGETVRQYAQK
(SEQ ID NO: 47)
PAEIVQER
(SEQ ID NO: 48)
PQGSIEALQAGERVK
(SEQ ID NO: 49)
QAASETLNTTTQTAQEK
(SEQ ID NO: 50)
SAGGTTASYVGEK
(SEQ ID NO: 51)
SLTSIGEK
(SEQ ID NO: 52)
TPQGSIEALQAGERVK
(SEQ ID NO: 53)
VTDHAAANVVGNK

Also, the mass spectroscopic data obtained for spot 6 on a mass spectrometer was analyzed by comparing the data against the Uniprot protein data, and as a result, the antigen in question was identified as Seed biotin-containing protein SBP65 (amino acid sequence: SEQ ID NO: 12, encoding nucleotide sequence: SEQ ID NO: 11).

Accordingly, in the present invention, the antigen in spot 6 can be any of (6A-a) to (6A-e) and (6B) as defined below:
(6A-a) a protein comprising an amino acid sequence with deletion, substitution, insertion or addition of one or several amino acids in SEQ ID NO: 12;
(6A-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 12;
(6A-c) a protein comprising an amino acid sequence encoded by a nucleotide sequence with deletion, substitution, insertion or addition of one or several nucleotides in SEQ ID NO: 11;
(6A-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 11;
(6A-e) a protein comprising an amino acid sequence encoded by a nucleic acid that hybridizes under stringent conditions with a nucleic acid having a nucleotide sequence complementary to the nucleotide sequence of SEQ ID NO: 11;
(6B) a protein comprising at least one amino acid sequence selected from the group consisting of SEQ ID NOs: 35-53, preferably a protein comprising at least 2, 3, 4, 5, 6, 7, 8, 9, 10, 11, 12, 13, 14, 15, 16, 17 or 18 or all sequences of the amino acid sequences; more preferably a protein comprising at least one amino acid sequence selected from the group consisting of SEQ ID NOs: 35-39, 41-44, 46, 48-50, 52 and 53, preferably a protein comprising at least 2, 3, 4, 5, 6, 7, 8, 9, 10, 11, 12, 13 or 14 or all sequences of the amino acid sequences. As referred to above, the amino acid sequence of any of SEQ ID NOs: 35-53 may be an amino acid sequence derived therefrom by deletion, substitution, insertion or addition of one or several amino acids.

The proteins of (6A-a) to (6A-e) and (6B) as defined above also include those proteins whose amino acid residues are modified by phosphorylation, sugar chain modification, aminoacylation, ring-opening, deamination or the like.

The proteins of (6A-a) to (6A-e) and (6B) as defined above can be proteins that are found in a protein spot with a molecular weight of around 50 to 100 kDa, preferably around 55 to 90 kDa, more preferably around 60 to 75 kDa and an isoelectric point of 5.0 to 10.0, preferably 6.0 to 8.0 on gels used in the two-dimensional electrophoresis performed under the conditions described above in the subsection titled “Identification of antigens”.

Preferably, a protein that is an antigen in spot 6 has activity equivalent to that of the protein having the amino acid sequence of SEQ ID NO: 12 (Seed biotin-containing protein SBP65) or causative of an allergy to soybean.

### (7) Antigen in Spot 7

As the result of sequence identification of the antigen in spot 7 by mass spectroscopy, the following amino acid sequences were detected.

(SEQ ID NO: 54)
AASNAPAPLAGVIFEPFQELKK
(SEQ ID NO: 55)
ASNAPAPLAGVIFEPFQELKK
(SEQ ID NO: 56)
FFKESSEEEREHAEQLIK
(SEQ ID NO: 57)
GHGVWHFDQK
(SEQ ID NO: 58)
IAEYVAQLRLVGK
(SEQ ID NO: 59)
KIAEYVAQLRLVGK
(SEQ ID NO: 60)
LLHDEDHV
(SEQ ID NO: 61)
SNAPAPLAGVIFEPFQELKK

Also, the mass spectroscopic data obtained for spot 7 on a mass spectrometer was analyzed by comparing the data against the Uniprot protein data, and as a result, the antigen in question was identified as Ferritin-2 (amino acid sequence: SEQ ID NO: 14, encoding nucleotide sequence: SEQ ID NO: 13).

Accordingly, in the present invention, the antigen in spot 7 can be any of (7A-a) to (7A-e) and (7B) as defined below:
(7A-a) a protein comprising an amino acid sequence with deletion, substitution, insertion or addition of one or several amino acids in SEQ ID NO: 14;
(7A-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 14;
(7A-c) a protein comprising an amino acid sequence encoded by a nucleotide sequence with deletion, substitution, insertion or addition of one or several nucleotides in SEQ ID NO: 13;
(7A-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 13;
(7A-e) a protein comprising an amino acid sequence encoded by a nucleic acid that hybridizes under stringent conditions with a nucleic acid having a nucleotide sequence complementary to the nucleotide sequence of SEQ ID NO: 13;
(7B) a protein comprising at least one amino acid sequence selected from the group consisting of SEQ ID NOs: 54-61, preferably a protein comprising at least 2, 3, 4, 5, 6 or 7 or all sequences of the amino acid sequences; more preferably a protein comprising at least one amino acid sequence selected from the group consisting of SEQ ID NOs: 54-59 and 61, preferably a protein comprising at least 2, 3, 4, 5 or 6 or all sequences of the amino acid sequences. As referred to above, the amino acid sequence of any of SEQ ID NOs: 54-61 may be an amino acid sequence derived therefrom by deletion, substitution, insertion or addition of one or several amino acids.

The proteins of (7A-a) to (7A-e) and (7B) as defined above also include those proteins whose amino acid residues are modified by phosphorylation, sugar chain modification, aminoacylation, ring-opening, deamination or the like.

The proteins of (7A-a) to (7A-e) and (7B) as defined above can be proteins that are found in a protein spot with a molecular weight of around 5 to 37 kDa, preferably around 10 to 30 kDa, more preferably around 15 to 25 kDa and an isoelectric point of 3.0 to 7.0, preferably 4.0 to 6.0 on gels used in the two-dimensional electrophoresis performed under the conditions described above in the subsection titled “Identification of antigens”.

Preferably, a protein that is an antigen in spot 7 has activity equivalent to that of the protein having the amino acid sequence of SEQ ID NO: 14 (Ferritin-2) or causative of an allergy to soybean.

### (8) Antigen in Spot 8

As the result of sequence identification of the antigen in spot 8 by mass spectroscopy, the following amino acid sequences were detected.

(SEQ ID NO: 62)
AGESESNLRK
(SEQ ID NO: 63)
GESEANVREIFDK
(SEQ ID NO: 64)
GILLYGPPGSGK
(SEQ ID NO: 65)
LAGESESNLRK
(SEQ ID NO: 66)
LSDDVDLERIAK
(SEQ ID NO: 67)
RELQETVQYPVEHPEK
(SEQ ID NO: 68)
TVFIIGATNRPDIID

Also, the mass spectroscopic data obtained for spot 8 on a mass spectrometer was analyzed by comparing the data against the Uniprot protein data, and as a result, the antigen in question was identified as Cell division cycle protein 48 homolog (amino acid sequence: SEQ ID NO: 16, encoding nucleotide sequence: SEQ ID NO: 15).

Accordingly, in the present invention, the antigen in spot 8 can be any of (8A-a) to (8A-e) and (8B) as defined below:
(8A-a) a protein comprising an amino acid sequence with deletion, substitution, insertion or addition of one or several amino acids in SEQ ID NO: 16;
(8A-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 16;
(8A-c) a protein comprising an amino acid sequence encoded by a nucleotide sequence with deletion, substitution, insertion or addition of one or several nucleotides in SEQ ID NO: 15;
(8A-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 15;
(8A-e) a protein comprising an amino acid sequence encoded by a nucleic acid that hybridizes under stringent conditions with a nucleic acid having a nucleotide sequence complementary to the nucleotide sequence of SEQ ID NO: 15;
(8B) a protein comprising at least one amino acid sequence selected from the group consisting of SEQ ID NOs: 62-68, preferably a protein comprising at least 2, 3, 4, 5 or 6 or all sequences of the amino acid sequences. As referred to above, the amino acid sequence of any of SEQ ID NOs: 62-68 may be an amino acid sequence derived therefrom by deletion, substitution, insertion or addition of one or several amino acids.

The proteins of (8A-a) to (8A-e) and (8B) as defined above also include those proteins whose amino acid residues are modified by phosphorylation, sugar chain modification, aminoacylation, ring-opening, deamination or the like.

The proteins of (8A-a) to (8A-e) and (8B) as defined above can be proteins that are found in a protein spot with a molecular weight of around 75 to 200 kDa, preferably around 100 to 170 kDa, more preferably around 100 to 150 kDa and an isoelectric point of 3.0 to 7.0, preferably 4.0 to 6.0 on gels used in the two-dimensional electrophoresis performed under the conditions described above in the subsection titled “Identification of antigens”.

Preferably, a protein that is an antigen in spot 8 has activity equivalent to that of the protein having the amino acid sequence of SEQ ID NO: 16 (Cell division cycle protein 48 homolog) or causative of an allergy to soybean.

### (1α) Antigen in Spot 1α

As the result of sequence identification of the antigen in spot 1α by mass spectroscopy, the following amino acid sequences were detected.

(SEQ ID NO: 71)
APQDLYESYYNANK
(SEQ ID NO: 72)
AVLFVPPK
(SEQ ID NO: 73)
DFASRIYDSVK
(SEQ ID NO: 74)
DIFLRELISNASDALDK
(SEQ ID NO: 75)
DIFYITGTSK
(SEQ ID NO: 76)
DNVDDVK
(SEQ ID NO: 77)
EDSKPETDAVNDDNDVK
(SEQ ID NO: 78)
EEAGEYLEESK
(SEQ ID NO: 79)
EQLENSPFLERLK
(SEQ ID NO: 80)
EVLGEGDNTK
(SEQ ID NO: 81)
EVTEEEYTK
(SEQ ID NO: 82)
FYHSLAK
(SEQ ID NO: 83)
IGAVPHGLSTDSDVVK
(SEQ ID NO: 84)
LGIIEDATNRNRLAK
(SEQ ID NO: 85)
NPEDEGVK
(SEQ ID NO: 86)
SGTSAFVEK
(SEQ ID NO: 87)
TSLDISPEATVEEEDDTEVEAESDAK
(SEQ ID NO: 90)
APQDLYESYYNANK
(SEQ ID NO: 91)
AVLFVPPK
(SEQ ID NO: 92)
DFASRIYDSVK
(SEQ ID NO: 93)
DIFLRELISNASDALDK
(SEQ ID NO: 94)
DIFYITGTSK
(SEQ ID NO: 95)
DNVDDVK
(SEQ ID NO: 96)
DVLGEGDNTK
(SEQ ID NO: 97)
EEAGEYLQESK
(SEQ ID NO: 98)
EQLENSPFLERLK
(SEQ ID NO: 99)
EVTEEEYTK
(SEQ ID NO: 100)
FYHSLAK
(SEQ ID NO: 101)
IGAVPHGLSTDSDVVK
(SEQ ID NO: 102)
LGIIEDATNRNRLAK
(SEQ ID NO: 103)
NPEDEGVK
(SEQ ID NO: 104)
SGTSAFVEK

Also, the mass spectroscopic data obtained for spot 1α on a mass spectrometer was analyzed by comparing the data against the NCBI protein data, and as a result, endoplasmin homolog isoform X2 (amino acid sequence: SEQ ID NO: 70, encoding nucleotide sequence: SEQ ID NO: 69) was identified as a protein comprising any of SEQ ID NOs: 71 to 87 and endoplasmin homolog isoform X2 (amino acid sequence: SEQ ID NO: 89, encoding nucleotide sequence: SEQ ID NO: 88) was identified as a protein comprising any of SEQ ID NOs: 90 to 104.

Accordingly, the antigen in spot 1α in the present invention can be any of proteins selected from the group consisting of the proteins as defined below in (1αA1-a) to (1αA1-e), (1αB1), (1αA2-a) to (1αA2-e), and (1αB2):
(1αA1-a) a protein comprising an amino acid sequence with deletion, substitution, insertion or addition of one or several amino acids in SEQ ID NO: 70;
(1αA1-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 70;
(1αA1-c) a protein comprising an amino acid sequence encoded by a nucleotide sequence with deletion, substitution, insertion or addition of one or several nucleotides in SEQ ID NO: 69;
(1αA1-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 69;
(1αA1-e) a protein comprising an amino acid sequence encoded by a nucleic acid that hybridizes under stringent conditions with a nucleic acid having a nucleotide sequence complementary to the nucleotide sequence of SEQ ID NO: 69;
(1αB1) a protein comprising at least one amino acid sequence selected from the group consisting of SEQ ID NOs: 71-87, preferably a protein comprising at least 2, 3, 4, 5, 6, 7, 8, 9, 10, 11, 12, 13, 14, 15, or 16 or all sequences of the amino acid sequences. As referred to above, the amino acid sequence of any of SEQ ID NOs: 71 to 87 may be an amino acid sequence derived therefrom by deletion, substitution, insertion or addition of one or several amino acids;
(1αA2-a) a protein comprising an amino acid sequence with deletion, substitution, insertion or addition of one or several amino acids in SEQ ID NO: 89;
(1αA2-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 89;
(1αA2-c) a protein comprising an amino acid sequence encoded by a nucleotide sequence with deletion, substitution, insertion or addition of one or several nucleotides in SEQ ID NO: 88;
(1αA2-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 88;
(1αA2-e) a protein comprising an amino acid sequence encoded by a nucleic acid that hybridizes under stringent conditions with a nucleic acid having a nucleotide sequence complementary to the nucleotide sequence of SEQ ID NO: 88;
(1αB2) a protein comprising at least one amino acid sequence selected from the group consisting of SEQ ID NOs: 90-104, preferably a protein comprising at least 2, 3, 4, 5, 6, 7, 8, 9, 10, 11, 12, 13, or 14 or all sequences of the amino acid sequences. As referred to above, the amino acid sequence of any of SEQ ID NOs: 90-104 may be an amino acid sequence derived therefrom by deletion, substitution, insertion or addition of one or several amino acids.

The protein selected from the group consisting of the proteins as defined above in (1αA1-a) to (1αA1-e), (1αB1), (1αA2-a) to (1αA2-e), and (1αB2) also include those proteins whose amino acid residues are modified by phosphorylation, sugar chain modification, aminoacylation, ring-opening, deamination or the like.

The protein selected from the group consisting of the proteins as defined above in (1αA1-a) to (1αA1-e), (1αB1), (1αA2-a) to (1αA2-e), and (1αB2) can be proteins that are found in a protein spot with a molecular weight of around 70 to 120 kDa, preferably around 80 to 110 kDa, and an isoelectric point of 2.5 to 6.5, preferably 3.0 to 6.0 on gels used in the two-dimensional electrophoresis performed under the conditions described above in the subsection titled “Identification of antigens”.

Preferably, a protein that is an antigen in spot 1α has activity equivalent to that of the protein having the amino acid sequence of SEQ ID NO: 70 or 89 (endoplasmin homolog isoform X2) or causative of an allergy to soybean.

### (2α) Antigen in Spot 2α

As the result of sequence identification of the antigen in spot 2α by mass spectroscopy, the following amino acid sequences were detected.

(SEQ ID NO: 107)
ADLVNNLGTIARSGTK
(SEQ ID NO: 108)
AVENSPFLEK
(SEQ ID NO: 109)
EDQLEYLEERRLK
(SEQ ID NO: 110)
EEYAAFYK
(SEQ ID NO: 111)
EGQNDIYYITGESK
(SEQ ID NO: 112)
EIFLRELISNASDALDK
(SEQ ID NO: 113)
FYEAFSK
(SEQ ID NO: 114)
HFSVEGQLEFK
(SEQ ID NO: 115)
IRFESLTDK
(SEQ ID NO: 116)
LAELLRYHSTK
(SEQ ID NO: 117)
LDESEDEK
(SEQ ID NO: 118)
LGIHEDSQNK
(SEQ ID NO: 119)
RAPFDLFDTRK
(SEQ ID NO: 120)
VEDVDEDK
(SEQ ID NO: 123)
ADLVNNLGTIARSGTK
(SEQ ID NO: 124)
AVENSPFLEK
(SEQ ID NO: 125)
EDQLEYLEERRLK
(SEQ ID NO: 126)
EEYSAFYK
(SEQ ID NO: 127)
EGQSDIYYITGESK
(SEQ ID NO: 128)
EIFLRELISNASDALDK
(SEQ ID NO: 129)
FYEAFSK
(SEQ ID NO: 130)
HFSVEGQLEFK
(SEQ ID NO: 131)
IAELLRYHSTK
(SEQ ID NO: 132)
IRFESLTDK
(SEQ ID NO: 133)
LDESEDEK
(SEQ ID NO: 134)
LGIHEDSQNK
(SEQ ID NO: 135)
RAPFDLFDTRK
(SEQ ID NO: 136)
SGDELTSLK
(SEQ ID NO: 137)
VEDVDEEK
(SEQ ID NO: 140)
ADLVNNLGTIARSGTK
(SEQ ID NO: 141)
AVENSPFLEK
(SEQ ID NO: 142)
EDQLEYLEERRLK
(SEQ ID NO: 143)
EEYAAFYK
(SEQ ID NO: 144)
EGQNDIYYITGESK
(SEQ ID NO: 145)
EIFLRELISNASDALDK
(SEQ ID NO: 146)
FYEAFSK
(SEQ ID NO: 147)
HFSVEGQLEFK
(SEQ ID NO: 148)
IRFESLTDK
(SEQ ID NO: 149)
LAELLRYHSTK
(SEQ ID NO: 150)
LDESEDEK
(SEQ ID NO: 151)
LGIHEDSQNK
(SEQ ID NO: 152)
RAPFDLFDTK
(SEQ ID NO: 153)
VEDVDEDK

Also, the mass spectroscopic data obtained for spot 2α on a mass spectrometer was analyzed by comparing the data against the NCBI protein data, and as a result, heat shock cognate protein 80 (amino acid sequence: SEQ ID NO: 106, encoding nucleotide sequence: SEQ ID NO: 105) was identified as a protein comprising any of SEQ ID NOs: 107 to 120, heat shock protein 90-1 (amino acid sequence: SEQ ID NO: 122, encoding nucleotide sequence: SEQ ID NO: 121) was identified as a protein comprising any of SEQ ID NOs: 123 to 137, and hypothetical protein GLYMA_02G302500 was identified as a protein comprising any of SEQ ID NOs: 140 to 153.

Accordingly, the antigen in spot 2α in the present invention can be any of proteins selected from the group consisting of the proteins as defined below in (2αA1-a) to (2αA1-e), (2αB1), (2αA2-a) to (2αA2-e), (2αB2), (2αA3-a) to (2αA3-e), and (2αB3):
(2αA1-a) a protein comprising an amino acid sequence with deletion, substitution, insertion or addition of one or several amino acids in SEQ ID NO: 106;
(2αA1-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 106;
(2αA1-c) a protein comprising an amino acid sequence encoded by a nucleotide sequence with deletion, substitution, insertion or addition of one or several nucleotides in SEQ ID NO: 105;
(2αA1-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 105;
(2αA1-e) a protein comprising an amino acid sequence encoded by a nucleic acid that hybridizes under stringent conditions with a nucleic acid having a nucleotide sequence complementary to the nucleotide sequence of SEQ ID NO: 105;
(2αB1) a protein comprising at least one amino acid sequence selected from the group consisting of SEQ ID NOs: 107-120, preferably a protein comprising at least 2, 3, 4, 5, 6, 7, 8, 9, 10, 11, 12, or 13 or all sequences of the amino acid sequences. As referred to above, the amino acid sequence of any of SEQ ID NOs: 107 to 120 may be an amino acid sequence derived therefrom by deletion, substitution, insertion or addition of one or several amino acids;
(2αA2-a) a protein comprising an amino acid sequence with deletion, substitution, insertion or addition of one or several amino acids in SEQ ID NO: 122;
(2αA2-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 122;
(2αA2-c) a protein comprising an amino acid sequence encoded by a nucleotide sequence with deletion, substitution, insertion or addition of one or several nucleotides in SEQ ID NO: 121;
(2αA2-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 121;
(2αA2-e) a protein comprising an amino acid sequence encoded by a nucleic acid that hybridizes under stringent conditions with a nucleic acid having a nucleotide sequence complementary to the nucleotide sequence of SEQ ID NO: 121;
(2αB2) a protein comprising at least one amino acid sequence selected from the group consisting of SEQ ID NOs: 123-137, preferably a protein comprising at least 2, 3, 4, 5, 6, 7, 8, 9, 10, 11, 12, 13, or 14 or all sequences of the amino acid sequences. As referred to above, the amino acid sequence of any of SEQ ID NOs: 123 to 137 may be an amino acid sequence derived therefrom by deletion, substitution, insertion or addition of one or several amino acids.
(2αA3-a) a protein comprising an amino acid sequence with deletion, substitution, insertion or addition of one or several amino acids in SEQ ID NO: 139;
(2αA3-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 139;
(2αA3-c) a protein comprising an amino acid sequence encoded by a nucleotide sequence with deletion, substitution, insertion or addition of one or several nucleotides in SEQ ID NO: 138;
(2αA3-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 138;
(2αA3-e) a protein comprising an amino acid sequence encoded by a nucleic acid that hybridizes under stringent conditions with a nucleic acid having a nucleotide sequence complementary to the nucleotide sequence of SEQ ID NO: 138;
(2αB3) a protein comprising at least one amino acid sequence selected from the group consisting of SEQ ID NOs: 140-153, preferably a protein comprising at least 2, 3, 4, 5, 6, 7, 8, 9, 10, 11, 12, or 13 or all sequences of the amino acid sequences. As referred to above, the amino acid sequence of any of SEQ ID NOs: 140 to 153 may be an amino acid sequence derived therefrom by deletion, substitution, insertion or addition of one or several amino acids.

The protein selected from the group consisting of the proteins as defined above in (2αA1-a) to (2αA1-e), (2αB1), (2αA2-a) to (2αA2-e), (2αB2), (2αA3-a) to (2αA3-e), and (2αB3) also include those proteins whose amino acid residues are modified by phosphorylation, sugar chain modification, aminoacylation, ring-opening, deamination or the like.

The protein selected from the group consisting of the proteins as defined above in (2αA1-a) to (2αA1-e), (2αB1), (2αA2-a) to (2αA2-e), (2αB2), (2αA3-a) to (2αA3-e), and (2αB3) can be proteins that are found in a protein spot with a molecular weight of around 50 to 120 kDa, preferably around 60 to 110 kDa, and an isoelectric point of 2.5 to 6.5, preferably 3.0 to 6.0 on gels used in the two-dimensional electrophoresis performed under the conditions described above in the subsection titled “Identification of antigens”.

Preferably, a protein that is an antigen in spot 2α has activity equivalent to that of the protein having the amino acid sequence of SEQ ID NO: 106 (heat shock cognate protein 80), the protein having the amino acid sequence of SEQ ID NO: 122 (heat shock protein 90-1), or the protein having the amino acid sequence of SEQ ID NO: 139 (hypothetical protein GLYMA_02G302500), or causative of an allergy to soybean.

### (4α) Antigen in Spot 4α

As the result of sequence identification of the antigen in spot 4α by mass spectroscopy, the following amino acid sequences were detected.

(SEQ ID NO: 156)
AGGALSIAFVSK
(SEQ ID NO: 157)
DGLIVSIGK

Also, the mass spectroscopic data obtained for spot 4α on a mass spectrometer was analyzed by comparing the data against the NCBI protein data, and as a result, embryo-specific urease isoform X1 (amino acid sequence: SEQ ID NO: 155, encoding nucleotide sequence: SEQ ID NO: 154) was identified as a protein comprising SEQ ID NO: 156 or 157.

Accordingly, the antigens in spot 4α in the present invention can be any protein selected from the group consisting of the proteins as defined below in (4αA-a) to (4αA-e) and (4αB):
(4αA-a) a protein comprising an amino acid sequence with deletion, substitution, insertion or addition of one or several amino acids in SEQ ID NO: 155;
(4αA-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 155;
(4αA-c) a protein comprising an amino acid sequence encoded by a nucleotide sequence with deletion, substitution, insertion or addition of one or several nucleotides in SEQ ID NO: 154;
(4αA-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 154;
(4αA-e) a protein comprising an amino acid sequence encoded by a nucleic acid that hybridizes under stringent conditions with a nucleic acid having a nucleotide sequence complementary to the nucleotide sequence of SEQ ID NO: 154;
(4αB) a protein comprising at least one amino acid sequence selected from the group consisting of SEQ ID NOs: 156 and 157, preferably a protein comprising both the amino acid sequences. As referred to above, the amino acid sequence of any of SEQ ID NOs: 156 and 157 may be an amino acid sequence derived therefrom by deletion, substitution, insertion or addition of one or several amino acids.

The proteins selected from the group consisting of (4αA-a) to (4αA-e) and (4αB) as defined above also include those proteins whose amino acid residues are modified by phosphorylation, sugar chain modification, aminoacylation, ring-opening, deamination or the like.

The proteins selected from the group consisting of (4αA-a) to (4αA-e) and (4αB) as defined above can be proteins that are found in a protein spot with a molecular weight of around 70 to 120 kDa, preferably around 80 to 110 kDa and an isoelectric point of 3.5 to 7.5, preferably 4.0 to 7.0 on gels used in the two-dimensional electrophoresis performed under the conditions described above in the subsection titled “Identification of antigens”.

Preferably, a protein that is an antigen in spot 4α has activity equivalent to that of the protein having the amino acid sequence of SEQ ID NO: 155 (embryo-specific urease isoform X1) or causative of an allergy to soybean.

### (5α) Antigen in Spot 5α

As the result of sequence identification of the antigen in spot 5α by mass spectroscopy, the following amino acid sequences were detected.

(SEQ ID NO: 160)
AFATYVQAK
(SEQ ID NO: 161)
QQEATTSLSSFTPDASSIASSIK

Also, the mass spectroscopic data obtained for spot 5α on a mass spectrometer was analyzed by comparing the data against the NCBI protein data, and as a result, alpha-1,4 glucan phosphorylase L isozyme, chloroplastic/amyloplastic (amino acid sequence: SEQ ID NO: 159, encoding nucleotide sequence: SEQ ID NO: 158) was identified as a protein comprising SEQ ID NO: 160 or 161.

Accordingly, the antigens in spot 5α in the present invention can be any protein selected from the group consisting of the proteins as defined below in (5αA-a) to (5αA-e) and (5αB):
(5αA-a) a protein comprising an amino acid sequence with deletion, substitution, insertion or addition of one or several amino acids in SEQ ID NO: 159;
(5αA-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 159;
(5αA-c) a protein comprising an amino acid sequence encoded by a nucleotide sequence with deletion, substitution, insertion or addition of one or several nucleotides in SEQ ID NO: 158;
(5αA-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 158;
(5αA-e) a protein comprising an amino acid sequence encoded by a nucleic acid that hybridizes under stringent conditions with a nucleic acid having a nucleotide sequence complementary to the nucleotide sequence of SEQ ID NO: 158;
(5αB) a protein comprising at least one amino acid sequence selected from the group consisting of SEQ ID NOs: 160 and 161, preferably a protein comprising both the amino acid sequences. As referred to above, the amino acid sequence of any of SEQ ID NOs: 160 and 161 may be an amino acid sequence derived therefrom by deletion, substitution, insertion or addition of one or several amino acids.

The proteins selected from the group consisting of (5αA-a) to (5αA-e) and (5αB) as defined above also include those proteins whose amino acid residues are modified by phosphorylation, sugar chain modification, aminoacylation, ring-opening, deamination or the like.

The proteins selected from the group consisting of (5αA-a) to (5αA-e) and (5αB) as defined above can be proteins that are found in a protein spot with a molecular weight of around 70 to 170 kDa, preferably around 80 to 160 kDa and an isoelectric point of 3.5 to 7.5, preferably 4.0 to 7.0 on gels used in the two-dimensional electrophoresis performed under the conditions described above in the subsection titled “Identification of antigens”.

Preferably, a protein that is an antigen in spot 5α has activity equivalent to that of the protein having the amino acid sequence of SEQ ID NO: 159 (alpha-1,4 glucan phosphorylase L isozyme, chloroplastic/amyloplastic) or causative of an allergy to soybean.

### (6α) Antigen in Spot 6α

As the result of sequence identification of the antigen in spot 6α by mass spectroscopy, the following amino acid sequences were detected.

(SEQ ID NO: 164)
FLNGEVGPK
(SEQ ID NO: 165)
LSVFDAAEK
(SEQ ID NO: 166)
LSVFDVAEK
(SEQ ID NO: 167)
TSLAPGSGVVTK
(SEQ ID NO: 170)
FLNGEVGPK
(SEQ ID NO: 171)
LSVFDVAEK
(SEQ ID NO: 172)
TSLAPGSGVVTK

Also, the mass spectroscopic data obtained for spot 6α on a mass spectrometer was analyzed by comparing the data against the NCBI protein data, and as a result, aconitate hydratase 1 (amino acid sequence: SEQ ID NO: 163, encoding nucleotide sequence: SEQ ID NO: 162) was identified as a protein comprising any of SEQ ID NOs: 164 to 167 and aconitate hydratase 1 (amino acid sequence: SEQ ID NO: 169, encoding nucleotide sequence: SEQ ID NO: 168) was identified as a protein comprising any of SEQ ID NOs: 170 to 172.

Accordingly, the antigen in spot 6α in the present invention can be any of proteins selected from the group consisting of the proteins as defined below in (6αA1-a) to (6αA1-e), (6αB1), (6αA2-a) to (6αA2-e), and (6αB2):
(6αA1-a) a protein comprising an amino acid sequence with deletion, substitution, insertion or addition of one or several amino acids in SEQ ID NO: 163;
(6αA1-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 163;
(6αA1-c) a protein comprising an amino acid sequence encoded by a nucleotide sequence with deletion, substitution, insertion or addition of one or several nucleotides in SEQ ID NO: 162;
(6αA1-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 162;
(6αA1-e) a protein comprising an amino acid sequence encoded by a nucleic acid that hybridizes under stringent conditions with a nucleic acid having a nucleotide sequence complementary to the nucleotide sequence of SEQ ID NO: 162;
(6αB1) a protein comprising at least one amino acid sequence selected from the group consisting of SEQ ID NOs: 164-167, preferably a protein comprising at least 2, 3, or all sequences of the amino acid sequences. As referred to above, the amino acid sequence of any of SEQ ID NOs: 164 to 167 may be an amino acid sequence derived therefrom by deletion, substitution, insertion or addition of one or several amino acids;
(6αA2-a) a protein comprising an amino acid sequence with deletion, substitution, insertion or addition of one or several amino acids in SEQ ID NO: 169;
(6αA2-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 169;
(6αA2-c) a protein comprising an amino acid sequence encoded by a nucleotide sequence with deletion, substitution, insertion or addition of one or several nucleotides in SEQ ID NO: 168;
(6αA2-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 168;
(6αA2-e) a protein comprising an amino acid sequence encoded by a nucleic acid that hybridizes under stringent conditions with a nucleic acid having a nucleotide sequence complementary to the nucleotide sequence of SEQ ID NO: 168;
(6αB2) a protein comprising at least one amino acid sequence selected from the group consisting of SEQ ID NOs: 170-172, preferably a protein comprising at least 2, or all sequences of the amino acid sequences. As referred to above, the amino acid sequence of any of SEQ ID NOs: 170 to 172 may be an amino acid sequence derived therefrom by deletion, substitution, insertion or addition of one or several amino acids.

The protein selected from the group consisting of the proteins as defined above in (6αA1-a) to (6αA1-e), (6αB1), (6αA2-a) to (6αA2-e), and (6αB2) also include those proteins whose amino acid residues are modified by phosphorylation, sugar chain modification, aminoacylation, ring-opening, deamination or the like.

The protein selected from the group consisting of the proteins as defined above in (6αA1-a) to (6αA1-e), (6αB1), (6αA2-a) to (6αA2-e), and (6αB2) can be proteins that are found in a protein spot with a molecular weight of around 70 to 120 kDa, preferably around 80 to 110 kDa, and an isoelectric point of 3.5 to 7.5, preferably 4.0 to 7.0 on gels used in the two-dimensional electrophoresis performed under the conditions described above in the subsection titled “Identification of antigens”.

Preferably, a protein that is an antigen in spot 6α has activity equivalent to that of the protein having the amino acid sequence of SEQ ID NO: 163 or 169 (aconitate hydratase 1) or causative of an allergy to soybean.

### (7α) Antigen in Spot 7α

As the result of sequence identification of the antigen in spot 7α by mass spectroscopy, the following amino acid sequences were detected.

(SEQ ID NO: 175)
AGITVIQIDEAALREGLPLRK
(SEQ ID NO: 176)
ALGVDTVPVLVGPVTYLLLSKPAK
(SEQ ID NO: 177)
DEAFFSGNAAALASRK
(SEQ ID NO: 178)
DEVEDLEK
(SEQ ID NO: 179)
ISEEEYVK
(SEQ ID NO: 180)
IVEVNALAK
(SEQ ID NO: 181)
LIRNELAK
(SEQ ID NO: 182)
LLSVFREGVK
(SEQ ID NO: 183)
LVVSTSSSLLHTAVDLVNETK
(SEQ ID NO: 184)
SFSLLSLLPK
(SEQ ID NO: 185)
SSPRVTNEAVQK
(SEQ ID NO: 186)
TLTSLNGVTAYGFDLVRGTNTLDLIK
(SEQ ID NO: 187)
YGAGIGPGVYDIHSPRIPPTEEIADRINK

Also, the mass spectroscopic data obtained for spot 7α on a mass spectrometer was analyzed by comparing the data against the NCBI protein data, and as a result, methionine synthase (amino acid sequence: SEQ ID NO: 174, encoding nucleotide sequence: SEQ ID NO: 173) was identified as a protein comprising any of SEQ ID NOs: 175 to 187.

Accordingly, the antigens in spot 7α in the present invention can be any protein selected from the group consisting of the proteins as defined below in (7αA-a) to (7αA-e) and (7αB):
(7αA-a) a protein comprising an amino acid sequence with deletion, substitution, insertion or addition of one or several amino acids in SEQ ID NO: 174;
(7αA-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 174;
(7αA-c) a protein comprising an amino acid sequence encoded by a nucleotide sequence with deletion, substitution, insertion or addition of one or several nucleotides in SEQ ID NO: 173;
(7αA-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 173;
(7αA-e) a protein comprising an amino acid sequence encoded by a nucleic acid that hybridizes under stringent conditions with a nucleic acid having a nucleotide sequence complementary to the nucleotide sequence of SEQ ID NO: 173;
(7αB) a protein comprising at least one amino acid sequence selected from the group consisting of SEQ ID NOs: 175-187, preferably a protein comprising at least 2, 3, 4, 5, 6, 7, 8, 9, 10, 11 or 12 or all sequences of the amino acid sequences. As referred to above, the amino acid sequence of any of SEQ ID NOs: 175-187 may be an amino acid sequence derived therefrom by deletion, substitution, insertion or addition of one or several amino acids.

The proteins selected from the group consisting of (7αA-a) to (7αA-e) and (7αB) as defined above also include those proteins whose amino acid residues are modified by phosphorylation, sugar chain modification, aminoacylation, ring-opening, deamination or the like.

The proteins selected from the group consisting of (7αA-a) to (7αA-e) and (7αB) as defined above can be proteins that are found in a protein spot with a molecular weight of around 50 to 120 kDa, preferably around 60 to 110 kDa and an isoelectric point of 3.5 to 7.5, preferably 4.0 to 7.0 on gels used in the two-dimensional electrophoresis performed under the conditions described above in the subsection titled “Identification of antigens”.

Preferably, a protein that is an antigen in spot 7α has activity equivalent to that of the protein having the amino acid sequence of SEQ ID NO: 174 (methionine synthase) or causative of an allergy to soybean.

### (8α) Antigen in Spot 8α

As the result of sequence identification of the antigen in spot 8α by mass spectroscopy, the following amino acid sequences were detected.

(SEQ ID NO: 190)
AIGIDRFGASAPAGRIYK
(SEQ ID NO: 191)
ALPTYTPESPADATRNLSQTNLNALAK
(SEQ ID NO: 192)
ANSYSVHGSALGAK
(SEQ ID NO: 193)
ATADAALVEK
(SEQ ID NO: 194)
DKPTLIK
(SEQ ID NO: 195)
GGYTISDNSTGNKPDVILIGTGSELEIAAK
(SEQ ID NO: 196)
LPQLPGTSIEGVEK
(SEQ ID NO: 197)
NGNNGYDDIRAAIK
(SEQ ID NO: 198)
SVNTIRFLAIDAVEK
(SEQ ID NO: 199)
VTTTIGYGSPNK

Also, the mass spectroscopic data obtained for spot 8α on a mass spectrometer was analyzed by comparing the data against the NCBI protein data, and as a result, transketolase, chloroplastic (amino acid sequence: SEQ ID NO: 189, encoding nucleotide sequence: SEQ ID NO: 188) was identified as a protein comprising any of SEQ ID NOs: 190 to 199.

Accordingly, the antigens in spot 8α in the present invention can be any protein selected from the group consisting of the proteins as defined below in (8αA-a) to (8αA-e) and (8αB):
(8αA-a) a protein comprising an amino acid sequence with deletion, substitution, insertion or addition of one or several amino acids in SEQ ID NO: 189;
(8αA-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 189;
(8αA-c) a protein comprising an amino acid sequence encoded by a nucleotide sequence with deletion, substitution, insertion or addition of one or several nucleotides in SEQ ID NO: 188;
(8αA-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 188;
(8αA-e) a protein comprising an amino acid sequence encoded by a nucleic acid that hybridizes under stringent conditions with a nucleic acid having a nucleotide sequence complementary to the nucleotide sequence of SEQ ID NO: 188;
(8αB) a protein comprising at least one amino acid sequence selected from the group consisting of SEQ ID NOs: 190-199, preferably a protein comprising at least 2, 3, 4, 5, 6, 7, 8 or 9 or all sequences of the amino acid sequences. As referred to above, the amino acid sequence of any of SEQ ID NOs: 190-199 may be an amino acid sequence derived therefrom by deletion, substitution, insertion or addition of one or several amino acids.

The proteins selected from the group consisting of (8αA-a) to (8αA-e) and (8αB) as defined above also include those proteins whose amino acid residues are modified by phosphorylation, sugar chain modification, aminoacylation, ring-opening, deamination or the like.

The proteins selected from the group consisting of (8αA-a) to (8αA-e) and (8αB) as defined above can be proteins that are found in a protein spot with a molecular weight of around 50 to 120 kDa, preferably around 60 to 110 kDa and an isoelectric point of 3.5 to 8.5, preferably 4.0 to 8.0 on gels used in the two-dimensional electrophoresis performed under the conditions described above in the subsection titled “Identification of antigens”.

Preferably, a protein that is an antigen in spot 8α has activity equivalent to that of the protein having the amino acid sequence of SEQ ID NO: 189 (transketolase, chloroplastic) or causative of an allergy to soybean.

### (9α) Antigen in Spot 9α

As the result of sequence identification of the antigen in spot 9α by mass spectroscopy, the following amino acid sequences were detected.

(SEQ ID NO: 202)
DLSSEEANQYLK

Also, the mass spectroscopic data obtained for spot 9α on a mass spectrometer was analyzed by comparing the data against the NCBI protein data, and as a result, lysine-tRNA ligase, cytoplasmic-like isoform X2 (amino acid sequence: SEQ ID NO: 201, encoding nucleotide sequence: SEQ ID NO: 200) was identified as a protein comprising SEQ ID NO: 202.

Accordingly, the antigens in spot 9α in the present invention can be any protein selected from the group consisting of the proteins as defined below in (9αA-a) to (9αA-e) and (9αB):
(9αA-a) a protein comprising an amino acid sequence with deletion, substitution, insertion or addition of one or several amino acids in SEQ ID NO: 201;
(9αA-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 201;
(9αA-c) a protein comprising an amino acid sequence encoded by a nucleotide sequence with deletion, substitution, insertion or addition of one or several nucleotides in SEQ ID NO: 200;
(9αA2-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 200;
(9αA-e) a protein comprising an amino acid sequence encoded by a nucleic acid that hybridizes under stringent conditions with a nucleic acid having a nucleotide sequence complementary to the nucleotide sequence of SEQ ID NO: 200;
(9αB) a protein comprising an amino acid sequence of SEQ ID NO: 202. As referred to above, the amino acid sequence(s) of SEQ ID NO: 202 may be an amino acid sequence(s) derived therefrom by deletion, substitution, insertion or addition of one or several amino acids.

The proteins selected from the group consisting of (9αA-a) to (9αA-e) and (9αB) as defined above also include those proteins whose amino acid residues are modified by phosphorylation, sugar chain modification, aminoacylation, ring-opening, deamination or the like.

The proteins selected from the group consisting of (9αA-a) to (9αA-e) and (9αB) as defined above can be proteins that are found in a protein spot with a molecular weight of around 50 to 120 kDa, preferably around 60 to 110 kDa and an isoelectric point of 3.5 to 8.5, preferably 4.0 to 8.0 on gels used in the two-dimensional electrophoresis performed under the conditions described above in the subsection titled “Identification of antigens”.

Preferably, a protein that is an antigen in spot 9α has activity equivalent to that of the protein having the amino acid sequence of SEQ ID NO: 201 (lysine-tRNA ligase, cytoplasmic-like isoform X2) or causative of an allergy to soybean.

### (10α) Antigen in Spot 10α

As the result of sequence identification of the antigen in spot 10α by mass spectroscopy, the following amino acid sequences were detected.

(SEQ ID NO: 205)
AAVEEGIVPGGGVALLYASSELDK
(SEQ ID NO: 206)
DRVTDALNATK
(SEQ ID NO: 207)
EGVITISDGK
(SEQ ID NO: 208)
GRNVVIEQSFGAPK
(SEQ ID NO: 209)
GVEELADAVK
(SEQ ID NO: 210)
IGGASEAEVGEK
(SEQ ID NO: 211)
IGVQIIQNALK
(SEQ ID NO: 212)
ISSINAIVK
(SEQ ID NO: 213)
LLEQNDPDLGYDAAK
(SEQ ID NO: 214)
LQTANFDQK
(SEQ ID NO: 215)
LSGGVAVLK
(SEQ ID NO: 216)
NIGASLVK
(SEQ ID NO: 217)
TGIIDPLK
(SEQ ID NO: 218)
TPVHTIASNAGVEGAVVVGK
(SEQ ID NO: 221)
AGIIDPLK
(SEQ ID NO: 222)
DRVTDALNATK
(SEQ ID NO: 223)
EGVITISDGK
(SEQ ID NO: 224)
GRNVVIEQSFGAPK
(SEQ ID NO: 225)
GVEELADAVK
(SEQ ID NO: 226)
IGGASEAEVGEK
(SEQ ID NO: 227)
IGVQIIQNALK
(SEQ ID NO: 228)
ISSINAIVK
(SEQ ID NO: 229)
LLEQENHDLGYDAAK
(SEQ ID NO: 230)
LQTANFDQK
(SEQ ID NO: 231)
LSGGVAVLK

Also, the mass spectroscopic data obtained for spot 10α on a mass spectrometer was analyzed by comparing the data against the NCBI protein data, and as a result, chaperonin CPN60-2, mitochondrial (amino acid sequence: SEQ ID NO: 204, encoding nucleotide sequence: SEQ ID NO: 203) was identified as a protein comprising any of SEQ ID NOs: 205 to 218 and chaperonin CPN60-2, mitochondrial-like isoform X1 (amino acid sequence: SEQ ID NO: 220, encoding nucleotide sequence: SEQ ID NO: 219) was identified as a protein comprising any of SEQ ID NOs: 221 to 231.

Accordingly, the antigen in spot 10α in the present invention can be any of proteins selected from the group consisting of the proteins as defined below in (10αA1-a) to (10αA1-e), (10αB1), (10αA2-a) to (10αA2-e), and (10αB2):
(10αA1-a) a protein comprising an amino acid sequence with deletion, substitution, insertion or addition of one or several amino acids in SEQ ID NO: 204;
(10αA1-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 204;
(10αA1-c) a protein comprising an amino acid sequence encoded by a nucleotide sequence with deletion, substitution, insertion or addition of one or several nucleotides in SEQ ID NO: 203;
(10αA1-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 203;
(10αA1-e) a protein comprising an amino acid sequence encoded by a nucleic acid that hybridizes under stringent conditions with a nucleic acid having a nucleotide sequence complementary to the nucleotide sequence of SEQ ID NO: 203;
(10αB1) a protein comprising at least one amino acid sequence selected from the group consisting of SEQ ID NOs: 205-218, preferably a protein comprising at least 2, 3, 4, 5, 6, 7, 8, 9, 10, 11, 12, or 13 or all sequences of the amino acid sequences. As referred to above, the amino acid sequence of any of SEQ ID NOs: 205 to 218 may be an amino acid sequence derived therefrom by deletion, substitution, insertion or addition of one or several amino acids;
(10αA2-a) a protein comprising an amino acid sequence with deletion, substitution, insertion or addition of one or several amino acids in SEQ ID NO: 220;
(10αA2-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 220;
(10αA2-c) a protein comprising an amino acid sequence encoded by a nucleotide sequence with deletion, substitution, insertion or addition of one or several nucleotides in SEQ ID NO: 219;
(10αA2-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 219;
(10αA2-e) a protein comprising an amino acid sequence encoded by a nucleic acid that hybridizes under stringent conditions with a nucleic acid having a nucleotide sequence complementary to the nucleotide sequence of SEQ ID NO: 219;
(10αB2) a protein comprising at least one amino acid sequence selected from the group consisting of SEQ ID NOs: 221-231, preferably a protein comprising at least 2, 3, 4, 5, 6, 7, 8, 9, or 10, or all sequences of the amino acid sequences. As referred to above, the amino acid sequence of any of SEQ ID NOs: 221 to 231 may be an amino acid sequence derived therefrom by deletion, substitution, insertion or addition of one or several amino acids.

The protein selected from the group consisting of the proteins as defined above in (10αA1-a) to
(10αA1-e), (10αB1), (10αA2-a) to (10αA2-e), and (10αB2) also include those proteins whose amino acid residues are modified by phosphorylation, sugar chain modification, aminoacylation, ring-opening, deamination or the like.

The protein selected from the group consisting of the proteins as defined above in (10αA1-a) to (10αA1-e), (10αB1), (10αA2-a) to (10αA2-e), and (10αB2) can be proteins that are found in a protein spot with a molecular weight of around 30 to 90 kDa, preferably around 40 to 80 kDa, and an isoelectric point of 3.5 to 7.5, preferably 4.0 to 7.0 on gels used in the two-dimensional electrophoresis performed under the conditions described above in the subsection titled “Identification of antigens”.

Preferably, a protein that is an antigen in spot 10α has activity equivalent to that of the protein having the amino acid sequence of SEQ ID NO: 204 (haperonin CPN60-2, mitochondrial) or the protein having the amino acid sequence of SEQ ID NO: 220 (chaperonin CPN60-2, mitochondrial-like isoform X1) or causative of an allergy to soybean.

### (11α) Antigen in Spot 11α

As the result of sequence identification of the antigen in spot 11α by mass spectroscopy, the following amino acid sequences were detected.

(SEQ ID NO: 234)
AIDVLNFTPLNGK
(SEQ ID NO: 235)
FNNVFVK
(SEQ ID NO: 236)
GFGFVNFANVDDAAK
(SEQ ID NO: 237)
SGAANVFIK

Also, the mass spectroscopic data obtained for spot 11α on a mass spectrometer was analyzed by comparing the data against the NCBI protein data, and as a result, polyadenylate-binding protein 8 isoform X3 (amino acid sequence: SEQ ID NO: 233, encoding nucleotide sequence: SEQ ID NO: 232) was identified as a protein comprising any of SEQ ID NOs: 234 to 237.

Accordingly, the antigens in spot 11α in the present invention can be any protein selected from the group consisting of the proteins as defined below in (11αA-a) to (11αA-e) and (11αB):
(11αA-a) a protein comprising an amino acid sequence with deletion, substitution, insertion or addition of one or several amino acids in SEQ ID NO: 233;
(11αA-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 233;
(11αA-c) a protein comprising an amino acid sequence encoded by a nucleotide sequence with deletion, substitution, insertion or addition of one or several nucleotides in SEQ ID NO: 232;
(11αA-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 232;
(11αA-e) a protein comprising an amino acid sequence encoded by a nucleic acid that hybridizes under stringent conditions with a nucleic acid having a nucleotide sequence complementary to the nucleotide sequence of SEQ ID NO: 232;
(11αB) a protein comprising at least one amino acid sequence selected from the group consisting of SEQ ID NOs: 234-237, preferably a protein comprising at least 2 or 3 or all sequences of the amino acid sequences. As referred to above, the amino acid sequence of any of SEQ ID NOs: 234-237 may be an amino acid sequence derived therefrom by deletion, substitution, insertion or addition of one or several amino acids.

The proteins selected from the group consisting of (11αA-a) to (11αA-e) and (11αB) as defined above also include those proteins whose amino acid residues are modified by phosphorylation, sugar chain modification, aminoacylation, ring-opening, deamination or the like.

The proteins selected from the group consisting of (11αA-a) to (11αA-e) and (11αB) as defined above can be proteins that are found in a protein spot with a molecular weight of around 30 to 90 kDa, preferably around 40 to 80 kDa and an isoelectric point of 3.5 to 8.5, preferably 4.0 to 8.0 on gels used in the two-dimensional electrophoresis performed under the conditions described above in the subsection titled “Identification of antigens”.

Preferably, a protein that is an antigen in spot 11α has activity equivalent to that of the protein having the amino acid sequence of SEQ ID NO: 233 (polyadenylate-binding protein 8 isoform X3) or causative of an allergy to soybean.

### (12α) Antigen in Spot 12α

As the result of sequence identification of the antigen in spot 12α by mass spectroscopy, the following amino acid sequences were detected.

(SEQ ID NO: 240)
VVGVPVTLNGDLK

Also, the mass spectroscopic data obtained for spot 12α on a mass spectrometer was analyzed by comparing the data against the NCBI protein data, and as a result, pyrophosphate-fructose 6-phosphate 1-phosphotransferase subunit alpha-like (amino acid sequence: SEQ ID NO: 239, encoding nucleotide sequence: SEQ ID NO: 238) was identified as a protein comprising SEQ ID NO: 240.

Accordingly, the antigens in spot 12α in the present invention can be any protein selected from the group consisting of the proteins as defined below in (12αA-a) to (12αA-e) and (12αB):
(12αA-a) a protein comprising an amino acid sequence with deletion, substitution, insertion or addition of one or several amino acids in SEQ ID NO: 239;
(12αA-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 239;
(12αA-c) a protein comprising an amino acid sequence encoded by a nucleotide sequence with deletion, substitution, insertion or addition of one or several nucleotides in SEQ ID NO: 238;
(12αA-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 238;
(12αA-e) a protein comprising an amino acid sequence encoded by a nucleic acid that hybridizes under stringent conditions with a nucleic acid having a nucleotide sequence complementary to the nucleotide sequence of SEQ ID NO: 238;
(12αB) a protein comprising an amino acid sequence of SEQ ID NO: 240. As referred to above, the amino acid sequence(s) of SEQ ID NO: 240 may be an amino acid sequence(s) derived therefrom by deletion, substitution, insertion or addition of one or several amino acids.

The proteins selected from the group consisting of (12αA-a) to (12αA-e) and (12αB) as defined above also include those proteins whose amino acid residues are modified by phosphorylation, sugar chain modification, aminoacylation, ring-opening, deamination or the like.

The proteins selected from the group consisting of (12αA-a) to (12αA-e) and (12αB) as defined above can be proteins that are found in a protein spot with a molecular weight of around 30 to 90 kDa, preferably around 40 to 80 kDa and an isoelectric point of 3.5 to 8.5, preferably 4.0 to 8.0 on gels used in the two-dimensional electrophoresis performed under the conditions described above in the subsection titled “Identification of antigens”.

Preferably, a protein that is an antigen in spot 12α has activity equivalent to that of the protein having the amino acid sequence of SEQ ID NO: 239 (pyrophosphate-fructose 6-phosphate 1-phosphotransferase subunit alpha-like) or causative of an allergy to soybean.

### (13α) Antigen in Spot 13α

As the result of sequence identification of the antigen in spot 13α by mass spectroscopy, the following amino acid sequences were detected.

(SEQ ID NO: 243)
AASILSSHDPPVVLAK
(SEQ ID NO: 244)
DGNVAPFVK
(SEQ ID NO: 245)
DLASQYDVRGYPTIK
(SEQ ID NO: 246)
EDIIEFIEK
(SEQ ID NO: 247)
EEQVPLIIIQHNDGK
(SEQ ID NO: 248)
EFVLTLDHSNFHDTVSK
(SEQ ID NO: 249)
FFKPNLEADHIPTWLK
(SEQ ID NO: 250)
FFNSPNAK
(SEQ ID NO: 251)
FSGEEFDNFSALAEK
(SEQ ID NO: 252)
FVEESSTPVVTVFNNDPSNHPFVAK
(SEQ ID NO: 255)
AASILSSHDPPIVLAK
(SEQ ID NO: 256)
DGHVAPFVK
(SEQ ID NO: 257)
DLASQYDVK
(SEQ ID NO: 258)
EDIIEFIEK
(SEQ ID NO: 259)
EEQVPLIIIQHNDGK
(SEQ ID NO: 260)
EFVLTLDHSNFHDTVSK
(SEQ ID NO: 261)
FFKPNLEADHIPTWLK
(SEQ ID NO: 262)
FFNSPNAK
(SEQ ID NO: 263)
FSGEEFDNFSALAEK
(SEQ ID NO: 264)
FVEESSTPVVTVFNNEPSNHPFVVK
(SEQ ID NO: 265)
GFPTINILRNGGK
(SEQ ID NO: 266)
GPREADGIVDYLK
(SEQ ID NO: 267)
LAPEYEK
(SEQ ID NO: 268)
LDATANDIPSETFDVQGYPTVYFRSASGK
(SEQ ID NO: 269)
LRSDYDFGHTLNAK
(SEQ ID NO: 270)
LSQYDGGRTK
(SEQ ID NO: 271)
QLAPILDEVAISYQNEADVVIAK
(SEQ ID NO: 272)
QQGVSFLVGDVESSQGAFQYFGLK
(SEQ ID NO: 273)
QSGPASTEIK
(SEQ ID NO: 274)
SADEATAFIGENK
(SEQ ID NO: 275)
SEPIPETNDEPVK
(SEQ ID NO: 276)
VAIVGVFPK
(SEQ ID NO: 277)
VVVGASLEDIVFK
(SEQ ID NO: 278)
YREAAEQYK

Also, the mass spectroscopic data obtained for spot 13α on a mass spectrometer was analyzed by comparing the data against the NCBI protein data, and as a result, protein disulfide isomerase-like protein precursor (amino acid sequence: SEQ ID NO: 242, encoding nucleotide sequence: SEQ ID NO: 241) was identified as a protein comprising any of SEQ ID NOs: 243 to 252 and protein disulfide-isomerase (amino acid sequence: SEQ ID NO: 254, encoding nucleotide sequence: SEQ ID NO: 253) was identified as a protein comprising any of SEQ ID NOs: 255 to 278.

Accordingly, the antigen in spot 13α in the present invention can be any of proteins selected from the group consisting of the proteins as defined below in (13αA1-a) to (13αA1-e), (13αB1), (13αA2-a) to (13αA2-e), and (13αB2):
(13αA1-a) a protein comprising an amino acid sequence with deletion, substitution, insertion or addition of one or several amino acids in SEQ ID NO: 242;
(13αA1-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 242;
(13αA1-c) a protein comprising an amino acid sequence encoded by a nucleotide sequence with deletion, substitution, insertion or addition of one or several nucleotides in SEQ ID NO: 241;
(13αA1-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 241;
(13αA1-e) a protein comprising an amino acid sequence encoded by a nucleic acid that hybridizes under stringent conditions with a nucleic acid having a nucleotide sequence complementary to the nucleotide sequence of SEQ ID NO: 241;
(13αB1) a protein comprising at least one amino acid sequence selected from the group consisting of SEQ ID NOs: 243-252, preferably a protein comprising at least 2, 3, 4, 5, 6, 7, 8, or 9, or all sequences of the amino acid sequences. As referred to above, the amino acid sequence of any of SEQ ID NOs: 243-252 may be an amino acid sequence derived therefrom by deletion, substitution, insertion or addition of one or several amino acids;
(13αA2-a) a protein comprising an amino acid sequence with deletion, substitution, insertion or addition of one or several amino acids in SEQ ID NO: 254;
(13αA2-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 254;
(13αA2-c) a protein comprising an amino acid sequence encoded by a nucleotide sequence with deletion, substitution, insertion or addition of one or several nucleotides in SEQ ID NO: 253;
(13αA2-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 253;
(13αA2-e) a protein comprising an amino acid sequence encoded by a nucleic acid that hybridizes under stringent conditions with a nucleic acid having a nucleotide sequence complementary to the nucleotide sequence of SEQ ID NO: 253;
(13αB2) a protein comprising at least one amino acid sequence selected from the group consisting of SEQ ID NOs: 255-278, preferably a protein comprising at least 2, 3, 4, 5, 6, 7, 8, 9, 10, 11, 12, 13, 14, 15, 16, 17, 18, 19, 20, 21, 22, or 23, or all sequences of the amino acid sequences. As referred to above, the amino acid sequence of any of SEQ ID NOs: 255-278 may be an amino acid sequence derived therefrom by deletion, substitution, insertion or addition of one or several amino acids.

The protein selected from the group consisting of the proteins as defined above in (13αA1-a) to (13αA1-e), (13αB1), (13αA2-a) to (13αA2-e), and (13αB2) also include those proteins whose amino acid residues are modified by phosphorylation, sugar chain modification, aminoacylation, ring-opening, deamination or the like.

The protein selected from the group consisting of the proteins as defined above in (13αA1-a) to (13αA1-e), (13αB1), (13αA2-a) to (13αA2-e), and (13αB2) can be proteins that are found in a protein spot with a molecular weight of around 30 to 90 kDa, preferably around 40 to 80 kDa, and an isoelectric point of 3.5 to 7.5, preferably 4.0 to 7.0 on gels used in the two-dimensional electrophoresis performed under the conditions described above in the subsection titled “Identification of antigens”.

Preferably, a protein that is an antigen in spot 13α has activity equivalent to that of the protein having the amino acid sequence of SEQ ID NO: 242 (protein disulfide isomerase-like protein precursor) or the protein having the amino acid sequence of SEQ ID NO: 254 (protein disulfide-isomerase) or causative of an allergy to soybean.

### (14α) Antigen in Spot 14α

As the result of sequence identification of the antigen in spot 14α by mass spectroscopy, the following amino acid sequences were detected.

(SEQ ID NO: 281)
DHSDVSNQLYANYAIGK
(SEQ ID NO: 282)
FQEIVNIRLGDGTTRRGQVLEVDGEK
(SEQ ID NO: 283)
FTTVQFTGEVLK
(SEQ ID NO: 284)
TLDQFYSRDASN
(SEQ ID NO: 287)
FQEIVNIRLGDGTTRRGQVLEVDGEK
(SEQ ID NO: 288)
FTTVQFTGEVLK
(SEQ ID NO: 289)
TLDQFYSRDAGN

Also, the mass spectroscopic data obtained for spot 14α on a mass spectrometer was analyzed by comparing the data against the NCBI protein data, and as a result, V-type proton ATPase subunit B2 (amino acid sequence: SEQ ID NO: 280, encoding nucleotide sequence: SEQ ID NO: 279) was identified as a protein comprising any of SEQ ID NOs: 281 to 284 and V-type proton ATPase subunit B2 (amino acid sequence: SEQ ID NO: 286, encoding nucleotide sequence: SEQ ID NO: 285) was identified as a protein comprising any of SEQ ID NOs: 287 to 289.

Accordingly, the antigen in spot 14α in the present invention can be any of proteins selected from the group consisting of the proteins as defined below in (14αA1-a) to (14αA1-e), (14αB1), (14αA2-a) to (14αA2-e), and (14αB2):
(14αA1-a) a protein comprising an amino acid sequence with deletion, substitution, insertion or addition of one or several amino acids in SEQ ID NO: 280;
(14αA1-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 280;
(14αA1-c) a protein comprising an amino acid sequence encoded by a nucleotide sequence with deletion, substitution, insertion or addition of one or several nucleotides in SEQ ID NO: 279;
(14αA1-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 279;
(14αA1-e) a protein comprising an amino acid sequence encoded by a nucleic acid that hybridizes under stringent conditions with a nucleic acid having a nucleotide sequence complementary to the nucleotide sequence of SEQ ID NO: 279;
(14αB1) a protein comprising at least one amino acid sequence selected from the group consisting of SEQ ID NOs: 281-284, preferably a protein comprising at least 2, 3, or all sequences of the amino acid sequences. As referred to above, the amino acid sequence of any of SEQ ID NOs: 281 to 284 may be an amino acid sequence derived therefrom by deletion, substitution, insertion or addition of one or several amino acids;
(14αA2-a) a protein comprising an amino acid sequence with deletion, substitution, insertion or addition of one or several amino acids in SEQ ID NO: 286;
(14αA2-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 286;
(14αA2-c) a protein comprising an amino acid sequence encoded by a nucleotide sequence with deletion, substitution, insertion or addition of one or several nucleotides in SEQ ID NO: 285;
(14αA2-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 285;
(14αA2-e) a protein comprising an amino acid sequence encoded by a nucleic acid that hybridizes under stringent conditions with a nucleic acid having a nucleotide sequence complementary to the nucleotide sequence of SEQ ID NO: 285;
(14αB2) a protein comprising at least one amino acid sequence selected from the group consisting of SEQ ID NOs: 287-289, preferably a protein comprising at least 2, or all sequences of the amino acid sequences. As referred to above, the amino acid sequence of any of SEQ ID NOs: 287 to 289 may be an amino acid sequence derived therefrom by deletion, substitution, insertion or addition of one or several amino acids.

The protein selected from the group consisting of the proteins as defined above in (14αA1-a) to (14αA1-e), (14αB1), (14αA2-a) to (14αA2-e), and (14αB2) also include those proteins whose amino acid residues are modified by phosphorylation, sugar chain modification, aminoacylation, ring-opening, deamination or the like.

The protein selected from the group consisting of the proteins as defined above in (14αA1-a) to (14αA1-e), (14αB1), (14αA2-a) to (14αA2-e), and (14αB2) can be proteins that are found in a protein spot with a molecular weight of around 30 to 90 kDa, preferably around 40 to 80 kDa, and an isoelectric point of 2.5 to 6.5, preferably 3.0 to 6.0 on gels used in the two-dimensional electrophoresis performed under the conditions described above in the subsection titled “Identification of antigens”.

Preferably, a protein that is an antigen in spot 14α has activity equivalent to that of the protein having the amino acid sequence of SEQ ID NO: 280 or 286 (V-type proton ATPase subunit B2) or causative of an allergy to soybean.

### (15α, 16α) Antigens in Spots 15α, 16α

As the result of sequence identification of the antigens in spots 15α and 16α by mass spectroscopy, the following amino acid sequences were detected.

(SEQ ID NO: 292)
AIGINVPRSRFLPVK
(SEQ ID NO: 293)
ARENPENPSIELGPEFK
(SEQ ID NO: 294)
ATSDLLLVQSDLYTLEDGFVIRNK
(SEQ ID NO: 295)
EINGPEDL
(SEQ ID NO: 296)
EYVFVANSDNLGAIVDLK
(SEQ ID NO: 297)
ILNHLIQNK
(SEQ ID NO: 298)
IQTPTDEVVVPYDTLAPTPEGSSEVK
(SEQ ID NO: 299)
LDALLSQGK
(SEQ ID NO: 300)
RLVEADALK
(SEQ ID NO: 301)
SAVAGLNEISENEK
(SEQ ID NO: 302)
SIPSIVELDSLK
(SEQ ID NO: 303)
VLQLETAAGAAIRFFDK
(SEQ ID NO: 304)
VSNFLGRFK
(SEQ ID NO: 307)
AIGINVPRSRFLPVK
(SEQ ID NO: 308)
ATSDLLLVQSDLYTLEDGFVIRNK
(SEQ ID NO: 309)
EYVFVANSDNLGAIVDLK
(SEQ ID NO: 310)
ILNHLIQNK
(SEQ ID NO: 311)
IQTPTDEVVVPYDTLAPTPDGSSDVK
(SEQ ID NO: 312)
LDALLSQGK
(SEQ ID NO: 313)
LEVPDGAVISDK
(SEQ ID NO: 314)
RLVEADALK
(SEQ ID NO: 315)
SAVAGLNEISESEK
(SEQ ID NO: 316)
SIPSIVELDSLK
(SEQ ID NO: 317)
VLQLETAAGAAIRFFDK
(SEQ ID NO: 318)
VSNFLGRFK

Also, the mass spectroscopic data obtained for spots 15α, 16α on a mass spectrometer was analyzed by comparing the data against the NCBI protein data, and as a result, UTP-glucose-1-phosphate uridylyltransferase (amino acid sequence: SEQ ID NO: 291, encoding nucleotide sequence: SEQ ID NO: 290) was identified as a protein comprising any of SEQ ID NOs: 292 to 304 and the hypothetical protein GLYMA_02G241100 (amino acid sequence: SEQ ID NO: 306, encoding nucleotide sequence: SEQ ID NO: 305) was identified as a protein comprising any of SEQ ID NOs: 307 to 318.

Accordingly, the antigen in spot 15α, 16α in the present invention can be any of proteins selected from the group consisting of the proteins as defined below in (15, 16αA1-a) to (15, 16αA1-e), (15, 16αB1), (15, 16αA2-a) to (15, 16αA2-e), and (15, 16αB2):
(15, 16αA1-a) a protein comprising an amino acid sequence with deletion, substitution, insertion or addition of one or several amino acids in SEQ ID NO: 291;
(15, 16αA1-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 291;
(15, 16αA1-c) a protein comprising an amino acid sequence encoded by a nucleotide sequence with deletion, substitution, insertion or addition of one or several nucleotides in SEQ ID NO: 290;
(15, 16αA1-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 290;
(15, 16αA1-e) a protein comprising an amino acid sequence encoded by a nucleic acid that hybridizes under stringent conditions with a nucleic acid having a nucleotide sequence complementary to the nucleotide sequence of SEQ ID NO: 290;
(15, 16αB1) a protein comprising at least one amino acid sequence selected from the group consisting of SEQ ID NOs: 292-304, preferably a protein comprising at least 2, 3, 4, 5, 6, 7, 8, 9, 10, 11, or 12 or all sequences of the amino acid sequences. As referred to above, the amino acid sequence of any of SEQ ID NOs: 292 to 304 may be an amino acid sequence derived therefrom by deletion, substitution, insertion or addition of one or several amino acids;
(15, 16αA2-a) a protein comprising an amino acid sequence with deletion, substitution, insertion or addition of one or several amino acids in SEQ ID NO: 306;
(15, 16αA2-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 306;
(15, 16αA2-c) a protein comprising an amino acid sequence encoded by a nucleotide sequence with deletion, substitution, insertion or addition of one or several nucleotides in SEQ ID NO: 305;
(15, 16αA2-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 305;
(15, 16αA2-e) a protein comprising an amino acid sequence encoded by a nucleic acid that hybridizes under stringent conditions with a nucleic acid having a nucleotide sequence complementary to the nucleotide sequence of SEQ ID NO: 305;
(15, 16αB2) a protein comprising at least one amino acid sequence selected from the group consisting of SEQ ID NOs: 307-318, preferably a protein comprising at least 2, 3, 4, 5, 6, 7, 8, 9, 10, or 11 or all sequences of the amino acid sequences. As referred to above, the amino acid sequence of any of SEQ ID NOs: 307 to 318 may be an amino acid sequence derived therefrom by deletion, substitution, insertion or addition of one or several amino acids.

The protein selected from the group consisting of the proteins as defined above in (15, 16αA1-a) to (15, 16αA1-e), (15, 16αB1), (15, 16αA2-a) to (15, 16αA2-e), and (15, 16αB2) also include those proteins whose amino acid residues are modified by phosphorylation, sugar chain modification, aminoacylation, ring-opening, deamination or the like.

The protein selected from the group consisting of the proteins as defined above in (15, 16αA1-a) to (15, 16αA1-e), (15, 16αB1), (15, 16αA2-a) to (15, 16αA2-e), and (15, 16αB2) can be proteins that are found in a protein spot with a molecular weight of around 20 to 90 kDa, preferably around 30 to 80 kDa, and an isoelectric point of 3.5 to 7.5, preferably 4.0 to 7.0 on gels used in the two-dimensional electrophoresis performed under the conditions described above in the subsection titled “Identification of antigens”.

Preferably, a protein that is an antigen in spot 15α or 16α has activity equivalent to that of the protein having the amino acid sequence of SEQ ID NO: 291 (UTP-glucose-1-phosphate uridylyltransferase) or the protein having the amino acid sequence of SEQ ID NO: 306 (hypothetical protein GLYMA_02G241100) or causative of an allergy to soybean.

### (17α) Antigen in Spot 17α

As the result of sequence identification of the antigen in spot 17α by mass spectroscopy, the following amino acid sequences were detected.

(SEQ ID NO: 321)
AVDGSFVFNK
(SEQ ID NO: 322)
FFIYVQNYNESDPK
(SEQ ID NO: 323)
VEFDEEGK
(SEQ ID NO: 324)
VIDLSVDLSAASAAEE
(SEQ ID NO: 325)
VVGVTSEGETAK
(SEQ ID NO: 328)
AVDGSYVFNK
(SEQ ID NO: 329)
FFIYVQNYNESDPK
(SEQ ID NO: 330)
VEFDEEGK
(SEQ ID NO: 331)
VIDLSVDLSAASAAEE
(SEQ ID NO: 332)
VVGVTSEGETAK

Also, the mass spectroscopic data obtained for spot 17α on a mass spectrometer was analyzed by comparing the data against the NCBI protein data, and as a result, guanosine nucleotide diphosphate dissociation inhibitor 2 (amino acid sequence: SEQ ID NO: 320, encoding nucleotide sequence: SEQ ID NO: 319) was identified as a protein comprising any of SEQ ID NOs: 321 to 325 and rab GDP dissociation inhibitor alpha-like (amino acid sequence: SEQ ID NO: 327, encoding nucleotide sequence: SEQ ID NO: 326) was identified as a protein comprising any of SEQ ID NOs: 328 to 332.

Accordingly, the antigen in spot 17α in the present invention can be any of proteins selected from the group consisting of the proteins as defined below in (17αA1-a) to (17αA1-e), (17αB1), (17αA2-a) to (17αA2-e), and (17αB2):
(17αA1-a) a protein comprising an amino acid sequence with deletion, substitution, insertion or addition of one or several amino acids in SEQ ID NO: 320;
(17αA1-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 320;
(17αA1-c) a protein comprising an amino acid sequence encoded by a nucleotide sequence with deletion, substitution, insertion or addition of one or several nucleotides in SEQ ID NO: 319;
(17αA1-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 319;
(17αA1-e) a protein comprising an amino acid sequence encoded by a nucleic acid that hybridizes under stringent conditions with a nucleic acid having a nucleotide sequence complementary to the nucleotide sequence of SEQ ID NO: 319;
(17αB1) a protein comprising at least one amino acid sequence selected from the group consisting of SEQ ID NOs: 321-325, preferably a protein comprising at least 2, 3, or 4 or all sequences of the amino acid sequences. As referred to above, the amino acid sequence of any of SEQ ID NOs: 321 to 325 may be an amino acid sequence derived therefrom by deletion, substitution, insertion or addition of one or several amino acids.
(17αA2-a) a protein comprising an amino acid sequence with deletion, substitution, insertion or addition of one or several amino acids in SEQ ID NO: 327;
(17αA2-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 327;
(17αA2-c) a protein comprising an amino acid sequence encoded by a nucleotide sequence with deletion, substitution, insertion or addition of one or several nucleotides in SEQ ID NO: 326;
(17αA2-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 326;
(17αA2-e) a protein comprising an amino acid sequence encoded by a nucleic acid that hybridizes under stringent conditions with a nucleic acid having a nucleotide sequence complementary to the nucleotide sequence of SEQ ID NO: 326;
(17αB2) a protein comprising at least one amino acid sequence selected from the group consisting of SEQ ID NOs: 328-332, preferably a protein comprising at least 2, 3, or 4 or all sequences of the amino acid sequences. As referred to above, the amino acid sequence of any of SEQ ID NOs: 328 to 332 may be an amino acid sequence derived therefrom by deletion, substitution, insertion or addition of one or several amino acids;

The protein selected from the group consisting of the proteins as defined above in (17αA1-a) to (17αA1-e), (17αB1), (17αA2-a) to (17αA2-e), and (17αB2) also include those proteins whose amino acid residues are modified by phosphorylation, sugar chain modification, aminoacylation, ring-opening, deamination or the like.

The protein selected from the group consisting of the proteins as defined above in (17αA1-a) to (17αA1-e), (17αB1), (17αA2-a) to (17αA2-e), and (17αB2) can be proteins that are found in a protein spot with a molecular weight of around 20 to 90 kDa, preferably around 30 to 80 kDa, and an isoelectric point of 3.5 to 7.5, preferably 4.0 to 7.0 on gels used in the two-dimensional electrophoresis performed under the conditions described above in the subsection titled “Identification of antigens”.

Preferably, a protein that is an antigen in spot 17α has activity equivalent to that of the protein having the amino acid sequence of SEQ ID NO: 320 (guanosine nucleotide diphosphate dissociation inhibitor 2) or the protein having the amino acid sequence of SEQ ID NO: 327 (rab GDP dissociation inhibitor alpha-like) or causative of an allergy to soybean.

### (18α) Antigen in Spot 18α

As the result of sequence identification of the antigen in spot 18α by mass spectroscopy, the following amino acid sequences were detected.

(SEQ ID NO: 335)
DFETRVETEGGRIRVLK
(SEQ ID NO: 336)
DNIVSSLDNVAK
(SEQ ID NO: 337)
GRAVLGLVSESETEK
(SEQ ID NO: 338)
LFDQQNEGSIFAISREQVRALAPTK
(SEQ ID NO: 339)
RPTISNGYGRLTEVGPDDDEK

Also, the mass spectroscopic data obtained for spot 18α on a mass spectrometer was analyzed by comparing the data against the NCBI protein data, and as a result, hypothetical protein GLYMA_10G028300 (amino acid sequence: SEQ ID NO: 334, encoding nucleotide sequence: SEQ ID NO: 333) was identified as a protein comprising any of SEQ ID NOs: 335 to 339.

Accordingly, the antigens in spot 18α in the present invention can be any protein selected from the group consisting of the proteins as defined below in (18αA-a) to (18αA-e) and (18αB):
(18αA-a) a protein comprising an amino acid sequence with deletion, substitution, insertion or addition of one or several amino acids in SEQ ID NO: 334;
(18αA-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 334;
(18αA-c) a protein comprising an amino acid sequence encoded by a nucleotide sequence with deletion, substitution, insertion or addition of one or several nucleotides in SEQ ID NO: 333;
(18αA-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 333;
(18αA-e) a protein comprising an amino acid sequence encoded by a nucleic acid that hybridizes under stringent conditions with a nucleic acid having a nucleotide sequence complementary to the nucleotide sequence of SEQ ID NO: 333;
(18αB) a protein comprising at least one amino acid sequence selected from the group consisting of SEQ ID NOs: 335-339, preferably a protein comprising at least 2, 3 or 4 or all sequences of the amino acid sequences. As referred to above, the amino acid sequence of any of SEQ ID NOs: 335-339 may be an amino acid sequence derived therefrom by deletion, substitution, insertion or addition of one or several amino acids.

The proteins selected from the group consisting of (18αA-a) to (18αA-e) and (18αB) as defined above also include those proteins whose amino acid residues are modified by phosphorylation, sugar chain modification, aminoacylation, ring-opening, deamination or the like.

The proteins selected from the group consisting of (18αA-a) to (18αA-e) and (18αB) as defined above can be proteins that are found in a protein spot with a molecular weight of around 30 to 90 kDa, preferably around 40 to 80 kDa and an isoelectric point of 3.5 to 8.5, preferably 4.0 to 8.0 on gels used in the two-dimensional electrophoresis performed under the conditions described above in the subsection titled “Identification of antigens”.

Preferably, a protein that is an antigen in spot 18α has activity equivalent to that of the protein having the amino acid sequence of SEQ ID NO: 334 (hypothetical protein GLYMA_10G028300) or causative of an allergy to soybean.

### (19α) Antigen in Spot 19α

As the result of sequence identification of the antigen in spot 19α by mass spectroscopy, the following amino acid sequences were detected.

(SEQ ID NO: 342)
AQFNIFSK
(SEQ ID NO: 343)
DFSTRVETEGGSIRVLK
(SEQ ID NO: 344)
DNIVSSLDNVAK
(SEQ ID NO: 345)
GRAVLGLVRESETEK
(SEQ ID NO: 346)
RPTFSNGYGRLTEVGPDDEK

Also, the mass spectroscopic data obtained for spot 19α on a mass spectrometer was analyzed by comparing the data against the NCBI protein data, and as a result, sucrose binding protein homolog S-64 (amino acid sequence: SEQ ID NO: 341, encoding nucleotide sequence: SEQ ID NO: 340) was identified as a protein comprising any of SEQ ID NOs: 342 to 346.

Accordingly, the antigens in spot 19α in the present invention can be any protein selected from the group consisting of the proteins as defined below in (19αA-a) to (19αA-e) and (19αB):
(19αA-a) a protein comprising an amino acid sequence with deletion, substitution, insertion or addition of one or several amino acids in SEQ ID NO: 341;
(19αA-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 341;
(19αA-c) a protein comprising an amino acid sequence encoded by a nucleotide sequence with deletion, substitution, insertion or addition of one or several nucleotides in SEQ ID NO: 340;
(19αA-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 340;
(19αA-e) a protein comprising an amino acid sequence encoded by a nucleic acid that hybridizes under stringent conditions with a nucleic acid having a nucleotide sequence complementary to the nucleotide sequence of SEQ ID NO: 340;
(19αB) a protein comprising at least one amino acid sequence selected from the group consisting of SEQ ID NOs: 342-346, preferably a protein comprising at least 2, 3 or 4 or all sequences of the amino acid sequences. As referred to above, the amino acid sequence of any of SEQ ID NOs: 342-346 may be an amino acid sequence derived therefrom by deletion, substitution, insertion or addition of one or several amino acids.

The proteins selected from the group consisting of (19αA-a) to (19αA-e) and (19αB) as defined above also include those proteins whose amino acid residues are modified by phosphorylation, sugar chain modification, aminoacylation, ring-opening, deamination or the like.

The proteins selected from the group consisting of (19αA-a) to (19αA-e) and (19αB) as defined above can be proteins that are found in a protein spot with a molecular weight of around 30 to 90 kDa, preferably around 40 to 80 kDa and an isoelectric point of 3.5 to 8.5, preferably 4.0 to 8.0 on gels used in the two-dimensional electrophoresis performed under the conditions described above in the subsection titled “Identification of antigens”.

Preferably, a protein that is an antigen in spot 19α has activity equivalent to that of the protein having the amino acid sequence of SEQ ID NO: 341 (sucrose binding protein homolog S-64) or causative of an allergy to soybean.

### (25α) Antigen in Spot 25α

As the result of sequence identification of the antigen in spot 25α by mass spectroscopy, the following amino acid sequences were detected.

(SEQ ID NO: 349)
DLFPNENELVVK
(SEQ ID NO: 350)
EIFSLRDTTQEDPREVTAAK
(SEQ ID NO: 351)
LHGGTPANFLDVGGNASENQVVEAFK
(SEQ ID NO: 352)
LNFDDNAAYRQK
(SEQ ID NO: 353)
SQILAGGRGLGTFK
(SEQ ID NO: 354)
TDQVEDIAGK
(SEQ ID NO: 355)
VPVVVRLEGTNVDQGK
(SEQ ID NO: 356)
VVDGLALK
(SEQ ID NO: 359)
DLFPNENELVVK
(SEQ ID NO: 360)
EIFALRDTTQEDPREVTAAK
(SEQ ID NO: 361)
LHGGTPANFLDVGGNASEGQVVEAFK
(SEQ ID NO: 362)
LNFDDNAAYRQK
(SEQ ID NO: 363)
SQILAGGRGLGTFK
(SEQ ID NO: 364)
TDQVEDIAGK
(SEQ ID NO: 365)
VVDGLALK

Also, the mass spectroscopic data obtained for spot 25α on a mass spectrometer was analyzed by comparing the data against the NCBI protein data, and as a result, succinyl-CoA ligase [ADP-forming]subunit beta, mitochondrial (amino acid sequence: SEQ ID NO: 348, encoding nucleotide sequence: SEQ ID NO: 347) was identified as a protein comprising any of SEQ ID NOs: 349 to 356 and succinyl-CoA ligase [ADP-forming]subunit beta, mitochondrial (amino acid sequence: SEQ ID NO: 358, encoding nucleotide sequence: SEQ ID NO: 357) was identified as a protein comprising any of SEQ ID NOs: 359 to 365.

Accordingly, the antigen in spot 25α in the present invention can be any of proteins selected from the group consisting of the proteins as defined below in (25αA1-a) to (25αA1-e), (25αB1), (25αA2-a) to (25αA2-e), and (25αB2):
(25αA1-a) a protein comprising an amino acid sequence with deletion, substitution, insertion or addition of one or several amino acids in SEQ ID NO: 348;
(25αA1-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 348;
(25αA1-c) a protein comprising an amino acid sequence encoded by a nucleotide sequence with deletion, substitution, insertion or addition of one or several nucleotides in SEQ ID NO: 347;
(25αA1-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 347;
(25αA1-e) a protein comprising an amino acid sequence encoded by a nucleic acid that hybridizes under stringent conditions with a nucleic acid having a nucleotide sequence complementary to the nucleotide sequence of SEQ ID NO: 347;
(25αB1) a protein comprising at least one amino acid sequence selected from the group consisting of SEQ ID NOs: 349-356, preferably a protein comprising at least 2, 3, 4, 5, 6, or 7 or all sequences of the amino acid sequences. As referred to above, the amino acid sequence of any of SEQ ID NOs: 349 to 356 may be an amino acid sequence derived therefrom by deletion, substitution, insertion or addition of one or several amino acids;
(25αA2-a) a protein comprising an amino acid sequence with deletion, substitution, insertion or addition of one or several amino acids in SEQ ID NO: 358;
(25αA2-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 358;
(25αA2-c) a protein comprising an amino acid sequence encoded by a nucleotide sequence with deletion, substitution, insertion or addition of one or several nucleotides in SEQ ID NO: 357;
(25αA2-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 357;
(25αA2-e) a protein comprising an amino acid sequence encoded by a nucleic acid that hybridizes under stringent conditions with a nucleic acid having a nucleotide sequence complementary to the nucleotide sequence of SEQ ID NO: 357;
(25αB2) a protein comprising at least one amino acid sequence selected from the group consisting of SEQ ID NOs: 359-365, preferably a protein comprising at least 2, 3, 4, 5, or 6 or all sequences of the amino acid sequences. As referred to above, the amino acid sequence of any of SEQ ID NOs: 359 to 365 may be an amino acid sequence derived therefrom by deletion, substitution, insertion or addition of one or several amino acids.

The protein selected from the group consisting of the proteins as defined above in (25αA1-a) to (25αA1-e), (25αB1), (25αA2-a) to (25αA2-e), and (25αB2) also include those proteins whose amino acid residues are modified by phosphorylation, sugar chain modification, aminoacylation, ring-opening, deamination or the like.

The protein selected from the group consisting of the proteins as defined above in (25αA1-a) to (25αA1-e), (25αB1), (25αA2-a) to (25αA2-e), and (25αB2) can be proteins that are found in a protein spot with a molecular weight of around 20 to 90 kDa, preferably around 30 to 80 kDa, and an isoelectric point of 3.5 to 7.5, preferably 4.0 to 7.0 on gels used in the two-dimensional electrophoresis performed under the conditions described above in the subsection titled “Identification of antigens”.

Preferably, a protein that is an antigen in spot 25α has activity equivalent to that of the protein having the amino acid sequence of SEQ ID NO: 348 or 358 (succinyl-CoA ligase [ADP-forming]subunit beta, mitochondrial) or causative of an allergy to soybean.

### (26α) Antigen in Spot 26α

As the result of sequence identification of the antigen in spot 26α by mass spectroscopy, the following amino acid sequences were detected.

(SEQ ID NO: 368)
AQGYSVGSSLVEEDK
(SEQ ID NO: 369)
ELDYLVGAVSNPK
(SEQ ID NO: 370)
FAADANSK
(SEQ ID NO: 371)
FATGTEAIAK
(SEQ ID NO: 372)
GVSLLLPTDVVIADK
(SEQ ID NO: 373)
GVTTIIGGGDSVAAVEK
(SEQ ID NO: 374)
IGVIESLLEK
(SEQ ID NO: 375)
LASLADLYVNDAFGTAHRAHASTEGVAK
(SEQ ID NO: 376)
LSLATTLLEK
(SEQ ID NO: 377)
LVTQLPEGGVLLLENVRFYK
(SEQ ID NO: 378)
NDPEFAK
(SEQ ID NO: 379)
QLPGVLALDDA
(SEQ ID NO: 380)
RPFAAIVGGSK
(SEQ ID NO: 381)
VILSSHLGRPK
(SEQ ID NO: 382)
YLTGYGAK
(SEQ ID NO: 385)
ELDYLVGAVSNPK
(SEQ ID NO: 386)
ELPGVLALDEAVPVAV
(SEQ ID NO: 387)
FAVGTEAIAK
(SEQ ID NO: 388)
GVSLLLPSDVVIADK
(SEQ ID NO: 389)
GVTTIIGGGDSVAAVEK
(SEQ ID NO: 390)
IGVIESLLEK
(SEQ ID NO: 391)
RPFAAIVGGSK
(SEQ ID NO: 392)
VILSSHLGRPK
(SEQ ID NO: 395)
AQGYSVGSSLVEEDK
(SEQ ID NO: 396)
ELDYLVGAVSNPK
(SEQ ID NO: 397)
FAADANSK
(SEQ ID NO: 398)
FATGTEAIAK
(SEQ ID NO: 399)
GVSLLLPTDVVIADK
(SEQ ID NO: 400)
GVTTIIGGGDSVAAVEK
(SEQ ID NO: 401)
IGVIESLLEK
(SEQ ID NO: 402)
LASLADLYVNDAFGTAHRAHASTEGVAK
(SEQ ID NO: 403)
LSLATTLLEK
(SEQ ID NO: 404)
LVAQLPEGGVLLLENVRFYK
(SEQ ID NO: 405)
NDPEFAK
(SEQ ID NO: 406)
QLPGVLALDDA
(SEQ ID NO: 407)
RPFAAIVGGSK
(SEQ ID NO: 408)
TFGEALDK
(SEQ ID NO: 409)
VILSSHLGRPK

Also, the mass spectroscopic data obtained for spot 26α on a mass spectrometer was analyzed by comparing the data against the NCBI protein data, and as a result, phosphoglycerate kinase, cytosolic (amino acid sequence: SEQ ID NO: 367, encoding nucleotide sequence: SEQ ID NO: 366) was identified as a protein comprising any of SEQ ID NOs: 368 to 382, phosphoglycerate kinase, cytosolic (amino acid sequence: SEQ ID NO: 384, encoding nucleotide sequence: SEQ ID NO: 383) was identified as a protein comprising any of SEQ ID NOs: 385 to 392, and phosphoglycerate kinase, cytosolic (amino acid sequence: SEQ ID NO: 394, encoding nucleotide sequence: SEQ ID NO: 393) was identified as a protein comprising any of SEQ ID NOs: 395 to 409.

Accordingly, the antigen in spot 26α in the present invention can be any of proteins selected from the group consisting of the proteins as defined below in (26αA1-a) to (26αA1-e), (26αB1), (26αA2-a) to (26αA2-e), (26αB2), (26αA3-a) to (26αA3-e), and (26αB3):
(26αA1-a) a protein comprising an amino acid sequence with deletion, substitution, insertion or addition of one or several amino acids in SEQ ID NO: 367;
(26αA1-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 367;
(26αA1-c) a protein comprising an amino acid sequence encoded by a nucleotide sequence with deletion, substitution, insertion or addition of one or several nucleotides in SEQ ID NO: 366;
(26αA1-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 366;
(26αA1-e) a protein comprising an amino acid sequence encoded by a nucleic acid that hybridizes under stringent conditions with a nucleic acid having a nucleotide sequence complementary to the nucleotide sequence of SEQ ID NO: 366;
(26αB1) a protein comprising at least one amino acid sequence selected from the group consisting of SEQ ID NOs: 368-382, preferably a protein comprising at least 2, 3, 4, 5, 6, 7, 8, 9, 10, 11, 12, 13, or 14 or all sequences of the amino acid sequences. As referred to above, the amino acid sequence of any of SEQ ID NOs: 368 to 382 may be an amino acid sequence derived therefrom by deletion, substitution, insertion or addition of one or several amino acids;
(26αA2-a) a protein comprising an amino acid sequence with deletion, substitution, insertion or addition of one or several amino acids in SEQ ID NO: 384;
(26αA2-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 384;
(26αA2-c) a protein comprising an amino acid sequence encoded by a nucleotide sequence with deletion, substitution, insertion or addition of one or several nucleotides in SEQ ID NO: 383;
(26αA2-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 383;
(26αA2-e) a protein comprising an amino acid sequence encoded by a nucleic acid that hybridizes under stringent conditions with a nucleic acid having a nucleotide sequence complementary to the nucleotide sequence of SEQ ID NO: 383;
(26αB2) a protein comprising at least one amino acid sequence selected from the group consisting of SEQ ID NOs: 385-392, preferably a protein comprising at least 2, 3, 4, 5, 6, or 7 or all sequences of the amino acid sequences. As referred to above, the amino acid sequence of any of SEQ ID NOs: 385 to 392 may be an amino acid sequence derived therefrom by deletion, substitution, insertion or addition of one or several amino acids.
(26αA3-a) a protein comprising an amino acid sequence with deletion, substitution, insertion or addition of one or several amino acids in SEQ ID NO: 394;
(26αA3-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 394;
(26αA3-c) a protein comprising an amino acid sequence encoded by a nucleotide sequence with deletion, substitution, insertion or addition of one or several nucleotides in SEQ ID NO: 393;
(26αA3-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 393;
(26αA3-e) a protein comprising an amino acid sequence encoded by a nucleic acid that hybridizes under stringent conditions with a nucleic acid having a nucleotide sequence complementary to the nucleotide sequence of SEQ ID NO: 393;
(26αB3) a protein comprising at least one amino acid sequence selected from the group consisting of SEQ ID NOs: 395-409, preferably a protein comprising at least 2, 3, 4, 5, 6, 7, 8, 9, 10, 11, 12, 13, or 14 or all sequences of the amino acid sequences. As referred to above, the amino acid sequence of any of SEQ ID NOs: 395 to 409 may be an amino acid sequence derived therefrom by deletion, substitution, insertion or addition of one or several amino acids.

The protein selected from the group consisting of the proteins as defined above in (26αA1-a) to (26αA1-e), (26αB1), (26αA2-a) to (26αA2-e), (26αB2), (26αA3-a) to (26αA3-e), and (26αB3) also include those proteins whose amino acid residues are modified by phosphorylation, sugar chain modification, aminoacylation, ring-opening, deamination or the like.

The protein selected from the group consisting of the proteins as defined above in (26αA1-a) to (26αA1-e), (26αB1), (26αA2-a) to (26αA2-e), (26αB2), (26αA3-a) to (26αA3-e), and (26αB3) can be proteins that are found in a protein spot with a molecular weight of around 20 to 90 kDa, preferably around 30 to 80 kDa, and an isoelectric point of 3.5 to 7.5, preferably 4.0 to 7.0 on gels used in the two-dimensional electrophoresis performed under the conditions described above in the subsection titled “Identification of antigens”.

Preferably, a protein that is an antigen in spot 25α has activity equivalent to that of the protein having the amino acid sequence of SEQ ID NO: 367, 384, or 394 (phosphoglycerate kinase, cytosolic) or causative of an allergy to soybean.

### (27α) Antigen in Spot 27α

As the result of sequence identification of the antigen in spot 27α by mass spectroscopy, the following amino acid sequences were detected.

(SEQ ID NO: 412)
FGVNEFVNPK
(SEQ ID NO: 413)
FITHTVPFSEINK
(SEQ ID NO: 414)
GTFYGNYKPRTDLPSVVEK
(SEQ ID NO: 415)
THPVNFLNERTLK
(SEQ ID NO: 416)
VNPAAPLDK
(SEQ ID NO: 419)
GTFYGHYRPRTDIPGVVEK
(SEQ ID NO: 420)
INPNAPLDK
(SEQ ID NO: 423)
FGVNEFVNPK
(SEQ ID NO: 424)
FITHTVPFSEINK
(SEQ ID NO: 425)
GTFYGNYKPRTDLPSVVEK
(SEQ ID NO: 426)
INPAAPLDK
(SEQ ID NO: 427)
TAPINFLNERTLK

Also, the mass spectroscopic data obtained for spot 27α on a mass spectrometer was analyzed by comparing the data against the NCBI protein data, and as a result, alcohol dehydrogenase 1 (amino acid sequence: SEQ ID NO: 411, encoding nucleotide sequence: SEQ ID NO: 410) was identified as a protein comprising any of SEQ ID NOs: 412 to 416, alcohol dehydrogenase 1 (amino acid sequence: SEQ ID NO: 418, encoding nucleotide sequence: SEQ ID NO: 417) was identified as a protein comprising any of SEQ ID NOs: 419 and 420, and alcohol dehydrogenase 1-like (amino acid sequence: SEQ ID NO: 422, encoding nucleotide sequence: SEQ ID NO: 421) was identified as a protein comprising any of SEQ ID NOs: 423 to 427.

Accordingly, the antigen in spot 27α in the present invention can be any of proteins selected from the group consisting of the proteins as defined below in (27αA1-a) to (27αA1-e), (27αB1), (27αA2-a) to (27αA2-e), (27αB2), (27αA3-a) to (27αA3-e), and (27αB3):
(27αA1-a) a protein comprising an amino acid sequence with deletion, substitution, insertion or addition of one or several amino acids in SEQ ID NO: 411;
(27αA1-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 411;
(27αA1-c) a protein comprising an amino acid sequence encoded by a nucleotide sequence with deletion, substitution, insertion or addition of one or several nucleotides in SEQ ID NO: 410;
(27αA1-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 410;
(27αA1-e) a protein comprising an amino acid sequence encoded by a nucleic acid that hybridizes under stringent conditions with a nucleic acid having a nucleotide sequence complementary to the nucleotide sequence of SEQ ID NO: 410;
(27αB1) a protein comprising at least one amino acid sequence selected from the group consisting of SEQ ID NOs: 412-416, preferably a protein comprising at least 2, 3, or 4 or all sequences of the amino acid sequences. As referred to above, the amino acid sequence of any of SEQ ID NOs: 412 to 416 may be an amino acid sequence derived therefrom by deletion, substitution, insertion or addition of one or several amino acids;
(27αA2-a) a protein comprising an amino acid sequence with deletion, substitution, insertion or addition of one or several amino acids in SEQ ID NO: 418;
(27αA2-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 418;
(27αA2-c) a protein comprising an amino acid sequence encoded by a nucleotide sequence with deletion, substitution, insertion or addition of one or several nucleotides in SEQ ID NO: 417;
(27αA2-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 417;
(27αA2-e) a protein comprising an amino acid sequence encoded by a nucleic acid that hybridizes under stringent conditions with a nucleic acid having a nucleotide sequence complementary to the nucleotide sequence of SEQ ID NO: 417;
(27αB2) a protein comprising at least one amino acid sequence selected from the group consisting of SEQ ID NOs: 419 and 420, preferably a protein comprising both the amino acid sequences. As referred to above, the amino acid sequence of any of SEQ ID NOs: 419, 420 may be an amino acid sequence derived therefrom by deletion, substitution, insertion or addition of one or several amino acids.
(27αA3-a) a protein comprising an amino acid sequence with deletion, substitution, insertion or addition of one or several amino acids in SEQ ID NO: 422;
(27αA3-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 422;
(27αA3-c) a protein comprising an amino acid sequence encoded by a nucleotide sequence with deletion, substitution, insertion or addition of one or several nucleotides in SEQ ID NO: 421;
(27αA3-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 421;
(27αA3-e) a protein comprising an amino acid sequence encoded by a nucleic acid that hybridizes under stringent conditions with a nucleic acid having a nucleotide sequence complementary to the nucleotide sequence of SEQ ID NO: 421;
(27αB3) a protein comprising at least one amino acid sequence selected from the group consisting of SEQ ID NOs: 423-427, preferably a protein comprising at least 2, 3, or 4 or all sequences of the amino acid sequences. As referred to above, the amino acid sequence of any of SEQ ID NOs: 423 to 427 may be an amino acid sequence derived therefrom by deletion, substitution, insertion or addition of one or several amino acids.

The protein selected from the group consisting of the proteins as defined above in (27αA1-a) to (27αA1-e), (27αB1), (27αA2-a) to (27αA2-e), (27αB2), (27αA3-a) to (27αA3-e), and (27αB3) also include those proteins whose amino acid residues are modified by phosphorylation, sugar chain modification, aminoacylation, ring-opening, deamination or the like.

The protein selected from the group consisting of the proteins as defined above in (27αA1-a) to (27αA1-e), (27αB1), (27αA2-a) to (27αA2-e), (27αB2), (27αA3-a) to (27αA3-e), and (27αB3) can be proteins that are found in a protein spot with a molecular weight of around 20 to 90 kDa, preferably around 30 to 80 kDa, and an isoelectric point of 3.5 to 7.5, preferably 4.0 to 7.0 on gels used in the two-dimensional electrophoresis performed under the conditions described above in the subsection titled “Identification of antigens”.

Preferably, a protein that is an antigen in spot 27α has activity equivalent to that of the protein having the amino acid sequence of SEQ ID NO: 411 or 418 (alcohol dehydrogenase 1) or the protein having the amino acid sequence of SEQ ID NO: 422 (alcohol dehydrogenase 1-like) or causative of an allergy to soybean.

### (28α) Antigen in Spot 28α

As the result of sequence identification of the antigen in spot 28α by mass spectroscopy, the following amino acid sequences were detected.

(SEQ ID NO: 430)
AGEYTDYAAQK
(SEQ ID NO: 431)
DIGDINRTNERDEGYDLHDEANFK
(SEQ ID NO: 432)
DLEDANRLRDNTFNTWK
(SEQ ID NO: 433)
EAVVGKPHHEEVYAK

Also, the mass spectroscopic data obtained for spot 28α on a mass spectrometer was analyzed by comparing the data against the NCBI protein data, and as a result, 35 kDa seed maturation protein isoform X1 (amino acid sequence: SEQ ID NO: 429, encoding nucleotide sequence: SEQ ID NO: 428) was identified as a protein comprising any of SEQ ID NO: 430 to 433.

Accordingly, the antigens in spot 28α in the present invention can be any protein selected from the group consisting of the proteins as defined below in (28αA-a) to (28αA-e) and (28αB):
(28αA-a) a protein comprising an amino acid sequence with deletion, substitution, insertion or addition of one or several amino acids in SEQ ID NO: 429;
(28αA-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 429;
(28αA-c) a protein comprising an amino acid sequence encoded by a nucleotide sequence with deletion, substitution, insertion or addition of one or several nucleotides in SEQ ID NO: 428;
(28αA-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 428;
(28αA-e) a protein comprising an amino acid sequence encoded by a nucleic acid that hybridizes under stringent conditions with a nucleic acid having a nucleotide sequence complementary to the nucleotide sequence of SEQ ID NO: 428;
(28αB) a protein comprising at least one amino acid sequence selected from the group consisting of SEQ ID NOs: 430-433, preferably a protein comprising at least 2 or 3 or all sequences of the amino acid sequences. As referred to above, the amino acid sequence of any of SEQ ID NOs: 430-433 may be an amino acid sequence derived therefrom by deletion, substitution, insertion or addition of one or several amino acids.

The proteins selected from the group consisting of (28αA-a) to (28αA-e) and (28αB) as defined above also include those proteins whose amino acid residues are modified by phosphorylation, sugar chain modification, aminoacylation, ring-opening, deamination or the like.

The proteins selected from the group consisting of (28αA-a) to (28αA-e) and (28αB) as defined above can be proteins that are found in a protein spot with a molecular weight of around 10 to 70 kDa, preferably around 20 to 60 kDa and an isoelectric point of 3.5 to 7.5, preferably 4.0 to 7.0 on gels used in the two-dimensional electrophoresis performed under the conditions described above in the subsection titled “Identification of antigens”.

Preferably, a protein that is an antigen in spot 28α has activity equivalent to that of the protein having the amino acid sequence of SEQ ID NO: 422 (35 kDa seed maturation protein isoform X1) or causative of an allergy to soybean.

(29α, 30α, 31α, 32α, 33α) Antigens in spots 29α to 33α

As the result of sequence identification of the antigens in spots 29α to 33α by mass spectroscopy, the following amino acid sequences were detected.

(SEQ ID NO: 436)
EASYDEIK
(SEQ ID NO: 437)
GILGYTEDDVVSTDFIGDSRSSIFDAK
(SEQ ID NO: 438)
VIISAPSK
(SEQ ID NO: 439)
VLPALNGK
(SEQ ID NO: 442)
EASYDEIK
(SEQ ID NO: 443)
GILGYTEDDVVSTDFVGDSRSSIFDAK
(SEQ ID NO: 444)
VIISAPSK
(SEQ ID NO: 445)
VLPALNGK
(SEQ ID NO: 448)
AGISLNDNFVK
(SEQ ID NO: 449)
GILGYTEDDVVSTDFVGDNRSSIFDAK
(SEQ ID NO: 450)
VLPALNGK
(SEQ ID NO: 451)
VVISAPSK

Also, the mass spectroscopic data obtained for spots 29α to 33α on a mass spectrometer was analyzed by comparing the data against the NCBI protein data, and as a result, glyceraldehyde-3-phosphate dehydrogenase isoform X1 (amino acid sequence: SEQ ID NO: 435, encoding nucleotide sequence: SEQ ID NO: 434) was identified as a protein comprising any of SEQ ID NO: 436 to 439, glyceraldehyde-3-phosphate dehydrogenase, cytosolic (amino acid sequence: SEQ ID NO: 441, encoding nucleotide sequence: SEQ ID NO: 440) was identified as a protein comprising any of SEQ ID NO: 442 to 445, and uncharacterized protein LOC100782924 (amino acid sequence: SEQ ID NO: 447, encoding nucleotide sequence: SEQ ID NO: 446) was identified as a protein comprising any of SEQ ID NO: 448 to 451.

Accordingly, the antigen in spot 29α to 33α in the present invention can be any of proteins selected from the group consisting of the proteins as defined below in (29-33αA1-a) to (29-33αA1-e), (29-33αB1), (29-33αA2-a) to (29-33αA2-e), (29-33αB2), (29-33αA3-a) to (29-33αA3-e), and (29-33αB3):
(29-33αA1-a) a protein comprising an amino acid sequence with deletion, substitution, insertion or addition of one or several amino acids in SEQ ID NO: 435;
(29-33αA1-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 435;
(29-33αA1-c) a protein comprising an amino acid sequence encoded by a nucleotide sequence with deletion, substitution, insertion or addition of one or several nucleotides in SEQ ID NO: 434;
(29-33αA1-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 434;
(29-33αA1-e) a protein comprising an amino acid sequence encoded by a nucleic acid that hybridizes under stringent conditions with a nucleic acid having a nucleotide sequence complementary to the nucleotide sequence of SEQ ID NO: 434;
(29-33αB1) a protein comprising at least one amino acid sequence selected from the group consisting of SEQ ID NOs: 436-439, preferably a protein comprising at least 2, 3, or all sequences of the amino acid sequences. As referred to above, the amino acid sequence of any of SEQ ID NOs: 436 to 439 may be an amino acid sequence derived therefrom by deletion, substitution, insertion or addition of one or several amino acids;
(29-33αA2-a) a protein comprising an amino acid sequence with deletion, substitution, insertion or addition of one or several amino acids in SEQ ID NO: 441;
(29-33αA2-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 441;
(29-33αA2-c) a protein comprising an amino acid sequence encoded by a nucleotide sequence with deletion, substitution, insertion or addition of one or several nucleotides in SEQ ID NO: 440;
(29-33αA2-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 440;
(29-33αA2-e) a protein comprising an amino acid sequence encoded by a nucleic acid that hybridizes under stringent conditions with a nucleic acid having a nucleotide sequence complementary to the nucleotide sequence of SEQ ID NO: 440;
(29-33αB2) a protein comprising at least one amino acid sequence selected from the group consisting of SEQ ID NOs: 442-445, preferably a protein comprising at least 2, 3, or all sequences of the amino acid sequences. As referred to above, the amino acid sequence of any of SEQ ID NOs: 442 to 445 may be an amino acid sequence derived therefrom by deletion, substitution, insertion or addition of one or several amino acids.
(29-33αA3-a) a protein comprising an amino acid sequence with deletion, substitution, insertion or addition of one or several amino acids in SEQ ID NO: 447;
(29-33αA3-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 447;
(29-33αA3-c) a protein comprising an amino acid sequence encoded by a nucleotide sequence with deletion, substitution, insertion or addition of one or several nucleotides in SEQ ID NO: 446;
(29-33αA3-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 446;
(29-33αA3-e) a protein comprising an amino acid sequence encoded by a nucleic acid that hybridizes under stringent conditions with a nucleic acid having a nucleotide sequence complementary to the nucleotide sequence of SEQ ID NO: 446;
(29-33αB3) a protein comprising at least one amino acid sequence selected from the group consisting of SEQ ID NOs: 448-451, preferably a protein comprising at least 2, 3, or all sequences of the amino acid sequences. As referred to above, the amino acid sequence of any of SEQ ID NOs: 448 to 451 may be an amino acid sequence derived therefrom by deletion, substitution, insertion or addition of one or several amino acids.

The protein selected from the group consisting of the proteins as defined above in (29-33αA1-a) to (29-33αA1-e), (29-33αB1), (29-33αA2-a) to (29-33αA2-e), (29-33αB2), (29-33αA3-a) to (29-33αA3-e), and (29-33αB3) also include those proteins whose amino acid residues are modified by phosphorylation, sugar chain modification, aminoacylation, ring-opening, deamination or the like.

The protein selected from the group consisting of the proteins as defined above in (29-33αA1-a) to (29-33αA1-e), (29-33αB1), (29-33αA2-a) to (29-33αA2-e), (29-33αB2), (29-33αA3-a) to (29-33αA3-e), and (29-33αB3) can be proteins that are found in a protein spot with a molecular weight of around 10 to 70 kDa, preferably around 20 to 60 kDa, and an isoelectric point of 3.5 to 8.5, preferably 4.0 to 8.0 on gels used in the two-dimensional electrophoresis performed under the conditions described above in the subsection titled “Identification of antigens”.

Preferably, a protein that is an antigen in any of spots 29α to 33α has activity equivalent to that of the protein having the amino acid sequence of SEQ ID NO: 435 (glyceraldehyde-3-phosphate dehydrogenase isoform X1), the protein having the amino acid sequence of SEQ ID NO: 441 (glyceraldehyde-3-phosphate dehydrogenase, cytosolic), or the protein having the amino acid sequence of SEQ ID NO: 447 (uncharacterized protein LOC100782924) or causative of an allergy to soybean.

### (34α) Antigen in Spot 34α

As the result of sequence identification of the antigen in spot 34α by mass spectroscopy, the following amino acid sequences were detected.

(SEQ ID NO: 454)
LFLEEIARDIQK
(SEQ ID NO: 457)
LFLEEIARDIQK
(SEQ ID NO: 458)
TTFDAVIHFAGLK

Also, the mass spectroscopic data obtained for spot 34α on a mass spectrometer was analyzed by comparing the data against the NCBI protein data, and as a result, bifunctional UDP-glucose 4-epimerase and UDP-xylose 4-epimerase 1-like (amino acid sequence: SEQ ID NO: 453, encoding nucleotide sequence: SEQ ID NO: 452) was identified as a protein comprising SEQ ID NO: 454 and uncharacterized protein LOC100803483 (amino acid sequence: SEQ ID NO: 456, encoding nucleotide sequence: SEQ ID NO: 455) was identified as a protein comprising SEQ ID NO: 457 or 458.

Accordingly, the antigen in spot 34α in the present invention can be any of proteins selected from the group consisting of the proteins as defined below in (34αA1-a) to (34αA1-e), (34αB1), (34αA2-a) to (34αA2-e), and (34αB2):
(34αA1-a) a protein comprising an amino acid sequence with deletion, substitution, insertion or addition of one or several amino acids in SEQ ID NO: 453;
(34αA1-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 453;
(34αA1-c) a protein comprising an amino acid sequence encoded by a nucleotide sequence with deletion, substitution, insertion or addition of one or several nucleotides in SEQ ID NO: 452;
(34αA1-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 452;
(34αA1-e) a protein comprising an amino acid sequence encoded by a nucleic acid that hybridizes under stringent conditions with a nucleic acid having a nucleotide sequence complementary to the nucleotide sequence of SEQ ID NO: 452;
(34αB1) a protein comprising an amino acid sequence of SEQ ID NOs: 454. As referred to above, the amino acid sequence of any of SEQ ID NOs: 464 may be an amino acid sequence derived therefrom by deletion, substitution, insertion or addition of one or several amino acids;
(34αA2-a) a protein comprising an amino acid sequence with deletion, substitution, insertion or addition of one or several amino acids in SEQ ID NO: 456;
(34αA2-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 456;
(34αA2-c) a protein comprising an amino acid sequence encoded by a nucleotide sequence with deletion, substitution, insertion or addition of one or several nucleotides in SEQ ID NO: 455;
(34αA2-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 455;
(34αA2-e) a protein comprising an amino acid sequence encoded by a nucleic acid that hybridizes under stringent conditions with a nucleic acid having a nucleotide sequence complementary to the nucleotide sequence of SEQ ID NO: 455;
(34αB2) a protein comprising at least one amino acid sequence selected from the group consisting of SEQ ID NOs: 457 and 458, preferably a protein comprising both the amino acid sequences. As referred to above, the amino acid sequence of any of SEQ ID NOs: 457, 458 may be an amino acid sequence derived therefrom by deletion, substitution, insertion or addition of one or several amino acids.

The protein selected from the group consisting of the proteins as defined above in (34αA1-a) to (34αA1-e), (34αB1), (34αA2-a) to (34αA2-e), and (34αB2) also include those proteins whose amino acid residues are modified by phosphorylation, sugar chain modification, aminoacylation, ring-opening, deamination or the like.

The protein selected from the group consisting of the proteins as defined above in (34αA1-a) to (34αA1-e), (34αB1), (34αA2-a) to (34αA2-e), and (34αB2) can be proteins that are found in a protein spot with a molecular weight of around 10 to 70 kDa, preferably around 20 to 60 kDa, and an isoelectric point of 5.5 to 10.5, preferably 6.0 to 10.0 on gels used in the two-dimensional electrophoresis performed under the conditions described above in the subsection titled “Identification of antigens”.

Preferably, a protein that is an antigen in spot 34α has activity equivalent to that of the protein having the amino acid sequence of SEQ ID NO: 453 (bifunctional UDP-glucose 4-epimerase and UDP-xylose 4-epimerase 1-like) or the protein having the amino acid sequence of SEQ ID NO: 456 (uncharacterized protein LOC100803483) or causative of an allergy to soybean.

### (35α) Antigen in Spot 35α

As the result of sequence identification of the antigen in spot 35α by mass spectroscopy, the following amino acid sequences were detected.

(SEQ ID NO: 461)
ARYDEIVK
(SEQ ID NO: 462)
DGQTREHALLAFTLGVK
(SEQ ID NO: 463)
EVSSYLK
(SEQ ID NO: 464)
FSEILTK
(SEQ ID NO: 465)
STTTGHLIYK
(SEQ ID NO: 466)
THINIVVIGHVDSGK
(SEQ ID NO: 469)
DGQTREHALLAFTLGVK
(SEQ ID NO: 470)
EVSSYLK
(SEQ ID NO: 471)
STTTGHLIYK
(SEQ ID NO: 474)
ARYDEIVK
(SEQ ID NO: 475)
DGQTREHALLSFTLGVK
(SEQ ID NO: 476)
EVSSYLK
(SEQ ID NO: 477)
STTTGHLIYK

Also, the mass spectroscopic data obtained for spot 35α on a mass spectrometer was analyzed by comparing the data against the NCBI protein data, and as a result, elongation factor 1-alpha (amino acid sequence: SEQ ID NO: 460, encoding nucleotide sequence: SEQ ID NO: 459) was identified as a protein comprising any of SEQ ID NOs: 461 to 466, elongation factor 1-alpha (amino acid sequence: SEQ ID NO: 468, encoding nucleotide sequence: SEQ ID NO: 467) was identified as a protein comprising any of SEQ ID NOs: 469 to 471, and elongation factor-1A isoform X1 (amino acid sequence: SEQ ID NO: 473, encoding nucleotide sequence: SEQ ID NO: 472) was identified as a protein comprising any of SEQ ID NOs: 474 to 477.

Accordingly, the antigen in spot 35α in the present invention can be any of proteins selected from the group consisting of the proteins as defined below in (35αA1-a) to (35αA1-e), (35αB1), (35αA2-a) to (35αA2-e), (35αB2), (35αA3-a) to (35αA3-e), and (35αB3):
(35αA1-a) a protein comprising an amino acid sequence with deletion, substitution, insertion or addition of one or several amino acids in SEQ ID NO: 460;
(35αA1-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 460;
(35αA1-c) a protein comprising an amino acid sequence encoded by a nucleotide sequence with deletion, substitution, insertion or addition of one or several nucleotides in SEQ ID NO: 459;
(35αA1-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 459;
(35αA1-e) a protein comprising an amino acid sequence encoded by a nucleic acid that hybridizes under stringent conditions with a nucleic acid having a nucleotide sequence complementary to the nucleotide sequence of SEQ ID NO: 459;
(35αB1) a protein comprising at least one amino acid sequence selected from the group consisting of SEQ ID NOs: 461-466, preferably a protein comprising at least 2, 3, 4, or 5 or all sequences of the amino acid sequences. As referred to above, the amino acid sequence of any of SEQ ID NOs: 461-466 may be an amino acid sequence derived therefrom by deletion, substitution, insertion or addition of one or several amino acids;
(35αA2-a) a protein comprising an amino acid sequence with deletion, substitution, insertion or addition of one or several amino acids in SEQ ID NO: 468;
(35αA2-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 468;
(35αA2-c) a protein comprising an amino acid sequence encoded by a nucleotide sequence with deletion, substitution, insertion or addition of one or several nucleotides in SEQ ID NO: 467;
(35αA2-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 467;
(35αA2-e) a protein comprising an amino acid sequence encoded by a nucleic acid that hybridizes under stringent conditions with a nucleic acid having a nucleotide sequence complementary to the nucleotide sequence of SEQ ID NO: 467;
(35αB2) a protein comprising at least one amino acid sequence selected from the group consisting of SEQ ID NOs: 469-471, preferably a protein comprising at least 2, or all sequences of the amino acid sequences. As referred to above, the amino acid sequence of any of SEQ ID NOs: 469 to 471 may be an amino acid sequence derived therefrom by deletion, substitution, insertion or addition of one or several amino acids.
(35αA3-a) a protein comprising an amino acid sequence with deletion, substitution, insertion or addition of one or several amino acids in SEQ ID NO: 473;
(35αA3-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 473;
(35αA3-c) a protein comprising an amino acid sequence encoded by a nucleotide sequence with deletion, substitution, insertion or addition of one or several nucleotides in SEQ ID NO: 472;
(35αA3-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 472;
(35αA3-e) a protein comprising an amino acid sequence encoded by a nucleic acid that hybridizes under stringent conditions with a nucleic acid having a nucleotide sequence complementary to the nucleotide sequence of SEQ ID NO: 472;
(35αB3) a protein comprising at least one amino acid sequence selected from the group consisting of SEQ ID NOs: 474-477, preferably a protein comprising at least 2, 3, or all sequences of the amino acid sequences. As referred to above, the amino acid sequence of any of SEQ ID NOs: 474 to 477 may be an amino acid sequence derived therefrom by deletion, substitution, insertion or addition of one or several amino acids.

The protein selected from the group consisting of the proteins as defined above in (35αA1-a) to (35αA1-e), (35αB1), (35αA2-a) to (35αA2-e), (35αB2), (35αA3-a) to (35αA3-e), and (35αB3) also include those proteins whose amino acid residues are modified by phosphorylation, sugar chain modification, aminoacylation, ring-opening, deamination or the like.

The protein selected from the group consisting of the proteins as defined above in (35αA1-a) to (35αA1-e), (35αB1), (35αA2-a) to (35αA2-e), (35αB2), (35αA3-a) to (35αA3-e), and (35αB3) can be proteins that are found in a protein spot with a molecular weight of around 20 to 90 kDa, preferably around 30 to 80 kDa, and an isoelectric point of 5.5 to 10.5, preferably 6.0 to 10.0 on gels used in the two-dimensional electrophoresis performed under the conditions described above in the subsection titled “Identification of antigens”.

Preferably, a protein that is an antigen in spot 35α has activity equivalent to that of the protein having the amino acid sequence of SEQ ID NO: 460 or 468 (elongation factor 1-alpha) or the protein having the amino acid sequence of SEQ ID NO: 473 (elongation factor-1A isoform X1) or causative of an allergy to soybean.

### (36α, 37α) Antigens in Spots 36α, 37α

As the result of sequence identification of the antigens in spots 36α and 37α by mass spectroscopy, the following amino acid sequences were detected.

(SEQ ID NO: 480)
EGSSIINTTSVNAYK
(SEQ ID NO: 481)
FPPQQQQTQPGK
(SEQ ID NO: 482)
GAIVAYTRGLALQLVSK
(SEQ ID NO: 483)
LLDYTSTK
(SEQ ID NO: 486)
EGATVAFTYVK
(SEQ ID NO: 487)
QVIDLVVK
(SEQ ID NO: 490)
EGSSIINTTSVNAYK
(SEQ ID NO: 491)
FPPQQQQTQPGK
(SEQ ID NO: 492)
GAIVAYTRGLALQLVSK
(SEQ ID NO: 493)
LLDYTSTK

Also, the mass spectroscopic data obtained for spots 36α and 37α on a mass spectrometer was analyzed by comparing the data against the NCBI protein data, and as a result, seed maturation protein PM34 (amino acid sequence: SEQ ID NO: 479, encoding nucleotide sequence: SEQ ID NO: 478) was identified as a protein comprising any of SEQ ID NOs: 480 to 483, uncharacterized protein LOC100809384 (amino acid sequence: SEQ ID NO: 485, encoding nucleotide sequence: SEQ ID NO: 484) was identified as a protein comprising SEQ ID NO: 486 or 487, and hypothetical protein GLYMA_10G155300 (amino acid sequence: SEQ ID NO: 489, encoding nucleotide sequence: SEQ ID NO: 488) was identified as a protein comprising any of SEQ ID NOs: 490 to 493.

Accordingly, the antigen in spot 36α, 37α in the present invention can be any of proteins selected from the group consisting of the proteins as defined below in (36, 37αA1-a) to (36, 37αA1-e), (36, 37αB1), (36, 37αA2-a) to (36, 37αA2-e), (36, 37αB2), (36, 37αA3-a) to (36, 37αA3-e), and (36, 37αB3):
(36, 37αA1-a) a protein comprising an amino acid sequence with deletion, substitution, insertion or addition of one or several amino acids in SEQ ID NO: 479;
(36, 37αA1-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 479;
(36, 37αA1-c) a protein comprising an amino acid sequence encoded by a nucleotide sequence with deletion, substitution, insertion or addition of one or several nucleotides in SEQ ID NO: 478;
(36, 37αA1-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 478;
(36, 37αA1-e) a protein comprising an amino acid sequence encoded by a nucleic acid that hybridizes under stringent conditions with a nucleic acid having a nucleotide sequence complementary to the nucleotide sequence of SEQ ID NO: 478;
(36, 37αB1) a protein comprising at least one amino acid sequence selected from the group consisting of SEQ ID NOs: 480-483, preferably a protein comprising at least 2, 3, or all sequences of the amino acid sequences. As referred to above, the amino acid sequence of any of SEQ ID NOs: 480 to 483 may be an amino acid sequence derived therefrom by deletion, substitution, insertion or addition of one or several amino acids;
(36, 37αA2-a) a protein comprising an amino acid sequence with deletion, substitution, insertion or addition of one or several amino acids in SEQ ID NO: 485;
(36, 37αA2-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 485;
(36, 37αA2-c) a protein comprising an amino acid sequence encoded by a nucleotide sequence with deletion, substitution, insertion or addition of one or several nucleotides in SEQ ID NO: 484;
(36, 37αA2-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 484;
(36, 37αA2-e) a protein comprising an amino acid sequence encoded by a nucleic acid that hybridizes under stringent conditions with a nucleic acid having a nucleotide sequence complementary to the nucleotide sequence of SEQ ID NO: 484;
(36, 37αB2) a protein comprising at least one amino acid sequence selected from the group consisting of SEQ ID NOs: 486 and 487, preferably a protein comprising both the amino acid sequences. As referred to above, the amino acid sequence of any of SEQ ID NOs: 486, 487 may be an amino acid sequence derived therefrom by deletion, substitution, insertion or addition of one or several amino acids.
(36, 37αA3-a) a protein comprising an amino acid sequence with deletion, substitution, insertion or addition of one or several amino acids in SEQ ID NO: 489;
(36, 37αA3-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 489;
(36, 37αA3-c) a protein comprising an amino acid sequence encoded by a nucleotide sequence with deletion, substitution, insertion or addition of one or several nucleotides in SEQ ID NO: 488;
(36, 37αA3-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 488;
(36, 37αA3-e) a protein comprising an amino acid sequence encoded by a nucleic acid that hybridizes under stringent conditions with a nucleic acid having a nucleotide sequence complementary to the nucleotide sequence of SEQ ID NO: 488;
(36, 37αB3) a protein comprising at least one amino acid sequence selected from the group consisting of SEQ ID NOs: 490-493, preferably a protein comprising at least 2, 3, or all sequences of the amino acid sequences. As referred to above, the amino acid sequence of any of SEQ ID NOs: 490 to 493 may be an amino acid sequence derived therefrom by deletion, substitution, insertion or addition of one or several amino acids.

The protein selected from the group consisting of the proteins as defined above in (36, 37αA1-a) to (36, 37αA1-e), (36, 37αB1), (36, 37αA2-a) to (36, 37αA2-e), (36, 37αB2), (36, 37αA3-a) to (36, 37αA3-e), and (36, 37αB3) also include those proteins whose amino acid residues are modified by phosphorylation, sugar chain modification, aminoacylation, ring-opening, deamination or the like.

The protein selected from the group consisting of the proteins as defined above in (36, 37αA1-a) to (36, 37αA1-e), (36, 37αB1), (36, 37αA2-a) to (36, 37αA2-e), (36, 37αB2), (36, 37αA3-a) to (36, 37αA3-e), and (36, 37αB3) can be proteins that are found in a protein spot with a molecular weight of around 10 to 70 kDa, preferably around 20 to 60 kDa, and an isoelectric point of 3.5 to 8.5, preferably 4.0 to 8.0 on gels used in the two-dimensional electrophoresis performed under the conditions described above in the subsection titled “Identification of antigens”.

Preferably, a protein that is an antigen in spot 36α or 37α has activity equivalent to that of the protein having the amino acid sequence of SEQ ID NO: 479 (seed maturation protein PM34), the protein having the amino acid sequence of SEQ ID NO: 485 (uncharacterized protein LOC100809384), or the protein having the amino acid sequence of SEQ ID NO: 489 (hypothetical protein GLYMA_10G155300) or causative of an allergy to soybean.

### (38α) Antigen in Spot 38α

As the result of sequence identification of the antigen in spot 38α by mass spectroscopy, the following amino acid sequences were detected.

(SEQ ID NO: 496)
AAGSTTAQYVGEK
(SEQ ID NO: 497)
ALATGWSAAHYSTEITVEGTK
(SEQ ID NO: 498)
AQVAGSTTVQYAGEK
(SEQ ID NO: 499)
DTIGRVDNITTPLAK
(SEQ ID NO: 500)
DTIGSVDHITTPLAEK
(SEQ ID NO: 501)
ERELASDRARSGNIVK
(SEQ ID NO: 502)
ERYERANQATSEAAK
(SEQ ID NO: 503)
GGGEEQGRRREEIGEENK
(SEQ ID NO: 504)
GLAAAAAGENAK
(SEQ ID NO: 505)
GRVVIESEK
(SEQ ID NO: 506)
GVAEYAGQK
(SEQ ID NO: 507)
LGSAAHTLVK
(SEQ ID NO: 508)
QSSLEEIFK
(SEQ ID NO: 509)
QSSLEEISK
(SEQ ID NO: 510)
REQKPLGSIIGESFQGVQEK
(SEQ ID NO: 511)
TGNETQDIGQK
(SEQ ID NO: 512)
VIVAGEGEK
(SEQ ID NO: 513)
VQQHQGEGGGGVLNAIGETIAEIAETTK

Also, the mass spectroscopic data obtained for spot 38α on a mass spectrometer was analyzed by comparing the data against the NCBI protein data, and as a result, seed biotin-containing protein SBP65-like isoform X1 (amino acid sequence: SEQ ID NO: 495, encoding nucleotide sequence: SEQ ID NO: 494) was identified as a protein comprising any of SEQ ID NOs: 496 to 513.

Accordingly, the antigens in spot 38α in the present invention can be any protein selected from the group consisting of the proteins as defined below in (38αA-a) to (38αA-e) and (38αB):
(38αA-a) a protein comprising an amino acid sequence with deletion, substitution, insertion or addition of one or several amino acids in SEQ ID NO: 495;
(38αA-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 495;
(38αA-c) a protein comprising an amino acid sequence encoded by a nucleotide sequence with deletion, substitution, insertion or addition of one or several nucleotides in SEQ ID NO: 494;
(38αA-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 494;
(38αA-e) a protein comprising an amino acid sequence encoded by a nucleic acid that hybridizes under stringent conditions with a nucleic acid having a nucleotide sequence complementary to the nucleotide sequence of SEQ ID NO: 494;
(38αB) a protein comprising at least one amino acid sequence selected from the group consisting of SEQ ID NOs: 496-513, preferably a protein comprising at least 2, 3, 4, 5, 6, 7, 8, 9, 10, 11, 12, 13, 14, 15, 16 or 17 or all sequences of the amino acid sequences. As referred to above, the amino acid sequence of any of SEQ ID NOs: 496-513 may be an amino acid sequence derived therefrom by deletion, substitution, insertion or addition of one or several amino acids.

The proteins selected from the group consisting of (38αA-a) to (38αA-e) and (38αB) as defined above also include those proteins whose amino acid residues are modified by phosphorylation, sugar chain modification, aminoacylation, ring-opening, deamination or the like.

The proteins selected from the group consisting of (38αA-a) to (38αA-e) and (38αB) as defined above can be proteins that are found in a protein spot with a molecular weight of around 70 to 170 kDa, preferably around 80 to 160 kDa and an isoelectric point of 5.5 to 10.5, preferably 6.0 to 10.0 on gels used in the two-dimensional electrophoresis performed under the conditions described above in the subsection titled “Identification of antigens”.

Preferably, a protein that is an antigen in spot 38α has activity equivalent to that of the protein having the amino acid sequence of SEQ ID NO: 495 (seed biotin-containing protein SBP65-like isoform X1) or causative of an allergy to soybean.

### (39α) Antigen in Spot 39α

As the result of sequence identification of the antigen in spot 39α by mass spectroscopy, the following amino acid sequences were detected.

(SEQ ID NO: 516)
ETLQEIIGSGAK

Also, the mass spectroscopic data obtained for spot 39α on a mass spectrometer was analyzed by comparing the data against the NCBI protein data, and as a result, ATP synthase subunit 0, mitochondrial-like (amino acid sequence: SEQ ID NO: 515, encoding nucleotide sequence: SEQ ID NO: 514) was identified as a protein comprising SEQ ID NO: 516.

Accordingly, the antigens in spot 39α in the present invention can be any protein selected from the group consisting of the proteins as defined below in (39αA-a) to (39αA-e) and (39αB):
(39αA-a) a protein comprising an amino acid sequence with deletion, substitution, insertion or addition of one or several amino acids in SEQ ID NO: 515;
(39αA-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 515;
(39αA-c) a protein comprising an amino acid sequence encoded by a nucleotide sequence with deletion, substitution, insertion or addition of one or several nucleotides in SEQ ID NO: 514;
(39αA-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 514;
(39αA-e) a protein comprising an amino acid sequence encoded by a nucleic acid that hybridizes under stringent conditions with a nucleic acid having a nucleotide sequence complementary to the nucleotide sequence of SEQ ID NO: 514;
(39αB) a protein comprising an amino acid sequence of SEQ ID NO: 516. As referred to above, the amino acid sequence(s) of SEQ ID NO: 516 may be an amino acid sequence(s) derived therefrom by deletion, substitution, insertion or addition of one or several amino acids.

The proteins selected from the group consisting of (39αA-a) to (39αA-e) and (39αB) as defined above also include those proteins whose amino acid residues are modified by phosphorylation, sugar chain modification, aminoacylation, ring-opening, deamination or the like.

The proteins selected from the group consisting of (39αA-a) to (39αA-e) and (39αB) as defined above can be proteins that are found in a protein spot with a molecular weight of around 5 to 60 kDa, preferably around 10 to 50 kDa and an isoelectric point of 5.5 to 10.5, preferably 6.0 to 10.0 on gels used in the two-dimensional electrophoresis performed under the conditions described above in the subsection titled “Identification of antigens”.

Preferably, a protein that is an antigen in spot 39α has activity equivalent to that of the protein having the amino acid sequence of SEQ ID NO: 515 (ATP synthase subunit 0, mitochondrial-like) or causative of an allergy to soybean.

### (40α, 56α) Antigens in Spots 40α, 56α

As the result of sequence identification of the antigens in spots 40α and 56α by mass spectroscopy, the following amino acid sequences were detected.

(SEQ ID NO: 519)
EVGQDIQSK
(SEQ ID NO: 520)
RTTVTATTATA

Also, the mass spectroscopic data obtained for spots 40α and 56α on a mass spectrometer was analyzed by comparing the data against the NCBI protein data, and as a result, P24 oleosin isoform A (amino acid sequence: SEQ ID NO: 518, encoding nucleotide sequence: SEQ ID NO: 517) was identified as a protein comprising SEQ ID NO: 519 or 520.

Accordingly, the antigens in spot 40α, 56α in the present invention can be any protein selected from the group consisting of the proteins as defined below in (40, 56αA-a) to (40, 56αA-e) and (40, 56αB):
(40, 56αA-a) a protein comprising an amino acid sequence with deletion, substitution, insertion or addition of one or several amino acids in SEQ ID NO: 518;
(40, 56αA-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 518;
(40, 56αA-c) a protein comprising an amino acid sequence encoded by a nucleotide sequence with deletion, substitution, insertion or addition of one or several nucleotides in SEQ ID NO: 517;
(40, 56αA-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 517;
(40, 56αA-e) a protein comprising an amino acid sequence encoded by a nucleic acid that hybridizes under stringent conditions with a nucleic acid having a nucleotide sequence complementary to the nucleotide sequence of SEQ ID NO: 517;
(40, 56αB) a protein comprising at least one amino acid sequence selected from the group consisting of SEQ ID NOs: 519 and 520, preferably a protein comprising both the amino acid sequences. As referred to above, the amino acid sequence of any of SEQ ID NOs: 519 and 520 may be an amino acid sequence derived therefrom by deletion, substitution, insertion or addition of one or several amino acids.

The proteins selected from the group consisting of (40, 56αA-a) to (40, 56αA-e) and (40, 56αB) as defined above also include those proteins whose amino acid residues are modified by phosphorylation, sugar chain modification, aminoacylation, ring-opening, deamination or the like.

The proteins selected from the group consisting of (40, 56αA-a) to (40, 56αA-e) and (40, 56αB) as defined above can be proteins that are found in a protein spot with a molecular weight of around 5 to 60 kDa, preferably around 10 to 50 kDa and an isoelectric point of 5.5 to 10.5, preferably 6.0 to 10.0 on gels used in the two-dimensional electrophoresis performed under the conditions described above in the subsection titled “Identification of antigens”.

Preferably, a protein that is an antigen in spot 40α or 56α has activity equivalent to that of the protein having the amino acid sequence of SEQ ID NO: 518 (P24 oleosin isoform A) or causative of an allergy to soybean.

### (41α) Antigen in Spot 41α

As the result of sequence identification of the antigen in spot 41α by mass spectroscopy, the following amino acid sequences were detected.

(SEQ ID NO: 523)
NAYGGSMK

Also, the mass spectroscopic data obtained for spot 41α on a mass spectrometer was analyzed by comparing the data against the NCBI protein data, and as a result, uncharacterized protein At3g49720-like isoform X2 (amino acid sequence: SEQ ID NO: 522, encoding nucleotide sequence: SEQ ID NO: 521) was identified as a protein comprising SEQ ID NO: 523.

Accordingly, the antigens in spot 41α in the present invention can be any protein selected from the group consisting of the proteins as defined below in (41αA-a) to (41αA-e) and (41αB):
(41αA-a) a protein comprising an amino acid sequence with deletion, substitution, insertion or addition of one or several amino acids in SEQ ID NO: 522;
(41αA-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 522;
(41αA-c) a protein comprising an amino acid sequence encoded by a nucleotide sequence with deletion, substitution, insertion or addition of one or several nucleotides in SEQ ID NO: 521;
(41αA-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 521;
(41αA-e) a protein comprising an amino acid sequence encoded by a nucleic acid that hybridizes under stringent conditions with a nucleic acid having a nucleotide sequence complementary to the nucleotide sequence of SEQ ID NO: 521;
(41αB) a protein comprising an amino acid sequence of SEQ ID NO: 523. As referred to above, the amino acid sequence of SEQ ID NO: 523 may be an amino acid sequence derived therefrom by deletion, substitution, insertion or addition of one or several amino acids.

The proteins selected from the group consisting of (41αA-a) to (41αA-e) and (41αB) as defined above also include those proteins whose amino acid residues are modified by phosphorylation, sugar chain modification, aminoacylation, ring-opening, deamination or the like.

The proteins selected from the group consisting of (41αA-a) to (41αA-e) and (41αB) as defined above can be proteins that are found in a protein spot with a molecular weight of around 5 to 60 kDa, preferably around 10 to 50 kDa and an isoelectric point of 5.5 to 10.5, preferably 6.0 to 10.0 on gels used in the two-dimensional electrophoresis performed under the conditions described above in the subsection titled “Identification of antigens”.

Preferably, a protein that is an antigen in spot 41α has activity equivalent to that of the protein having the amino acid sequence of SEQ ID NO: 515 (ATP synthase subunit 0, mitochondrial-like) or causative of an allergy to soybean.

### (42α, 43α) Antigen in Spots 42α, 43α

As the result of sequence identification of the antigens in spots 42α and 43α by mass spectroscopy, the following amino acid sequences were detected.

(SEQ ID NO: 526)
ALEQLQDAIAK
(SEQ ID NO: 527)
HHQTYITNYNK
(SEQ ID NO: 528)
NLAPVREGGGEPPK
(SEQ ID NO: 529)
NVRPDYLK
(SEQ ID NO: 530)
RLVVETTANQDPLVTK
(SEQ ID NO: 531)
YASEVYEK
(SEQ ID NO: 534)
ALEQLQDAVAK
(SEQ ID NO: 535)
HHQTYITNFNK
(SEQ ID NO: 536)
NLAPVREGGGEPPK
(SEQ ID NO: 537)
NVRPDYLK
(SEQ ID NO: 538)
RLVVETTANQDPLVTK
(SEQ ID NO: 539)
YASEVYEK

Also, the mass spectroscopic data obtained for spots 42α and 43α on a mass spectrometer was analyzed by comparing the data against the NCBI protein data, and as a result, superoxide dismutase [Mn], mitochondrial (amino acid sequence: SEQ ID NO: 525, encoding nucleotide sequence: SEQ ID NO: 524) was identified as a protein comprising any of SEQ ID NOs: 526 to 531 and hypothetical protein GLYMA_04G221300 (amino acid sequence: SEQ ID NO: 533, encoding nucleotide sequence: SEQ ID NO: 532) was identified as a protein comprising any of SEQ ID NOs: 534 to 539.

Accordingly, the antigen in spot 42α, 43α in the present invention can be any of proteins selected from the group consisting of the proteins as defined below in (42, 43αA1-a) to (42, 43αA1-e), (42, 43αB1), (42, 43αA2-a) to (42, 43αA2-e), and (42, 43αB2):
(42, 43αA1-a) a protein comprising an amino acid sequence with deletion, substitution, insertion or addition of one or several amino acids in SEQ ID NO: 525;
(42, 43αA1-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 525;
(42, 43αA1-c) a protein comprising an amino acid sequence encoded by a nucleotide sequence with deletion, substitution, insertion or addition of one or several nucleotides in SEQ ID NO: 524;
(42, 43αA1-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 524;
(42, 43αA1-e) a protein comprising an amino acid sequence encoded by a nucleic acid that hybridizes under stringent conditions with a nucleic acid having a nucleotide sequence complementary to the nucleotide sequence of SEQ ID NO: 524;
(42, 43αB1) a protein comprising at least one amino acid sequence selected from the group consisting of SEQ ID NOs: 526-531, preferably a protein comprising at least 2, 3, 4, or 5 or all sequences of the amino acid sequences. As referred to above, the amino acid sequence of any of SEQ ID NOs: 526 to 531 may be an amino acid sequence derived therefrom by deletion, substitution, insertion or addition of one or several amino acids;
(42, 43αA2-a) a protein comprising an amino acid sequence with deletion, substitution, insertion or addition of one or several amino acids in SEQ ID NO: 533;
(42, 43αA2-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 533;
(42, 43αA2-c) a protein comprising an amino acid sequence encoded by a nucleotide sequence with deletion, substitution, insertion or addition of one or several nucleotides in SEQ ID NO: 532;
(42, 43αA2-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 532;
(42, 43αA2-e) a protein comprising an amino acid sequence encoded by a nucleic acid that hybridizes under stringent conditions with a nucleic acid having a nucleotide sequence complementary to the nucleotide sequence of SEQ ID NO: 532;
(42, 43αB2) a protein comprising at least one amino acid sequence selected from the group consisting of SEQ ID NOs: 534-539, preferably a protein comprising at least 2, 3, 4, or 5 or all sequences of the amino acid sequences. As referred to above, the amino acid sequence of any of SEQ ID NOs: 534 to 539 may be an amino acid sequence derived therefrom by deletion, substitution, insertion or addition of one or several amino acids.

The protein selected from the group consisting of the proteins as defined above in (42, 43αA1-a) to (42, 43αA1-e), (42, 43αB1), (42, 43αA2-a) to (42, 43αA2-e), and (42, 43αB2) also include those proteins whose amino acid residues are modified by phosphorylation, sugar chain modification, aminoacylation, ring-opening, deamination or the like.

The protein selected from the group consisting of the proteins as defined above in (42, 43αA1-a) to (42, 43αA1-e), (42, 43αB1), (42, 43αA2-a) to (42, 43αA2-e), and (42, 43αB2) can be proteins that are found in a protein spot with a molecular weight of around 5 to 60 kDa, preferably around 10 to 50 kDa, and an isoelectric point of 5.5 to 10.5, preferably 6.0 to 10.0 on gels used in the two-dimensional electrophoresis performed under the conditions described above in the subsection titled “Identification of antigens”.

Preferably, a protein that is an antigen in spot 42α or 43α has activity equivalent to that of the protein having the amino acid sequence of SEQ ID NO: 525 (superoxide dismutase [Mn], mitochondrial) or the protein having the amino acid sequence of SEQ ID NO: 533 (hypothetical protein GLYMA_04G221300) or causative of an allergy to soybean.

### (45α) Antigen in Spot 45α

As the result of sequence identification of the antigen in spot 45α by mass spectroscopy, the following amino acid sequences were detected.

(SEQ ID NO: 542)
HVPGFIERAEELK
(SEQ ID NO: 543)
RFALLVEDLK
(SEQ ID NO: 544)
VANVESGGEFTISSAEEIIK
(SEQ ID NO: 545)
YTNALGLELDLTDK
(SEQ ID NO: 548)
HVPGFIERAEELK
(SEQ ID NO: 549)
RFALLVEDLK
(SEQ ID NO: 550)
VANVESGGEFTISSAEEIIK
(SEQ ID NO: 551)
YTNALGLELDLTDK

Also, the mass spectroscopic data obtained for spot 45α on a mass spectrometer was analyzed by comparing the data against the NCBI protein data, and as a result, uncharacterized protein LOC100499771 (amino acid sequence: SEQ ID NO: 541, encoding nucleotide sequence: SEQ ID NO: 540) was identified as a protein comprising any of SEQ ID NOs: 542 to 545 and hypothetical protein GLYMA_09G192800 (amino acid sequence: SEQ ID NO: 547, encoding nucleotide sequence: SEQ ID NO: 546) was identified as a protein comprising any of SEQ ID NOs: 548 to 551.

Accordingly, the antigen in spot 45α in the present invention can be any of proteins selected from the group consisting of the proteins as defined below in (45αA1-a) to (45αA1-e), (45αB1), (45αA2-a) to (45αA2e), and (45αB2):
(45αA1-a) a protein comprising an amino acid sequence with deletion, substitution, insertion or addition of one or several amino acids in SEQ ID NO: 541;
(45αA1-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 541;
(45αA1-c) a protein comprising an amino acid sequence encoded by a nucleotide sequence with deletion, substitution, insertion or addition of one or several nucleotides in SEQ ID NO: 540;
(45αA1-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 540;
(45αA1-e) a protein comprising an amino acid sequence encoded by a nucleic acid that hybridizes under stringent conditions with a nucleic acid having a nucleotide sequence complementary to the nucleotide sequence of SEQ ID NO: 540;
(45αB1) a protein comprising at least one amino acid sequence selected from the group consisting of SEQ ID NOs: 542-545, preferably a protein comprising at least 2, 3, or all sequences of the amino acid sequences. As referred to above, the amino acid sequence of any of SEQ ID NOs: 542 to 545 may be an amino acid sequence derived therefrom by deletion, substitution, insertion or addition of one or several amino acids;
(45αA2-a) a protein comprising an amino acid sequence with deletion, substitution, insertion or addition of one or several amino acids in SEQ ID NO: 547;
(45αA2-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 547;
(45αA2-c) a protein comprising an amino acid sequence encoded by a nucleotide sequence with deletion, substitution, insertion or addition of one or several nucleotides in SEQ ID NO: 546;
(45αA2-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 546;
(45αA2-e) a protein comprising an amino acid sequence encoded by a nucleic acid that hybridizes under stringent conditions with a nucleic acid having a nucleotide sequence complementary to the nucleotide sequence of SEQ ID NO: 546;
(45αB2) a protein comprising at least one amino acid sequence selected from the group consisting of SEQ ID NOs: 548-551, preferably a protein comprising at least 2, 3, or all sequences of the amino acid sequences. As referred to above, the amino acid sequence of any of SEQ ID NOs: 548 to 551 may be an amino acid sequence derived therefrom by deletion, substitution, insertion or addition of one or several amino acids.

The protein selected from the group consisting of the proteins as defined above in (45αA1-a) to (45αA1-e), (45αB1), (45αA2-a) to (45αA2-e), and (45αB2) also include those proteins whose amino acid residues are modified by phosphorylation, sugar chain modification, aminoacylation, ring-opening, deamination or the like.

The protein selected from the group consisting of the proteins as defined above in (45αA1-a) to (45αA1-e), (45αB1), (45αA2-a) to (45αA2-e), and (45αB2) can be proteins that are found in a protein spot with a molecular weight of around 2 to 50 kDa, preferably around 5 to 40 kDa, and an isoelectric point of 3.5 to 7.5, preferably 4.0 to 7.0 on gels used in the two-dimensional electrophoresis performed under the conditions described above in the subsection titled “Identification of antigens”.

Preferably, a protein that is an antigen in spot 45α has activity equivalent to that of the protein having the amino acid sequence of SEQ ID NO: 541 (uncharacterized protein LOC100499771) or the protein having the amino acid sequence of SEQ ID NO: 547 (hypothetical protein GLYMA_09G192800) or causative of an allergy to soybean.

### (46α) Antigen in Spot 46α

As the result of sequence identification of the antigen in spot 46α by mass spectroscopy, the following amino acid sequences were detected.

(SEQ ID NO: 554)
DNVEYLAK

Also, the mass spectroscopic data obtained for spot 46α on a mass spectrometer was analyzed by comparing the data against the NCBI protein data, and as a result, seed maturation protein PM22 (amino acid sequence: SEQ ID NO: 553, encoding nucleotide sequence: SEQ ID NO: 552) was identified as a protein comprising SEQ ID NO: 554.

Accordingly, the antigens in spot 46α in the present invention can be any protein selected from the group consisting of the proteins as defined below in (46αA-a) to (46αA-e) and (46αB):
(46αA-a) a protein comprising an amino acid sequence with deletion, substitution, insertion or addition of one or several amino acids in SEQ ID NO: 553;
(46αA-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 553;
(46αA-c) a protein comprising an amino acid sequence encoded by a nucleotide sequence with deletion, substitution, insertion or addition of one or several nucleotides in SEQ ID NO: 552;
(46αA-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 552;
(46αA-e) a protein comprising an amino acid sequence encoded by a nucleic acid that hybridizes under stringent conditions with a nucleic acid having a nucleotide sequence complementary to the nucleotide sequence of SEQ ID NO: 552;
(46αB) a protein comprising an amino acid sequence of SEQ ID NO: 554. As referred to above, the amino acid sequence(s) of SEQ ID NO: 554 may be an amino acid sequence(s) derived therefrom by deletion, substitution, insertion or addition of one or several amino acids.

The proteins selected from the group consisting of (46αA-a) to (46αA-e) and (46αB) as defined above also include those proteins whose amino acid residues are modified by phosphorylation, sugar chain modification, aminoacylation, ring-opening, deamination or the like.

The proteins selected from the group consisting of (46αA-a) to (46αA-e) and (46αB) as defined above can be proteins that are found in a protein spot with a molecular weight of around 2 to 50 kDa, preferably around 5 to 40 kDa and an isoelectric point of 3.5 to 7.5, preferably 4.0 to 7.0 on gels used in the two-dimensional electrophoresis performed under the conditions described above in the subsection titled “Identification of antigens”.

Preferably, a protein that is an antigen in spot 46α has activity equivalent to that of the protein having the amino acid sequence of SEQ ID NO: 553 (seed maturation protein PM22) or causative of an allergy to soybean.

### (47α) Antigen in Spot 47α

As the result of sequence identification of the antigen in spot 47α by mass spectroscopy, the following amino acid sequences were detected.

(SEQ ID NO: 557)
LPTDESLLTQIK
(SEQ ID NO: 558)
NGYIVIK
(SEQ ID NO: 561)
NGYIVIK
(SEQ ID NO: 562)
TYPQQAGTIRK
(SEQ ID NO: 563)
VVEVSTSK

Also, the mass spectroscopic data obtained for spot 47α on a mass spectrometer was analyzed by comparing the data against the NCBI protein data, and as a result, eukaryotic translation initiation factor 5A-2 (amino acid sequence: SEQ ID NO: 556, encoding nucleotide sequence: SEQ ID NO: 555) was identified as a protein comprising SEQ ID NO: 557 or 558 and uncharacterized protein LOC100305510 (amino acid sequence: SEQ ID NO: 560, encoding nucleotide sequence: SEQ ID NO: 559) was identified as a protein comprising any of SEQ ID NOs: 561 to 563.

Accordingly, the antigen in spot 47α in the present invention can be any of proteins selected from the group consisting of the proteins as defined below in (47αA1-a) to (47αA1-e), (47αB1), (47αA2-a) to (47αA2-e), and (47αB2):
(47αA1-a) a protein comprising an amino acid sequence with deletion, substitution, insertion or addition of one or several amino acids in SEQ ID NO: 556;
(47αA1-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 556;
(47αA1-c) a protein comprising an amino acid sequence encoded by a nucleotide sequence with deletion, substitution, insertion or addition of one or several nucleotides in SEQ ID NO: 555;
(47αA1-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 555;
(47αA1-e) a protein comprising an amino acid sequence encoded by a nucleic acid that hybridizes under stringent conditions with a nucleic acid having a nucleotide sequence complementary to the nucleotide sequence of SEQ ID NO: 555;
(47αB1) a protein comprising at least one amino acid sequence selected from the group consisting of SEQ ID NOs: 557 and 558, preferably a protein comprising both the amino acid sequences. As referred to above, the amino acid sequence of any of SEQ ID NOs: 557, 558 may be an amino acid sequence derived therefrom by deletion, substitution, insertion or addition of one or several amino acids;
(47αA2-a) a protein comprising an amino acid sequence with deletion, substitution, insertion or addition of one or several amino acids in SEQ ID NO: 560;
(47αA2-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 560;
(47αA2-c) a protein comprising an amino acid sequence encoded by a nucleotide sequence with deletion, substitution, insertion or addition of one or several nucleotides in SEQ ID NO: 559;
(47αA2-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 559;
(47αA2-e) a protein comprising an amino acid sequence encoded by a nucleic acid that hybridizes under stringent conditions with a nucleic acid having a nucleotide sequence complementary to the nucleotide sequence of SEQ ID NO: 559;
(47αB2) a protein comprising at least one amino acid sequence selected from the group consisting of SEQ ID NOs: 561-563, preferably a protein comprising at least 2, or all sequences of the amino acid sequences. As referred to above, the amino acid sequence of any of SEQ ID NOs: 561-563 may be an amino acid sequence derived therefrom by deletion, substitution, insertion or addition of one or several amino acids.

The protein selected from the group consisting of the proteins as defined above in (47αA1-a) to (47αA1-e), (47αB1), (47αA2-a) to (47αA2-e), and (47αB2) also include those proteins whose amino acid residues are modified by phosphorylation, sugar chain modification, aminoacylation, ring-opening, deamination or the like.

The protein selected from the group consisting of the proteins as defined above in (47αA1-a) to (47αA1-e), (47αB1), (47αA2-a) to (47αA2-e), and (47αB2) can be proteins that are found in a protein spot with a molecular weight of around 2 to 50 kDa, preferably around 5 to 40 kDa, and an isoelectric point of 3.5 to 7.5, preferably 4.0 to 7.0 on gels used in the two-dimensional electrophoresis performed under the conditions described above in the subsection titled “Identification of antigens”.

Preferably, a protein that is an antigen in spot 47α has activity equivalent to that of the protein having the amino acid sequence of SEQ ID NO: 556 (eukaryotic translation initiation factor 5A-2) or the protein having the amino acid sequence of SEQ ID NO: 560 (uncharacterized protein LOC100305510) or causative of an allergy to soybean.

### (48α) Antigen in Spot 48α

As the result of sequence identification of the antigen in spot 48α by mass spectroscopy, the following amino acid sequences were detected.

(SEQ ID NO: 566)
AEFPIFDK
(SEQ ID NO: 567)
DARGNNVNLADYK
(SEQ ID NO: 568)
DGNVVDRYAPTTSPLSIEK
(SEQ ID NO: 569)
GGLFGDSIK
(SEQ ID NO: 570)
VDVNGDNAAPLYK

Also, the mass spectroscopic data obtained for spot 48α on a mass spectrometer was analyzed by comparing the data against the NCBI protein data, and as a result, uncharacterized protein LOC100306570 (amino acid sequence: SEQ ID NO: 565, encoding nucleotide sequence: SEQ ID NO: 564) was identified as a protein comprising any of SEQ ID NOs: 566 to 570.

Accordingly, the antigens in spot 48α in the present invention can be any protein selected from the group consisting of the proteins as defined below in (48αA-a) to (48αA-e) and (48αB):
(48αA-a) a protein comprising an amino acid sequence with deletion, substitution, insertion or addition of one or several amino acids in SEQ ID NO: 565;
(48αA-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 565;
(48αA-c) a protein comprising an amino acid sequence encoded by a nucleotide sequence with deletion, substitution, insertion or addition of one or several nucleotides in SEQ ID NO: 564;
(48αA-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 564;
(48αA-e) a protein comprising an amino acid sequence encoded by a nucleic acid that hybridizes under stringent conditions with a nucleic acid having a nucleotide sequence complementary to the nucleotide sequence of SEQ ID NO: 564;
(48αB) a protein comprising at least one amino acid sequence selected from the group consisting of SEQ ID NOs: 566-570, preferably a protein comprising at least 2, 3 or 4 or all sequences of the amino acid sequences. As referred to above, the amino acid sequence of any of SEQ ID NOs: 566-570 may be an amino acid sequence derived therefrom by deletion, substitution, insertion or addition of one or several amino acids.

The proteins selected from the group consisting of (48αA-a) to (48αA-e) and (48αB) as defined above also include those proteins whose amino acid residues are modified by phosphorylation, sugar chain modification, aminoacylation, ring-opening, deamination or the like.

The proteins selected from the group consisting of (48αA-a) to (48αA-e) and (48αB) as defined above can be proteins that are found in a protein spot with a molecular weight of around 2 to 50 kDa, preferably around 5 to 40 kDa and an isoelectric point of 3.5 to 8.5, preferably 4.0 to 8.0 on gels used in the two-dimensional electrophoresis performed under the conditions described above in the subsection titled “Identification of antigens”.

Preferably, a protein that is an antigen in spot 48α has activity equivalent to that of the protein having the amino acid sequence of SEQ ID NO: 565 (uncharacterized protein LOC100306570) or causative of an allergy to soybean.

### (50α) Antigen in Spot 50α

As the result of sequence identification of the antigen in spot 50α by mass spectroscopy, the following amino acid sequences were detected.

(SEQ ID NO: 573)
AADYLHNYQGDNTTAPSQPAESK
(SEQ ID NO: 574)
LVAEAAQSALK
(SEQ ID NO: 575)
QGIGQYVDK
(SEQ ID NO: 576)
VADAAGDLLDAAGK

Also, the mass spectroscopic data obtained for spot 50α on a mass spectrometer was analyzed by comparing the data against the NCBI protein data, and as a result, uncharacterized protein LOC100813859 (amino acid sequence: SEQ ID NO: 572, encoding nucleotide sequence: SEQ ID NO: 571) was identified as a protein comprising any of SEQ ID NOs: 573 to 576.

Accordingly, the antigens in spot 50α in the present invention can be any protein selected from the group consisting of the proteins as defined below in (50αA-a) to (50αA-e) and (50αB):
(50αA-a) a protein comprising an amino acid sequence with deletion, substitution, insertion or addition of one or several amino acids in SEQ ID NO: 572;
(50αA-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 572;
(50αA-c) a protein comprising an amino acid sequence encoded by a nucleotide sequence with deletion, substitution, insertion or addition of one or several nucleotides in SEQ ID NO: 571;
(50αA-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 571;
(50αA-e) a protein comprising an amino acid sequence encoded by a nucleic acid that hybridizes under stringent conditions with a nucleic acid having a nucleotide sequence complementary to the nucleotide sequence of SEQ ID NO: 571;
(50αB) a protein comprising at least one amino acid sequence selected from the group consisting of SEQ ID NOs: 573-576, preferably a protein comprising at least 2 or 3 or all sequences of the amino acid sequences. As referred to above, the amino acid sequence of any of SEQ ID NOs: 573-576 may be an amino acid sequence derived therefrom by deletion, substitution, insertion or addition of one or several amino acids.

The proteins selected from the group consisting of (50αA-a) to (50αA-e) and (50αB) as defined above also include those proteins whose amino acid residues are modified by phosphorylation, sugar chain modification, aminoacylation, ring-opening, deamination or the like.

The proteins selected from the group consisting of (50αA-a) to (50αA-e) and (50αB) as defined above can be proteins that are found in a protein spot with a molecular weight of around 2 to 50 kDa, preferably around 5 to 40 kDa and an isoelectric point of 2.5 to 6.5, preferably 3.0 to 6.0 on gels used in the two-dimensional electrophoresis performed under the conditions described above in the subsection titled “Identification of antigens”.

Preferably, a protein that is an antigen in spot 50α has activity equivalent to that of the protein having the amino acid sequence of SEQ ID NO: 572 (uncharacterized protein LOC100813859) or causative of an allergy to soybean.

### (51α) Antigen in Spot 51α

As the result of sequence identification of the antigen in spot 51α by mass spectroscopy, the following amino acid sequences were detected.

(SEQ ID NO: 579)
EAAESTLSAYK
(SEQ ID NO: 580)
LLDSRLIPSASSGDSK
(SEQ ID NO: 581)
QAFDEAIAELDTLGEESYK
(SEQ ID NO: 582)
VSAAADNEELTVEERNLLSVAYK

Also, the mass spectroscopic data obtained for spot 51α on a mass spectrometer was analyzed by comparing the data against the NCBI protein data, and as a result, 14-3-3-like protein (amino acid sequence: SEQ ID NO: 578, encoding nucleotide sequence: SEQ ID NO: 577) was identified as a protein comprising any of SEQ ID NOs: 579 to 582.

Accordingly, the antigens in spot 51α in the present invention can be any protein selected from the group consisting of the proteins as defined below in (51αA-a) to (51αA-e) and (51αB):
(51αA-a) a protein comprising an amino acid sequence with deletion, substitution, insertion or addition of one or several amino acids in SEQ ID NO: 578;
(51αA-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 578;
(51αA-c) a protein comprising an amino acid sequence encoded by a nucleotide sequence with deletion, substitution, insertion or addition of one or several nucleotides in SEQ ID NO: 577;
(51αA-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 577;
(51αA-e) a protein comprising an amino acid sequence encoded by a nucleic acid that hybridizes under stringent conditions with a nucleic acid having a nucleotide sequence complementary to the nucleotide sequence of SEQ ID NO: 577;
(51αB) a protein comprising at least one amino acid sequence selected from the group consisting of SEQ ID NOs: 579-582, preferably a protein comprising at least 2 or 3 or all sequences of the amino acid sequences. As referred to above, the amino acid sequence of any of SEQ ID NOs: 579-582 may be an amino acid sequence derived therefrom by deletion, substitution, insertion or addition of one or several amino acids.

The proteins selected from the group consisting of (51αA-a) to (51αA-e) and (51αB) as defined above also include those proteins whose amino acid residues are modified by phosphorylation, sugar chain modification, aminoacylation, ring-opening, deamination or the like.

The proteins selected from the group consisting of (51αA-a) to (51αA-e) and (51αB) as defined above can be proteins that are found in a protein spot with a molecular weight of around 5 to 60 kDa, preferably around 10 to 50 kDa and an isoelectric point of 2.5 to 6.5, preferably 3.0 to 6.0 on gels used in the two-dimensional electrophoresis performed under the conditions described above in the subsection titled “Identification of antigens”.

Preferably, a protein that is an antigen in spot 51α has activity equivalent to that of the protein having the amino acid sequence of SEQ ID NO: 578 (14-3-3-like protein) or causative of an allergy to soybean.

### (52α) Antigen in Spot 52α

As the result of sequence identification of the antigen in spot 52α by mass spectroscopy, the following amino acid sequences were detected.

(SEQ ID NO: 585)
APGGAPANVAIAVSRLGGK
(SEQ ID NO: 586)
GSVDAFHVNTVDTTGAGDSFVGALLAK
(SEQ ID NO: 587)
IVDDQSILEDEPRLREVLK
(SEQ ID NO: 588)
VSDAELEFLTGSDK

Also, the mass spectroscopic data obtained for spot 52α on a mass spectrometer was analyzed by comparing the data against the NCBI protein data, and as a result, probable fructokinase-4 (amino acid sequence: SEQ ID NO: 584, encoding nucleotide sequence: SEQ ID NO: 583) was identified as a protein comprising any of SEQ ID NOs: 585 to 588.

Accordingly, the antigens in spot 52α in the present invention can be any protein selected from the group consisting of the proteins as defined below in (52αA-a) to (52αA-e) and (52αB):
(52αA-a) a protein comprising an amino acid sequence with deletion, substitution, insertion or addition of one or several amino acids in SEQ ID NO: 584;
(52αA-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 584;
(52αA-c) a protein comprising an amino acid sequence encoded by a nucleotide sequence with deletion, substitution, insertion or addition of one or several nucleotides in SEQ ID NO: 583;
(52αA-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 583;
(52αA-e) a protein comprising an amino acid sequence encoded by a nucleic acid that hybridizes under stringent conditions with a nucleic acid having a nucleotide sequence complementary to the nucleotide sequence of SEQ ID NO: 583;
(52αB) a protein comprising at least one amino acid sequence selected from the group consisting of SEQ ID NOs: 585-588, preferably a protein comprising at least 2 or 3 or all sequences of the amino acid sequences. As referred to above, the amino acid sequence of any of SEQ ID NOs: 585-588 may be an amino acid sequence derived therefrom by deletion, substitution, insertion or addition of one or several amino acids.

The proteins selected from the group consisting of (52αA-a) to (52αA-e) and (52αB) as defined above also include those proteins whose amino acid residues are modified by phosphorylation, sugar chain modification, aminoacylation, ring-opening, deamination or the like.

The proteins selected from the group consisting of (52αA-a) to (52αA-e) and (52αB) as defined above can be proteins that are found in a protein spot with a molecular weight of around 5 to 60 kDa, preferably around 10 to 50 kDa and an isoelectric point of 3.5 to 7.5, preferably 4.0 to 7.0 on gels used in the two-dimensional electrophoresis performed under the conditions described above in the subsection titled “Identification of antigens”.

Preferably, a protein that is an antigen in spot 52α has activity equivalent to that of the protein having the amino acid sequence of SEQ ID NO: 584 (probable fructokinase-4) or causative of an allergy to soybean.

### (53α) Antigen in Spot 53α

As the result of sequence identification of the antigen in spot 53α by mass spectroscopy, the following amino acid sequences were detected.

(SEQ ID NO: 591)
AEFVDIINAATVK
(SEQ ID NO: 592)
ELAAQPDVDGFLVGGASLK
(SEQ ID NO: 593)
FFVGGNWK
(SEQ ID NO: 594)
IVTTLNEAK
(SEQ ID NO: 595)
VATPAQAQEVHADLRK
(SEQ ID NO: 596)
VAYALQQGLK
(SEQ ID NO: 597)
VPGEDVVEVVVSPPFVFLPFVK
(SEQ ID NO: 600)
ELAAQPDVDGFLVGGASLKPEFVDIINAATVK
(SEQ ID NO: 601)
FFVGGNWK
(SEQ ID NO: 602)
IVTTLNEAK
(SEQ ID NO: 603)
VATPAQAQEVHADLRK
(SEQ ID NO: 604)
VAYALQQGLK

Also, the mass spectroscopic data obtained for spot 53α on a mass spectrometer was analyzed by comparing the data against the NCBI protein data, and as a result, triosephosphate isomerase isoform X1 (amino acid sequence: SEQ ID NO: 590, encoding nucleotide sequence: SEQ ID NO: 589) was identified as a protein comprising any of SEQ ID NOs: 591 to 597 and triosephosphate isomerase, cytosolic (amino acid sequence: SEQ ID NO: 599, encoding nucleotide sequence: SEQ ID NO: 598) was identified as a protein comprising any of SEQ ID NOs: 600 to 604.

Accordingly, the antigen in spot 53α in the present invention can be any of proteins selected from the group consisting of the proteins as defined below in (53αA1-a) to (53αA1-e), (53αB1), (53αA2-a) to (53αA2-e), and (53αB2):
(53αA1-a) a protein comprising an amino acid sequence with deletion, substitution, insertion or addition of one or several amino acids in SEQ ID NO: 590;
(53αA1-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 590;
(53αA1-c) a protein comprising an amino acid sequence encoded by a nucleotide sequence with deletion, substitution, insertion or addition of one or several nucleotides in SEQ ID NO: 589;
(53αA1-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 589;
(53αA1-e) a protein comprising an amino acid sequence encoded by a nucleic acid that hybridizes under stringent conditions with a nucleic acid having a nucleotide sequence complementary to the nucleotide sequence of SEQ ID NO: 589;
(53αB1) a protein comprising at least one amino acid sequence selected from the group consisting of SEQ ID NOs: 591-597, preferably a protein comprising at least 2, 3, 4, 5, or 6 or all sequences of the amino acid sequences. As referred to above, the amino acid sequence of any of SEQ ID NOs: 591 to 597 may be an amino acid sequence derived therefrom by deletion, substitution, insertion or addition of one or several amino acids;
(53αA2-a) a protein comprising an amino acid sequence with deletion, substitution, insertion or addition of one or several amino acids in SEQ ID NO: 599;
(53αA2-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 599;
(53αA2-c) a protein comprising an amino acid sequence encoded by a nucleotide sequence with deletion, substitution, insertion or addition of one or several nucleotides in SEQ ID NO: 598;
(53αA2-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 598;
(53αA2-e) a protein comprising an amino acid sequence encoded by a nucleic acid that hybridizes under stringent conditions with a nucleic acid having a nucleotide sequence complementary to the nucleotide sequence of SEQ ID NO: 598;
(53αB2) a protein comprising at least one amino acid sequence selected from the group consisting of SEQ ID NOs: 600-604, preferably a protein comprising at least 2, 3, or 4 or all sequences of the amino acid sequences. As referred to above, the amino acid sequence of any of SEQ ID NOs: 600 to 604 may be an amino acid sequence derived therefrom by deletion, substitution, insertion or addition of one or several amino acids.

The protein selected from the group consisting of the proteins as defined above in (53αA1-a) to (53αA1-e), (53αB1), (53αA2-a) to (53αA2-e), and (53αB2) also include those proteins whose amino acid residues are modified by phosphorylation, sugar chain modification, aminoacylation, ring-opening, deamination or the like.

The protein selected from the group consisting of the proteins as defined above in (53αA1-a) to (53αA1-e), (53αB1), (53αA2-a) to (53αA2-e), and (53αB2) can be proteins that are found in a protein spot with a molecular weight of around 5 to 60 kDa, preferably around 10 to 50 kDa, and an isoelectric point of 3.5 to 8.5, preferably 4.0 to 8.0 on gels used in the two-dimensional electrophoresis performed under the conditions described above in the subsection titled “Identification of antigens”.

Preferably, a protein that is an antigen in spot 53α has activity equivalent to that of the protein having the amino acid sequence of SEQ ID NO: 590 (triosephosphate isomerase isoform X1) or the protein having the amino acid sequence of SEQ ID NO: 599 (triosephosphate isomerase, cytosolic) or causative of an allergy to soybean.

### (54α) Antigen in Spot 54α

As the result of sequence identification of the antigen in spot 54α by mass spectroscopy, the following amino acid sequences were detected.

(SEQ ID NO: 607)
FDGENVANPPSPDEDNK
(SEQ ID NO: 608)
LVSWDAVSSRLEQAK
(SEQ ID NO: 609)
QVVGTELDGK
(SEQ ID NO: 610)
SLEEIIVTSYNK
(SEQ ID NO: 611)
TYVENLK
(SEQ ID NO: 614)
QIVGTELDGK
(SEQ ID NO: 615)
SLEEIIVTSYNK
(SEQ ID NO: 616)
TYVENLK

Also, the mass spectroscopic data obtained for spot 54α on a mass spectrometer was analyzed by comparing the data against the NCBI protein data, and as a result, superoxide dismutase [Fe], chloroplastic isoform X1 (amino acid sequence: SEQ ID NO: 606, encoding nucleotide sequence: SEQ ID NO: 605) as a protein comprising any of SEQ ID NOs: 607 to 611 and iron-superoxide dismutase (amino acid sequence: SEQ ID NO: 613, encoding nucleotide sequence: SEQ ID NO: 612) was identified as a protein comprising any of SEQ ID NOs: 614 to 616.

Accordingly, the antigen in spot 54α in the present invention can be any of proteins selected from the group consisting of the proteins as defined below in (54αA1-a) to (54αA1-e), (54αB1), (54αA2-a) to (54αA2-e), and (54αB2):
(54αA1-a) a protein comprising an amino acid sequence with deletion, substitution, insertion or addition of one or several amino acids in SEQ ID NO: 606;
(54αA1-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 606;
(54αA1-c) a protein comprising an amino acid sequence encoded by a nucleotide sequence with deletion, substitution, insertion or addition of one or several nucleotides in SEQ ID NO: 605;
(54αA1-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 605;
(54αA1-e) a protein comprising an amino acid sequence encoded by a nucleic acid that hybridizes under stringent conditions with a nucleic acid having a nucleotide sequence complementary to the nucleotide sequence of SEQ ID NO: 605;
(54αB1) a protein comprising at least one amino acid sequence selected from the group consisting of SEQ ID NOs: 607-611, preferably a protein comprising at least 2, 3, or 4 or all sequences of the amino acid sequences. As referred to above, the amino acid sequence of any of SEQ ID NOs: 607-611 may be an amino acid sequence derived therefrom by deletion, substitution, insertion or addition of one or several amino acids;
(54αA2-a) a protein comprising an amino acid sequence with deletion, substitution, insertion or addition of one or several amino acids in SEQ ID NO: 613;
(54αA2-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 613;
(54αA2-c) a protein comprising an amino acid sequence encoded by a nucleotide sequence with deletion, substitution, insertion or addition of one or several nucleotides in SEQ ID NO: 612;
(54αA2-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 612;
(54αA2-e) a protein comprising an amino acid sequence encoded by a nucleic acid that hybridizes under stringent conditions with a nucleic acid having a nucleotide sequence complementary to the nucleotide sequence of SEQ ID NO: 612;
(54αB2) a protein comprising at least one amino acid sequence selected from the group consisting of SEQ ID NOs: 614-616, preferably a protein comprising at least 2, or all sequences of the amino acid sequences. As referred to above, the amino acid sequence of any of SEQ ID NOs: 614 to 616 may be an amino acid sequence derived therefrom by deletion, substitution, insertion or addition of one or several amino acids.

The protein selected from the group consisting of the proteins as defined above in (54αA1-a) to (54αA1-e), (54αB1), (54αA2-a) to (54αA2-e), and (54αB2) also include those proteins whose amino acid residues are modified by phosphorylation, sugar chain modification, aminoacylation, ring-opening, deamination or the like.

The protein selected from the group consisting of the proteins as defined above in (54αA1-a) to (54αA1-e), (54αB1), (54αA2-a) to (54αA2-e), and (54αB2) can be proteins that are found in a protein spot with a molecular weight of around 5 to 60 kDa, preferably around 10 to 50 kDa, and an isoelectric point of 3.5 to 8.5, preferably 4.0 to 8.0 on gels used in the two-dimensional electrophoresis performed under the conditions described above in the subsection titled “Identification of antigens”.

Preferably, a protein that is an antigen in spot 54α has activity equivalent to that of the protein having the amino acid sequence of SEQ ID NO: 606 (superoxide dismutase [Fe], chloroplastic isoform X1) or the protein having the amino acid sequence of SEQ ID NO: 613 (iron-superoxide dismutase) or causative of an allergy to soybean.

### (55α) Antigen in Spot 55α

As the result of sequence identification of the antigen in spot 55α by mass spectroscopy, the following amino acid sequences were detected.

(SEQ ID NO: 619)
DYAGDAAQNVK
(SEQ ID NO: 620)
DYASDAAQK
(SEQ ID NO: 621)
DYASDATDAAK
(SEQ ID NO: 622)
DYASDAVQK
(SEQ ID NO: 623)
DYASDTAQRTK
(SEQ ID NO: 624)
DYASDTAQTSK
(SEQ ID NO: 625)
DYASEASDVAQNTK
(SEQ ID NO: 626)
DYATDAAQK
(SEQ ID NO: 627)
DYVGDAAQRSK
(SEQ ID NO: 628)
EASDYASETAK
(SEQ ID NO: 629)
EYAGDVALNAK
(SEQ ID NO: 630)
EYVGDAAQK
(SEQ ID NO: 631)
GAAEYASDAAQRTK
(SEQ ID NO: 632)
GYVGDAAQK
(SEQ ID NO: 633)
LQDIASEAGQYSAEK
(SEQ ID NO: 634)
LSEGLGFK
(SEQ ID NO: 635)
VSDYATDTAQK
(SEQ ID NO: 638)
DYAGDAVQNIK
(SEQ ID NO: 639)
DYASDAAQRTK
(SEQ ID NO: 640)
DYASDATDAAK
(SEQ ID NO: 641)
DYASDTAQRTK
(SEQ ID NO: 642)
DYVGDAAQK
(SEQ ID NO: 643)
EASDYASETAQK
(SEQ ID NO: 644)
EASEYASDAAQRTK
(SEQ ID NO: 645)
EYAGDVAQNAK
(SEQ ID NO: 646)
GYVGDAAQK
(SEQ ID NO: 647)
LQDIASEAGQYSTEK
(SEQ ID NO: 648)
LTEGLGFK
(SEQ ID NO: 649)
VSDYAGDAAQK

Also, the mass spectroscopic data obtained for spot 55α on a mass spectrometer was analyzed by comparing the data against the NCBI protein data, and as a result, 51 kDa seed maturation protein precursor (amino acid sequence: SEQ ID NO: 618, encoding nucleotide sequence: SEQ ID NO: 617) was identified as a protein comprising any of SEQ ID NOs: 619 to 635 and Lea protein precursor (amino acid sequence: SEQ ID NO: 637, encoding nucleotide sequence: SEQ ID NO: 636) was identified as a protein comprising any of SEQ ID NOs: 638 to 649.

Accordingly, the antigen in spot 55α in the present invention can be any of proteins selected from the group consisting of the proteins as defined below in (55αA1-a) to (55αA1-e), (55αB1), (55αA2-a) to (55αA2-e), and (55αB2):
(55αA1-a) a protein comprising an amino acid sequence with deletion, substitution, insertion or addition of one or several amino acids in SEQ ID NO: 618;
(55αA1-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 618;
(55αA1-c) a protein comprising an amino acid sequence encoded by a nucleotide sequence with deletion, substitution, insertion or addition of one or several nucleotides in SEQ ID NO: 617;
(55αA1-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 617;
(55αA1-e) a protein comprising an amino acid sequence encoded by a nucleic acid that hybridizes under stringent conditions with a nucleic acid having a nucleotide sequence complementary to the nucleotide sequence of SEQ ID NO: 617;
(55αB1) a protein comprising at least one amino acid sequence selected from the group consisting of SEQ ID NOs: 619-635, preferably a protein comprising at least 2, 3, 4, 5, 6, 7, 8, 9, 10, 11, 12, 13, 14, 15, or 16 or all sequences of the amino acid sequences. As referred to above, the amino acid sequence of any of SEQ ID NOs: 619 to 635 may be an amino acid sequence derived therefrom by deletion, substitution, insertion or addition of one or several amino acids;
(55αA2-a) a protein comprising an amino acid sequence with deletion, substitution, insertion or addition of one or several amino acids in SEQ ID NO: 637;
(55αA2-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 637;
(55αA2-c) a protein comprising an amino acid sequence encoded by a nucleotide sequence with deletion, substitution, insertion or addition of one or several nucleotides in SEQ ID NO: 636;
(55αA2-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 636;
(55αA2-e) a protein comprising an amino acid sequence encoded by a nucleic acid that hybridizes under stringent conditions with a nucleic acid having a nucleotide sequence complementary to the nucleotide sequence of SEQ ID NO: 636;
(55αB2) a protein comprising at least one amino acid sequence selected from the group consisting of SEQ ID NOs: 638-649, preferably a protein comprising at least 2, 3, 4, 5, 6, 7, 8, 9, 10, or 11 or all sequences of the amino acid sequences. As referred to above, the amino acid sequence of any of SEQ ID NOs: 638 to 649 may be an amino acid sequence derived therefrom by deletion, substitution, insertion or addition of one or several amino acids.

The protein selected from the group consisting of the proteins as defined above in (55αA1-a) to (55αA1-e), (55αB1), (55αA2-a) to (55αA2-e), and (55αB2) also include those proteins whose amino acid residues are modified by phosphorylation, sugar chain modification, aminoacylation, ring-opening, deamination or the like.

The protein selected from the group consisting of the proteins as defined above in (55αA1-a) to (55αA1-e), (55αB1), (55αA2-a) to (55αA2-e), and (55αB2) can be proteins that are found in a protein spot with a molecular weight of around 30 to 90 kDa, preferably around 40 to 80 kDa, and an isoelectric point of 5.5 to 10.5, preferably 6.0 to 10.0 on gels used in the two-dimensional electrophoresis performed under the conditions described above in the subsection titled “Identification of antigens”.

Preferably, a protein that is an antigen in spot 55α has activity equivalent to that of the protein having the amino acid sequence of SEQ ID NO: 618 (51 kDa seed maturation protein precursor) or the protein having the amino acid sequence of SEQ ID NO: 637 (Lea protein precursor) or causative of an allergy to soybean.

By stating herein “deletion, substitution, insertion or addition of one or several amino acids” in relation to amino acid sequence, it is meant that in an amino acid sequence of interest, one or several amino acids (e.g., 30%, preferably 25%, 20%, 15%, 10%, 5%, 3%, 2%, or 1% of amino acids with respect to the total length of the amino acid sequence) are deleted, one or several amino acids are substituted by any other amino acids, any other amino acids are inserted, and/or any other amino acids are added.

Among the aforementioned modifications, substitution is preferably conservative substitution. The “conservative substitution” refers to the substitution of a certain amino acid residue by a different amino acid residue having similar physicochemical characteristics, and can be any type of substitution as long as it does not substantially change the characteristics of the structure of the original sequence for example, any type of substitution is acceptable as long as any substituted amino acids do not disrupt the helical structure of the original sequence or other secondary structures that characterize the original sequence. The following gives examples of separate groups of amino acid residues that are conservatively substitutable with each other, but substitutable amino acid residues are not limited to the examples given below.
Group A: leucine, isoleucine, valine, alanine, methionine
Group B: aspartic acid, glutamic acid
Group C: asparagine, glutamine
Group D: lysine, arginine
Group E: serine, threonine
Group F: phenylalanine, tyrosine

In the case of non-conservative substitution, one member belonging to one of the aforementioned groups can be replaced with a member belong to any other group. For example, in order to eliminate the possibility of unwanted sugar-chain modification, amino acids of group B, D or E as listed above may be substituted by those of any other group. Also, cysteines may be deleted or substituted by any other amino acids to prevent them from being folded into a protein in its tertiary structure. Also, in order to maintain the balance between hydrophilicity and hydrophobicity or to increase hydrophilicity for the purpose of facilitating synthesis, any amino acids may be substituted in consideration of the hydropathy scales of amino acids, which are a measure of the hydrophilic and hydrophobic properties of amino acids (J. Kyte and R. Doolittle, J. Mol. Biol., Vol. 157, p. 105-132, 1982).

As referred to herein, the percent identity between two amino acid sequences can be determined by visual inspection and mathematical calculation. Alternatively, the percent identity can be determined using a computer program. Examples of such computer programs include BLAST FASTA (Altschul, et al., J. Mol. Biol., 215: 403-410 (1990)), and ClustalW. In particular, various conditions (parameters) for identity searches with the BLAST program are described in Altschul, et al. (Nucl. Acids. Res., 25, p. 3389-3402, 1997), and are publicly available on the websites of the National Center for Biotechnology Information (NCBI) and DNA Data Bank of Japan (DDBJ) (Altschul, et al., BLAST Handbook, Altschul, et al., NCB/NLM/NIH Bethesda, MD 20894). Also, the percent identity can be determined using a genetic information processing software program, such as GENETYX Ver.7 (Genetyx Corporation), DINASIS Pro (Hitachi Software Engineering Co., Ltd.), or Vector NTI (Infomax Inc.).

By stating herein “deletion, substitution, insertion or addition of one or several nucleotides” in relation to nucleotide sequence, it is meant that in a nucleotide sequence of interest, one or several nucleotides (e.g., 30%, preferably 25%, 20%, 15%, 10%, 5%, 3%, 2% or 1%, of nucleotides with respect to the total length of the nucleotide sequence) are deleted, one or several nucleotides are substituted by any other nucleotides, any other nucleotides are inserted, and/or any other nucleotides are added. It is preferable that such a nucleotide deletion, substitution, insertion or addition should not give rise to a frame shift in an amino acid coding sequence.

As referred to herein, the percent identity between two nucleotide sequences can be determined by visual inspection and mathematical calculation. Alternatively, the percent identity can be determined using a computer program. Examples of such sequence comparison computer programs include the BLASTN program, version 2.2.7, available on the website of the National Library of Medicine (http://www.ncbi.nlm.nih.gov/blast/bl2seq/bls.html) (Altschul, et al. (1990) J. Mol. Biol., 215: 403-10), or the WU-BLAST 2.0 algorithm. Standard default parameter settings for WU-BLAST 2.0 are found and available on the following website: http://blast.wustl.edu.

As referred to herein, “under stringent conditions” means that hybridization takes place under moderately or highly stringent conditions. To be specific, the moderately stringent conditions can be easily determined by those having ordinary skill in the art on the basis of, for example, the length of DNA. Basic conditions are described in Sambrook, et al., Molecular Cloning: A Laboratory Manual, 3rd ed., ch.6-7, Cold Spring Harbor Laboratory Press, 2001. The moderately stringent conditions include hybridization under the conditions of preferably 1×SSC to 6×SSC at 42° C. to 55° C., more preferably 1×SSC to 3×SSC at 45° C. to 50° C., most preferably 2×SSC at 50° C. In the case of using a hybridization solution containing, for example, about 50% formamide, a temperature around 5 to 15° C. lower than the foregoing should be adopted. Washing is also carried out under the conditions of 0.5×SSC to 6×SSC at 40° C. to 60° C. In the process of hybridization and washing, generally 0.05% to 0.2% SDS, preferably about 0.1% SDS, may be added. Likewise, the highly stringent conditions can be easily determined by those having ordinary skill in the art on the basis of, for example, the length of DNA. Generally, the highly stringent (high stringent) conditions include hybridization and/or washing at a higher temperature and/or a lower salt concentration than those adopted under the moderately stringent conditions. For example, hybridization is carried out under the conditions of 0.1×SSC to 2×SSC at 55° C. to 65° C., more preferably 0.1×SSC to 1×SSC at 60° C. to 65° C., most preferably 0.2×SSC at 63° C. Washing is carried out under the conditions of 0.2×SSC to 2×SSC at 50° C. to 68° C., more preferably 0.2×SSC at 60 to 65° C.

Antigens may be obtained by separating and purifying them from soybean using a combination of protein purification methods well known to those skilled in the art. Also, antigens may be obtained by expressing them as recombinant proteins using a genetic recombination technique well known to those skilled in the art and by separating and purifying them using protein purification methods well known to those skilled in the art.

Exemplary protein purification methods include: solubility-based purification methods such as salt precipitation and solvent precipitation; purification methods based on molecular weight difference, such as dialysis, ultrafiltration, gel filtration and SDS-PAGE; charge-based purification methods such as ion exchange chromatography and hydroxylapatite chromatography; specific affinity-based purification methods such as affinity chromatography; purification methods based on hydrophobicity difference, such as reverse-phase high-performance liquid chromatography; and purification methods based on isoelectric point difference, such as isoelectric focusing.

Preparation of a protein by a genetic recombination technique is carried out by preparing an expression vector comprising an antigen-encoding nucleic acid, introducing the expression vector into appropriate host cells by gene transfer or genetic transformation, culturing the host cells under suitable conditions for expression of a recombinant protein, and recovering the recombinant protein expressed in the host cells.

The “vector” refers to a nucleic acid that can be used to introduce a nucleic acid attached thereto into host cells. The “expression vector” is a vector that can induce the expression of a protein encoded by a nucleic acid introduced therethrough. Exemplary vectors include plasmid vectors and viral vectors. Those skilled in the art can select an appropriate expression vector for the expression of a recombinant protein depending on the type of host cells to be used.

The “host cells” refers to cells that undergo gene transfer or genetic transformation by a vector. The host cells can be appropriately selected by those skilled in the art depending on the type of the vector to be used.

The host cells can be derived from prokaryotes such as E. coli. When prokaryotic cells like E coli. are used as host cells, the antigen of the present invention may be designed to contain an N-terminal methionine residue in order to facilitate the expression of a recombinant protein in the prokaryotic cells. The N-terminal methionine can be cleaved from the recombinant protein after expression. Also, the host cells may be cells derived from eukaryotes, such as single-cell eukaryotes like yeast, plant cells and animal cells (e.g., human cells, monkey cells, hamster cells, rat cells, murine cells or insect cells) or silkworm.

Gene transfer or genetic transformation of an expression vector into host cells can be carried out as appropriate by following a technique known to those skilled in the art. Those skilled in the art can make possible the expression of a recombinant protein by selecting suitable conditions for the expression of the recombinant protein as appropriate depending on the type of host cells and culturing the host cells under the selected conditions. Then, those skilled in the art can homogenize the host cells having the expressed recombinant protein, and separate and purify an antigen expressed as the recombinant protein from the resulting homogenate by using an appropriate combination of such protein purification methods as mentioned above.

### Diagnosis Kit and Method

The present invention provides a method for providing an indicator for diagnosing an allergy to a soybean in a subject, the method comprising the steps of:
(i) contacting a sample obtained from the subject with an antigen, wherein the sample is a solution comprising an Ig antibody;
(ii) detecting binding between an IgE antibody present in the sample from the subject and the antigen; and
(iii) when the binding between the IgE antibody in the subject and the antigen is detected, an indicator of the fact that the subject is allergic to a soybean is provided;
wherein the antigen is at least one of proteins as defined above in any of (1) to (8) and (1α) to (55α).

The sample obtained from a subject is a solution containing an Ig antibody, preferably an IgE antibody, as collected from the subject. Examples of such solutions include blood, saliva, sputum, snivel, urine, sweat, and tear. The sample obtained from the subject may be subjected to pretreatment for increasing the concentration of an Ig antibody in the sample before being contacted with an antigen. The pretreatment of a sample may involve, for example, collection of the serum from the blood. In a particularly preferred mode, the step (i) mentioned above is carried out by contacting an IgE antibody present in the serum obtained from a subject with an antigen.

Detection of contact and binding between the sample obtained from a subject and an antigen can be carried out by using a known method. Examples of such methods that can be used include detection by ELISA (Enzyme-Linked Immunosorbent Assay), sandwich immunoassay, immunoblotting, or immunoprecipitation.

These are all techniques for detecting binding between an antigen and an IgE antibody from a subject by contacting and binding the IgE antibody from a subject with the antigen, allowing an enzymatically labelled secondary antibody to act on the IgE antibody specifically bound to the antigen, and adding an enzyme substrate (generally, chromogenic or luminescent reagent) to detect an enzymatic reaction product. Also, a method for detecting a fluorescently labeled secondary antibody can be used. Furthermore, a method for detecting antigen-antibody binding as a refraction angle using surface plasmon resonance can be used.

The antigen may be provided as an isolated antigen in a state immobilized on a carrier. In this case, the steps (i) and (ii) mentioned above can be carried out using ELISA, sandwich immunoassay, or the like. Also, the step (i) mentioned above can be carried out by contacting the sample obtained from a subject with an antigen-immobilized surface. The isolated antigen may be obtained by separating and purifying it from soybean using a combination of protein purification methods well known to those skilled in the art, or by preparing it using a genetic recombination technique.

The antigen may be detected by immunoblotting in a state separated by two-dimensional electrophoresis. The two-dimensional electrophoresis is a technique for separating a protein sample by performing isoelectric focusing in the first dimension and performing SDS-PAGE (SDS-polyacrylamide gel electrophoresis) in the second dimension. The conditions for two-dimensional electrophoresis are not particularly limited as long as the conditions permit the separation of the antigen of the present invention. For example, the conditions for two-dimensional electrophoresis as described above in the subsection titled “Identification of antigens” can be adopted. Also, the electrophoresis conditions may be defined by reference to the descriptions in Patent Literatures 1 to 4 mentioned above. For example, two-dimensional electrophoresis can be carried out under the conditions that satisfy at least one selected from the group consisting of the following requirements:
(A) the isoelectric focusing gels used in the first dimension should have a gel-strip length of 5 to 10 cm and a gel pH range of 3 to 10, and the pH gradient of the gels in the direction of electrophoresis should be as follows:
where the gel-strip length up to pH 5 is taken as “a”, that length from pH 5 to 7 as “b”, and that length above pH 7 as “c”, the relations “a<b” and “b>c” are satisfied;
(B) in the case of (A), when the total gel-strip length is taken as 1, “a” should be in the range of 0.15 to 0.3, “b” should be in the range of 0.4 to 0.7, and “c” should be in the range of 0.15 to 0.3;
(C) in the first dimensional isoelectric focusing, a constant voltage step should be performed by applying a constant voltage ranging from 100 V to 600 V per gel strip containing a sample, and after the electrophoresis variation width during electrophoresis for 30 minutes falls within the range of 5 μA, a voltage-increasing step should be started at which the voltage is increased from the aforementioned constant voltage;
(D) in the case of (C), the final voltage at the voltage-increasing step should be in the range of 3000 V to 6000 V;
(E) the isoelectric focusing gels used in the first dimension should have a longitudinal gel-strip length of 5 to 10 cm, and the electrophoresis gels used in the second dimension should have a gel concentration at the distal end in the direction of electrophoresis, which is in the range of 3 to 6%; and
(F) in the case of (E), the electrophoresis gels used in the second dimension should have a gel concentration at the proximal end in the direction of electrophoresis, which is set to a higher value than that at the distal end in the direction of electrophoresis.

The aforementioned antigens (1) to (8) and (1α) to (55α) are antigens that specifically bind to IgE antibodies from patients with allergy to soybean. Therefore, when binding between an IgE antibody from a subject and the antigen is detected, an indicator of the fact that the subject is allergic to soybean is provided.

The present invention further provides a kit for diagnosing an allergy to a soybean, comprising at least one of the aforementioned antigens (1) to (8) and (1α) to (55α). The diagnosis kit of this invention may be used in the aforementioned method for providing an indicator for diagnosing an allergy to a soybean or in a diagnosis method as described later. The diagnosis kit of this invention may comprise not only the at least one of the aforementioned antigens (1) to (8) and (1α) to (55α), but also an anti-IgE antibody labeled with an enzyme and a chromogenic or luminescent substrate serving as a substrate for the enzyme. Also, a fluorescent-labeled anti-IgE antibody may be used. In the diagnosis kit of this invention, the antigen may be provided in a state immobilized on a carrier. The diagnosis kit of this invention may also be provided together with instructions on the procedure for diagnosis or a package containing said instructions.

The present invention further provides a composition for diagnosing an allergy to a soybean, comprising at least one of the aforementioned antigens (1) to (8) and (1α) to (55α). The diagnosis composition of this invention can be used in a diagnosis method as described below. The diagnosis composition of this invention may further comprise a pharmaceutically acceptable carrier and/or additives commonly used with the antigen of this invention depending on the need.

In one mode, the present invention provides a method for diagnosing an allergy to a soybean in a subject, the method comprising:
(i) contacting a sample obtained from the subject with an antigen;
(ii) detecting binding between an IgE antibody present in the sample from the subject and the antigen; and
(iii) when the binding between the IgE antibody in the subject and the antigen is detected, diagnosing the subject as being allergic to a soybean;
wherein the antigen is at least one of proteins as defined above in any of (1) to (8) and (1α) to (55α). In this method, the steps (i) and (ii) are performed as described above regarding the corresponding steps of the method for providing an indicator for diagnosing an allergy to a soybean.

In another mode, the present invention provides a method for diagnosing an allergy to a soybean in a subject, the method comprising administering to the subject at least one of the aforementioned antigens (1) to (8) and (1α) to (55α). This method may be performed in the form of a skin test characterized by applying the antigen onto the skin. Examples of the skin test include various forms of tests, such as: a prick test in which a diagnosis composition is applied onto the skin and then a tiny prick to such an extent as not to provoke bleeding is made in the skin to allow an antigen to penetrate the skin, thereby observing a skin reaction; a scratch test in which a diagnosis composition is applied onto the skin and then the skin is lightly scratched to observe a reaction; a patch test in which a diagnosis composition in the form of cream, ointment, etc. is applied onto the skin to observe a reaction; and an intracutaneous test in which an antigen is administered intracutaneously to observe a reaction. If a skin reaction such as swelling occurs in a skin portion to which the antigen has been applied, the subject of interest is diagnosed as having an allergy to a soybean. The amount of the antigen to be applied to the skin in such tests can be, for example, not more than 100 μg per dose.

In yet another mode, the present invention provides at least one of the aforementioned antigens (1) to (8) and (1α) to (55α), intended for use in the diagnosis of an allergy to a soybean.

In still another mode, the present invention provides use of at least one of the aforementioned antigens (1) to (8) and (1α) to (55α) for the production of a diagnostic agent for an allergy to a soybean.

### Pharmaceutical Composition and Treatment Method

The present invention provides a pharmaceutical composition comprising at least one of the aforementioned antigens (1) to (8) and (1α) to (55α). In one mode, the aforementioned pharmaceutical composition is used for the treatment and diagnosis of an allergy to a soybean.

The present invention also provides a method for treating an allergy to a soybean, the method comprising administering at least one of the aforementioned antigens (1) to (8) and (1α) to (55α) to a patient in need of a treatment for an allergy to a soybean.

In another mode, the present invention provides at least one of the aforementioned antigens (1) to (8) and (1α) to (55α), intended for use in the treatment for an allergy to a soybean. In yet another mode, the present invention provides use of at least one of the aforementioned antigens (1) to (8) and (1α) to (55α) for the production of a therapeutic agent for an allergy to a soybean.

In the process of allergy treatment, a hyposensitization therapy aiming to induce immunological tolerance by administering an antigen to a patient is often adopted. The at least one of the aforementioned antigens (1) to (8) and (1α) to (55α) can be used as an active ingredient for a hyposensitization therapy or a load test for an allergy to a soybean. Here, the antigen protein to be used in the hyposensitization therapy and the load test may be a protein that has been expressed and purified and may be a protein that has been expressed in food such as rice, such as rice-based vaccine expressing pollen allergens.

The pharmaceutical composition of the present invention can be administered by common administration routes. Examples of common administration routes include oral, sublingual, percutaneous, intracutaneous, subcutaneous, intravascular, intranasal, intramuscular, and intraperitoneal administrations.

The pharmaceutical composition of the present invention can be used as a pharmaceutical composition to which a commonly used pharmaceutically acceptable adjuvant or excipient or any other additives (e.g., stabilizer, solubilizer, emulsifier, buffer, preservative, colorant) are added by a conventional method together with the antigen of this invention depending on the need. The dosage form of the pharmaceutical composition can be selected by those skilled in the art as appropriate depending on the administration route. The pharmaceutical composition can be in the form of, for example, tablet, capsule, troche, sublingual tablet, parenteral injection, intranasal spray, poultice, solution, cream, or lotion. The administration dose, frequency and/or period of the pharmaceutical composition of this invention can be selected by a physician as appropriate depending on the administration route and the patient's condition and characteristics such as age and body weight. For example, the pharmaceutical composition may be administered to an adult patient at a dose of not more than 100 μg per dose. The administration interval can be, for example, once daily, once weekly, twice weekly, once per three months or so. The administration period can be, for example, several weeks to several years. The pharmaceutical composition may be administered in such a manner that the dose is increased in incremental steps over the administration period.

### Tester

The present invention provides a tester comprising an antibody for at least one of the aforementioned antigens (1) to (8) and (1α) to (55α).

The antibody can be prepared by a conventional method. For example, the antibody may be prepared by immunizing a mammal such as rabbit with one of the aforementioned antigens (1) to (8) and (1α) to (55α).

The antibody may be an Ig antibody, a polyclonal antibody, a monoclonal antibody, or an antigen-binding fragment thereof (e.g., Fab, F(ab′)₂, Fab′).

Further, in the aforementioned tester, the antibody may be provided in a form bound to a carrier. The type of the carrier is not particularly limited as long as it is usable for detection of binding between an antibody and an antigen. Any given carrier known to those skilled in the art can be used.

In one mode, the aforementioned tester is used to determine the presence or absence of an antigen in foods or in products of interest in a food production line. The tester may also be used for quality inspection of production lines and pre-shipment products by manufacturers, or may be used for self-checking of the presence or absence of an antigen in a food of interest by consumers.

Examples of a method for determining the presence or absence of an antigen include a method in which a prepared tester comprising an Ig antibody is contacted with a sample obtained from a food, etc., ELISA or the like method is used to detect whether there is a binding between the Ig antibody and an antigen in the sample, and if the binding between the Ig antibody and the antigen is detected, it is determined that the antibody remains in the food, etc. of interest.

### Allergen-Free Food and the Like

The present invention provides a soybean or processed product of soybean in which at least one of the aforementioned antigens (1) to (8) and (1α) to (55α) is eliminated or reduced.

The method for eliminating or reducing the antigen of the present invention in a soybean or processed products of soybean is not limited. The elimination or reduction of the antigen may be conducted by any method, as long as the method permits the elimination or reduction of the antigen.

For example, the soybean of the present invention whose antigen is eliminated or reduced may be obtained by preparing a soybean in which the expression of the antigen of the present invention is knocked out, using a gene knock-out technique.

An antigen of the present invention may be the artefact that an antigen of the present invention assumed the removal or a reduced soybean raw material as for the removal or the reduced processed products of soybean. In the case of using an ordinary soybean as a source ingredient, a treatment for removing or reducing the antigen of this invention is performed before or after preparation of a processed product of soybean. The methods for removing or reducing the antigen of the present invention include a method to remove protein component in a high pressure treatment and food such as elution with the neutral salt solution or the high temperature steam with an antigen of the present invention in the processed products of soybean which assumed a normal soybean raw material.

### EXAMPLES

The following describes examples of the present invention. The technical scope of this invention is not limited by these examples.

### Example 1: Confirmation of a Protein Pattern

Proteins contained in soybean were investigated using a two-dimensional electrophoresis method described below.

### Protein Extraction

Commercially available dry soybeans (several beans) were powdered. 500 μl of water was added to 50 mg of the powder and shaking extraction was conducted using a vortex mixer at 25° C. for 10 minutes. After the shaking extraction, a liquid protein extract was collected.

Thereafter, the precipitation procedure was repeated twice using a 2D-CleanUP Kit (produced by GE). In the first round of precipitation, the collected liquid protein extract was precipitated by adding TCA (trichloroacetic acid) thereto and the precipitated product produced by this procedure (TCA-precipitated product) was collected. In the second round of precipitation, the TCA-precipitated product collected above was further precipitated by adding acetone thereto and the precipitated product (sample) produced by this procedure was collected.

Preparation of a sample solution Part of the collected sample (50 μg on a protein weight basis) was dissolved in 150 μL of a DeStreak Rehydration Solution (produced by GE), which is a swelling buffer for first-dimensional isoelectric focusing gels, thereby obtaining a sample solution for first-dimensional isoelectric focusing (sample solution for swelling). The constituents of the DeStreak Rehydration Solution are as mentioned below.
7M thiourea
2M urea
4% (w/v) of CHAPS (3-[(3-cholamidopropyl)dimethylammonio]propanesulfonate)
0.5% (v/v) IPG buffer; produced by GE
Moderate amount of BPB (bromophenol blue)

Penetration of the sample into first-dimensional isoelectric focusing gels First-dimensional isoelectric focusing gel strips (Immobiline Drystrip IPG gels (pH3-10NL); produced by GE) were immersed in 140 μL of the foregoing sample solution for first-dimensional isoelectric focusing (sample solution for swelling) and impregnated with the solution at room temperature overnight.

In this example, an IPGphor electrophoresis system produced by GE was used.

An electrophoresis tray was filled with silicone oil. Filter paper moisten with water was positioned at both ends of the gel strips impregnated with the sample, and the gel strips were set in the electrophoresis tray such that the gel strips were covered with silicone oil. Electrodes were placed on the gel strips with the filter paper intervening therebetween.

The maximum current of the isoelectric focusing system was set to 75 μA per gel strip, and the first-dimensional isoelectric focusing was carried out according to the following voltage program: (1) a constant voltage step was performed at a constant voltage of 300 V until the volt-hours reached 750 Vhr (the current variation width during electrophoresis for 30 minutes before the end of this step was 5 μA); (2) the voltage was increased gradually to 1000 V for 300 Vhr; (3) the voltage was further increased gradually to 5000 V for 4500 Vhr; and then (4) the voltage was held at a constant voltage of 5000 V until the total Vhr reached 12000.

### SDS Equilibration of Isoelectric Focusing Gels

After the aforementioned first-dimensional isoelectric focusing was done, the gel strips were taken out of the isoelectric focusing system, immersed in an equilibration buffer containing a reducing agent, and shaken at room temperature for 15 minutes. The constituents of the equilibration buffer containing the reducing agent are as mentioned below.
100 mM Tris-HCl (pH 8.0)
6M urea
30% (v/v) glycerol
2% (w/v) SDS
1% (w/v) DTT

Next, the equilibration buffer containing the reducing agent was removed, and then the gel strips were immersed in an equilibration buffer containing an alkylating agent and shaken at room temperature for 15 minutes to obtain SDS-equilibrated gels. The constituents of the equilibration buffer containing the alkylating agent are as mentioned below.
100 mM Tris-HCl (pH 8.0)
6M urea
30% (v/v) glycerol
2% (w/v) SDS
2.5% (w/v) iodoacetamide

### Second-Dimensional SDS-PAGE

In this example, the XCell SureLock Mini-Cell electrophoresis system produced by Life Technologies was used. The second-dimensional electrophoresis gels used were NuPAGE 4-12% Bis-Tris Gels produced by Life Technologies. Also, an electrophoresis buffer composed of the following constituents was prepared and used.
50 mM MOPS
50 mM Tris base
0.1% (w/v) SDS
1 mM EDTA

Further, an agarose solution for gel adhesion was used in this example, which was prepared by dissolving 0.5% (w/v) Agarose S (produced by Nippon Gene Co., Ltd.) and a moderate amount of BPB (bromophenol blue) in the electrophoresis buffer.

SDS-PAGE wells were washed well with the electrophoresis buffer, and then the buffer used for the washing was removed. Next, the washed wells were charged with the fully dissolved agarose solution for gel adhesion. Next, the SDS-equilibrated gel strips were immersed in agarose and closely adhered to second-dimensional electrophoresis gels using tweezers. After it was confirmed that agarose was fully fixed with the gels being closely adhered to each other, electrophoresis was performed at a constant voltage of 200 V for about 45 minutes.

### Fluorescent Staining of Gels

The gels were fluorescently stained with SYPRO Ruby (produced by Life Technologies).

First, an airtight container to be used was washed well in advance with 98% (v/v) ethanol. The electrophoresed second-dimensional electrophoresis gel strips were taken out of the SDS-PAGE system, placed onto the washed airtight container, and treated twice by immersion in 50% (v/v) methanol and 7% (v/v) aqueous solution containing acetic acid for 30 minutes. Then, a further immersion treatment was done for 10 minutes, with the solution being replaced by water. Next, the second-dimensional electrophoresis gel strips were immersed in 40 mL of SYPRO Ruby and shaken at room temperature overnight. Thereafter, the SYPRO Ruby was removed, and then the second-dimensional electrophoresis gel strips were washed with water and shaken in 10% (v/v) methanol and 7% (v/v) aqueous solution containing acetic acid for 30 minutes. Further shaking was done for at least 30 minutes, with the solution being replaced by water.

### Analysis

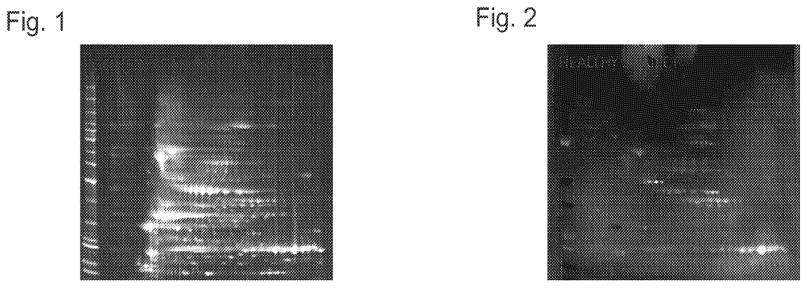

The second-dimensional electrophoresis gels obtained through the foregoing series of treatments were subjected to fluorescent image scanning on Typhoon 9400 (produced by GE). The results of the two-dimensional electrophoresis are shown in FIG. 1. Molecular weight marker bands are found at the left of the panels. The positions of the bands denote particular molecular weights (in KDa).

### Example 2: Identification of Antigens by Immunoblotting (1)

Identification of antigens by immunoblotting was carried out by taking all the steps up to the step of “Second-dimensional SDS-PAGE” as described above in Example 1, followed by the steps of “Transfer to membrane”, “Immunoblotting” and “Analysis” as described below.

### Transfer to Membrane

Transfer to membrane was done using the following transfer system and transfer buffer. Transfer system: XCell SureLock Mini-Cell and XCell II Blot Module (produced by Life Technologies) Transfer buffer: NuPAGE Transfer Buffer (X20) (produced by Life Technologies), used in a form diluted 20-fold with milliQ water.

To be specific, proteins in the two-dimensional electrophoresis gels were transferred to a membrane (PVDF membrane) according to the following procedure.
(1) The PVDF membrane was immersed in 100% methanol followed by milliQ water, and then moved into the transfer buffer to hydrophilize the PVDF membrane.
(2) After sponge, filter paper, the gels treated by second-dimensional SDS-PAGE, the hydrophilized PVDF membrane, filter paper, and sponge were put in place in this order, the transfer system was energized at a constant voltage of 30 V for one hour.

### Immunoblotting

Immunoblotting of the membrane was carried out using, as a primary antibody, a serum sample from a patient with a soybean allergy or a serum sample from a healthy subject.

Immunoblotting of the membrane was carried out according to the following procedure.
(1) The transferred membrane was shaken in a 5% skim milk/PBST solution (a PBS buffer containing 0.3% Tween 20 nonionic surfactant) at room temperature for one hour.
(2) The membrane was left to stand in a solution of 3% primary antibody serum in 5% skim milk/PBST at room temperature for one hour.
(3) The membrane was washed with a PBST solution (5 min.×3 times).
(4) The membrane was left to stand in a 1:2500 dilution of the secondary antibody, anti-human IgE-HRP (horseradish peroxidase), with a 3% skim milk/PBST solution at room temperature for one hour.
(5) The membrane was washed with a PBST solution (5 min.×3 times).
(6) The membrane was left to stand in Pierce Western Blotting Substrate Plus (produced by Thermo Fisher Scientific) for 5 minutes.

### Analysis

The membrane obtained through the foregoing series of treatments was subjected to fluorescent image scanning on Typhoon 9400 (produced by GE).

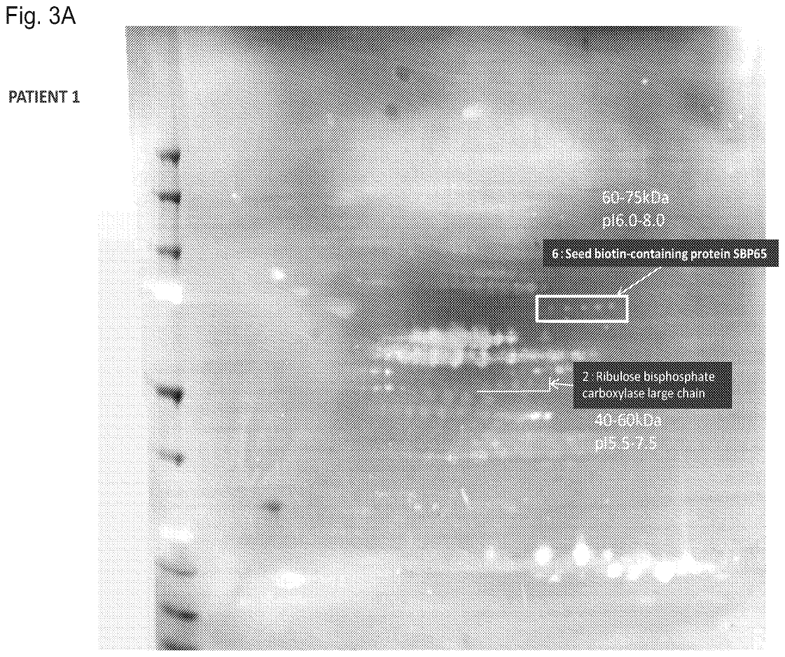

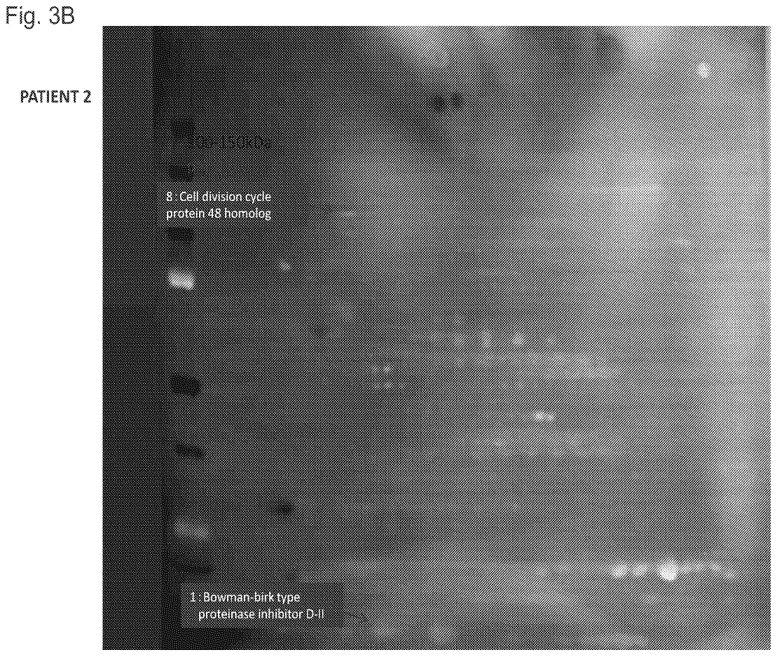

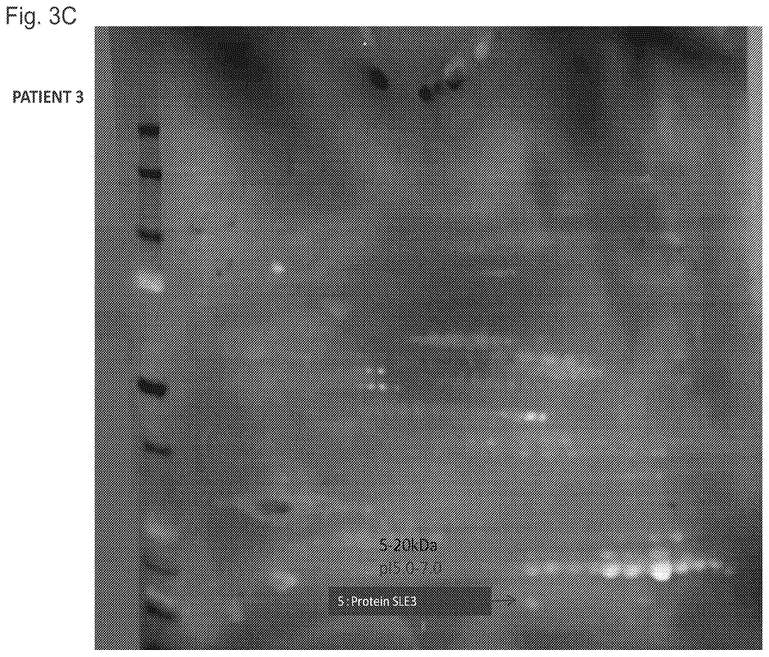

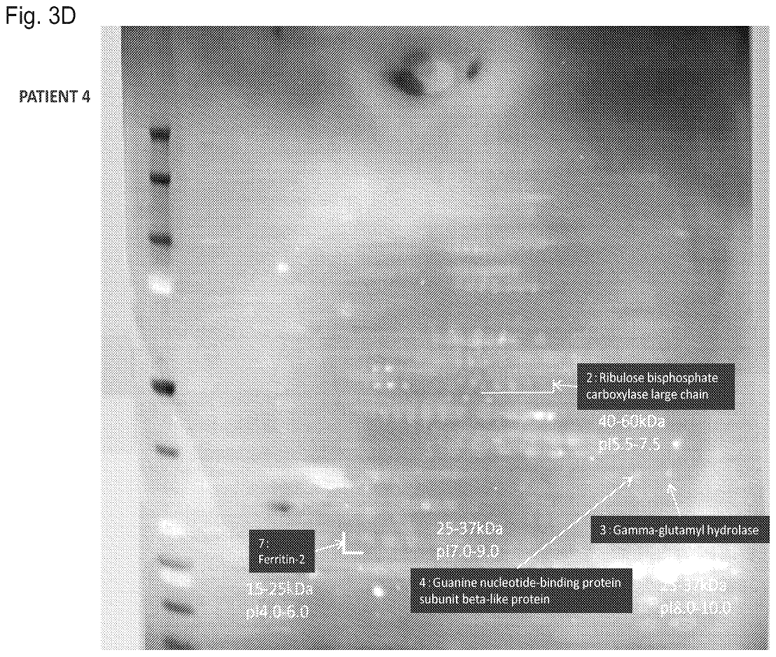

The immunoblots obtained with the serum from the soybean-allergic patient were compared with those obtained with the control serum from the healthy subject. By immunoblotting using the serum from soybean-allergic Patient 1, Patient 2, Patient 3, and Patient 4, 8 spots were detected (FIGS. 3A to D), which are different from the spots detected when the serum of the healthy subject was used (FIG. 2) and different from those of the known soybean allergen proteins.

The molecular weights and isoelectric points of the 8 spots are as follows.
Spot 1: Molecular weight 5 to 20 kDa, pl 4.0 to 6.0
Spot 2: Molecular weight 40 to 60 kDa, pl 5.5 to 7.5
Spot 3: Molecular weight 25 to 37 kDa, pl 8.0 to 10.0
Spot 4: Molecular weight 25 to 37 kDa, pl 7.0 to 9.0
Spot 5: Molecular weight 5 to 20 kDa, pl 5.0 to 7.0
Spot 6: Molecular weight 60 to 75 kDa, pl 6.0 to 8.0
Spot 7: Molecular weight 15 to 25 kDa, pl 4.0 to 6.0
Spot 8: Molecular weight 100 to 150 kDa, pl 4.0 to 6.0

### Example 3: Mass Spectrometry and Identification of Antigens (1)

The amino acid sequences of the antigens that form the above eight protein spots were identified by mass spectroscopy.

To be specific, protein extraction and mass spectroscopy were done by the following procedure.
(1) Soybeans were subjected to protein extraction, two-dimensional electrophoresis and transfer to membrane by following the procedures described in Example 1 and 2, and the resulting membrane was stained by shaking in a solution of 0.008% Direct blue in 40% ethanol and 10% acetic acid.
(2) Then, the membrane was decolorized by repeating a 5-minute treatment with 40% ethanol and 10% acetic acid three times, washed with water for 5 minutes, and then dried by air.
(3) A protein spot of interest was cut out with a clean cutter blade and put into a centrifugal tube. The cut membrane was subjected to hydrophilization with 50 μL of methanol, followed by washing with 100 μL of water twice and then centrifugal cleaning. Thereafter, 20 μL of 20 mM NH₄HCO₃ and 50% acetonitrile were added.
(4) 1 μL of 1 pmol/μL lysyl endopeptidase (produced by WAKO) was added, and the solution was left to stand at 37° C. for 60 minutes and then collected in a new centrifugal tube. After 20 μL of 20 mM NH₄HCO₃ and 70% acetonitrile were added to the membrane, the membrane was immersed therein at room temperature for 10 minutes, and the resulting solution was further collected. The solution was dissolved with 0.1% formic acid and L of 4% acetonitrile and transferred to a tube.
(5) The collected solution was dried under reduced pressure, dissolved with 15 μL of solution A (a 0.1% formic acid/4% acetonitrile solution), and analyzed by mass spectroscopy (ESI-TOF5600, produced by AB Sciex).
(6) Identification of proteins based on the MS data obtained with the mass spectrometer was done by searching the NCBI database.

### Results

The mass spectrometry of the spots resulted in the detection of the following amino acid sequences.
Spot 1: the amino acid sequences set forth in SEQ ID NO: 17 to 20
Spot 2: the amino acid sequences set forth in SEQ ID NO: 21 to 28
Spot 3: the amino acid sequences set forth in SEQ ID NO: 29, 30
Spot 4: the amino acid sequences set forth in SEQ ID NO: 31, 32
Spot 5: the amino acid sequences set forth in SEQ ID NO: 33, 34
Spot 6: the amino acid sequences set forth in SEQ ID NO: 35 to 53
Spot 7: the amino acid sequences set forth in SEQ ID NO: 54 to 61
Spot 8: the amino acid sequences set forth in SEQ ID NO: 62 to 68

Furthermore, the spots were identified as the following proteins by the analysis of the mass data obtained from the mass spectrometer for the spots at NCBI.
Spot 1: Bowman-Birk type proteinase inhibitor D-II (UniProt accession number P01064, amino acid sequence: SEQ ID NO: 2, encoding nucleotide sequence: SEQ ID NO: 1)
Spot 2: Ribulose bisphosphate carboxylase large chain (UniProt accession number P27066, amino acid sequence: SEQ ID NO: 4, encoding nucleotide sequence: SEQ ID NO: 3)
Spot 3: Gamma-glutamyl hydrolase (UniProt accession number P93164, amino acid sequence: SEQ ID NO: 6, encoding nucleotide sequence: SEQ ID NO: 5)
Spot 4: Guanine nucleotide-binding protein subunit beta-like protein (UniProt accession number Q39836, amino acid sequence: SEQ ID NO: 8, encoding nucleotide sequence: SEQ ID NO: 7)
Spot 5: Protein SLE3 (UniProt accession number C6T0L2, amino acid sequence: SEQ ID NO: 10, encoding nucleotide sequence: SEQ ID NO: 9)
Spot 6: Seed biotin-containing protein SBP65 (UniProt accession number Q39846, amino acid sequence: SEQ ID NO: 12, encoding nucleotide sequence: SEQ ID NO: 11)
Spot 7: Ferritin-2 (UniProt accession number Q941C4, amino acid sequence: SEQ ID NO: 14, encoding nucleotide sequence: SEQ ID NO: 13)
Spot 8: Cell division cycle protein 48 homolog (UniProt accession number P54774, amino acid sequence: SEQ ID NO: 16, encoding nucleotide sequence: SEQ ID NO: 15)

### Example 4: Identification of Antigens by Immunoblotting (2)

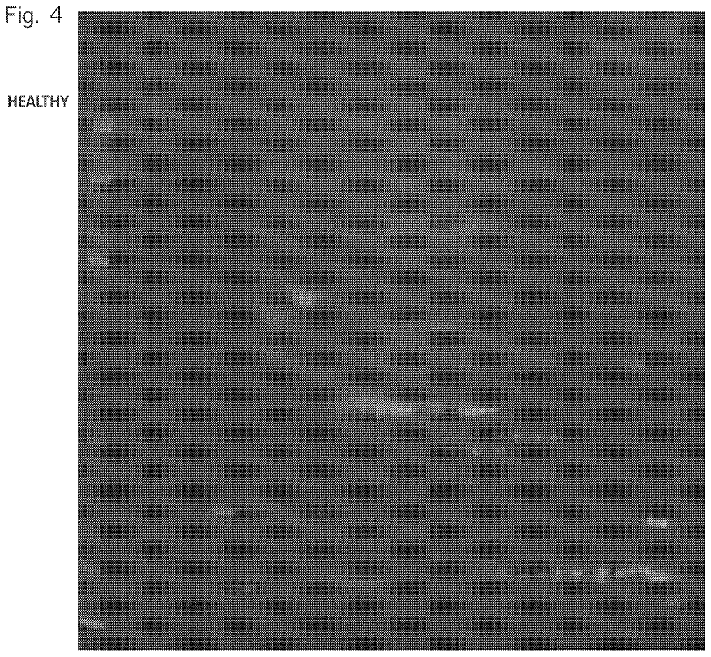

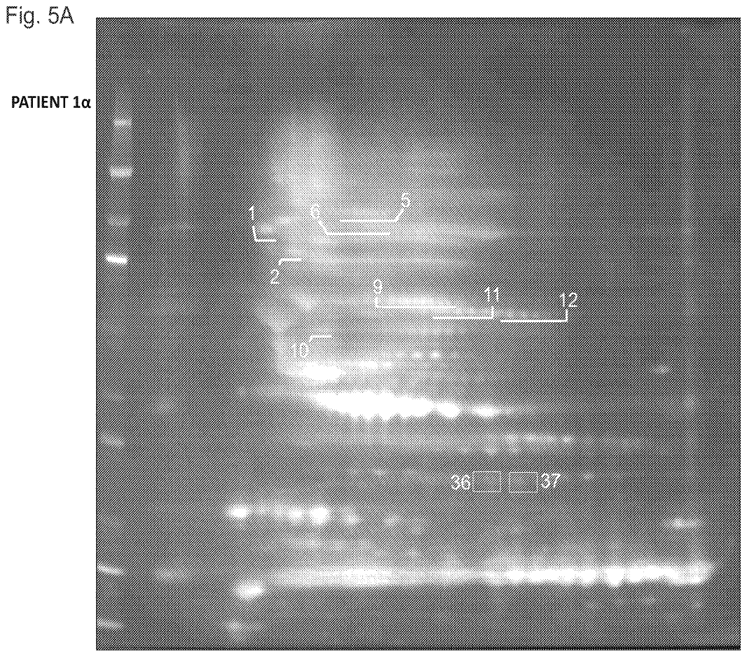

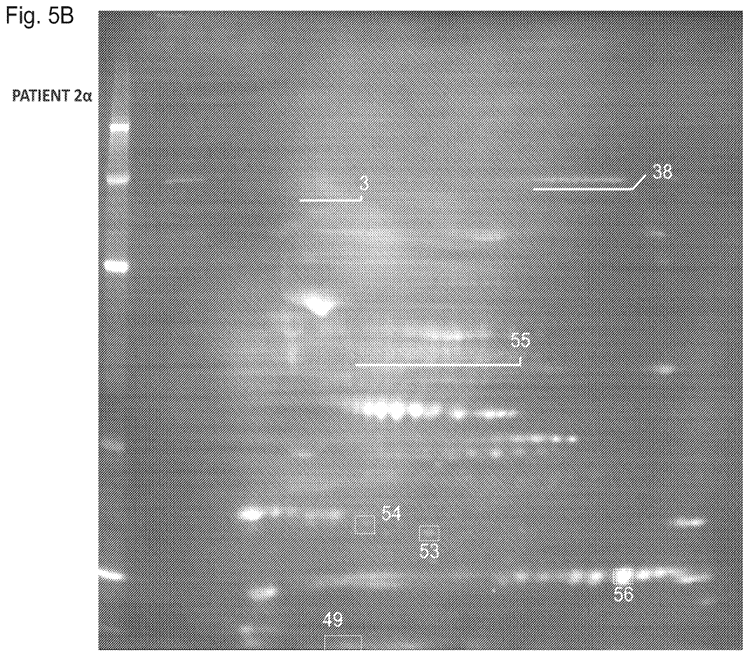

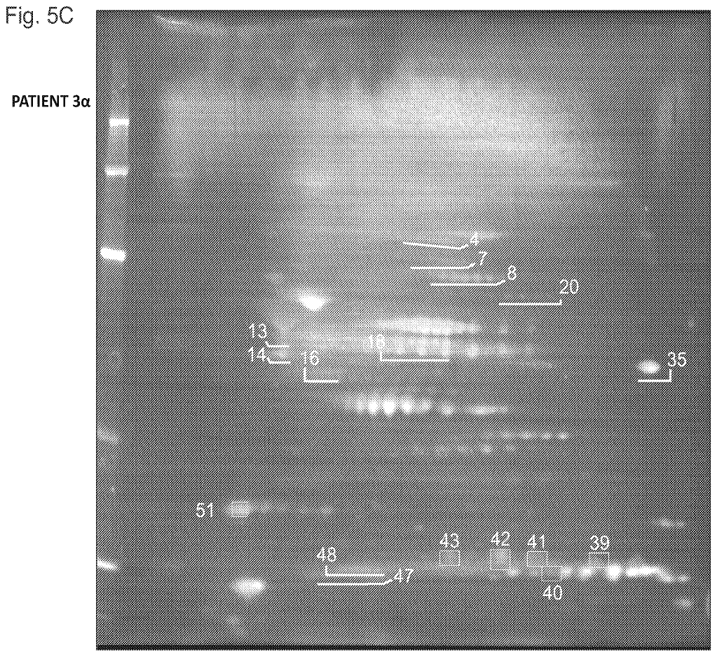

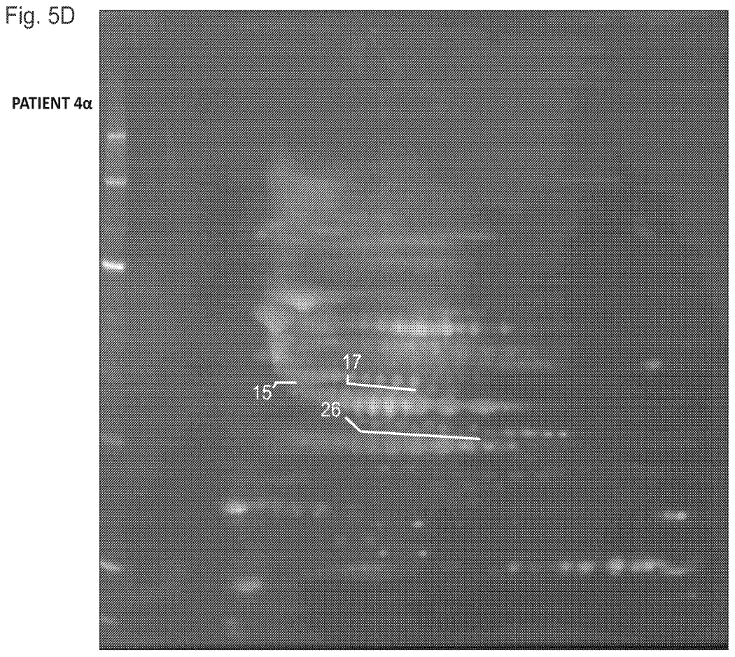

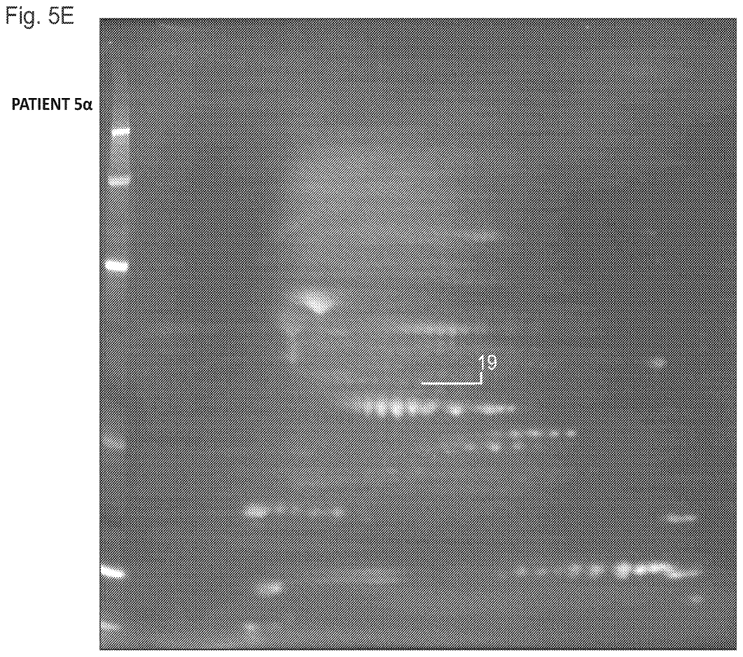

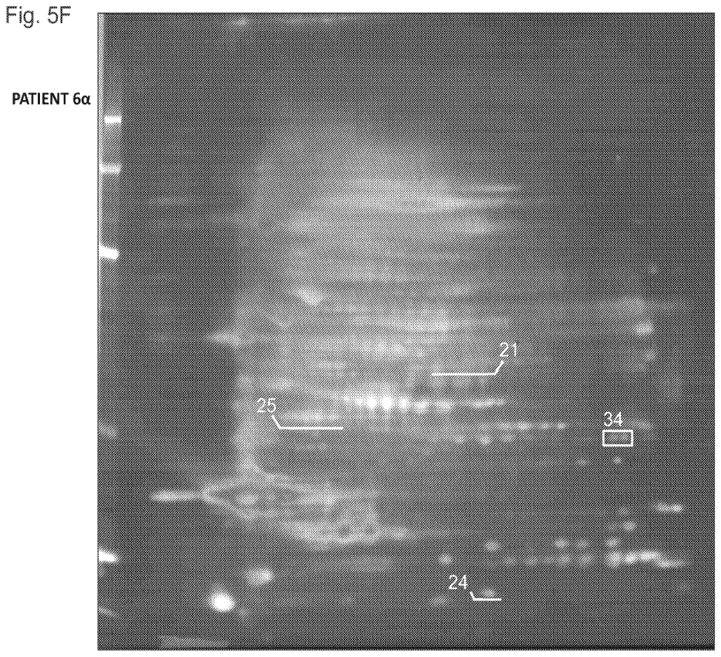

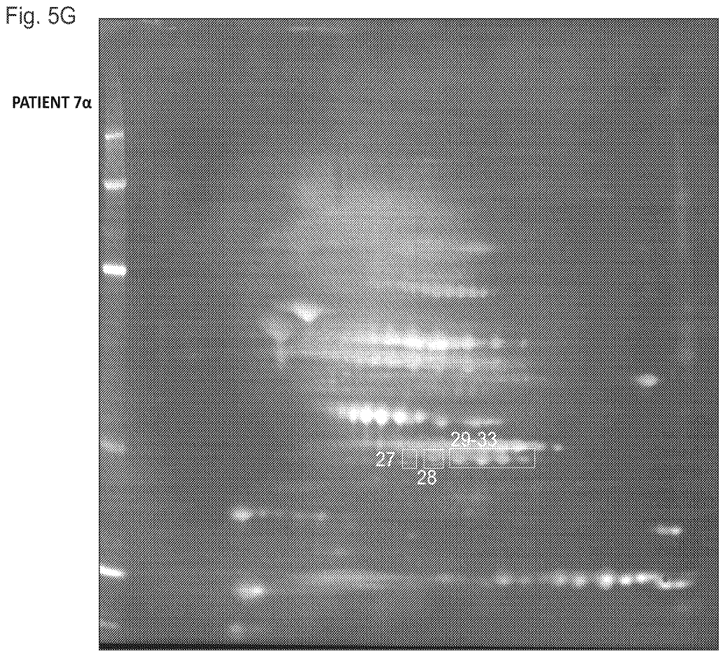

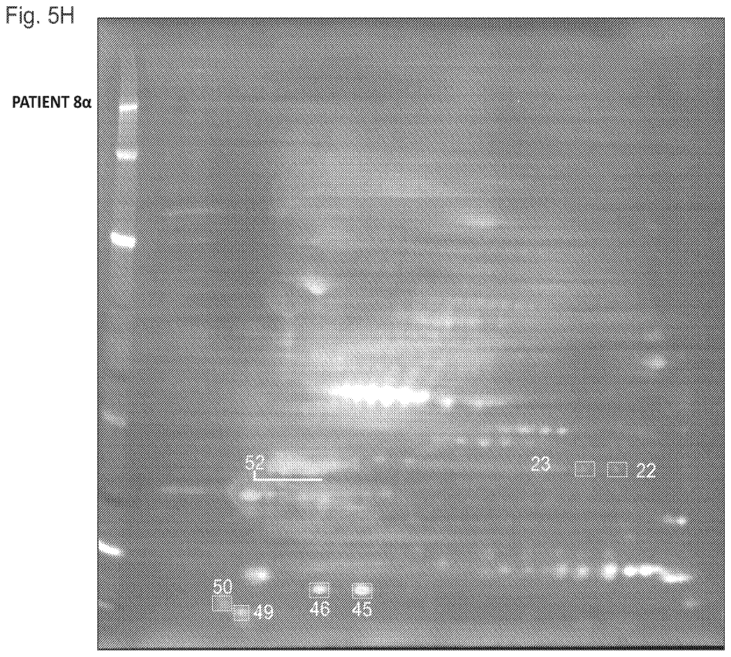

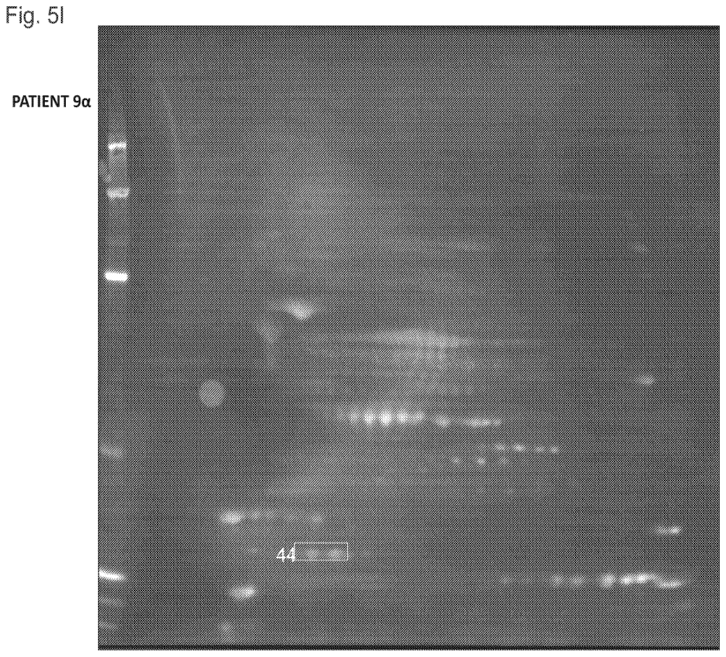

The immunoblots obtained with the serum from the soybean-allergic patient were compared with those obtained with the control serum from the healthy subject, in the same manner as in Example 2. By immunoblotting using the serum from soybean-allergic Patient 1α, Patient 2α, Patient 3α, Patient 4α, Patient 5α, Patient 6α, Patient 7α, Patient 8α, and Patient 9α, 56 spots were detected (FIGS. 5A to I), which are different from the spots detected when the serum of the healthy subject was used (FIG. 4) and different from those of the known soybean allergen proteins.

The molecular weights and isoelectric points of the 56 spots are as follows.
Spot 1α: Molecular weight 80 to 110 kDa, pl 3.0 to 6.0
Spot 2α: Molecular weight 60 to 110 kDa, pl 3.0 to 6.0
Spot 3α: Molecular weight 100 to 150 kDa, pl 4.0 to 6.0
Spot 4α: Molecular weight 80 to 110 kDa, pl 4.0 to 7.0
Spot 5α: Molecular weight 80 to 160 kDa, pl 4.0 to 7.0
Spot 6α: Molecular weight 80 to 110 kDa, pl 4.0 to 7.0
Spot 7α: Molecular weight 60 to 110 kDa, pl 4.0 to 7.0
Spot 8α: Molecular weight 60 to 110 kDa, pl 4.0 to 8.0
Spot 9α: Molecular weight 60 to 110 kDa, pl 4.0 to 8.0
Spot 10α: Molecular weight 40 to 80 kDa, pl 4.0 to 7.0
Spot 11α: Molecular weight 40 to 80 kDa, pl 4.0 to 8.0
Spot 12α: Molecular weight 40 to 80 kDa, pl 4.0 to 8.0
Spot 13α: Molecular weight 40 to 80 kDa, pl 4.0 to 7.0
Spot 14α: Molecular weight 40 to 80 kDa, pl 3.0 to 6.0
Spot 15α, 16α: Molecular weight 30 to 80 kDa, pl 4.0 to 7.0
Spot 17α: Molecular weight 30 to 80 kDa, pl 4.0 to 7.0
Spot 18α: Molecular weight 40 to 80 kDa, pl 4.0 to 8.0
Spot 19α: Molecular weight 40 to 80 kDa, pl 4.0 to 8.0
Spot 20α: Molecular weight 60 to 75 kDa, pl 6.0 to 8.0
Spot 21α: Molecular weight 40 to 60 kDa, pl 5.0 to 8.0
Spot 22α: Molecular weight 25 to 37 kDa, pl 8.0 to 10.0
Spot 23α: Molecular weight 25 to 37 kDa, pl 7.0 to 9.0
Spot 24α: Molecular weight 5 to 20 kDa, pl 5.0 to 7.0
Spot 25α: Molecular weight 30 to 80 kDa, pl 4.0 to 7.0
Spot 26α: Molecular weight 30 to 80 kDa, pl 4.0 to 7.0
Spot 27α: Molecular weight 30 to 80 kDa, pl 4.0 to 7.0
Spot 28α: Molecular weight 20 to 60 kDa, pl 4.0 to 7.0
Spot 29α, 30α, 31α, 32α, 33α: Molecular weight 20 to 60 kDa, pl 4.0 to 8.0
Spot 34α: Molecular weight 20 to 60 kDa, pl 6.0 to 10.0
Spot 35α: Molecular weight 30 to 80 kDa, pl 6.0 to 10.0
Spot 36α, 37α: Molecular weight 20 to 60 kDa, pl 4.0 to 8.0
Spot 38α: Molecular weight 80 to 160 kDa, pl 6.0 to 10.0
Spot 39α: Molecular weight 10 to 50 kDa, pl 6.0 to 10.0
Spot 40α, 56α: Molecular weight 10 to 50 kDa, pl 6.0 to 10.0
Spot 41α: Molecular weight 10 to 50 kDa, pl 6.0 to 10.0
Spot 42α, 43α: Molecular weight 10 to 50 kDa, pl 6.0 to 10.0
Spot 44α: Molecular weight 10 to 50 kDa, pl 4.0 to 7.0
Spot 45α: Molecular weight 5 to 40 kDa, pl 4.0 to 7.0
Spot 46α: Molecular weight 5 to 40 kDa, pl 4.0 to 7.0
Spot 47α: Molecular weight 5 to 40 kDa, pl 4.0 to 7.0
Spot 48α: Molecular weight 5 to 40 kDa, pl 4.0 to 8.0
Spot 49α: Molecular weight 5 to 20 kDa, pl 4.0 to 7.0
Spot 50α: Molecular weight 5 to 40 kDa, pl 3.0 to 6.0
Spot 51α: Molecular weight 10 to 50 kDa, pl 3.0 to 6.0
Spot 52α: Molecular weight 20 to 60 kDa, pl 4.0 to 7.0
Spot 53α: Molecular weight 10 to 50 kDa, pl 4.0 to 8.0
Spot 54α: Molecular weight 10 to 50 kDa, pl 4.0 to 8.0
Spot 55α: Molecular weight 40 to 80 kDa, pl 6.0 to 10.0

### Example 5: Mass Spectrometry and Identification of Antigens (2)

The amino acid sequences of the antigens that form the 56 protein spots were identified by mass spectroscopy, in the same manner as in Example 3.

The mass spectrometry of the spots resulted in the detection of the following amino acid sequences. Furthermore, the spots were identified as the following proteins by the analysis of the mass data obtained from the mass spectrometer for the spots at NCBI.

### Spot 1α:

The amino acid sequences set forth in SEQ ID NOs: 71 to 87 were detected by the mass spectrometry. The protein identified from this mass data was endoplasmin homolog isoform X2 (NCBI accession number XP_006596543.1, amino acid sequence: SEQ ID NO: 70, encoding nucleotide sequence: SEQ ID NO: 69).

The amino acid sequences set forth in SEQ ID NOs: 90 to 104 were detected by the mass spectrometry. The protein identified from this mass data was endoplasmin homolog isoform X2 (NCBI accession number XP_006601356.1, amino acid sequence: SEQ ID NO: 89, encoding nucleotide sequence: SEQ ID NO: 88).

### Spot 2α

The amino acid sequences set forth in SEQ ID NOs: 107 to 120 were detected by the mass spectrometry. The protein identified from this mass data was heat shock cognate protein 80 (NCBI accession number XP_003544594.1, amino acid sequence: SEQ ID NO: 106, encoding nucleotide sequence: SEQ ID NO: 105).

The amino acid sequences set forth in SEQ ID NOs: 123 to 137 were detected by the mass spectrometry. The protein identified from this mass data was heat shock protein 90-1 (NCBI accession number NP_001236612.1, amino acid sequence: SEQ ID NO: 122, encoding nucleotide sequence: SEQ ID NO: 121).

The amino acid sequences set forth in SEQ ID NOs: 140 to 153 were detected by the mass spectrometry. The protein identified from this mass data was hypothetical protein GLYMA_02G302500 (NCBI accession number KRH73936.1, amino acid sequence: SEQ ID NO: 139, encoding nucleotide sequence: SEQ ID NO: 138).

### Spot 3α:

The amino acid sequences set forth in SEQ ID NOs: 62 to 68 were detected by the mass spectrometry, as in Spot 8 of Example 3. The protein identified from this mass data was Cell division cycle protein 48 homolog (UniProt accession number P54774, amino acid sequence: SEQ ID NO: 16, encoding nucleotide sequence: SEQ ID NO: 15).

### Spot 4α:

The amino acid sequences set forth in SEQ ID NOs: 156, 157 were detected by the mass spectrometry. The protein identified from this mass data was embryo-specific urease isoform X1 (NCBI accession number XP_014630847.1, amino acid sequence: SEQ ID NO: 155, encoding nucleotide sequence: SEQ ID NO: 154).

### Spot 5α:

The amino acid sequences set forth in SEQ ID NOs: 160, 161 were detected by the mass spectrometry. The protein identified from this mass data was alpha-1,4 glucan phosphorylase L isozyme, chloroplastic/amyloplastic (NCBI accession number XP_006603904.1, amino acid sequence: SEQ ID NO: 159, encoding nucleotide sequence: SEQ ID NO: 158).

### Spot 6α:

The amino acid sequences set forth in SEQ ID NOs: 164 to 167 were detected by the mass spectrometry. The protein identified from this mass data was aconitate hydratase 1 (NCBI accession number XP_003537655.1, amino acid sequence: SEQ ID NO: 163, encoding nucleotide sequence: SEQ ID NO: 162).

The amino acid sequences set forth in SEQ ID NOs: 170 to 172 were detected by the mass spectrometry. The protein identified from this mass data was aconitate hydratase 1 (NCBI accession number XP_003517155.1, amino acid sequence: SEQ ID NO: 169, encoding nucleotide sequence: SEQ ID NO: 168).

### Spot 7α:

The amino acid sequences set forth in SEQ ID NOs: 175 to 187 were detected by the mass spectrometry. The protein identified from this mass data was methionine synthase (NCBI accession number NP_001235794.1, amino acid sequence: SEQ ID NO: 174, encoding nucleotide sequence: SEQ ID NO: 173).

### Spot 8α:

The amino acid sequences set forth in SEQ ID NOs: 190 to 199 were detected by the mass spectrometry. The protein identified from this mass data was transketolase, chloroplastic (NCBI accession number XP_003521870.1, amino acid sequence: SEQ ID NO: 189, encoding nucleotide sequence: SEQ ID NO: 188).

### Spot 9α:

The amino acid sequence set forth in SEQ ID NO: 202 was detected by the mass spectrometry. The protein identified from this mass data was lysine-tRNA ligase, cytoplasmic-like isoform X2 (NCBI accession number XP_014625509.1, amino acid sequence: SEQ ID NO: 201, encoding nucleotide sequence: SEQ ID NO: 200).

### Spot 10α

The amino acid sequences set forth in SEQ ID NOs: 205 to 218 were detected by the mass spectrometry. The protein identified from this mass data was chaperonin CPN60-2, mitochondrial (NCBI accession number XP_003556325.1, amino acid sequence: SEQ ID NO: 204, encoding nucleotide sequence: SEQ ID NO: 203).

The amino acid sequences set forth in SEQ ID NOs: 221 to 231 were detected by the mass spectrometry. The protein identified from this mass data was chaperonin CPN60-2, mitochondrial-like isoform X1 (NCBI accession number XP_003535967.1, amino acid sequence: SEQ ID NO: 220, encoding nucleotide sequence: SEQ ID NO: 219).

### Spot 11α:

The amino acid sequences set forth in SEQ ID NOs: 234 to 237 were detected by the mass spectrometry. The protein identified from this mass data was polyadenylate-binding protein 8 isoform X3 (NCBI accession number XP_006578034.1, amino acid sequence: SEQ ID NO: 233, encoding nucleotide sequence: SEQ ID NO: 232).

### Spot 12α:

The amino acid sequence set forth in SEQ ID NO: 240 was detected by the mass spectrometry. The protein identified from this mass data was pyrophosphate-fructose 6-phosphate 1-phosphotransferase subunit alpha-like (NCBI accession number XP_003555655.1, amino acid sequence: SEQ ID NO: 239, encoding nucleotide sequence: SEQ ID NO: 238).

### Spot 13α:

The amino acid sequences set forth in SEQ ID NOs: 243 to 252 were detected by the mass spectrometry. The protein identified from this mass data was protein disulfide isomerase-like protein precursor (NCBI accession number NP_001238009.1, amino acid sequence: SEQ ID NO: 242, encoding nucleotide sequence: SEQ ID NO: 241).

The amino acid sequences set forth in SEQ ID NOs: 255 to 278 were detected by the mass spectrometry. The protein identified from this mass data was protein disulfide-isomerase (NCBI accession number XP_006581588.1, amino acid sequence: SEQ ID NO: 254, encoding nucleotide sequence: SEQ ID NO: 253).

### Spot 14α:

The amino acid sequences set forth in SEQ ID NOs: 281 to 284 were detected by the mass spectrometry. The protein identified from this mass data was V-type proton ATPase subunit B 2 (NCBI accession number XP_003552473.1, amino acid sequence: SEQ ID NO: 280, encoding nucleotide sequence: SEQ ID NO: 279).

The amino acid sequences set forth in SEQ ID NOs: 287 to 289 were detected by the mass spectrometry. The protein identified from this mass data was V-type proton ATPase subunit B 2 (NCBI accession number XP_006605857.1, amino acid sequence: SEQ ID NO: 286, encoding nucleotide sequence: SEQ ID NO: 275).

### Spots 15α, 16α:

The amino acid sequences set forth in SEQ ID NOs: 292 to 304 were detected by the mass spectrometry. The protein identified from this mass data was UTP-glucose-1-phosphate uridylyltransferase (NCBI accession number XP_003544964.1, amino acid sequence: SEQ ID NO: 291, encoding nucleotide sequence: SEQ ID NO: 290).

The amino acid sequences set forth in SEQ ID NOs: 307 to 318 were detected by the mass spectrometry. The protein identified from this mass data was hypothetical protein GLYMA_02G241100 (NCBI accession number KRH72929.1, amino acid sequence: SEQ ID NO: 306, encoding nucleotide sequence: SEQ ID NO: 305).

### Spot 17α:

The amino acid sequences set forth in SEQ ID NOs: 321 to 325 were detected by the mass spectrometry. The protein identified from this mass data was guanosine nucleotide diphosphate dissociation inhibitor 2 (NCBI accession number XP_003531293.1, amino acid sequence: SEQ ID NO: 320, encoding nucleotide sequence: SEQ ID NO: 319).

The amino acid sequences set forth in SEQ ID NOs: 328 to 332 were detected by the mass spectrometry. The protein identified from this mass data was rab GDP dissociation inhibitor alpha-like (NCBI accession number NP_001276273.1, amino acid sequence: SEQ ID NO: 327, encoding nucleotide sequence: SEQ ID NO: 326).

### Spot 18α:

The amino acid sequences set forth in SEQ ID NOs: 335 to 339 were detected by the mass spectrometry. The protein identified from this mass data was hypothetical protein GLYMA_10G028300 (NCBI accession number KRH32039.1, amino acid sequence: SEQ ID NO: 334, encoding nucleotide sequence: SEQ ID NO: 333).

### Spot 19α:

The amino acid sequences set forth in SEQ ID NOs: 342 to 346 were detected by the mass spectrometry. The protein identified from this mass data was sucrose binding protein homolog S-64 (NCBI accession number AAF05723.1, amino acid sequence: SEQ ID NO: 341, encoding nucleotide sequence: SEQ ID NO: 340).

### Spot 20α:

The amino acid sequences set forth in SEQ ID NOs: 35 to 53 were detected by the mass spectrometry, as in Spot 6 of Example 3. The protein identified from this mass data was Seed biotin-containing protein SBP65 (UniProt accession number Q39846, amino acid sequence: SEQ ID NO: 12, encoding nucleotide sequence: SEQ ID NO: 11).

### Spot 21α:

The amino acid sequences set forth in SEQ ID NOs: 21 to 28 were detected by the mass spectrometry, as in Spot 2 of Example 3. The protein identified from this mass data was Ribulose bisphosphate carboxylase large chain (UniProt accession number P27066, amino acid sequence: SEQ ID NO: 4, encoding nucleotide sequence: SEQ ID NO: 3).

### Spot 22α:

The amino acid sequences set forth in SEQ ID NOs: 29, 30 were detected by the mass spectrometry, as in Spot 3 of Example 3. The protein identified from this mass data was Gamma-glutamyl hydrolase (UniProt accession number P93164, amino acid sequence: SEQ ID NO: 6, encoding nucleotide sequence: SEQ ID NO: 5).

### Spot 23α:

The amino acid sequences set forth in SEQ ID NOs: 31, 32 were detected by the mass spectrometry, as in Spot 4 of Example 3. The protein identified from this mass data was Guanine nucleotide-binding protein subunit beta-like protein (UniProt accession number Q39836, amino acid sequence: SEQ ID NO: 8, encoding nucleotide sequence: SEQ ID NO: 7).

### Spot 24α:

The amino acid sequences set forth in SEQ ID NOs: 33, 34 were detected by the mass spectrometry, as in Spot 5 of Example 3. The protein identified from this mass data was Protein SLE3 (UniProt accession number C6T0L2, amino acid sequence: SEQ ID NO: 10, encoding nucleotide sequence: SEQ ID NO: 9).

### Spot 25α:

The amino acid sequences set forth in SEQ ID NOs: 349 to 356 were detected by the mass spectrometry. The protein identified from this mass data was succinyl-CoA ligase [ADP-forming]subunit beta, mitochondrial (NCBI accession number XP_003537078.1, amino acid sequence: SEQ ID NO: 348, encoding nucleotide sequence: SEQ ID NO: 347).

The amino acid sequences set forth in SEQ ID NOs: 359 to 365 were detected by the mass spectrometry. The protein identified from this mass data was succinyl-CoA ligase [ADP-forming]subunit beta, mitochondrial (NCBI accession number XP_003542431.1, amino acid sequence: SEQ ID NO: 358, encoding nucleotide sequence: SEQ ID NO: 357).

### Spot 26α:

The amino acid sequences set forth in SEQ ID NOs: 368 to 382 were detected by the mass spectrometry. The protein identified from this mass data was phosphoglycerate kinase, cytosolic (NCBI accession number XP_003546821.1, amino acid sequence: SEQ ID NO: 367, encoding nucleotide sequence: SEQ ID NO: 366).

The amino acid sequences set forth in SEQ ID NOs: 385 to 392 were detected by the mass spectrometry. The protein identified from this mass data was phosphoglycerate kinase, cytosolic (NCBI accession number XP_003531482.1, amino acid sequence: SEQ ID NO: 384, encoding nucleotide sequence: SEQ ID NO: 383).

The amino acid sequences set forth in SEQ ID NOs: 395 to 409 were detected by the mass spectrometry. The protein identified from this mass data was phosphoglycerate kinase, cytosolic (NCBI accession number XP_003531483.1, amino acid sequence: SEQ ID NO: 394, encoding nucleotide sequence: SEQ ID NO: 393).

### Spot 27α:

The amino acid sequences set forth in SEQ ID NOs: 412 to 416 were detected by the mass spectrometry. The protein identified from this mass data was alcohol dehydrogenase 1 (NCBI accession number XP_003526673.1, amino acid sequence: SEQ ID NO: 411, encoding nucleotide sequence: SEQ ID NO: 410).

The amino acid sequences set forth in SEQ ID NOs: 419, 420 were detected by the mass spectrometry. The protein identified from this mass data was alcohol dehydrogenase 1 (NCBI accession number XP_003545664.3, amino acid sequence: SEQ ID NO: 418, encoding nucleotide sequence: SEQ ID NO: 417).

The amino acid sequences set forth in SEQ ID NOs: 423 to 427 were detected by the mass spectrometry. The protein identified from this mass data was alcohol dehydrogenase 1-like (NCBI accession number XP_003544738.1, amino acid sequence: SEQ ID NO: 422, encoding nucleotide sequence: SEQ ID NO: 421).

### Spot 28α:

The amino acid sequences set forth in SEQ ID NOs: 430 to 433 were detected by the mass spectrometry. The protein identified from this mass data was 35 kDa seed maturation protein isoform X1 (NCBI accession number XP_006576267.1, amino acid sequence: SEQ ID NO: 429, encoding nucleotide sequence: SEQ ID NO: 428).

### Spots 29α, 30α, 31α, 32α, 33α:

The amino acid sequences set forth in SEQ ID NOs: 436 to 439 were detected by the mass spectrometry. The protein identified from this mass data was glyceraldehyde-3-phosphate dehydrogenase isoform X1 (NCBI accession number XP_014631598.1, amino acid sequence: SEQ ID NO: 435, encoding nucleotide sequence: SEQ ID NO: 434).

The amino acid sequences set forth in SEQ ID NOs: 442 to 445 were detected by the mass spectrometry. The protein identified from this mass data was glyceraldehyde-3-phosphate dehydrogenase, cytosolic (NCBI accession number XP_006578692.1, amino acid sequence: SEQ ID NO: 441, encoding nucleotide sequence: SEQ ID NO: 440).

The amino acid sequences set forth in SEQ ID NOs: 448 to 451 were detected by the mass spectrometry. The protein identified from this mass data was uncharacterized protein LOC100782924 (NCBI accession number NP_001240046.1, amino acid sequence: SEQ ID NO: 447, encoding nucleotide sequence: SEQ ID NO: 446).

### Spot 34α:

The amino acid sequence set forth in SEQ ID NO: 454 was detected by the mass spectrometry. The protein identified from this mass data was bifunctional UDP-glucose 4-epimerase and UDP-xylose 4-epimerase 1-like (NCBI accession number XP_003532591.1, amino acid sequence: SEQ ID NO: 453, encoding nucleotide sequence: SEQ ID NO: 452).

The amino acid sequences set forth in SEQ ID NOs: 457, 458 were detected by the mass spectrometry. The protein identified from this mass data was uncharacterized protein LOC100803483 (NCBI accession number NP_001241075.1, amino acid sequence: SEQ ID NO: 456, encoding nucleotide sequence: SEQ ID NO: 455).

### Spot 35α:

The amino acid sequences set forth in SEQ ID NOs: 461 to 466 were detected by the mass spectrometry. The protein identified from this mass data was elongation factor 1-alpha (NCBI accession number XP_003553292.1, amino acid sequence: SEQ ID NO: 460, encoding nucleotide sequence: SEQ ID NO: 459).

The amino acid sequences set forth in SEQ ID NOs: 469 to 471 were detected by the mass spectrometry. The protein identified from this mass data was elongation factor 1-alpha (NCBI accession number XP_003547695.1, amino acid sequence: SEQ ID NO: 468, encoding nucleotide sequence: SEQ ID NO: 467).

The amino acid sequences set forth in SEQ ID NOs: 474 to 477 were detected by the mass spectrometry. The protein identified from this mass data was elongation factor-1A isoform X1 (NCBI accession number XP_006600131.1, amino acid sequence: SEQ ID NO: 473, encoding nucleotide sequence: SEQ ID NO: 472).

### Spots 36α, 37α:

The amino acid sequences set forth in SEQ ID NOs: 480 to 483 were detected by the mass spectrometry. The protein identified from this mass data was seed maturation protein PM34 (NCBI accession number NP_001238285.1, amino acid sequence: SEQ ID NO: 479, encoding nucleotide sequence: SEQ ID NO: 478).

The amino acid sequences set forth in SEQ ID NOs: 486, 487 were detected by the mass spectrometry. The protein identified from this mass data was uncharacterized protein LOC100809384 (NCBI accession number NP_001241158.1, amino acid sequence: SEQ ID NO: 485, encoding nucleotide sequence: SEQ ID NO: 484).

The amino acid sequences set forth in SEQ ID NOs: 490 to 493 were detected by the mass spectrometry. The protein identified from this mass data was hypothetical protein GLYMA_10G155300 (NCBI accession number KRH33956.1, amino acid sequence: SEQ ID NO: 489, encoding nucleotide sequence: SEQ ID NO: 488).

### Spot 38α

The amino acid sequences set forth in SEQ ID NOs: 496 to 513 were detected by the mass spectrometry. The protein identified from this mass data was seed biotin-containing protein SBP65-like isoform X1 (NCBI accession number XP_003526237.1, amino acid sequence: SEQ ID NO: 495, encoding nucleotide sequence: SEQ ID NO: 494).

### Spot 39α:

The amino acid sequence set forth in SEQ ID NO: 516 was detected by the mass spectrometry. The protein identified from this mass data was ATP synthase subunit 0, mitochondrial-like (NCBI accession number XP_003546884.1, amino acid sequence: SEQ ID NO: 515, encoding nucleotide sequence: SEQ ID NO: 514).

### Spots 40α, 56α:

The amino acid sequences set forth in SEQ ID NOs: 519, 520 were detected by the mass spectrometry. The protein identified from this mass data was P24 oleosin isoform A (NCBI accession number XP_003553822.1, amino acid sequence: SEQ ID NO: 518, encoding nucleotide sequence: SEQ ID NO: 517).

### Spot 41α:

The amino acid sequence set forth in SEQ ID NO: 523 was detected by the mass spectrometry. The protein identified from this mass data was uncharacterized protein At3g49720-like isoform X2 (NCBI accession number XP_006600908.1, amino acid sequence: SEQ ID NO: 522, encoding nucleotide sequence: SEQ ID NO: 521).

### Spots 42α, 43α

The amino acid sequences set forth in SEQ ID NOs: 526 to 531 were detected by the mass spectrometry. The protein identified from this mass data was superoxide dismutase [Mn], mitochondrial (NCBI accession number XP_003526813.1, amino acid sequence: SEQ ID NO: 525, encoding nucleotide sequence: SEQ ID NO: 524).

The amino acid sequences set forth in SEQ ID NOs: 534 to 539 were detected by the mass spectrometry. The protein identified from this mass data was hypothetical protein GLYMA_04G221300 (NCBI accession number KRH64185.1, amino acid sequence: SEQ ID NO: 533, encoding nucleotide sequence: SEQ ID NO: 532).

### Spot 44α:

The amino acid sequences set forth in SEQ ID NOs: 54 to 61 were detected by the mass spectrometry, as in Spot 7 of Example 3. The protein identified from this mass data was Ferritin-2 (UniProt accession number Q94|C4, amino acid sequence: SEQ ID NO: 14, encoding nucleotide sequence: SEQ ID NO: 13).

### Spot 45α:

The amino acid sequences set forth in SEQ ID NOs: 542 to 545 were detected by the mass spectrometry. The protein identified from this mass data was uncharacterized protein LOC100499771 (NCBI accession number NP_001235797.1, amino acid sequence: SEQ ID NO: 541, encoding nucleotide sequence: SEQ ID NO: 540).

The amino acid sequences set forth in SEQ ID NOs: 548 to 551 were detected by the mass spectrometry. The protein identified from this mass data was hypothetical protein GLYMA_09G192800 (NCBI accession number KRH39322.1, amino acid sequence: SEQ ID NO: 547, encoding nucleotide sequence: SEQ ID NO: 546).

### Spot 46α:

The amino acid sequence set forth in SEQ ID NO: 554 was detected by the mass spectrometry. The protein identified from this mass data was seed maturation protein PM22 (NCBI accession number NP_001237935.1, amino acid sequence: SEQ ID NO: 553, encoding nucleotide sequence: SEQ ID NO: 552).

### Spot 47α:

The amino acid sequences set forth in SEQ ID NOs: 557, 558 were detected by the mass spectrometry. The protein identified from this mass data was eukaryotic translation initiation factor 5A-2 (NCBI accession number XP_003549690.1, amino acid sequence: SEQ ID NO: 556, encoding nucleotide sequence: SEQ ID NO: 555).

The amino acid sequences set forth in SEQ ID NOs: 561 to 563 were detected by the mass spectrometry. The protein identified from this mass data was uncharacterized protein LOC100305510 (NCBI accession number NP_001236395.1, amino acid sequence: SEQ ID NO: 560, encoding nucleotide sequence: SEQ ID NO: 559).

### Spot 48α:

The amino acid sequences set forth in SEQ ID NOs: 566 to 570 were detected by the mass spectrometry. The protein identified from this mass data was uncharacterized protein LOC100306570 (NCBI accession number NP_001236895.1, amino acid sequence: SEQ ID NO: 565, encoding nucleotide sequence: SEQ ID NO: 564).

### Spot 49α:

The amino acid sequences set forth in SEQ ID NOs: 17 to 20 were detected by the mass spectrometry, as in Spot 1 of Example 3. The protein identified from this mass data was Bowman-Birk type proteinase inhibitor D-II (UniProt accession number P01064, amino acid sequence: SEQ ID NO: 2, encoding nucleotide sequence: SEQ ID NO: 1).

### Spot 50α:

The amino acid sequences set forth in SEQ ID NOs: 573 to 576 were detected by the mass spectrometry. The protein identified from this mass data was uncharacterized protein LOC100813859 (NCBI accession number NP_001239816.1, amino acid sequence: SEQ ID NO: 572, encoding nucleotide sequence: SEQ ID NO: 571).

### Spot 51α:

The amino acid sequences set forth in SEQ ID NOs: 579 to 582 were detected by the mass spectrometry. The protein identified from this mass data was 14-3-3-like protein (NCBI accession number XP_003526571.1, amino acid sequence: SEQ ID NO: 578, encoding nucleotide sequence: SEQ ID NO: 577).

### Spot 52α:

The amino acid sequences set forth in SEQ ID NOs: 585 to 588 were detected by the mass spectrometry. The protein identified from this mass data was probable fructokinase-4 (NCBI accession number XP_003543564.1, amino acid sequence: SEQ ID NO: 584, encoding nucleotide sequence: SEQ ID NO: 583).

### Spot 53α:

The amino acid sequences set forth in SEQ ID NOs: 591 to 597 were detected by the mass spectrometry. The protein identified from this mass data was triosephosphate isomerase isoform X1 (NCBI accession number XP_006593480.2, amino acid sequence: SEQ ID NO: 590, encoding nucleotide sequence: SEQ ID NO: 589).

The amino acid sequences set forth in SEQ ID NOs: 600 to 604 were detected by the mass spectrometry. The protein identified from this mass data was triosephosphate isomerase, cytosolic (NCBI accession number XP_003547334.1, amino acid sequence: SEQ ID NO: 599, encoding nucleotide sequence: SEQ ID NO: 598).

### Spot 54α:

The amino acid sequences set forth in SEQ ID NOs: 607 to 611 were detected by the mass spectrometry. The protein identified from this mass data was superoxide dismutase [Fe], chloroplastic isoform X1 (NCBI accession number XP_014627751.1, amino acid sequence: SEQ ID NO: 606, encoding nucleotide sequence: SEQ ID NO: 605).

The amino acid sequences set forth in SEQ ID NOs: 614 to 616 were detected by the mass spectrometry. The protein identified from this mass data was iron-superoxide dismutase (NCBI accession number NP_001237901.1, amino acid sequence: SEQ ID NO: 613, encoding nucleotide sequence: SEQ ID NO: 612).

### Spot 55α:

The amino acid sequences set forth in SEQ ID NOs: 619 to 635 were detected by the mass spectrometry. The protein identified from this mass data was 51 kDa seed maturation protein precursor (NCBI accession number NP_001238138.1, amino acid sequence: SEQ ID NO: 618, encoding nucleotide sequence: SEQ ID NO: 617).

The amino acid sequences set forth in SEQ ID NOs: 638 to 649 were detected by the mass spectrometry. The protein identified from this mass data was Lea protein precursor (NCBI accession number NP_001238201.1, amino acid sequence: SEQ ID NO: 637, encoding nucleotide sequence: SEQ ID NO: 636).

### INDUSTRIAL APPLICABILITY

The present invention can provide novel antigens of an allergy to soybean, methods and kits for diagnosing an allergy to soybean, pharmaceutical compositions comprising such an antigen, and soybeans or processed products of soybean in which such an antigen is eliminated or reduced.

1.-9. (canceled)

10. A method for detecting binding between a soybean antigen and an antibody that specifically binds to the soybean antigen present in a sample,
wherein the method uses a kit that comprises the soybean antigen,
wherein the soybean antigen is a Ribulose bisphosphate carboxylase large chain antigen comprising the amino acid sequence of SEQ ID NO: 4,
wherein the soybean antigen specifically binds to IgE antibody from a patient having allergy to soybean, wherein the soybean antigen is immobilized to a carrier or a surface,
wherein the method comprises:
(i) contacting the antibody in the sample with the soybean antigen from the kit, and
(ii) detecting binding between said soybean antigen and the antibody in the sample.

11. A method for detecting binding between a soybean antigen and an antibody that specifically binds to the soybean antigen present in a sample,
wherein the method uses a kit that comprises the soybean antigen,
wherein the soybean antigen is a Ribulose bisphosphate carboxylase large chain antigen comprising the amino acid sequence of SEQ ID NO: 4,
wherein the soybean antigen specifically binds to IgE antibody from a patient having allergy to soybean, wherein the kit further comprises a reagent that detects binding between said soybean antigen and said antibody;
wherein the method comprises:
(i) contacting the antibody in the sample with the soybean antigen from the kit, and
(ii) detecting binding between said soybean antigen and the antibody in the sample.

12. The method of claim 11, wherein the reagent comprises an enzymatically labelled secondary antibody against said antibody that specifically binds to said soybean antigen.

13. The method of claim 12, wherein the kit further comprises an enzyme substrate for an enzyme in the enzymatically labelled secondary antibody.

14. The method of claim 11, wherein the reagent comprises a fluorescently labelled secondary antibody against the antibody that specifically binds to said soybean antigen.

15. A method for detecting binding between a soybean antigen and an antibody that specifically binds to the soybean antigen present in a sample,
wherein the method uses a kit that comprises the soybean antigen,
wherein the soybean antigen is a elongation factor 1-alpha antigen comprising the amino acid sequence of SEQ ID NO: 460 or SEQ ID NO: 468,
wherein the soybean antigen specifically binds to IgE antibody from a patient having allergy to soybean, wherein the soybean antigen is immobilized to a carrier or a surface,
wherein the method comprises:
(i) contacting the antibody in the sample with the soybean antigen from the kit, and
(ii) detecting binding between said soybean antigen and the antibody in the sample.

16. A method for detecting binding between a soybean antigen and an antibody that specifically binds to the soybean antigen present in a sample,
wherein the method uses a kit that comprises the soybean antigen,
wherein the soybean antigen is a elongation factor 1-alpha antigen comprising the amino acid sequence of SEQ ID NO: 460 or SEQ ID NO: 468,
wherein the soybean antigen specifically binds to IgE antibody from a patient having allergy to soybean, wherein the kit further comprises a reagent that detects binding between said soybean antigen and said antibody;
wherein the method comprises:
(i) contacting the antibody in the sample with the soybean antigen from the kit, and
(ii) detecting binding between said soybean antigen and the antibody in the sample.

17. The method of claim 16, wherein the reagent comprises an enzymatically labelled secondary antibody against said antibody that specifically binds to said soybean antigen.

18. The method of claim 17, wherein the kit further comprises an enzyme substrate for an enzyme in the enzymatically labelled secondary antibody.

19. The method of claim 16, wherein the reagent comprises a fluorescently labelled secondary antibody against the antibody that specifically binds to said soybean antigen.


SEQUENCE LISTING (649 sequences)

SEQ ID NO: 1 - DNA, 249 bp, Glycine max
  source 1..249: mol_type = other DNA
  CDS 1..249
atgtgtattctgagcttcctcaaaagtgatcagtcatcaagttatgatgatgatgagtattcaaaaccatgctgtgatctctgcatgtgcacacgctcaatgcctcctcaatgcagctgtgaagatattaggctgaattcatgccactcagattgtaagagctgtatgtgcacacgctcacagccaggacagtgtcgttgtcttgacaccaacgacttctgctacaaaccttgcaagtccagagatgac

SEQ ID NO: 2 - protein, 83 aa, Glycine max
MCILSFLKSDQSSSYDDDEYSKPCCDLCMCTRSMPPQCSCEDIRLNSCHSDCKSCMCTRSQPGQCRCLDTNDFCYKPCKSRDD

SEQ ID NO: 3 - DNA, 1425 bp, Glycine max
  source 1..1425: mol_type = other DNA
  CDS 1..1425
atgtcaccacaaacagagactaaagcaagtgttgggttcaaagctggtgttaaagattataaattgacttattatactcctgactatgaaaccaaagatactgatatcttggcagcattccgagtaactcctcaaccaggagttccgcctgaagaagcaggtgccgcggtagccgccgaatcttctactggtacatggacaactgtgtggaccgatgggcttaccagtcttgatcgttacaaagggcgatgctacggccttgaacctgttgctggggaagaaaatcaatatattgcttatgtagcttatcccttagacctttttgaagaaggttctgttactaacatgtttacttccattgtcggtaatgtatttgggttcaaggccctgcgtgctctacgtctggaggatttgcgaatccctactgcttatattaaaactttccaaggtccgcctcatggcatccaagttgagagagataaattgaacaagtatggtcgtcccctattaggatgtactattaaacctaaattggggttatccgctaagaattatggtagagctgtttatgaatgtcttcgtgggggacttgattttaccaaagatgatgaaaatgtgaattcccaaccatttatgcgttggagagaccgtttcttattttgtgccgaagccatttttaaatcacaggctgaaacaggtgaaatcaaagggcattacttgaatgcaactgcgggtacatgcgaagaaatgatgaaaagagctgtatttgccagagaattaggcgttcctatcgtaatgcatgattatttaacggggggattcactgcaaatactagcttggctcattattgccgagataatggtctacttcttcatatacaccgtgcaatgcatgcagttatcgacagacaaaagaatcatggtatgcactttcgtgtactagctaaagcattacgtttgtctggtggagatcatgttcacgccggtaccgtagtaggtaaacttgaaggggaaagagaaatcactttaggttttgttgatttactacgtgatgattttgttgaaaaagatcgaagtcgcggtatttatttcactcaggattgggtttctctaccaggtgttttgcctgtagcttcgggaggtattcacgtttggcatatgcctgctctgaccgagatctttggggatgattctgtactccaatttggcggaggaactttaggacacccttggggaaatgcaccaggtgctgtagctaatcgagtagctcttgaagcatgtgtgcaggctcgaaatgaaggacgcgatcttgctcgtgaaggtaatgaaattatccgtgaggctagcaaatggagtcctgaattagctgctgcttgtgaagtatggaaggagatcaaatttgaattcgaagcaatggatactttg

SEQ ID NO: 4 - protein, 475 aa, Glycine max
MSPQTETKASVGFKAGVKDYKLTYYTPDYETKDTDILAAFRVTPQPGVPPEEAGAAVAAESSTGTWTTVWTDGLTSLDRYKGRCYGLEPVAGEENQYIAYVAYPLDLFEEGSVTNMFTSIVGNVFGFKALRALRLEDLRIPTAYIKTFQGPPHGIQVERDKLNKYGRPLLGCTIKPKLGLSAKNYGRAVYECLRGGLDFTKDDENVNSQPFMRWRDRFLFCAEAIFKSQAETGEIKGHYLNATAGTCEEMMKRAVFARELGVPIVMHDYLTGGFTANTSLAHYCRDNGLLLHIHRAMHAVIDRQKNHGMHFRVLAKALRLSGGDHVHAGTVVGKLEGEREITLGFVDLLRDDFVEKDRSRGIYFTQDWVSLPGVLPVASGGIHVWHMPALTEIFGDDSVLQFGGGTLGHPWGNAPGAVANRVALEACVQARNEGRDLAREGNEIIREASKWSPELAAACEVWKEIKFEFEAMDTL

SEQ ID NO: 5 - DNA, 1026 bp, Glycine max
  source 1..1026: mol_type = other DNA
  CDS 1..1026
atgccaaacgacagcgttctgagtcttttcttcttcgttaccctcttcacatgccttctctctgctacttcccacgatgaccatattttcctaccgagtcaactccatgacgatgactcagtgagttgcactgcaaccgacccaagccttaactacaagccggtgatcggaatcctgacccaccccggcgacggtgcctctggccggctcagcaacgccaccggggtttcttacatcgccgcctcctacgttaaattcgtggagtccggtggtgctagagttattccccttatctacaatgagtcacctgagaaccttaacaagaagctcgatttggtcaatggagtgctcttcactggtgggtgggctgtttctggtccatatttggatactcttggaaacatttttaagaaagctttagagagaaatgatgctggagaccatttcccagtaattgccttcaacttaggtggaaacctcgtaataaggatcgtaagcgagcaaactgatattcttgaaccatttactgcgtcgagtttgccaagttctcttgtattgtggaacgaagcaaatgctaaaggaagtctatttcaaagatttccttcagatttgttgacacagctgaagacagattgtctcgtcctgcacaatcatcgttatgccatatccccacgcaagcttcaatataataccaagttatctgacttttttgagattctggcaacatctggggatagagatggaaagactttcgtttccacagcacgtggtaggaaatatcccgtgactgtcaacctatggcaaccagagaaaaatgcgtttgagtgggctacatctttgaaagcccctcacacagaggatgcaattcgggttactcagtccacagcaaactttttcataagtgaagctaggaaatccacaaacacacctgatgctcaaaaagtgcgagatagtctcatctacaattacaaacctacttttggtggaacggctgggaaaggatatgatcaagtgtatctctttgaa

SEQ ID NO: 6 - protein, 342 aa, Glycine max
MPNDSVLSLFFFVTLFTCLLSATSHDDHIFLPSQLHDDDSVSCTATDPSLNYKPVIGILTHPGDGASGRLSNATGVSYIAASYVKFVESGGARVIPLIYNESPENLNKKLDLVNGVLFTGGWAVSGPYLDTLGNIFKKALERNDAGDHFPVIAFNLGGNLVIRIVSEQTDILEPFTASSLPSSLVLWNEANAKGSLFQRFPSDLLTQLKTDCLVLHNHRYAISPRKLQYNTKLSDFFEILATSGDRDGKTFVSTARGRKYPVTVNLWQPEKNAFEWATSLKAPHTEDAIRVTQSTANFFISEARKSTNTPDAQKVRDSLIYNYKPTFGGTAGKGYDQVYLFE

SEQ ID NO: 7 - DNA, 975 bp, Glycine max
  source 1..975: mol_type = other DNA
  CDS 1..975
atggcggaaggactcgtcctgaagggaaccatgcgcgcccacaccgacgtcgtgacggcgattgccactcccatcgacaactccgacatgatcgtgacggcgtcgcgcgacagatccatcattctctggcacctcaccaaggaggacaagacctacggtgtcccccgccgccgcctcaccggacattctcacttcgtccaggacgtcgtcctctcatccgacggtcagttcgccctctccggctcctgggacggcgagctccgcctctgggacctcgcggctggcacctctgcccgccgcttcgttggccacaccaaggacgtgctctccgtggcgttctccatcgacaaccgtcagatcgtgtcggcctctcgtgaccgcacgatcaagctgtggaacaccctgggtgagtgcaagtacacaatccaagatggcgatgcgcattcggattgggtaagttgcgtccgtttcagccctagcactcttcagccaaccattgtttctgcttcatgggacaggaccgttaaggtttggaacctgaccaactgcaagctgaggaacacccttgctggacacaatgggtatgtgaatactgttgctgtttcccctgatggctctctctgtgccagtggtggcaaagatggggttattcttctgtgggatttggctgagggtaagcgtctttactctctcgatgctggctcaatcatccatgcactctgcttcagccccagcaggtactggctctgcgccgccaccgagcagagcatcaagatctgggatttggagagcaagagtatcgttgaggatttgaaggttgacctcaagactgaggctgatgccacctccggtggtggtaacgccaacaagaagaaggttatttattgcacaagtttgaactggagtgcggatggaagcactttgtttagtggctataccgatggtgtggccagagtttgggctattggacgttat

SEQ ID NO: 8 - protein, 325 aa, Glycine max
MAEGLVLKGTMRAHTDVVTAIATPIDNSDMIVTASRDRSIILWHLTKEDKTYGVPRRRLTGHSHFVQDVVLSSDGQFALSGSWDGELRLWDLAAGTSARRFVGHTKDVLSVAFSIDNRQIVSASRDRTIKLWNTLGECKYTIQDGDAHSDWVSCVRFSPSTLQPTIVSASWDRTVKVWNLTNCKLRNTLAGHNGYVNTVAVSPDGSLCASGGKDGVILLWDLAEGKRLYSLDAGSIIHALCFSPSRYWLCAATEQSIKIWDLESKSIVEDLKVDLKTEADATSGGGNANKKKVIYCTSLNWSADGSTLFSGYTDGVARVWAIGRY

SEQ ID NO: 9 - DNA, 303 bp, Glycine max
  source 1..303: mol_type = other DNA
  CDS 1..303
atggcatctcatcaacaaaacaagcaagagcttgatgaaagggcaaggcaaggagagactgttgttccgggtggcactggtggcaagagccttgaggctcagcaacatcttgctgaaggaaggagcaggggagggaaaacaaggaaggagcagctggggacagaagggtaccatgaaatgggacgcaaaggtgggttgagcactatggacaagtcaggagaagaacgtgctcaagaggaagccattgacatcgatgagtccaagttcaggactggtaataaccaagacaagaaccagaacaag

SEQ ID NO: 10 - protein, 101 aa, Glycine max
MASHQQNKQELDERARQGETVVPGGTGGKSLEAQQHLAEGRSRGGKTRKEQLGTEGYHEMGRKGGLSTMDKSGEERAQEEAIDIDESKFRTGNNQDKNQNK

SEQ ID NO: 11 - DNA, 1929 bp, Glycine max
  source 1..1929: mol_type = other DNA
  CDS 1..1929
atggcgtctgaacaattggctcgcagagagaacaccaccaccgagaaggagattcacgttgagaaacacagggttcccaagatggccacccacttcgagcaccttgcggagcaagctaaagaatctgacatcaccgctgggaaggatactccgcagggtagcatagaagccttgcaagctggagagagggtcaaagatcatgcaggaaaagccatgggagatattggtggacgtgggaaagccagagagactcacgagctgggtgcacactttgaatccctggctgacaaagtaactgaccatgcagcagccaatgtagttggaaacaaagagagtcaaagagaagctcgtggtggtgttcgtgatgttgggaagttcgaaatgagaactgaagggggagaaaaaggaaacaaggatagaccagaactgaaaacacgaaccagagaggtaataggaagaacggagaaagaaagaggaagagaaagtggaggacaagtagtggcagagaaaggaagagaaacagaaactgcaaggggaagggttggtgctgaaaatgaaggtgcaagaacaaccgctgttataacttgcacgttggaaaaaggtggtggtactcagaaaccaataagagaagaagagagagagagtgaaagtgaaagatcagcgtgggaacagatttccaactacagtgatcaagccacacaaggtgtgaaggaaaagtacgagagagcaaagcaagcagcaagcgagacactgaacaccacaacacaaaccgcacaagagaaaagcgcacaggcaaagaatcttgcagcacaagcaaaggacgcaacgttagagaagggtcagcaaggctatgctgtgacaaaggacaccatttcaagtgcagcgaaaaccgcttcggagaaaactgcgccagtggcagagaaggccaaagactacactcttcaggctgcggagaaggccaagagtgcgggtggcacaacggcgagttacgtcggagagaaagcggtgcaggccaaggatgtcgccgtggagagtgggaagagtgcggctgggtatgctgccaaggtggctgctgatttgagggacaaggccaccgcggtggggtgggccgcggcccatttttcggcggaaaagacggtggaagggaccaaggccgccgcccatgttgtcgagggagcggccgggtatgccggacataaggccgcggagcttgcttcgatgtcggccggtgctgttaaagggctggctgcttctgctggtgagactgctaaggaatacaccgcaaagaagaaagaggaggcacagcgggaattggaggccaaaaaaccatctcaacctcaggaagctgaagagaggccatcagaaggaattggcgaaactgtgaggcagtatgcacaaaaaccaaagccaagtgaaggaaaccctcagaaggagggaaccggaagcatcgtgtttacagcaataggcgaaacagtaagcagtgccggtgagaaggtaaagaaaccgtttaaaaacactatgggaggagaaagtgaaggaggaggagggaaagaagagggaaagagtgtcattggtaaaagtttgaccagcattggtgagaagctgggtgatgcaaaacagagagaggagctgttagacaacgtcactggcaacatcaccgagggaggaggcgaggtgttgggagcagtaggtgaaaccgttgctgagattggacagaacatgatgaagccagcggagatagttcaggaacgcgctcacgttcgacaagcgggtggggtcttggatgccattggtgaaaccattgctgagattgctgaaacgactcgagtcatggtttctggagaggacgagagagtgctgaggcagagcgtggtgttggagactcgcgtaactggtcgtgccaagcatgaagaagggtcgcacggtgct

SEQ ID NO: 12 - protein, 643 aa, Glycine max
MASEQLARRENTTTEKEIHVEKHRVPKMATHFEHLAEQAKESDITAGKDTPQGSIEALQAGERVKDHAGKAMGDIGGRGKARETHELGAHFESLADKVTDHAAANVVGNKESQREARGGVRDVGKFEMRTEGGEKGNKDRPELKTRTREVIGRTEKERGRESGGQVVAEKGRETETARGRVGAENEGARTTAVITCTLEKGGGTQKPIREEERESESERSAWEQISNYSDQATQGVKEKYERAKQAASETLNTTTQTAQEKSAQAKNLAAQAKDATLEKGQQGYAVTKDTISSAAKTASEKTAPVAEKAKDYTLQAAEKAKSAGGTTASYVGEKAVQAKDVAVESGKSAAGYAAKVAADLRDKATAVGWAAAHFSAEKTVEGTKAAAHVVEGAAGYAGHKAAELASMSAGAVKGLAASAGETAKEYTAKKKEEAQRELEAKKPSQPQEAEERPSEGIGETVRQYAQKPKPSEGNPQKEGTGSIVFTAIGETVSSAGEKVKKPFKNTMGGESEGGGGKEEGKSVIGKSLTSIGEKLGDAKQREELLDNVTGNITEGGGEVLGAVGETVAEIGQNMMKPAEIVQERAHVRQAGGVLDAIGETIAEIAETTRVMVSGEDERVLRQSVVLETRVTGRAKHEEGSHGA

SEQ ID NO: 13 - DNA, 771 bp, Glycine max
  misc_feature 242: note = n is a, c, g, or t
  source 1..771: mol_type = other DNA
  CDS 1..771
atggccctttcttgctccaaagttttaagcttttatctctcgcccgttgtgggtggtggtgatgttccgaaaaagctcactttttcttcttttttgggtctttctaagggtgttggtggaagcaggagctcgagggtgtgtgctgcttcaaatgcacccgcaccactcgctggggtcatatttgaaccatttcaagagctcaagaaggattatcttgctgttccgattgcacacaatgtttncttggctcgtcagaactatgcagatgactctgaatctgcaatcaacgagcagatcaatgtggaatacaacgtttcctatgtgtaccacgccttgtttgcgtacttcgacagagacaacatagctctcaaaggacttgcaaagttcttcaaggaatcaagtgaagaagaaagagagcatgccgagcagcttataaagtatcagaacattcgtggtggacgagtggtgctgcacccaatcacaagtcctccctcggaatttgagcattcggaaaaaggggatgccttgtatgccatggaactagctttgtctttggagaagttgacgaatgagaaacttctgcacgttcacagtgtggcagaacgtaacaatgatcctcaaytdgcagacttcattgagagcgagttcttgtatgagcaggttaaatcgattaaaaagattgcagagtatgtggctcaactgagattagttggaaagggtcatggtgtgtggcactttgatcaaaagcttcttcacgatgaagatcatgta

SEQ ID NO: 14 - protein, 257 aa, Glycine max
  SITE 81: note = misc_feature - The 'Xaa' at location 81 stands for Tyr, Cys, Ser, or Phe.
  SITE 208: note = misc_feature - The 'Xaa' at location 208 stands for Leu, or Phe.
MALSCSKVLSFYLSPVVGGGDVPKKLTFSSFLGLSKGVGGSRSSRVCAASNAPAPLAGVIFEPFQELKKDYLAVPIAHNVXLARQNYADDSESAINEQINVEYNVSYVYHALFAYFDRDNIALKGLAKFFKESSEEEREHAEQLIKYQNIRGGRVVLHPITSPPSEFEHSEKGDALYAMELALSLEKLTNEKLLHVHSVAERNNDPQXADFIESEFLYEQVKSIKKIAEYVAQLRLVGKGHGVWHFDQKLLHDEDHV

SEQ ID NO: 15 - DNA, 2421 bp, Glycine max
  source 1..2421: mol_type = other DNA
  CDS 1..2421
atgtctcagcaaggcgaatcatcggacccgaaatcggggaagaaggacttctcaaccgcgattctggaacgcaaaaagtcgccgaaccggcttgtcgtggatgaggctgtcaacgacgacaactccgtggtcaccatgcatccccaaaccatggaaaagcttcagcttttccgaggggacactatcctcatcaagggtaagaaacggaaggataccatttgcatagctcttgctgatgagaactgtgaggagccgaagatcaggatgaataaagttgtgaggtcaaatttgcgggttcggcttggagatgttgtgtctgtgcaccagtgcccggatgttaaatatgggaaacgtgtgcacattctgcctattgatgataccattgaaggtgtcactggcaatctgtttgatgctttcttgaaaccctatttcttggaagcttaccgtcctgtcagaaaaggagatctatttcttgtccggggagggatgagaagtgtagagttcaaggtggttgaaaccgatcctggagaatattgtgttgttgctccagacactgaaattttctgtgagggggagcctttgaaaagagaagatgaagagagactggatgaagtcgggtatgatgatgtgggtggtgtcaggaaacaaatggctcagattcgtgagttggttgagcttccattgaggcatccacagctttttaaatcaattggtgtgaaaccacccaaaggaattttactttatggacccccaggttctgggaagacattaatagcaagagctgtagctaatgaaactggagcttttttcttttgtattaacggaccagagattatgtccaaacttgctggagagagtgaaagcaatctcaggaaagcgtttgaagaagcagaaaagaatgcaccatccattatcttcattgatgaaattgattccattgcccccaagcgggagaagacacatggtgaagttgaaaggaggattgtttcacaacttttgactcttatggatggattaaaatctcgtgctcatgttattgttattggagctactaatcgtccaaatagcattgacccagcattgagaaggtttggtagatttgatagggaaattgatattggtgttcccgatgaagttgggagacttgaagtccttcgcatacatactaaaaacatgaagctatctgatgatgttgatttagagagaattgcaaaagatactcatgggtatgttggtgctgaccttgctgccctttgcactgaagctgctttgcaatgcattagggagaagatggatgtaattgacttggaggatgagactattgatgcagaagtactgaattccatggcagttacaaatgagcatttccagactgcccttggaacaagcaacccatcagctttacgtgaaactgttgttgaagtgcctaatgtcagctgggaagacattggaggccttgaaaatgtcaagcgtgaactgcaagagactgtacaatatccagttgagcacccagaaaagtttgagaagtttggtatgtctccatctaaaggagtacttttctatggtcctccaggatgtgggaaaactttgttagccaaagcaattgctaatgaatgtcaagctaacttcatcagtgtgaaaggtccagaattgcttacaatgtggtttggtgaaagtgaagctaatgttagagaaatttttgacaaggccagacaatcggctccatgtgtcctcttctttgatgagcttgactctattgccactcagagaggaagcagtgttggggatgctggtggtgcggctgatagagttctaaatcaacttttgactgaaatggatggcatgtctgccaaaaagactgtgttcataattggggcaactaacagacctgacatcattgacccagcacttcttcgaccaggccgtctggatcaattgatctatattcctctccctgatgaggattcccggcatcaaatctttaaagcctgcttgaggaagtcacctatcgcaaaaaatgttgatttgagagcattggctaggcacactcaagggttcagtggtgctgatattacagaaatatgccagcgagcatgcaaatatgccataagagaaaacatagagaaggacattgagcgagagaggaagagtagggaaaaccctgaggccatggatgaagataccgtcgacgacgaagttgcggagatcaaggctgctcattttgaggagtcaatgaagtttgcacggaggagtgttagtgatgctgatattcgcaagtatcaggcatttgctcagaccttacagcaatcaagagggtttggaagtgaatttaggtttccagagtctggagataggactactactggatctgacccctttgcagcttctgctggtggggctgacgaagatgatctatatagt

SEQ ID NO: 16 - protein, 807 aa, Glycine max
MSQQGESSDPKSGKKDFSTAILERKKSPNRLVVDEAVNDDNSVVTMHPQTMEKLQLFRGDTILIKGKKRKDTICIALADENCEEPKIRMNKVVRSNLRVRLGDVVSVHQCPDVKYGKRVHILPIDDTIEGVTGNLFDAFLKPYFLEAYRPVRKGDLFLVRGGMRSVEFKVVETDPGEYCVVAPDTEIFCEGEPLKREDEERLDEVGYDDVGGVRKQMAQIRELVELPLRHPQLFKSIGVKPPKGILLYGPPGSGKTLIARAVANETGAFFFCINGPEIMSKLAGESESNLRKAFEEAEKNAPSIIFIDEIDSIAPKREKTHGEVERRIVSQLLTLMDGLKSRAHVIVIGATNRPNSIDPALRRFGRFDREIDIGVPDEVGRLEVLRIHTKNMKLSDDVDLERIAKDTHGYVGADLAALCTEAALQCIREKMDVIDLEDETIDAEVLNSMAVTNEHFQTALGTSNPSALRETVVEVPNVSWEDIGGLENVKRELQETVQYPVEHPEKFEKFGMSPSKGVLFYGPPGCGKTLLAKAIANECQANFISVKGPELLTMWFGESEANVREIFDKARQSAPCVLFFDELDSIATQRGSSVGDAGGAADRVLNQLLTEMDGMSAKKTVFIIGATNRPDIIDPALLRPGRLDQLIYIPLPDEDSRHQIFKACLRKSPIAKNVDLRALARHTQGFSGADITEICQRACKYAIRENIEKDIERERKSRENPEAMDEDTVDDEVAEIKAAHFEESMKFARRSVSDADIRKYQAFAQTLQQSRGFGSEFRFPESGDRTTTGSDPFAASAGGADEDDLYS

SEQ ID NO: 17 - protein, 13 aa, Glycine max
DQSSSYDDDEYSK

SEQ ID NO: 18 - protein, 15 aa, Glycine max
KSDQSSSYDDDEYSK

SEQ ID NO: 19 - protein, 14 aa, Glycine max
SDQSSSYDDDEYSK

SEQ ID NO: 20 - protein, 9 aa, Glycine max
SYDDDEYSK

SEQ ID NO: 21 - protein, 18 aa, Glycine max
ALRALRLEDLRIPTAYIK

SEQ ID NO: 22 - protein, 18 aa, Glycine max
ALRLSGGDHVHAGTVVGK

SEQ ID NO: 23 - protein, 22 aa, Glycine max
LEGEREITLGFVDLLRDDFVEK

SEQ ID NO: 24 - protein, 14 aa, Glycine max
LRLEDLRIPTAYIK

SEQ ID NO: 25 - protein, 11 aa, Glycine max
LTYYTPDYETK

SEQ ID NO: 26 - protein, 11 aa, Glycine max
TDGLTSLDRYK

SEQ ID NO: 27 - protein, 14 aa, Glycine max
TFQGPPHGIQVERD

SEQ ID NO: 28 - protein, 15 aa, Glycine max
TFQGPPHGIQVERDK

SEQ ID NO: 29 - protein, 24 aa, Glycine max
APHTEDAIRVTQSTANFFISEARK

SEQ ID NO: 30 - protein, 23 aa, Glycine max
FVESGGARVIPLIYNESPENLNK

SEQ ID NO: 31 - protein, 7 aa, Glycine max
IWDLESK

SEQ ID NO: 32 - protein, 7 aa, Glycine max
SIVEDLK

SEQ ID NO: 33 - protein, 21 aa, Glycine max
QELDERARQGETVVPGGTGGK

SEQ ID NO: 34 - protein, 17 aa, Glycine max
SGEERAQEEAIDIDESK

SEQ ID NO: 35 - protein, 16 aa, Glycine max
AAAHVVEGAAGYAGHK

SEQ ID NO: 36 - protein, 11 aa, Glycine max
AKDYTLQAAEK

SEQ ID NO: 37 - protein, 17 aa, Glycine max
ARETHELGAHFESLADK

SEQ ID NO: 38 - protein, 15 aa, Glycine max
ATAVGWAAAHFSAEK

SEQ ID NO: 39 - protein, 17 aa, Glycine max
DTPQGSIEALQAGERVK

SEQ ID NO: 40 - protein, 9 aa, Glycine max
DYTLQAAEK

SEQ ID NO: 41 - protein, 10 aa, Glycine max
EEAQRELEAK

SEQ ID NO: 42 - protein, 21 aa, Glycine max
EGTGSIVFTAIGETVSSAGEK

SEQ ID NO: 43 - protein, 14 aa, Glycine max
ERGRESGGQVVAEK

SEQ ID NO: 44 - protein, 11 aa, Glycine max
GLAASAGETAK

SEQ ID NO: 45 - protein, 9 aa, Glycine max
GQQGYAVTK

SEQ ID NO: 46 - protein, 26 aa, Glycine max
KPSQPQEAEERPSEGIGETVRQYAQK

SEQ ID NO: 47 - protein, 8 aa, Glycine max
PAEIVQER

SEQ ID NO: 48 - protein, 15 aa, Glycine max
PQGSIEALQAGERVK

SEQ ID NO: 49 - protein, 17 aa, Glycine max
QAASETLNTTTQTAQEK

SEQ ID NO: 50 - protein, 13 aa, Glycine max
SAGGTTASYVGEK

SEQ ID NO: 51 - protein, 8 aa, Glycine max
SLTSIGEK

SEQ ID NO: 52 - protein, 16 aa, Glycine max
TPQGSIEALQAGERVK

SEQ ID NO: 53 - protein, 13 aa, Glycine max
VTDHAAANVVGNK

SEQ ID NO: 54 - protein, 22 aa, Glycine max
AASNAPAPLAGVIFEPFQELKK

SEQ ID NO: 55 - protein, 21 aa, Glycine max
ASNAPAPLAGVIFEPFQELKK

SEQ ID NO: 56 - protein, 18 aa, Glycine max
FFKESSEEEREHAEQLIK

SEQ ID NO: 57 - protein, 10 aa, Glycine max
GHGVWHFDQK

SEQ ID NO: 58 - protein, 13 aa, Glycine max
IAEYVAQLRLVGK

SEQ ID NO: 59 - protein, 14 aa, Glycine max
KIAEYVAQLRLVGK

SEQ ID NO: 60 - protein, 8 aa, Glycine max
LLHDEDHV

SEQ ID NO: 61 - protein, 20 aa, Glycine max
SNAPAPLAGVIFEPFQELKK

SEQ ID NO: 62 - protein, 10 aa, Glycine max
AGESESNLRK

SEQ ID NO: 63 - protein, 13 aa, Glycine max
GESEANVREIFDK

SEQ ID NO: 64 - protein, 12 aa, Glycine max
GILLYGPPGSGK

SEQ ID NO: 65 - protein, 11 aa, Glycine max
LAGESESNLRK

SEQ ID NO: 66 - protein, 12 aa, Glycine max
LSDDVDLERIAK

SEQ ID NO: 67 - protein, 16 aa, Glycine max
RELQETVQYPVEHPEK

SEQ ID NO: 68 - protein, 15 aa, Glycine max
TVFIIGATNRPDIID

SEQ ID NO: 69 - DNA, 2448 bp, Glycine max
  source 1..2448: mol_type = other DNA
  CDS 1..2448
atgaggaagtggacggttgcttccgctctccttcttctctctctcctctttctctttgcagatcaaggtcgaaagtttcaggcgaatgccgagggagattccgacgagcttgtagatccacccaaggtggaggacaagatcggtgctgtgcctcatggcctttcaaccgattcagatgtcgtcaagagggaggcggagtcgatctcgaagagatctctccggagcaacgcggagaagtttgagttccaagcggaagtgtcgcggcttatggatatcatcatcaattctctctacagtaacaaggatatcttcctcagagagttgatttccaatgcttctgatgctttggacaagattaggttcctctccctcacggataaggaggtgttgggagaaggtgataacaccaagcttgatattcagattaagttggacaaggaaaagaaaattctctcaattcgagacagaggtatcggtatgacgaaggaggatttgataaagaatttggggacaatagcgaaatctggaacttctgcatttgttgagaagatgcaaacaagtggagatctcaatctgattgggcagtttggagtcggcttctactctgtttatcttgtggccgactatgtcgaagttattagcaagaataacgatgataagcagtatgtatgggaatcaaaagctgatggagcattcgctatttcagaagatacatggaatgaaccacttggacgaggaactgagattagattacacctcaaggaagaggctggggaatacttggaggagtctaaactgaaagaattggtgaagagatactctgaatttattaacttccctatctatatttgggcaagcaaagaagttgatgtggaggttcccgctgatgaagatgattccagtgatgaagaggattcatctgaaagcagctccaaggaggaaagcgaagatgaagatgctgataaaagtgaagatgaagaaaaaaagcctaagacaaagacagtgaaggaaacaacttatgagtgggaacttttaaatgatgtgaaagctatatggctgaggaatccaaaagaggttacagaagaagaatacaccaaattctaccactctcttgccaaggatttcagtgatgagaaacctttagcatggagtcatttcactgctgagggtgatgttgagttcaaggcagtcctgtttgtaccacctaaggcacctcaagatttatatgagagctattataacgcaaacaaatcgaacctgaagttgtatgttagacgtgtgttcatttcagatgaatttaatgaactgttgcccaagtacctaaactttttgctgggtcttgttgattctgacactttgccacttaatgtatcacgagaaatgcttcaacaacatagtagtctgaagacaatcaagaagaaacttattaggaaagcccttgatatgatccgcaggattgctgatgaggatcctgacgagtcaactgacaaagaaaagaaagatgcatcctctgacaatgatgagaagaaaggtcaatattctaagttctggaatgagtttggtaaatccattaaacttggtatcatcgaggatgccaccaacagaaatcgtttggcaaaactgctcagatttgagagcaccaagtctgagggtaaattgacatctctggatcagtacatatctagaatgaaaactgggcagaaggatatcttttacataactggaaccagcaaagaacaactggaaaactctcccttccttgagcggcttaagaagaaaaattttgaggttattttcttcacggatccagttgatgagtacttgatgcaatacctgatggattatgaagacaagaagttccaaaatgtgtccaaggagggtttgaaacttgggaaagattcaaaagacaaggaactgaaggaatcttttaaggatcttaccaagtggtggaaaactgctctttccaaagacaatgttgatgatgtgaagatttccaaccgattagataacactccttgtgtagtggtgacatcaaaatttggttggagtgctaacatggagagaatcatgcagtctcaaactttgtcagatgctagcaagcaagcatacatgcgcggcaagagggttcttgagatcaatcctaggcaccctattatcaaggagctccgagagagagtagtaaagaaccctgaggatgagggtgtgaagcatacagcacagctgatgtaccagactgcactctttgaaagcggcttcctgcttgatgatcccaaggattttgcttcccggatttatgattcagtgaagactagcctggacatcagtcctgaagcaacagttgaggaggaagatgacactgaagtagaggctgaaagtgacgcgaaagaagattcaaagcctgaaactgatgctgttaacgatgataatgatgtcaaggacgagttgtag

SEQ ID NO: 70 - protein, 815 aa, Glycine max
MRKWTVASALLLLSLLFLFADQGRKFQANAEGDSDELVDPPKVEDKIGAVPHGLSTDSDVVKREAESISKRSLRSNAEKFEFQAEVSRLMDIIINSLYSNKDIFLRELISNASDALDKIRFLSLTDKEVLGEGDNTKLDIQIKLDKEKKILSIRDRGIGMTKEDLIKNLGTIAKSGTSAFVEKMQTSGDLNLIGQFGVGFYSVYLVADYVEVISKNNDDKQYVWESKADGAFAISEDTWNEPLGRGTEIRLHLKEEAGEYLEESKLKELVKRYSEFINFPIYIWASKEVDVEVPADEDDSSDEEDSSESSSKEESEDEDADKSEDEEKKPKTKTVKETTYEWELLNDVKAIWLRNPKEVTEEEYTKFYHSLAKDFSDEKPLAWSHFTAEGDVEFKAVLFVPPKAPQDLYESYYNANKSNLKLYVRRVFISDEFNELLPKYLNFLLGLVDSDTLPLNVSREMLQQHSSLKTIKKKLIRKALDMIRRIADEDPDESTDKEKKDASSDNDEKKGQYSKFWNEFGKSIKLGIIEDATNRNRLAKLLRFESTKSEGKLTSLDQYISRMKTGQKDIFYITGTSKEQLENSPFLERLKKKNFEVIFFTDPVDEYLMQYLMDYEDKKFQNVSKEGLKLGKDSKDKELKESFKDLTKWWKTALSKDNVDDVKISNRLDNTPCVVVTSKFGWSANMERIMQSQTLSDASKQAYMRGKRVLEINPRHPIIKELRERVVKNPEDEGVKHTAQLMYQTALFESGFLLDDPKDFASRIYDSVKTSLDISPEATVEEEDDTEVEAESDAKEDSKPETDAVNDDNDVKDEL

SEQ ID NO: 71 - protein, 14 aa, Glycine max
APQDLYESYYNANK

SEQ ID NO: 72 - protein, 8 aa, Glycine max
AVLFVPPK

SEQ ID NO: 73 - protein, 11 aa, Glycine max
DFASRIYDSVK

SEQ ID NO: 74 - protein, 17 aa, Glycine max
DIFLRELISNASDALDK

SEQ ID NO: 75 - protein, 10 aa, Glycine max
DIFYITGTSK

SEQ ID NO: 76 - protein, 7 aa, Glycine max
DNVDDVK

SEQ ID NO: 77 - protein, 17 aa, Glycine max
EDSKPETDAVNDDNDVK

SEQ ID NO: 78 - protein, 11 aa, Glycine max
EEAGEYLEESK

SEQ ID NO: 79 - protein, 13 aa, Glycine max
EQLENSPFLERLK

SEQ ID NO: 80 - protein, 10 aa, Glycine max
EVLGEGDNTK

SEQ ID NO: 81 - protein, 9 aa, Glycine max
EVTEEEYTK

SEQ ID NO: 82 - protein, 7 aa, Glycine max
FYHSLAK

SEQ ID NO: 83 - protein, 16 aa, Glycine max
IGAVPHGLSTDSDVVK

SEQ ID NO: 84 - protein, 15 aa, Glycine max
LGIIEDATNRNRLAK

SEQ ID NO: 85 - protein, 8 aa, Glycine max
NPEDEGVK

SEQ ID NO: 86 - protein, 9 aa, Glycine max
SGTSAFVEK

SEQ ID NO: 87 - protein, 26 aa, Glycine max
TSLDISPEATVEEEDDTEVEAESDAK

SEQ ID NO: 88 - DNA, 2442 bp, Glycine max
  source 1..2442: mol_type = other DNA
  CDS 1..2442
atgaggaagtggacggttgcttccgctctccttcttctctctctcctctttctctttgcagatcaaggtcgaaagtttcaggcgaatgccgagggagattccgacgagcttgtagatcctccgaaggtggaggacaagatcggtgctgtgcctcatggcctttccaccgattcagatgtcgtcaagagggagtcggagtcgatctcgaagagatctctccggagcaatgcggagaagtttgagttccaagcggaagtgtcacggcttatggatattatcatcaattctctctacagtaacaaggatatcttcctcagagagttgatttccaatgcttctgatgctttggacaagattaggttcctttccctcacggataaggacgttttgggagaaggtgataacaccaagcttgatattcagattaagttggacaaggaaaagaaaattctctcaattcgagacagaggtattggtatgacgaaggaggatttgataaagaatttggggacaatagcaaaatctggaacttctgcatttgttgagaagatgcaaacaagtggagatctcaatctgattgggcagtttggagtcggcttctactctgtttatctcgtggctgactatgtcgaagtcattagcaagaataatgatgataaacagtatgtatgggaatcaaaagctgatggagcatttgcaatttcagaagatacatggaatgaacctctagggcgtggaactgagattagattacacctcaaggaagaggctggggagtacttgcaggagtctaaactgaaagaattggtgaagagatactctgaatttattaacttccctatctatatttgggcaagcaaagaggttgatgtggaggttcccgctgatgaagatgattccagtgatgaagaggattcatctgaaagcagctccaaggaggaaagcgaagatgaagatgctgataaaagtgaagacgaagaaaaaaagcccaagacaaagacggtgaaggaaacaacttatgaatgggaacttttaaatgatgtgaaagctatatggctgaggaatccaaaggaggttacagaagaagaatacaccaaattctaccactctcttgccaaggatttcagtgatgagaaacctttagcatggagtcatttcactgctgagggcgatgtcgagttcaaggcagtcctgtttgtaccacctaaggcacctcaagatttatacgagagctattataacgcaaacaaatccaacctgaagttgtatgttagacgtgtgttcatttcagatgaatttaatgaactgttgcctaagtacctaaactttttgctgggtcttgttgattctgacacattgccacttaatgtatcgcgagaaatgcttcagcaacatagtagtctaaagacaatcaagaagaaacttattaggaaagcccttgatatgatccgtaggattgctgatgaggatcctgatgagtccactgacaaagaaaagaaagatacgtcatctgacaataatgagaagaaaggtcaatattccaagttctggaatgagtttggtaaatccattaaacttggtatcatcgaggatgccaccaacagaaatcgtttggcaaaactgctcagatttgagagcaccaagtctgagggtaaattgacatctctggatcagtacatatctagaatgaaagctgggcagaaggatatcttttacataactggaaccagcaaagaacaactggaaaactctccattccttgagcggcttaagaagaaaaattttgaggttattttcttcacggatccagttgatgaatacttgatgcaatacctgatggattatgaagacaagaagttccaaaatgtgtccaaggagggtttgaaactcgggaaagattcaaaagacaaggaactgaaggaatccttcaaggaccttaccaagtggtggaaaactgctctttccaaagacaatgttgatgatgtgaagatttccaaccgattggacaacactccttgtgtagtggtaacatcaaaatttggttggagtgctaacatggagagaatcatgcagtctcaaactttgtctgatgctagcaagcaagcatacatgcgcggcaagagggttcttgaggtcaatcctaggcaccctattatcaaggagctccgagagagagtagtaaagaaccctgaggatgagggtgtgaaacatacagcacagttgatgtaccagaccgcactcttcgaaagtggcttcctgcttgatgatcccaaggattttgcttcccgtatttatgattcagttaagaccagcttggacatcagtcctgaagcaacagttgaggaggaagatgacaccgaagtagaggctgaaagtgattcaaagcctgaagctgatgcggttcatgatggtgatgttgatgatgccaaggacgagttgtag

SEQ ID NO: 89 - protein, 813 aa, Glycine max
MRKWTVASALLLLSLLFLFADQGRKFQANAEGDSDELVDPPKVEDKIGAVPHGLSTDSDVVKRESESISKRSLRSNAEKFEFQAEVSRLMDIIINSLYSNKDIFLRELISNASDALDKIRFLSLTDKDVLGEGDNTKLDIQIKLDKEKKILSIRDRGIGMTKEDLIKNLGTIAKSGTSAFVEKMQTSGDLNLIGQFGVGFYSVYLVADYVEVISKNNDDKQYVWESKADGAFAISEDTWNEPLGRGTEIRLHLKEEAGEYLQESKLKELVKRYSEFINFPIYIWASKEVDVEVPADEDDSSDEEDSSESSSKEESEDEDADKSEDEEKKPKTKTVKETTYEWELLNDVKAIWLRNPKEVTEEEYTKFYHSLAKDFSDEKPLAWSHFTAEGDVEFKAVLFVPPKAPQDLYESYYNANKSNLKLYVRRVFISDEFNELLPKYLNFLLGLVDSDTLPLNVSREMLQQHSSLKTIKKKLIRKALDMIRRIADEDPDESTDKEKKDTSSDNNEKKGQYSKFWNEFGKSIKLGIIEDATNRNRLAKLLRFESTKSEGKLTSLDQYISRMKAGQKDIFYITGTSKEQLENSPFLERLKKKNFEVIFFTDPVDEYLMQYLMDYEDKKFQNVSKEGLKLGKDSKDKELKESFKDLTKWWKTALSKDNVDDVKISNRLDNTPCVVVTSKFGWSANMERIMQSQTLSDASKQAYMRGKRVLEVNPRHPIIKELRERVVKNPEDEGVKHTAQLMYQTALFESGFLLDDPKDFASRIYDSVKTSLDISPEATVEEEDDTEVEAESDSKPEADAVHDGDVDDAKDEL

SEQ ID NO: 90 - protein, 14 aa, Glycine max
APQDLYESYYNANK

SEQ ID NO: 91 - protein, 8 aa, Glycine max
AVLFVPPK

SEQ ID NO: 92 - protein, 11 aa, Glycine max
DFASRIYDSVK

SEQ ID NO: 93 - protein, 17 aa, Glycine max
DIFLRELISNASDALDK

SEQ ID NO: 94 - protein, 10 aa, Glycine max
DIFYITGTSK

SEQ ID NO: 95 - protein, 7 aa, Glycine max
DNVDDVK

SEQ ID NO: 96 - protein, 10 aa, Glycine max
DVLGEGDNTK

SEQ ID NO: 97 - protein, 11 aa, Glycine max
EEAGEYLQESK

SEQ ID NO: 98 - protein, 13 aa, Glycine max
EQLENSPFLERLK

SEQ ID NO: 99 - protein, 9 aa, Glycine max
EVTEEEYTK

SEQ ID NO: 100 - protein, 7 aa, Glycine max
FYHSLAK

SEQ ID NO: 101 - protein, 16 aa, Glycine max
IGAVPHGLSTDSDVVK

SEQ ID NO: 102 - protein, 15 aa, Glycine max
LGIIEDATNRNRLAK

SEQ ID NO: 103 - protein, 8 aa, Glycine max
NPEDEGVK

SEQ ID NO: 104 - protein, 9 aa, Glycine max
SGTSAFVEK

SEQ ID NO: 105 - DNA, 2103 bp, Glycine max
  source 1..2103: mol_type = other DNA
  CDS 1..2103
atggcttcggagactgagacgttcgcttttcaggccgagatcaaccagctcctcagtctaataatcaacaccttctacagcaacaaggagatctttcttcgtgagctcatcagcaatgcctctgacgctttggacaagattcgatttgagagtcttacagacaagagcaagcttgatgctcagccagagttgttcattcacattattcctgacaagaccaacaacacattgtctatcattgacagtggtattggcatgactaaggctgatttggtgaataacctcggtactattgcaaggtctgggaccaaggaattcatggaagccctggctgctggtgctgacgtgagcatgattgggcagttcggtgttggtttctactcggcttatcttgtagctgataaggttattgttaccacaaagcacaatgatgatgaacagtatgtctgggagtcccatgccggtggctcatttactgtgacaagagatacatctggtgagaaccttggtaggggaacaaagataactttgtttcttaaggaagatcaacttgagtatcttgaggaacgccgtctgaaggatttgataaagaagcattctgaattcattagctatccaatttctctctggattgagaagactactgaaaaggagatttctgatgatgaggatgaggaagagaagaaggatgaggagggtaaagttgaagatgtggatgaagataaggagaaggaggagaagaaaaagaagaccattaaggaggtatctcacgagtggtcattggtgaacaagcagaagcctatttggatgaggaaacctgaagagattacgaaagaagaatatgctgctttctacaagagtcttaccaatgattgggaagagcatttggctgttaagcacttttctgttgagggtcagctggagtttaaggctgtcctctttatccccaagagggcaccttttgatctctttgacaccaggaagaagcctaacaacatcaaactgtatgtccgccgtgtcttcatcatggacaactgtgaggagttgatgcctgaatatcttagctttgttaagggtattgtggattctgaggatcttcctctcaacatttctagagaaatgctgcagcagaacaagatcctgaaagtcatcaggaagaacttggtcaagaagtgcattgagatgttctttgaaattgctgaaaacaaggaagactataacaagttttatgaagccttctctaagaacctgaaacttggtattcatgaggattctcagaacaagacaaagctagctgaattgctcaggtatcactccactaagagtggtgatgagatgaccagcctcaaggactatgttaccaggatgaaggaaggacagaatgacatctactacattactggtgaaagcaagaaagctgtcgagaattcccccttccttgaaaagcttaagaagaagggatacgaggttctctacatggttgatgctattgatgagtatgctgttggccagcttaaggaatttgagggaaagaagttggtctctgctaccaaggaaggcctaaaacttgatgagagcgaagacgagaagaaaaagaaggaagaactaaaggataaatttgagggtctttgccatgtgatcaaggatgtgttgggtgacaaggtggagaaagttgtggtgtctgatcgtgttgtggattctccttgctgtctggtgacaggtgaatatgggtggacagccaacatggaaaggattatgaaagcacaggcattgagggacagcagcatggctggatacatgtcaagcaagaagacgatggagattaaccctgagaaccccatcatggaggagctgaggaagcgagcagatgctgataagaatgacaagtctgtgaaggatctcgtgctcttgctctttgagactgctcttctaacttctgggttcagccttgatgatcccaacactttcggtaacaggattcacaggatgctgaagcttggactgagcattgatgaagatgctggtgaagcagatgctgacatgcctcctcttgaggacgctgatgcagatgctgagggtagcaagatggaagaagttgattaa

SEQ ID NO: 106 - protein, 700 aa, Glycine max
MASETETFAFQAEINQLLSLIINTFYSNKEIFLRELISNASDALDKIRFESLTDKSKLDAQPELFIHIIPDKTNNTLSIIDSGIGMTKADLVNNLGTIARSGTKEFMEALAAGADVSMIGQFGVGFYSAYLVADKVIVTTKHNDDEQYVWESHAGGSFTVTRDTSGENLGRGTKITLFLKEDQLEYLEERRLKDLIKKHSEFISYPISLWIEKTTEKEISDDEDEEEKKDEEGKVEDVDEDKEKEEKKKKTIKEVSHEWSLVNKQKPIWMRKPEEITKEEYAAFYKSLTNDWEEHLAVKHFSVEGQLEFKAVLFIPKRAPFDLFDTRKKPNNIKLYVRRVFIMDNCEELMPEYLSFVKGIVDSEDLPLNISREMLQQNKILKVIRKNLVKKCIEMFFEIAENKEDYNKFYEAFSKNLKLGIHEDSQNKTKLAELLRYHSTKSGDEMTSLKDYVTRMKEGQNDIYYITGESKKAVENSPFLEKLKKKGYEVLYMVDAIDEYAVGQLKEFEGKKLVSATKEGLKLDESEDEKKKKEELKDKFEGLCHVIKDVLGDKVEKVVVSDRVVDSPCCLVTGEYGWTANMERIMKAQALRDSSMAGYMSSKKTMEINPENPIMEELRKRADADKNDKSVKDLVLLLFETALLTSGFSLDDPNTFGNRIHRMLKLGLSIDEDAGEADADMPPLEDADADAEGSKMEEVD

SEQ ID NO: 107 - protein, 16 aa, Glycine max
ADLVNNLGTIARSGTK

SEQ ID NO: 108 - protein, 10 aa, Glycine max
AVENSPFLEK

SEQ ID NO: 109 - protein, 13 aa, Glycine max
EDQLEYLEERRLK

SEQ ID NO: 110 - protein, 8 aa, Glycine max
EEYAAFYK

SEQ ID NO: 111 - protein, 14 aa, Glycine max
EGQNDIYYITGESK

SEQ ID NO: 112 - protein, 17 aa, Glycine max
EIFLRELISNASDALDK

SEQ ID NO: 113 - protein, 7 aa, Glycine max
FYEAFSK

SEQ ID NO: 114 - protein, 11 aa, Glycine max
HFSVEGQLEFK

SEQ ID NO: 115 - protein, 9 aa, Glycine max
IRFESLTDK

SEQ ID NO: 116 - protein, 11 aa, Glycine max
LAELLRYHSTK

SEQ ID NO: 117 - protein, 8 aa, Glycine max
LDESEDEK

SEQ ID NO: 118 - protein, 10 aa, Glycine max
LGIHEDSQNK

SEQ ID NO: 119 - protein, 11 aa, Glycine max
RAPFDLFDTRK

SEQ ID NO: 120 - protein, 8 aa, Glycine max
VEDVDEDK

SEQ ID NO: 121 - DNA, 2109 bp, Glycine max
  source 1..2109: mol_type = other DNA
  CDS 1..2109
atggcggagacagagacgttcgcattccaagcagagatcaaccagctcctcagtctcatcatcaacaccttctactccaacaaggagattttccttcgtgaactcatcagcaacgcctccgatgctttggacaagattcgatttgagagtttgacagataagagcaagctcgatgctcagccagagttgttcattcatattattccagacaagactaacaatacactgaccattgttgacagtggtattggcatgaccaaagctgatttggtgaacaacttgggtacaattgctagatctggtaccaaggaattcatggaagccttggctgctggtgctgatgttagtatgattggacagtttggtgtggggttctactctgcatatcttgttgctgagaaggtcattgttacaaccaaacacaatgatgatgaacagtatgtttgggagtctcaagctggaggttctttcactgtcaccagggataacactggtgaggttcttggcaggggtactaagatcactctctacctcaaggaagatcagcttgagtaccttgaggagcgtcgcttgaaggatctgatcaagaagcattctgagttcattagctacccaatttctctttggattgagaagactactgagaaggagatctctgacgatgaggatgaggaagagaagaaggatgaggagggtaaggtggaggatgttgacgaagagaaggagaaggaagaaaaaaagaaaaagaagatcaaggaagtgtcacatgaatggtccttggtgaacaagcagaagcccatttggatgaggaagcctgaggagatcacaaaagaggagtattctgctttctacaagagtcttaccaatgactgggaagagcatttggctgtcaagcacttctctgttgagggtcagttggagtttaaggctatactctttgttcccaagagggcaccttttgacctctttgacacaaggaagaagcctaacaacattaagctctatgtccgccgtgtctttattatggacaactgtgaggagttgattcctgagtatttgggctttgtcaagggtattgttgattctgaagatctcccactcaacatttcaagagaaatgttgcaacagaacaagatcttgaaggtcatccgtaagaacttggtcaagaagtgccttgagctcttctttgaaattgcagagaacaaggaggattacaacaagttctatgaggctttctctaagaacttaaagcttggtatccatgaggattcccagaacaagggaaagattgctgaattgctcaggtaccactccaccaagagtggtgatgagctgacaagcctcaaggattatgtgaccaggatgaaggaaggtcagagtgacatctactacatcaccggtgaaagcaagaaggctgttgagaactccccattcttggagaagttgaagaagaaagggtatgaggttcttttcatggttgatgccattgatgaatatgctgttggccagctcaaggaatttgagggcaagaagcttgtctctgccaccaaggagggtctcaaacttgacgagagtgaagatgagaagaagaagcaagaggagttgaaggagaagtttgacaacctatgcaaggtgatcaaggatgttttgggtgacaaggttgagaaggttgtagtctctgaccgtgttgttgattcaccatgctgtctggtgactggagaatacggttggactgcaaacatggaaaggatcatgaaagctcaagccctaagggacaacagcatggcaggatacatgtcaagcaagaagacaatggagatcaaccctgagaacccaatcatggaggagctcaggaagcgtgcggatgctgacaagaacgacaaatccgtgaaggaccttgttctgttgctgttcgagactgctcttctcacttctggtttcagccttgacgaacccaacacttttggaaacaggatccacagaatgttgaagcttggactgagcatcgacgaggatgcagctgaagctgatgctgatgctgatatgcccccactggaggaggaggctgaagctgatgctgagggcagcaaaatggaggaggttgattaa

SEQ ID NO: 122 - protein, 702 aa, Glycine max
MAETETFAFQAEINQLLSLIINTFYSNKEIFLRELISNASDALDKIRFESLTDKSKLDAQPELFIHIIPDKTNNTLTIVDSGIGMTKADLVNNLGTIARSGTKEFMEALAAGADVSMIGQFGVGFYSAYLVAEKVIVTTKHNDDEQYVWESQAGGSFTVTRDNTGEVLGRGTKITLYLKEDQLEYLEERRLKDLIKKHSEFISYPISLWIEKTTEKEISDDEDEEEKKDEEGKVEDVDEEKEKEEKKKKKIKEVSHEWSLVNKQKPIWMRKPEEITKEEYSAFYKSLTNDWEEHLAVKHFSVEGQLEFKAILFVPKRAPFDLFDTRKKPNNIKLYVRRVFIMDNCEELIPEYLGFVKGIVDSEDLPLNISREMLQQNKILKVIRKNLVKKCLELFFEIAENKEDYNKFYEAFSKNLKLGIHEDSQNKGKIAELLRYHSTKSGDELTSLKDYVTRMKEGQSDIYYITGESKKAVENSPFLEKLKKKGYEVLFMVDAIDEYAVGQLKEFEGKKLVSATKEGLKLDESEDEKKKQEELKEKFDNLCKVIKDVLGDKVEKVVVSDRVVDSPCCLVTGEYGWTANMERIMKAQALRDNSMAGYMSSKKTMEINPENPIMEELRKRADADKNDKSVKDLVLLLFETALLTSGFSLDEPNTFGNRIHRMLKLGLSIDEDAAEADADADMPPLEEEAEADAEGSKMEEVD

SEQ ID NO: 123 - protein, 16 aa, Glycine max
ADLVNNLGTIARSGTK

SEQ ID NO: 124 - protein, 10 aa, Glycine max
AVENSPFLEK

SEQ ID NO: 125 - protein, 13 aa, Glycine max
EDQLEYLEERRLK

SEQ ID NO: 126 - protein, 8 aa, Glycine max
EEYSAFYK

SEQ ID NO: 127 - protein, 14 aa, Glycine max
EGQSDIYYITGESK

SEQ ID NO: 128 - protein, 17 aa, Glycine max
EIFLRELISNASDALDK

SEQ ID NO: 129 - protein, 7 aa, Glycine max
FYEAFSK

SEQ ID NO: 130 - protein, 11 aa, Glycine max
HFSVEGQLEFK

SEQ ID NO: 131 - protein, 11 aa, Glycine max
IAELLRYHSTK

SEQ ID NO: 132 - protein, 9 aa, Glycine max
IRFESLTDK

SEQ ID NO: 133 - protein, 8 aa, Glycine max
LDESEDEK

SEQ ID NO: 134 - protein, 10 aa, Glycine max
LGIHEDSQNK

SEQ ID NO: 135 - protein, 11 aa, Glycine max
RAPFDLFDTRK

SEQ ID NO: 136 - protein, 9 aa, Glycine max
SGDELTSLK

SEQ ID NO: 137 - protein, 8 aa, Glycine max
VEDVDEEK

SEQ ID NO: 138 - DNA, 2109 bp, Glycine max
  source 1..2109: mol_type = other DNA
  CDS 1..2109
atggcttcggagactgagacgtttgcttttcaggccgagatcaaccagctcctcagtctaatcatcaacaccttctacagcaacaaggagatctttcttcgtgagctcatcagcaatgcctctgatgctttggacaagattcgatttgagagtcttacagacaagagcaagcttgatgctcagccagagttgttcattcacattattcctgacaagaccaacaacacattgtctatcattgacagtggtattggcatgactaaggccgatttggtgaataacctcggtactattgcaaggtctgggaccaaggaattcatggaagccctggctgctggtgctgatgtgagcatgattggacagtttggtgttggtttctactcggcttatcttgtagctgacaaggttattgttaccacaaagcacaatgatgatgagcagtatgtctgggagtcccatgctggtggctcatttactgtgacaagggatacatctggtgagaaccttggtaggggaacaaagataactttgtttctcaaggaagatcaacttgagtatcttgaggaacgccgtctgaaggatttgataaagaagcattctgaattcattagctatccaatttctctttggattgagaagaccactgaaaaggagatttctgatgatgaggacgaggaagagaagaaggatgaggagggtaaagttgaagatgtggatgaagataaggagaaggaggagaagaaaaagaagaccattaaggaggtatctcatgagtggtcattggtgaacaagcagaagcctatttggatgaggaaacctgaagagatcacaaaagaagaatatgctgctttctacaagagtcttaccaatgattgggaagagcatttggctgttaagcacttttctgttgagggtcagctggagtttaaggctgtcctctttatccccaagagggcaccttttgatctttttgacactaagaagaagcctaacaacatcaaactgtatgtccgccgtgttttcatcatggacaactgtgaggagttgatgccagaatatcttagctttgttaagggtattgtggattctgaggatcttcctctcaacatttctagagaaatgctgcagcagaacaagatcctgaaagtcatcaggaagaacttggtcaagaagtgcattgagatgttctttgaaattgctgagaacaaggaagactataacaagttttatgaagccttctctaagaatctgaaacttggtattcatgaggattctcagaacaagacaaaacttgctgaattgctcaggtatcactccaccaagagtggtgatgagatgaccagcctcaaggactatgttaccaggatgaaggaagggcagaatgacatctactacattactggtgaaagcaagaaagctgttgagaattcccctttccttgaaaagcttaagaagaaggggttcgaggttctctacatggttgatgctattgacgagtatgctgttggtcagcttaaggaatttgagggaaagaagttggtctctgctactaaggaaggcctgaaacttgacgagagcgaagacgaaaagaaaaagaaggaagaattgaaggagaaatttgagggtctctgccatgtgatcaaggatgtgttgggtgacaaggtggagaaagttgtggtgtctgatcgtgttgtggattctccttgctgtctcgtgactggtgaatatgggtggacagccaacatggaaaggattatgaaagcacaggcattgagggacagcagcatggctggatacatgtcgagcaagaagaccatggagattaaccctgagaaccccatcatggatgaactgaggaagcgagctgatgctgacaagaatgacaaatctgtgaaggatctcgttctcctactctttgagactgctcttctaacttctgggttcagccttgatgatcccaacactttcggtaacaggattcacaggatgctgaagcttggactgagcattgatgaagatgctggtgaagcagatgctgacatgcctcctctcgaggacgccgatgctgatgccgatgctgagggtagcaagatggaagaagttgattaa

SEQ ID NO: 139 - protein, 702 aa, Glycine max
MASETETFAFQAEINQLLSLIINTFYSNKEIFLRELISNASDALDKIRFESLTDKSKLDAQPELFIHIIPDKTNNTLSIIDSGIGMTKADLVNNLGTIARSGTKEFMEALAAGADVSMIGQFGVGFYSAYLVADKVIVTTKHNDDEQYVWESHAGGSFTVTRDTSGENLGRGTKITLFLKEDQLEYLEERRLKDLIKKHSEFISYPISLWIEKTTEKEISDDEDEEEKKDEEGKVEDVDEDKEKEEKKKKTIKEVSHEWSLVNKQKPIWMRKPEEITKEEYAAFYKSLTNDWEEHLAVKHFSVEGQLEFKAVLFIPKRAPFDLFDTKKKPNNIKLYVRRVFIMDNCEELMPEYLSFVKGIVDSEDLPLNISREMLQQNKILKVIRKNLVKKCIEMFFEIAENKEDYNKFYEAFSKNLKLGIHEDSQNKTKLAELLRYHSTKSGDEMTSLKDYVTRMKEGQNDIYYITGESKKAVENSPFLEKLKKKGFEVLYMVDAIDEYAVGQLKEFEGKKLVSATKEGLKLDESEDEKKKKEELKEKFEGLCHVIKDVLGDKVEKVVVSDRVVDSPCCLVTGEYGWTANMERIMKAQALRDSSMAGYMSSKKTMEINPENPIMDELRKRADADKNDKSVKDLVLLLFETALLTSGFSLDDPNTFGNRIHRMLKLGLSIDEDAGEADADMPPLEDADADADAEGSKMEEVD

SEQ ID NO: 140 - protein, 16 aa, Glycine max
ADLVNNLGTIARSGTK

SEQ ID NO: 141 - protein, 10 aa, Glycine max
AVENSPFLEK

SEQ ID NO: 142 - protein, 13 aa, Glycine max
EDQLEYLEERRLK

SEQ ID NO: 143 - protein, 8 aa, Glycine max
EEYAAFYK

SEQ ID NO: 144 - protein, 14 aa, Glycine max
EGQNDIYYITGESK

SEQ ID NO: 145 - protein, 17 aa, Glycine max
EIFLRELISNASDALDK

SEQ ID NO: 146 - protein, 7 aa, Glycine max
FYEAFSK

SEQ ID NO: 147 - protein, 11 aa, Glycine max
HFSVEGQLEFK

SEQ ID NO: 148 - protein, 9 aa, Glycine max
IRFESLTDK

SEQ ID NO: 149 - protein, 11 aa, Glycine max
LAELLRYHSTK

SEQ ID NO: 150 - protein, 8 aa, Glycine max
LDESEDEK

SEQ ID NO: 151 - protein, 10 aa, Glycine max
LGIHEDSQNK

SEQ ID NO: 152 - protein, 10 aa, Glycine max
RAPFDLFDTK

SEQ ID NO: 153 - protein, 8 aa, Glycine max
VEDVDEDK

SEQ ID NO: 154 - DNA, 2109 bp, Glycine max; identical to SEQ ID NO: 138
  source 1..2109: mol_type = other DNA
  CDS 1..2109

SEQ ID NO: 155 - protein, 702 aa, Glycine max; identical to SEQ ID NO: 139

SEQ ID NO: 156 - protein, 12 aa, Glycine max
AGGALSIAFVSK

SEQ ID NO: 157 - protein, 9 aa, Glycine max
DGLIVSIGK

SEQ ID NO: 158 - DNA, 2946 bp, Glycine max
  source 1..2946: mol_type = other DNA
  CDS 1..2946
atggcttcttcttccacgacaatgcggttctccgcggcctccaccggcgccgcggcggcgcttcctcgccggagctccgtcgccggattcatcggcgtcgcggccagatcctccgccaaatcgaggctccgcttcatcggaaggaacgcgaacttgagcctccggatgaggaggatgagctctttctccgtcgtgaagtgcgtctccggaagcgaggccaaggtgcaggataccgtggccaaacaacaagaagctacaacctctctcagctccttcacgccagatgcttcatctattgcctcaagtattaaataccatgcagagttcactccattgttttctccagagaattttgatctaccacaggcttttcttgcaactgcacaaagtgttcgagattctctcattataaactggaatgctacctatgattactatgagaaactgaatgttaagcaggcatattacctttcgatggaatttttgcagggtagagcattattaaatgcaataggcaatctggagctaactggtccctatgctgaggctttgagcaagctcgggcataaattagaaaatgtggcataccaggagccagatgctgcacttggaaatggtggtcttgggcgtcttgcttcatgtttcttggactctttggcaaccttgaactatccagcttggggttatggattgagatataagtatggtttatttaaacagcgaataaccaaagatgggcaggaggaagttgctgaagattggcttgagatgggcaatccctgggagattattagaaatgatgtttcatatcctgtaaagttctatggcaaggttgtttcagggtcagatggtaaaaaacattggattggaggagaagatataaaagctgttgcacatgatgttcccataccaggatataaaactaagactacaatcaacttgagactttggtctacaaaagctgcatcagaagaatttgatttatctgcttttaatgctggaaggcacactgaagcatctgaggctctagcaaatgctgaaaagatatgctatatactctaccctggggatgaaccaatagagggcaagatccttcgcctcaagcaacaatatactttatgttctgcttctcttcaagacatcattgctcgttttgagagaagatcaggagcaaatgttaattgggaagagtttcccgagaaggttgcagtgcagatgaatgatactcacccaacgttgtgcatcccagagctcatgagaattttaatagatgtaaagggcttaaattggaaggatgcttggaatattacacaaaggactgtagcatatacaaaccacactgttcttccagaggcattagagaaatggagcttggatcttatgcagaaactgcttccacgccatattgagataatagaaatgattgacgaggagctggttcggaccataattgcagaatatggcacagaaaattcagacttgctggagaaaaaattgaaggaaatgagaatactagaaaatgtcgaattgactgcagaatttgcagatatactggttaagtccaaagaggccattgatattcctagtgaggagctgcaaagttctgaacaagcagaagcagaagacgaaaaagatgatgatgaagtggaagcagttgctaaaaagaatggaacagatgaaagttccattgaggatgagaaagaagagttgcctgagccagtaccagaaccaccaaaattggttcgtatggcaaatttgtgtgttgtgggtggtcatgcagtgaatggggttgcagagatacatagtgaaattgtaaaagatgatgttttcaatgcattttataagttgtggcccgagaagttccaaaacaaaacaaatggtgtgacaccaaggagatggatcaggttctgcaatccagatctgagtaaaataataaccgagtggataggtactgaggattgggtcctaaatactggaaaattggccgagctacggaagtttgtagacaatgaggatctacaagtgcagtggagggaagcaaagaggagtaacaaagttaaagttgcagcattcatcagagaaaaaacagggtattctgtcagtcctgatgcaatgtttgacatccaggtgaagagaatccatgaatataagcgacaattaatgaacatatttggaattgtttaccgatacaagaagatgaaagaaatgagtgcggcagaaagggaagcaaattttgttcctagagtttgtatatttgggggaaaagcatttgctacatatgtgcaagccaagagaattgtgaaatttattacagatgttggggctacagtaaatcatgaccctgaaataggagatctacttaaggttgtttttgtccctgattataatgtcagtgttgccgagatgcttattccagcaagtgaattgtcacaacatatcagtactgctggaatggaggcaagtggaactagcaatatgaaatttgcaatgaatggctgcatattgattggaactttagatggggccaatgttgaaataagggaagaggttggtgcagacaacttcttcctctttggtgccaaggctcatgaaattgctgggctgaggaaagaaagagcagagggaaagtttgtgcctgatccacgatttgaagaagttaaagaatttgtcagaagtggcatttttggatcttacaactatgacgaattgatgggatccttagaaggaaatgaaggttttggtcgagcagattatttccttgtagggaaagactttcccagctacatagaatgccaagagaaggttgatgaagcataccgtgaccaaactaaatggacaagaatgtcaatattgaacacagcaggttcatacaagtttagcagtgaccgcacgattcatgaatatgcccgagagatatggaacattgaaccagtccaattgccatag

SEQ ID NO: 159 - protein, 981 aa, Glycine max
MASSSTTMRFSAASTGAAAALPRRSSVAGFIGVAARSSAKSRLRFIGRNANLSLRMRRMSSFSVVKCVSGSEAKVQDTVAKQQEATTSLSSFTPDASSIASSIKYHAEFTPLFSPENFDLPQAFLATAQSVRDSLIINWNATYDYYEKLNVKQAYYLSMEFLQGRALLNAIGNLELTGPYAEALSKLGHKLENVAYQEPDAALGNGGLGRLASCFLDSLATLNYPAWGYGLRYKYGLFKQRITKDGQEEVAEDWLEMGNPWEIIRNDVSYPVKFYGKVVSGSDGKKHWIGGEDIKAVAHDVPIPGYKTKTTINLRLWSTKAASEEFDLSAFNAGRHTEASEALANAEKICYILYPGDEPIEGKILRLKQQYTLCSASLQDIIARFERRSGANVNWEEFPEKVAVQMNDTHPTLCIPELMRILIDVKGLNWKDAWNITQRTVAYTNHTVLPEALEKWSLDLMQKLLPRHIEIIEMIDEELVRTIIAEYGTENSDLLEKKLKEMRILENVELTAEFADILVKSKEAIDIPSEELQSSEQAEAEDEKDDDEVEAVAKKNGTDESSIEDEKEELPEPVPEPPKLVRMANLCVVGGHAVNGVAEIHSEIVKDDVFNAFYKLWPEKFQNKTNGVTPRRWIRFCNPDLSKIITEWIGTEDWVLNTGKLAELRKFVDNEDLQVQWREAKRSNKVKVAAFIREKTGYSVSPDAMFDIQVKRIHEYKRQLMNIFGIVYRYKKMKEMSAAEREANFVPRVCIFGGKAFATYVQAKRIVKFITDVGATVNHDPEIGDLLKVVFVPDYNVSVAEMLIPASELSQHISTAGMEASGTSNMKFAMNGCILIGTLDGANVEIREEVGADNFFLFGAKAHEIAGLRKERAEGKFVPDPRFEEVKEFVRSGIFGSYNYDELMGSLEGNEGFGRADYFLVGKDFPSYIECQEKVDEAYRDQTKWTRMSILNTAGSYKFSSDRTIHEYAREIWNIEPVQLP

SEQ ID NO: 160 - protein, 9 aa, Glycine max
AFATYVQAK

SEQ ID NO: 161 - protein, 23 aa, Glycine max
QQEATTSLSSFTPDASSIASSIK

SEQ ID NO: 162 - DNA, 2706 bp, Glycine max
  source 1..2706: mol_type = other DNA
  CDS 1..2706
atggcgactgagaatcctttcaatagcattttgacgacgctcgaaaagcccggtggtgctggcgagtttggaaagtactttagcttgcctgctctcaacgatcgtagaatcgatagacttccttactctgtgaggatacttttggaatctgcaatccggaactgtgatgagtttcaagttaagagtaacgacgttgaaaaaattattgattgggagaatacctctccgaagctggtggagattccattcaagccagctagagtgcttctccaggattttactggcgtgcctgctgttgtcgatcttgcttgcatgcgagatgccatgaacaaacttggtggtgattctaataaaattaatcccttggtaccagttgatcttgtcattgatcactctgttcaagttgatgtggcaagatcagaaaatgctgtccaggcaaatatggaacttgaattccaaagaaacaaggaaagatttggtttcctcaaatggggttctaatgcattcaataatatgcttgtcgttcctcctggatcagggatagttcaccaggttaatttagaataccttggtagagttgtgttcaacacaaatggtgtgctttatcctgacagcgttgttggaactgattcacacacaactatgattgatggcctgggtgttgctggatggggagttggtggaatagaagcagaagctgcaatgcttggccaacccatgagcatggtcctaccgggtgtcgttgggttcaaattattaggcaaactacgagatggtgtcacagctactgacttggtgttgactgttacacaaatgctaagaaagcatggggttgttggcaagtttgtagagttttatggggaaggcatgagtgaactgtctttggcagatcgtgccaccatagcaaacatgtctcctgagtatggtgcaaccatgggcttctttcctgtggatcatgtcactttgcaatatttgagactgactggcagaagtgatgagactgtttctatgatagaatcgtacttacgagcaaataagatgtttgtggattacagcgagccccaagtggagagagtgtactcatcgtatttggaactcaatcttgaggatgtagaaccctgtgtctcaggtccaaaaaggcctcatgaccgtgtccctttgagagaaatgaaggtagactggcatgcctgcctcaacaataaagttggattcaagggttttgctgtaccaaaagaatctcagaataaggttgcagagttcacattccaagggacaccagcacatcttaggcacggtgatgttgttatagctgctatcaccagttgtacaaatacttcaaatcctagcgtaatgcttggagctgcattggttgcaaagaaagcatgtgaattgggattgcaggtgaagccatggattaaaacaagccttgccccaggttctggtgttgtgactaagtatttgcagaggagtggcttgcagaagtatttgaatgagctagggtttaatatagttgggtatggatgcaccacatgcattggaaattcaggggatattaatgaagctgttgcatctgcaattactgaaaatgatatagtagctgcagctgtactgtctggaaataggaactttgagggccgagttcacccattaacaagagctaattatcttgcttctcctcctcttgttgttgcctatgcccttgctggcacagtggacattgactttgacactgaacccattgggatagggaaggatggcacaaagatcttcttcagggatatttggccatccagtgaagaaatagcaaatgttgtacaatcaagtgtgctgcctgctatgtttagggacacatataatgcgatcacccaagggaatcccatgtggaataatttatctgttccaactggcactctttatgcttgggaccctacatcaacttatattcatgagccaccttattttagagatatgagcatgtctcccccaggctctcatggagtgaaagatgcttactgtttgctcaactttggagacagtatcacaactgaccacatctctccagctggcagcatacataaggacagtcctgcagccagataccttatagaacgtggggttgatagacgggacttcaactcctatggaagtcgccgtggtaatgatgaggtgatggcccgaggaacttttgccaatattcgcattgtcaacaaatttttgaatggagaagttggacctaaaactattcatattccttctggggagaagttatcagtctttgatgctgcagagaaatacaagagtgaggggcatgatatgattatcttggctggtgctgagtatgggagtggaagctctcgtgattgggctgcaaaggggccaatgctactgggcgtgaaagctgtcatagcaaaaagttttgaaaggattcaccgaagtaatttggtgggaatgggtatcattccattgtgttttaagcccggggatgatgctgattctcttggattaactggccatgaacgttacaccattgatctaccaagcaatgtgaatgaaatacgacccggccaagatgtcacagtggtgacagatgccgggaaatcatttgtatctacactgagatttgatacagaggttgaactagcttacttcaaccatggaggcatcttacaatatgtcatcagaaacatggtcaatgctaaacattga

SEQ ID NO: 163 - protein, 901 aa, Glycine max
MATENPFNSILTTLEKPGGAGEFGKYFSLPALNDRRIDRLPYSVRILLESAIRNCDEFQVKSNDVEKIIDWENTSPKLVEIPFKPARVLLQDFTGVPAVVDLACMRDAMNKLGGDSNKINPLVPVDLVIDHSVQVDVARSENAVQANMELEFQRNKERFGFLKWGSNAFNNMLVVPPGSGIVHQVNLEYLGRVVFNTNGVLYPDSVVGTDSHTTMIDGLGVAGWGVGGIEAEAAMLGQPMSMVLPGVVGFKLLGKLRDGVTATDLVLTVTQMLRKHGVVGKFVEFYGEGMSELSLADRATIANMSPEYGATMGFFPVDHVTLQYLRLTGRSDETVSMIESYLRANKMFVDYSEPQVERVYSSYLELNLEDVEPCVSGPKRPHDRVPLREMKVDWHACLNNKVGFKGFAVPKESQNKVAEFTFQGTPAHLRHGDVVIAAITSCTNTSNPSVMLGAALVAKKACELGLQVKPWIKTSLAPGSGVVTKYLQRSGLQKYLNELGFNIVGYGCTTCIGNSGDINEAVASAITENDIVAAAVLSGNRNFEGRVHPLTRANYLASPPLVVAYALAGTVDIDFDTEPIGIGKDGTKIFFRDIWPSSEEIANVVQSSVLPAMFRDTYNAITQGNPMWNNLSVPTGTLYAWDPTSTYIHEPPYFRDMSMSPPGSHGVKDAYCLLNFGDSITTDHISPAGSIHKDSPAARYLIERGVDRRDFNSYGSRRGNDEVMARGTFANIRIVNKFLNGEVGPKTIHIPSGEKLSVFDAAEKYKSEGHDMIILAGAEYGSGSSRDWAAKGPMLLGVKAVIAKSFERIHRSNLVGMGIIPLCFKPGDDADSLGLTGHERYTIDLPSNVNEIRPGQDVTVVTDAGKSFVSTLRFDTEVELAYFNHGGILQYVIRNMVNAKH

SEQ ID NO: 164 - protein, 9 aa, Glycine max
FLNGEVGPK

SEQ ID NO: 165 - protein, 9 aa, Glycine max
LSVFDAAEK

SEQ ID NO: 166 - protein, 9 aa, Glycine max
LSVFDVAEK

SEQ ID NO: 167 - protein, 12 aa, Glycine max
TSLAPGSGVVTK

SEQ ID NO: 168 - DNA, 2706 bp, Glycine max
  source 1..2706: mol_type = other DNA
  CDS 1..2706
atggcgactgagaaccctttcaatagcattttgaggacgctcgagaagcccggcggcgctggcgagtttggaaagtactttagcttgcccgctctcaacgatcctagaatcgatagacttccttactctgtgaggatacttttggaatctgcaatccgtaactgtgatgagtttcaagttaagagtaacgacattgaaaaaattattgattgggagaatacctctcccaagctggtggagattccattcaagccagctagagtgcttctgcaggactttactggggtgcctgctgtggttgatcttgcttgcatgcgagatgcaatgaacaaacttggtggtgattctaataaaattaaccccttggtaccagttgatcttgtcattgatcactctgttcaagttgatgtggcaagatcagaaaatgctgtccaggcaaatatggaacttgaattccaaagaaacaaggaaagatttggtttcctcaaatggggttctaatgcattcaataatatgcttgtcgttcctcctggatcagggatagttcatcaggttaatttagaataccttggtagagttgtgttcaacacaaatggtgtgctttatccagacagcgttgtcggaactgattcacacactactatgatagatggcctgggtgttgctggatggggagttggtggaatagaagcagaagctgcaatgcttggccagcccatgagcatggtcctaccgggtgttgttgggttcaaattattaggcaaactacgagatggtgtcacagctactgacttggtgttgactgttactcaaatgctaagaaagcatggggttgttggcaagtttgtagagttttatggggaaggcatgagtgaactgtctttggcagatcgtgccactatagcaaacatgtctcctgagtatggtgcaaccatgggcttctttcctgtggatcatgtcactttgcaatatttgagactgactggcagaagtgatgagactgtttctatgatagaatcctatttacgagcaaataagatgtttgtggattatagcgagccccaagtggagagagtgtactcatcctatttggaactcaatcttgaggatgtagagccctgtgtctcaggtccaaaaaggcctcacgaccgtgtccctttgagagaaatgaaggtagactggcatgcctgcctcaacaataaagttggatttaagggttttgctgtatcaaaagaatctcagaataaggttgcagagttcacattccaagggacaccagcacatcttaggcatggtgatgttgttatagctgctatcaccagttgtacaaatacttcaaatcctagcgtaatgcttggagctgcattggttgcaaagaaagcatgtgaattgggattgcaggtgaagccatggattaaaacaagtcttgccccaggttctggtgttgtaactaagtatttgcagaggagtggcttgcagaagtatttgaatgagctagggtttaatatagttgggtatggatgcaccacatgcattggaaattcaggggatattaatgaagctgttgcatctgcgattaccgaaaatgatatagtagctgcagctgtattgtctggaaataggaactttgagggccgagttcacccattaacaagagctaattatcttgcttctcctcctcttgttgttgcctatgcccttgctggcacagtggacattgactttgacactgaacccattgggatagggaaggatggaacagagatcttcttcaaggatatttggccatccagtgaagaaatagcaaatgttgtacaatcaagtgtgctgcctgatatgtttagggacacatataatgcaatcacccaagggaatcccatgtggaataatttatctgttccaacgggcactctttatgcttgggaccctacatcgacttatattcatgagccaccttattttagagatatgagcatgtctcccccaggctctcatggagtgaaggatgcttactgtttgctcaactttggagacagtatcacaactgaccacatctctccagctggcagcatacataaggacagtcctgcggccagataccttatagaacgtggggttgatagacgggacttcaactcctatggaagtcgccgtggtaatgatgaggtgatggcccgaggaacgtttgccaatattcgcattgtcaacaaatttttgaatggagaagttggacctaaaactattcatattccttctggggagaagttgtcagtctttgacgttgcagagaaatacaagagtgaagggcatgatatgattatcttggctggtgctgagtatgggagtggaagctctcgtgattgggctgcaaaggggccaatgctactgggcgtgaaagctgtcatagcaaaaagttttgaaaggattcaccgaagtaatttggtgggaatgggtatcattcctttgtgttttaagcccggggaggatgctgattctcttggattaactggtcaagaacgttacaccattgatctaccaagcaatgtgaatgaaatacgacccggtcaagatgtcacagtggtgacagacaccgggaaatcatttgtatctacattgagatttgatactgaggttgaactagcttacttcaaccatggaggtatcttacaatatgtcatcagaaacttgatcaatgctaaacattga

SEQ ID NO: 169 - protein, 901 aa, Glycine max
MATENPFNSILRTLEKPGGAGEFGKYFSLPALNDPRIDRLPYSVRILLESAIRNCDEFQVKSNDIEKIIDWENTSPKLVEIPFKPARVLLQDFTGVPAVVDLACMRDAMNKLGGDSNKINPLVPVDLVIDHSVQVDVARSENAVQANMELEFQRNKERFGFLKWGSNAFNNMLVVPPGSGIVHQVNLEYLGRVVFNTNGVLYPDSVVGTDSHTTMIDGLGVAGWGVGGIEAEAAMLGQPMSMVLPGVVGFKLLGKLRDGVTATDLVLTVTQMLRKHGVVGKFVEFYGEGMSELSLADRATIANMSPEYGATMGFFPVDHVTLQYLRLTGRSDETVSMIESYLRANKMFVDYSEPQVERVYSSYLELNLEDVEPCVSGPKRPHDRVPLREMKVDWHACLNNKVGFKGFAVSKESQNKVAEFTFQGTPAHLRHGDVVIAAITSCTNTSNPSVMLGAALVAKKACELGLQVKPWIKTSLAPGSGVVTKYLQRSGLQKYLNELGFNIVGYGCTTCIGNSGDINEAVASAITENDIVAAAVLSGNRNFEGRVHPLTRANYLASPPLVVAYALAGTVDIDFDTEPIGIGKDGTEIFFKDIWPSSEEIANVVQSSVLPDMFRDTYNAITQGNPMWNNLSVPTGTLYAWDPTSTYIHEPPYFRDMSMSPPGSHGVKDAYCLLNFGDSITTDHISPAGSIHKDSPAARYLIERGVDRRDFNSYGSRRGNDEVMARGTFANIRIVNKFLNGEVGPKTIHIPSGEKLSVFDVAEKYKSEGHDMIILAGAEYGSGSSRDWAAKGPMLLGVKAVIAKSFERIHRSNLVGMGIIPLCFKPGEDADSLGLTGQERYTIDLPSNVNEIRPGQDVTVVTDTGKSFVSTLRFDTEVELAYFNHGGILQYVIRNLINAKH

SEQ ID NO: 170 - protein, 9 aa, Glycine max
FLNGEVGPK

SEQ ID NO: 171 - protein, 9 aa, Glycine max
LSVFDVAEK

SEQ ID NO: 172 - protein, 12 aa, Glycine max
TSLAPGSGVVTK

SEQ ID NO: 173 - DNA, 2292 bp, Glycine max
  source 1..2292: mol_type = other DNA
  CDS 1..2292
atggcatctcacatcgttggatacccccgtatgggtcccaagagagagctcaagttcgctctcgagtctttctgggatggcaagagcagcgccgaggatttgcagaaggtgtcttctgatctcagggcatccatctggaagcagatggctgatgctgggatcaagtacatccccagcaacactttctctcactatgaccaggttctcgacgccaccgccaccctcggtgccgttccaccaaggtacggctggaccggcggcgagattgggtttgatacctacttctccatggccagaggtaatgctaccgtgccagctatggagatgaccaagtggttcgacaccaactaccactttattgtccctgaattgggccctgatgtgaacttcacctatgcttctcacaaggctgttgatgaatacaaggaggccaaggcgcttggagtggataccgttccggtcctcgttggccctgttacatacctgttgctctccaagcctgccaagggagttgagaaatccttttctctcctctctctccttcccaaggttcttgctgtctacaaggaagttattgctgaccttaaggcagctggtgcttcatggattcagtttgatgagcctacccttgtcttggaccttgagtctcacaagttgcaagcattcactgacgcatatgcagaacttgcgcctgctttgtctggtttgaatgttcttgttgagacctactttgctgacatccctgctgaggcatacaagaccctcacatctctgaatggcgtcactgcatatggatttgatttggtccgtggaaccaacactcttgatttgatcaagggtggatttcccagcggaaaatacctctttgctggagtggttgatggaaggaacatctgggccaatgaccttgctgcttctctcactaccttgcagggtcttgagggcattgtgggcaaagataagcttgttgtgtccacctcctcctcccttcttcacactgctgttgatctagttaacgagaccaagttggatgatgagatcaagtcatggctagcttttgctgcccaaaaaattgttgaagttaacgcattggctaaagcattgtctggccacaaggatgaggccttcttctctggtaatgctgctgctctggcttcaaggaagtcttctccaagagtgaccaacgaggctgttcagaaggctgctgctgcattgaagggttcagatcatcgccgtgcaacaaatgtcagtgccagactggattctcaacaaaagaagctcaaccttccaatcctgccaaccaccactattggatccttccctcagactgtagaactgaggagggtacgccgtgaattcaaggctaacaagatctccgaggaagagtatgtaaagtcaattaaggaggaaattcgcaaagttgttgagcttcaagaagagcttgatattgatgttcttgttcatggagaaccagagagaaatgatatggttgagtacttcggtgaacaattgtcaggctttgccttcaccgttaatgggtgggtgcaatcctatggttcccgttgcgtgaagccaccgatcatctatggtgatgtgagccgcccaaagccaatgaccgtcttctggtcatctctggctcagagctttaccaagcgcccaatgaagggaatgcttaccggtcctgttaccattctcaactggtcctttgttagaaatgaccaacctagatctgagaccacctaccagattgctttgtctatcaaggacgaagtggaagaccttgaaaaggctggcatcactgttatccaaattgatgaagctgctttgagagagggtcttccactgaggaaatcagagcaagctcactacttggactgggctgtccatgccttcagaatcaccaatgttggtgtccaggataccacccagatccacactcacatgtgctactcgaatttcaacgacatcatccactccatcatcgacatggacgccgatgttatcaccattgagaactctcgctccgacgagaagcttctgtcagtcttccgcgaaggtgtgaagtatggtgctggaattggccctggtgtctatgacatccactccccaagaataccaccaactgaagaaattgctgacagaatcaacaagatgctggcagtgctcgagaagaacatcttgtgggtgaaccctgactgtgggctcaagacccgtaagtacactgaggtgaagccagccctcacaaacatggttgccgcagcaaaactcatccgcaacgaacttgccaagtga

SEQ ID NO: 174 - protein, 763 aa, Glycine max
MASHIVGYPRMGPKRELKFALESFWDGKSSAEDLQKVSSDLRASIWKQMADAGIKYIPSNTFSHYDQVLDATATLGAVPPRYGWTGGEIGFDTYFSMARGNATVPAMEMTKWFDTNYHFIVPELGPDVNFTYASHKAVDEYKEAKALGVDTVPVLVGPVTYLLLSKPAKGVEKSFSLLSLLPKVLAVYKEVIADLKAAGASWIQFDEPTLVLDLESHKLQAFTDAYAELAPALSGLNVLVETYFADIPAEAYKTLTSLNGVTAYGFDLVRGTNTLDLIKGGFPSGKYLFAGVVDGRNIWANDLAASLTTLQGLEGIVGKDKLVVSTSSSLLHTAVDLVNETKLDDEIKSWLAFAAQKIVEVNALAKALSGHKDEAFFSGNAAALASRKSSPRVTNEAVQKAAAALKGSDHRRATNVSARLDSQQKKLNLPILPTTTIGSFPQTVELRRVRREFKANKISEEEYVKSIKEEIRKVVELQEELDIDVLVHGEPERNDMVEYFGEQLSGFAFTVNGWVQSYGSRCVKPPIIYGDVSRPKPMTVFWSSLAQSFTKRPMKGMLTGPVTILNWSFVRNDQPRSETTYQIALSIKDEVEDLEKAGITVIQIDEAALREGLPLRKSEQAHYLDWAVHAFRITNVGVQDTTQIHTHMCYSNFNDIIHSIIDMDADVITIENSRSDEKLLSVFREGVKYGAGIGPGVYDIHSPRIPPTEEIADRINKMLAVLEKNILWVNPDCGLKTRKYTEVKPALTNMVAAAKLIRNELAK

SEQ ID NO: 175 - protein, 21 aa, Glycine max
AGITVIQIDEAALREGLPLRK

SEQ ID NO: 176 - protein, 24 aa, Glycine max
ALGVDTVPVLVGPVTYLLLSKPAK

SEQ ID NO: 177 - protein, 16 aa, Glycine max
DEAFFSGNAAALASRK

SEQ ID NO: 178 - protein, 8 aa, Glycine max
DEVEDLEK

SEQ ID NO: 179 - protein, 8 aa, Glycine max
ISEEEYVK

SEQ ID NO: 180 - protein, 9 aa, Glycine max
IVEVNALAK

SEQ ID NO: 181 - protein, 8 aa, Glycine max
LIRNELAK

SEQ ID NO: 182 - protein, 10 aa, Glycine max
LLSVFREGVK

SEQ ID NO: 183 - protein, 21 aa, Glycine max
LVVSTSSSLLHTAVDLVNETK

SEQ ID NO: 184 - protein, 10 aa, Glycine max
SFSLLSLLPK

SEQ ID NO: 185 - protein, 12 aa, Glycine max
SSPRVTNEAVQK

SEQ ID NO: 186 - protein, 26 aa, Glycine max
TLTSLNGVTAYGFDLVRGTNTLDLIK

SEQ ID NO: 187 - protein, 29 aa, Glycine max
YGAGIGPGVYDIHSPRIPPTEEIADRINK

SEQ ID NO: 188 - DNA, 2196 bp, Glycine max
  source 1..2196: mol_type = other DNA
  CDS 1..2196
atggcttcttccacttcctctatctctctctcccaagccctcctccgccccaccaccacctctctcccctcctcctcctcctccctccgcctcacccccactctccgccctaccccacccgccctctccctccgccgccgcctcaccacctccatccgcgccgccgcctccgtcaaaaccgtcgagaaggcaaccgcggacgccgcgctggtcgaaaaatccgtcaacacgatccgcttcctcgccatcgacgccgtcgagaaggcgaattcgggtcacccggggctccccatgggatgcgctccgatgggtcacgtgctatatgatgaaaccatgaagtataaccctaagaaccctttctggttcaaccgcgatcgcttcgttctctccgctgggcacgggtgcatgcttcagtacgcgcttctgcacctcgctgggttcgatagcgtcaaggaagaggatttgagggaatttcgccaatgggggagcagaactccgggacaccctgagaattttgagactccgggaattgaagttacaacaggtcctcttggtcaggggattgccaatgctgttggtttggcccttgcagagaagcacttggctgcaagatacaacaagcctgacaatgagattgttgaccattacacgtatgctattctgggtgatggttgtcaaatggagggaattgcaaatgaagcatgctcacttgctggccactggggactggggaagctgatagcattttatgatgacaatcatatttctattgatggtaacacagagattgcgttcaccgagagtgttgatagtcgttttgagggacttgggtggcatgttatttgggtaaagaatggaaataatggctatgatgatattcgtgctgccattaaggaagcaaaagctgtcaaagacaaacccacattaatcaaggtcacaacaaccattggttatggttctcctaacaaggctaactcctacagtgtgcatggaagtgcactgggtgccaaagaagttgatgccaccaggcagaaccttggatggtcacatgagccattccacgtgcctgaggatgtcaaaaagcattggagtcgccacacccctgagggtgctgcacttgaagctgagtggaatgctaagtttgctgagtatgaaaagaaatacaaggaggaggctgcagaattgaaatctattatcaatggtgaattccctgctggttgggagaaagcacttccgacatacactccagagagcccagcggatgccaccagaaacctgtctcaaacaaaccttaatgcccttgcaaaggttcttcccggtctgcttggtggcagtgcagatcttgcttcttccaacatgaccttgctcaaaatgttcggggacttccaaaaggatactccagcagagcgtaatgttagattcggtgttagagaacacggaatgggagctatctgcaacggcattgctcttcacagccctggactgattccatattgtgcaaccttctttgtattcactgactacatgagaggtgccataaggctttctgcgctgtctgaggctggggttatttatgtcatgacccatgattcaataggacttggagaagatgggccaacccaccagccaattgagcacctagcaagcttccgggcaatgccaaacattttgatgcttcgtcccgccgacggtaacgaaacagccggagcatacaaagtggccgtgctcaacaggaagagaccctccattcttgccctatccaggcaaaaactgccccagcttcccggaacttccattgaaggagttgaaaagggtggttacaccatttcggacaactccactggcaacaagcctgatgtcattttgatcggaactggttcggaattggaaatcgctgccaaagctgctgatgacctaaggaaggaagggaaggctgttagagttgtttcccttgtttcttgggaactttttgatgagcaatcagaagcctacaaggagagtgttttccctgctgctgtttcagccagagttagcattgaggcaggatcaacatttgggtgggagaaaattgttggagcaaagggaaaagcaataggcattgatcgttttggagctagtgctccagctggaagaatatacaaagaatttggtatcactaaggaagctgttgttgctgcagctaaagagcttatctag

SEQ ID NO: 189 - protein, 731 aa, Glycine max
MASSTSSISLSQALLRPTTTSLPSSSSSLRLTPTLRPTPPALSLRRRLTTSIRAAASVKTVEKATADAALVEKSVNTIRFLAIDAVEKANSGHPGLPMGCAPMGHVLYDETMKYNPKNPFWFNRDRFVLSAGHGCMLQYALLHLAGFDSVKEEDLREFRQWGSRTPGHPENFETPGIEVTTGPLGQGIANAVGLALAEKHLAARYNKPDNEIVDHYTYAILGDGCQMEGIANEACSLAGHWGLGKLIAFYDDNHISIDGNTEIAFTESVDSRFEGLGWHVIWVKNGNNGYDDIRAAIKEAKAVKDKPTLIKVTTTIGYGSPNKANSYSVHGSALGAKEVDATRQNLGWSHEPFHVPEDVKKHWSRHTPEGAALEAEWNAKFAEYEKKYKEEAAELKSIINGEFPAGWEKALPTYTPESPADATRNLSQTNLNALAKVLPGLLGGSADLASSNMTLLKMFGDFQKDTPAERNVRFGVREHGMGAICNGIALHSPGLIPYCATFFVFTDYMRGAIRLSALSEAGVIYVMTHDSIGLGEDGPTHQPIEHLASFRAMPNILMLRPADGNETAGAYKVAVLNRKRPSILALSRQKLPQLPGTSIEGVEKGGYTISDNSTGNKPDVILIGTGSELEIAAKAADDLRKEGKAVRVVSLVSWELFDEQSEAYKESVFPAAVSARVSIEAGSTFGWEKIVGAKGKAIGIDRFGASAPAGRIYKEFGITKEAVVAAAKELI

SEQ ID NO: 190 - protein, 18 aa, Glycine max
AIGIDRFGASAPAGRIYK

SEQ ID NO: 191 - protein, 27 aa, Glycine max
ALPTYTPESPADATRNLSQTNLNALAK

SEQ ID NO: 192 - protein, 14 aa, Glycine max
ANSYSVHGSALGAK

SEQ ID NO: 193 - protein, 10 aa, Glycine max
ATADAALVEK

SEQ ID NO: 194 - protein, 7 aa, Glycine max
DKPTLIK

SEQ ID NO: 195 - protein, 30 aa, Glycine max
GGYTISDNSTGNKPDVILIGTGSELEIAAK

SEQ ID NO: 196 - protein, 14 aa, Glycine max
LPQLPGTSIEGVEK

SEQ ID NO: 197 - protein, 14 aa, Glycine max
NGNNGYDDIRAAIK

SEQ ID NO: 198 - protein, 15 aa, Glycine max
SVNTIRFLAIDAVEK

SEQ ID NO: 199 - protein, 12 aa, Glycine max
VTTTIGYGSPNK

SEQ ID NO: 200 - DNA, 1797 bp, Glycine max
  source 1..1797: mol_type = other DNA
  CDS 1..1797
atggaagttccttcggaggtaccatctactgacactggcaccagcaccggcaccggcgaaaccatgagcaagaatgcgttgaagcgggagctgaagaacaaacagagagaagaagaaaggaagcgcaaggatgaggagaaggccaaaaaggcagctgaagcacaacaggcaaaggataacaaatctgcaccagctgatgatgaggatatggatccaactcaataccttgagaacaggctaaagtatcttgcagttcaaaagacagaagggaagaacccgtatcctcataaattctttgtcactatgtctattgatcaatacatcaaggaatatggaggtttaagcaacgggcaacacattgaggatgtctctgtgtctttggctggccggatcatgcacaaacgcacctctggttctaagcttgtcttttatgacctgcacggtggtggttgcaaggtccaggttatggctgatgctagtaaatcagacttggatgaggctggattttccaaattccattctaatgtgaagcgcggtgacatagttggtgtcactgggttcccaggcaaaagtaagaagggcgaacttagtatttttcccaagaattttgtgtcgctgtctcattgtttgcacatgatgccaaggcaaaagtctgctgctgctgctattgataatagaaatccatggataccgggaagtactaggaatcctgaaacatatattttgaaggatcaggaaactagatatcggttacgccatttggatttgatgcttaatccagaggttcgagacatattcaagaccaggtctaaaatcatttcttacattaggaggttccttgatgaccttgatttcttggaggttgaaacacctatgatgaatatgattgctggtggagctgcagcccgtccatttgtaacacatcacaatgatcttaacatgaggttattcatgaggattgctccagaactgtatcttaaggagttggttgttggtggactggatcgtgtttatgaaattggtaaacaatttaggaatgagggcatagatttgactcataatcctgagtttactacctgtgagttctatatggcttataaggactacaatgacttaatggatctaacagagcaaatgttgagtggtatggttaaggagcttacgaaaggcagctataaaatcaagtatcatgcagatgggattgataaggaacctattgaaattgactttactacaccttttagaaggattgacatgattgaagagttagagaagatggcaggtctaagtattcccaaagacttgtcaagtgaggaagccaatcaatatttgaaggatgcatgcgtgaagtatgagatcaaatgtcctccccctgagacaactgctcgtttgttggataaacttgtgggtcactttttggaagaaacgtgtgtaaatcctacattcatcaaaaaccatcctgagatcatgagtcctctagcaaagtggcacagatcaaaaccaggcctgactgaacgttttgaattgtttgttaataaacgtgaactttgcaatgcatatactgaattgaatgaccctgtagtacaacgacagagatttgctgaacaactcaaggatcggcaatctggcgatgatgaagcaatggccttagatgaaacattttgtacggctctagaatatggtttaccacctactggtggttggggtttgggcattgatcggttgaccatgttactgactgattcacagaatattaaggaggttcttctcttccctgccatgaaacctcaagactga

SEQ ID NO: 201 - protein, 598 aa, Glycine max
MEVPSEVPSTDTGTSTGTGETMSKNALKRELKNKQREEERKRKDEEKAKKAAEAQQAKDNKSAPADDEDMDPTQYLENRLKYLAVQKTEGKNPYPHKFFVTMSIDQYIKEYGGLSNGQHIEDVSVSLAGRIMHKRTSGSKLVFYDLHGGGCKVQVMADASKSDLDEAGFSKFHSNVKRGDIVGVTGFPGKSKKGELSIFPKNFVSLSHCLHMMPRQKSAAAAIDNRNPWIPGSTRNPETYILKDQETRYRLRHLDLMLNPEVRDIFKTRSKIISYIRRFLDDLDFLEVETPMMNMIAGGAAARPFVTHHNDLNMRLFMRIAPELYLKELVVGGLDRVYEIGKQFRNEGIDLTHNPEFTTCEFYMAYKDYNDLMDLTEQMLSGMVKELTKGSYKIKYHADGIDKEPIEIDFTTPFRRIDMIEELEKMAGLSIPKDLSSEEANQYLKDACVKYEIKCPPPETTARLLDKLVGHFLEETCVNPTFIKNHPEIMSPLAKWHRSKPGLTERFELFVNKRELCNAYTELNDPVVQRQRFAEQLKDRQSGDDEAMALDETFCTALEYGLPPTGGWGLGIDRLTMLLTDSQNIKEVLLFPAMKPQD

SEQ ID NO: 202 - protein, 12 aa, Glycine max
DLSSEEANQYLK

SEQ ID NO: 203 - DNA, 1728 bp, Glycine max
  source 1..1728: mol_type = other DNA
  CDS 1..1728
atgtaccgctttgcaacctccctcgcctccaaagctagaattgccaggagcagcactcaacagattggaagtagggtcacttggaacaggaattatgcggccaaagacattaagtttggtgtggaggctcgggctctgatgcttaagggtgtcgaagagctcgctgatgccgttaaagtaacaatgggccctaaggggcgtaatgtggttattgagcaaagttttggagcacctaaagtgacgaaagatggagtaaccgttgctaagagcattgaattcaaggacaaagtcaagaatatcggtgccagtcttgtaaagcaggttgctaatgctactaatgacgtggctggtgatggaaccacttgtgctacaattcttactaaagcaatatttacggaaggatgtaaatcagttgcagctggaatgaatgcgatggacctgagacgaggtataaatatggctgttgatgctgtggtgacaaatctgaaaagcagagcacgtatgattagcacttctgaagaaattgcccaggttggaacaatatctgccaatggggagagggaaattggtgagttaattgcaaaagctatggagaaagttggcaaagagggtgtcatcacaatctcagatggcaagacattgtacaatgagttggaagtcgttgaaggaatgaagctcgacaggggatacatttctccatatttcataacaaaccagaagaaccagaaatgtgaacttgaggatcctctcattataatccatgaaaagaaaatctcaagcataaatgctatcgttaaagtattagagttagctttgaagagacaaagatctttattgattgtcgccgaggatgtggaaagtgatgcccttgcaactcttattctaaataaacttcgtgctggaatcaaggtatgtgccatcaaagctcctggttttggtgaaaaccgtaaatctggtctccaggatcttgctgttcttacaggaggtcaacttatcaccgaagagcttggtttgaatcttgaaaaagtagacctggacttatttggctcttgcaagaagataacaatttctaaagatgacactgtcatccttgatggagctggtgataagaaagcaattgaggaaagatgtgagcagattagatcagcaattgaaaatagcacttcagattatgacaaggaaaagttacaggaaagattagccaagctttctgggggtgttgcagtactgaagattggaggagccagtgaggctgaagttggtgagaagaaggatagagtgacagatgccttaaatgcaaccaaggcagctgtagaggagggcatagtacctggtggtggtgttgctcttctttatgcatcgagtgagttggataagcttcaaactgccaactttgatcaaaagataggtgtccagatcatccaaaatgctttgaagactcctgtgcacacaattgcttcaaatgcaggagttgagggtgctgttgttgttggcaaactattggaacagaatgatcctgatcttggatatgatgcagccaaaggtgaatatgttgatatggttaaaactggaattattgatccattgaaggttattaggaccgccttggttgatgcagccagtgtgtcatctttgatgacaacaactgaagctgttgtgtctgaactcccgaaggacgataaagatgttcctgccatgggtggtggcatgggtggcatggattattaa

SEQ ID NO: 204 - protein, 575 aa, Glycine max
MYRFATSLASKARIARSSTQQIGSRVTWNRNYAAKDIKFGVEARALMLKGVEELADAVKVTMGPKGRNVVIEQSFGAPKVTKDGVTVAKSIEFKDKVKNIGASLVKQVANATNDVAGDGTTCATILTKAIFTEGCKSVAAGMNAMDLRRGINMAVDAVVTNLKSRARMISTSEEIAQVGTISANGEREIGELIAKAMEKVGKEGVITISDGKTLYNELEVVEGMKLDRGYISPYFITNQKNQKCELEDPLIIIHEKKISSINAIVKVLELALKRQRSLLIVAEDVESDALATLILNKLRAGIKVCAIKAPGFGENRKSGLQDLAVLTGGQLITEELGLNLEKVDLDLFGSCKKITISKDDTVILDGAGDKKAIEERCEQIRSAIENSTSDYDKEKLQERLAKLSGGVAVLKIGGASEAEVGEKKDRVTDALNATKAAVEEGIVPGGGVALLYASSELDKLQTANFDQKIGVQIIQNALKTPVHTIASNAGVEGAVVVGKLLEQNDPDLGYDAAKGEYVDMVKTGIIDPLKVIRTALVDAASVSSLMTTTEAVVSELPKDDKDVPAMGGGMGGMDY

SEQ ID NO: 205 - protein, 24 aa, Glycine max
AAVEEGIVPGGGVALLYASSELDK

SEQ ID NO: 206 - protein, 11 aa, Glycine max
DRVTDALNATK

SEQ ID NO: 207 - protein, 10 aa, Glycine max
EGVITISDGK

SEQ ID NO: 208 - protein, 14 aa, Glycine max
GRNVVIEQSFGAPK

SEQ ID NO: 209 - protein, 10 aa, Glycine max
GVEELADAVK

SEQ ID NO: 210 - protein, 12 aa, Glycine max
IGGASEAEVGEK

SEQ ID NO: 211 - protein, 11 aa, Glycine max
IGVQIIQNALK

SEQ ID NO: 212 - protein, 9 aa, Glycine max
ISSINAIVK

SEQ ID NO: 213 - protein, 15 aa, Glycine max
LLEQNDPDLGYDAAK

SEQ ID NO: 214 - protein, 9 aa, Glycine max
LQTANFDQK

SEQ ID NO: 215 - protein, 9 aa, Glycine max
LSGGVAVLK

SEQ ID NO: 216 - protein, 8 aa, Glycine max
NIGASLVK

SEQ ID NO: 217 - protein, 8 aa, Glycine max
TGIIDPLK

SEQ ID NO: 218 - protein, 20 aa, Glycine max
TPVHTIASNAGVEGAVVVGK

SEQ ID NO: 219 - DNA, 1725 bp, Glycine max
  source 1..1725: mol_type = other DNA
  CDS 1..1725
atgtatcgctttgcttctaacctcgcttccaaagcaaggattgctaggagtagcagccaccagattggaagtaggctgagttcgagcagaaactatgcggctaaagacattagatttggtgtggaggctcgggcactgatgcttaagggtgttgaagagctagctgatgctgtcaaagtaaccatgggtcccaaggggcgtaatgttgtgattgagcaaagttttggtgcccctaaagtaaccaaagatggagtaactgttgcaaagagcattgaattcaaggataaagttaagaatgttggtgccagtcttgtaaagcaggttgcaaatgctactaatgatgtggctggtgatggaaccacgtgcgctacagtccttacacgagcaatatttactgaaggctgcaaatcagttgcggctggaatgaatgcaatggacctgaggcgtggtataagtatggctgtcgatgctgtggtgacaaacttgaaaagcagagcaaggatgattagcacatctgaagaaatagcacaggtagggacaatatctgctaacggagagagagaaattggtgagttgattgcaaaagctatggagaaagttggcaaagagggagtaatcacaatttcagatggcaaaaccttatataatgagttggaagttgttgaaggaatgaagctcgacaggggctacatatctccttatttcattacaaatgataagaaccagaaatgtgaacttgaagatcccctcattctaattcatgagaagaaaatctcgagtataaatgctatagttaaagtcttagagttggctttgaagagacaaagatctttattgattattgccgaggatgtggaaagtgatgcacttgcaactcttattctaaataagcttcgggctggaattaaggtatgtgctattaaagcccctggttttggcgaaaataggaaagccaatcttcaagatcttgcagttctcacaggaggagcacttattactgaagagcttggcttgaagcttgaaaaagtggatttggatatgcttggcacctgtaaaaaaataacagtttctaaggatgacactgtcattcttgacggagcaggtgacaagaaagcacttgaggaaagatgcgagcagattaggtctgcaattgaaaacagcacctcagattatgacaaggaaaagttacaggaaagattagccaagctttctggtggtgttgccgtgcttaagattggaggagccagtgaagctgaagttggtgagaagaaggatagagtgacagatgccttaaatgcaactaaggctgctgtagaggaaggcattgtatcaggtggtggtgttgctcttctatatgcgtcaaaagagttggataaacttcaaactgctaactttgatcagaagattggtgtccagattatccaaaatgctttaaagacacctgtgctcacaattgcatcaaatgcgggtgttgagggtgcagttgttgttggcaaactattggaacaggagaatcatgatctaggatatgatgcagccaaaggtgaatatgttgatatggttaaagctggaatcatagaccctttgaaggtgattaggacagccttggttgatgcagccagtgtatcatctttgatgacaacaactgaagctgttgtgtctgaacttccaaacgatgataaagatactccagctatggctggtggcatgggtggcatgggttat

SEQ ID NO: 220 - protein, 575 aa, Glycine max
MYRFASNLASKARIARSSSHQIGSRLSSSRNYAAKDIRFGVEARALMLKGVEELADAVKVTMGPKGRNVVIEQSFGAPKVTKDGVTVAKSIEFKDKVKNVGASLVKQVANATNDVAGDGTTCATVLTRAIFTEGCKSVAAGMNAMDLRRGISMAVDAVVTNLKSRARMISTSEEIAQVGTISANGEREIGELIAKAMEKVGKEGVITISDGKTLYNELEVVEGMKLDRGYISPYFITNDKNQKCELEDPLILIHEKKISSINAIVKVLELALKRQRSLLIIAEDVESDALATLILNKLRAGIKVCAIKAPGFGENRKANLQDLAVLTGGALITEELGLKLEKVDLDMLGTCKKITVSKDDTVILDGAGDKKALEERCEQIRSAIENSTSDYDKEKLQERLAKLSGGVAVLKIGGASEAEVGEKKDRVTDALNATKAAVEEGIVSGGGVALLYASKELDKLQTANFDQKIGVQIIQNALKTPVLTIASNAGVEGAVVVGKLLEQENHDLGYDAAKGEYVDMVKAGIIDPLKVIRTALVDAASVSSLMTTTEAVVSELPNDDKDTPAMAGGMGGMGY

SEQ ID NO: 221 - protein, 8 aa, Glycine max
AGIIDPLK

SEQ ID NO: 222 - protein, 11 aa, Glycine max
DRVTDALNATK

SEQ ID NO: 223 - protein, 10 aa, Glycine max
EGVITISDGK

SEQ ID NO: 224 - protein, 14 aa, Glycine max
GRNVVIEQSFGAPK

SEQ ID NO: 225 - protein, 10 aa, Glycine max
GVEELADAVK

SEQ ID NO: 226 - protein, 12 aa, Glycine max
IGGASEAEVGEK

SEQ ID NO: 227 - protein, 11 aa, Glycine max
IGVQIIQNALK

SEQ ID NO: 228 - protein, 9 aa, Glycine max
ISSINAIVK

SEQ ID NO: 229 - protein, 15 aa, Glycine max
LLEQENHDLGYDAAK

SEQ ID NO: 230 - protein, 9 aa, Glycine max
LQTANFDQK

SEQ ID NO: 231 - protein, 9 aa, Glycine max
LSGGVAVLK

SEQ ID NO: 232 - DNA, 1869 bp, Glycine max
  source 1..1869: mol_type = other DNA
  CDS 1..1869
atggctcaagttcttgagaaccccaccattgacgcggcggcttccggcgcgaacccctcgctgacgaccatctcgttgtacgtcggggatcttcaccacgacgtcaacgatccgcagctgtatgatctcttcaaccaggttgcccaagtcgtttccgttcgaatttgcagggacgtggccacccaacaatcccttggttacggctacgtcaatttcagcaacgctcacgatgccgcaaaagcaattgatgtgctgaatttcactccactgaacggcaaaatcattaggataatgtactctattcgggaccctagtgctcggaagagtggtgcagctaatgttttcataaagaatttggacaaggcgattgatcacaaggctttgtacgatacattttcggcttttgggaatattctatcttgcaaggttgcgacagatgcttctggccagtctaaaggccatggttttgtgcagtttgagagtgaagaatctgcacaaaatgcaattgacaagttaaacggcatgctgatcaatgataagcaggtctttgtgggtcctttcctgcggaagcaggatagagagagtgctctcagtggcacaaagtttaataacgttttcgtgaaaaacctgttggattcaatgactgaggctgacttggagagaatttttggagaatatggagctattactagcgctgtagtgatgagagatgtagatggtaaatcaaaaggttttggttttgtcaattttgcaaatgtagacgatgctgctaaagctgtggaggctcttaatggaaagaattttgatggtaaggagtggtatgttggaaaagcccagaaaaagtctgaaagggagcttgaactgaaagggcaacatgaacagattacaaaggagactgttgataaatatcatggtacaaacttgtatatcaagaacttggatgatagtgttggtgatgaagaactcatggaactgttctctgagtttggtacgataacctcatgcaaggttatgcgagatccaaatggaattagtagaggatctggatttgtttcattttcaattgctgagggagcaactcgggctcttggtgagatgaatggtaaaatggttgctggcaaacctctctatgttgcccttgcacagagaaaagaagatagaagagcaaggttacaggcacaattttcacaatcgaggcctgctgcaataacccctaatgtttcacctcgaatgcctctataccctctaggggctcctgcgataggacaacaatttttgtatgggcaagcagctccagccacgatacctcaacattttgttcctggaatgaggccaggtggtgctccaaacttctatgttccactggttcagcagggtcaacagggacagcgcccaggaggacaccgaggatcaggtcctatgcaacagctccagcagccattgccattgatgcaccaacagatgcttccaaggggacatgtctatcgatatccttctggctccaatatgcaaaatattcaactggcaggagtacctggaggaatggtcacttatggcattggtcaacaaatgcccacgcaggccctggcctcggcccttgccaatgctactcctgaacagcagaggactatgcttggtgaagctttatacccgttagtagataagctggagcatgaagcagcagcaaaggttactggtatgctcttagagatggatcagcctgaggtattacatctgattgagtcaccggatgctctcaaggctaaagttgttgaggccatggatgtgttgaagaaagttacacagcagcaatcaaacagcccagccgatcaactggcttcactctctctgaatgataatgtcgagtct

SEQ ID NO: 233 - protein, 623 aa, Glycine max
MAQVLENPTIDAAASGANPSLTTISLYVGDLHHDVNDPQLYDLFNQVAQVVSVRICRDVATQQSLGYGYVNFSNAHDAAKAIDVLNFTPLNGKIIRIMYSIRDPSARKSGAANVFIKNLDKAIDHKALYDTFSAFGNILSCKVATDASGQSKGHGFVQFESEESAQNAIDKLNGMLINDKQVFVGPFLRKQDRESALSGTKFNNVFVKNLLDSMTEADLERIFGEYGAITSAVVMRDVDGKSKGFGFVNFANVDDAAKAVEALNGKNFDGKEWYVGKAQKKSERELELKGQHEQITKETVDKYHGTNLYIKNLDDSVGDEELMELFSEFGTITSCKVMRDPNGISRGSGFVSFSIAEGATRALGEMNGKMVAGKPLYVALAQRKEDRRARLQAQFSQSRPAAITPNVSPRMPLYPLGAPAIGQQFLYGQAAPATIPQHFVPGMRPGGAPNFYVPLVQQGQQGQRPGGHRGSGPMQQLQQPLPLMHQQMLPRGHVYRYPSGSNMQNIQLAGVPGGMVTYGIGQQMPTQALASALANATPEQQRTMLGEALYPLVDKLEHEAAAKVTGMLLEMDQPEVLHLIESPDALKAKVVEAMDVLKKVTQQQSNSPADQLASLSLNDNVES

SEQ ID NO: 234 - protein, 13 aa, Glycine max
AIDVLNFTPLNGK

SEQ ID NO: 235 - protein, 7 aa, Glycine max
FNNVFVK

SEQ ID NO: 236 - protein, 15 aa, Glycine max
GFGFVNFANVDDAAK

SEQ ID NO: 237 - protein, 9 aa, Glycine max
SGAANVFIK

SEQ ID NO: 238 - DNA, 1851 bp, Glycine max
  source 1..1851: mol_type = other DNA
  CDS 1..1851
atggattctgattatggtattcccagggaactctcagatcttcaaaagattcggtctttgtaccagccagagctccctccttgtctccagggaaccactgtgagggttgaatttggtgacgcaaccaccactgctgaccccactgatgcactcaccgtctgcagggcttttcctaacacttatggacaccctttggctcactttctcagggccactgccaaagtccctgatgctcagatcataactgagttccctcctattagggtggggattgtcttttgtgggagacaatctcctggagggcataatgtcatttggggtatccacaatgctctcaaaattcacaatcccaacagtgttttgcttggatttcttggtggttcagaaggtctatttgcccagaaaactctggagataactgacgatattctttcgacgtacaaaaatcaaggtggttatgatttgcttgggcggacgaaggatcaaattaggaccacagaacaagttaatgctgcacttgctgcatgcaacaatttgaaacttgatggccttgtcataatcggaggggtgacatcaaacacagatgctgcacagcttgctgaaacatttgttgtagcaaaatgccccacaaaggtggttggtgttcctgtcactctgaatggagatcttaaaaaccagtttgtagaaactaatgttggttttgatacaatttgtaaggttaattctcagctcatcagcaatgtctgcactgatgctctctctgcagagaagtattattatttcatccgtcttatgggacgcaaggcatcacacgttgctttggaatgcaccctccagtcccatccaaatatggtaattcttggtgaggaggttgctgcatcaaagcttaccctttctgaaattgcaaaacagattactgatgcaattcaggctagagcagagcaagacaaataccatggagtcattctccttccagaagggctgatagagagtattcctgaagtgtatgccctcttgaaggaaattcatggtttactcagacaaggtgttgaagttgacaagatatcttctcagctaacaccatgggcatctgctttatttgaatttctgccaccctttattaggagacagttgctcctttaccctgaatccgacgactcagcacagttatcacagattgaaactgagaaattactagcatatcttgtggaagtcgagattaacaagcgtctgaaagaaggcacatacaaggggaagaaattcaatgccatttgccacttttttggttatcaagctcgtggatctctgccatcaaaatttgactgtgattacgcatatgtccttgggcacatctgctaccatatactggccgctggtctaaatggatacatggcaactttaactaacctgaagaatccagtaaacaagtggaaatgtggggctgctccaattgcatctatgatgactgtaaagcgctggtctccaaatccaggagctacatcaattggaaaacctgctattcatccagctactgtggacttgagaggcaaagcatatgagctgttgagacaaaaggcacaaagctttcttatggatgacatctacagaaatccaggtccacttcagtttgatggtcccggcgcagatgccaaggttataactctttctgttgaagaccaggactacatgggccgcattaagaaacttcaggagtaccttgaacaggttcgaaccattgtcaagccaggatgccctcaggaggttttgaaagctgctctaagcgtcatggcgtcggttaccgaagttctagctgcaatgtcaacatcttataccaacaccctggcatccctg

SEQ ID NO: 239 - protein, 617 aa, Glycine max
MDSDYGIPRELSDLQKIRSLYQPELPPCLQGTTVRVEFGDATTTADPTDALTVCRAFPNTYGHPLAHFLRATAKVPDAQIITEFPPIRVGIVFCGRQSPGGHNVIWGIHNALKIHNPNSVLLGFLGGSEGLFAQKTLEITDDILSTYKNQGGYDLLGRTKDQIRTTEQVNAALAACNNLKLDGLVIIGGVTSNTDAAQLAETFVVAKCPTKVVGVPVTLNGDLKNQFVETNVGFDTICKVNSQLISNVCTDALSAEKYYYFIRLMGRKASHVALECTLQSHPNMVILGEEVAASKLTLSEIAKQITDAIQARAEQDKYHGVILLPEGLIESIPEVYALLKEIHGLLRQGVEVDKISSQLTPWASALFEFLPPFIRRQLLLYPESDDSAQLSQIETEKLLAYLVEVEINKRLKEGTYKGKKFNAICHFFGYQARGSLPSKFDCDYAYVLGHICYHILAAGLNGYMATLTNLKNPVNKWKCGAAPIASMMTVKRWSPNPGATSIGKPAIHPATVDLRGKAYELLRQKAQSFLMDDIYRNPGPLQFDGPGADAKVITLSVEDQDYMGRIKKLQEYLEQVRTIVKPGCPQEVLKAALSVMASVTEVLAAMSTSYTNTLASL

SEQ ID NO: 240 - protein, 13 aa, Glycine max
VVGVPVTLNGDLK

SEQ ID NO: 241 - DNA, 1575 bp, Glycine max
  source 1..1575: mol_type = other DNA
  CDS 1..1575
atgcccaaattttttcatagcattttcagaggaacaatggcgggtagggtttcgacgtgcttcttcttcgtgtttgctctttctctccttcttccttttcagatctccgctgaggaatcatcggagaaagagttcgtactcacattggatcactctaactttcacgacactgtcagcaagcacgatttcatcgtcgtcgagttctacgctccatggtgtggccactgtaagaagcttgctcccgagtatgagaaagctgcttctatcttgagtagtcatgatcctccggttgttttggctaaaattgatgccaatgaggagaagaacaaagaccttgcatctcaatatgatgttaggggatacccaacaattaagattctgagaaatgggggaaagaatgttcaggaatacaaaggtccccgtgaagcagatggcatcgttgactatttgaagaaacaaagtggtcctgcttcaactgaaattaaatctgctgatgaagctactgctttcattggtgaaaataaagttgctattgttggagttttcccgaaattttctggagaggagtttgacaacttttctgccttagcagagaagttgcgttctgactatgacttcggtcacactctgaatgccaaacaccttccaagaggagagtcatcagtttctggtcccgtagttaggttgttcaagccattcgatgaactttttgttgacttccaggatttcaatgtggaagctctagagaaatttgttgaggaatctagtacccctgttgttactgtctttaacaatgatccgagcaatcaccctttcgttgccaaattcttcaacagtcccaatgcaaaggcaatgttgttcatcaacttcactgccgaaggtgctgaatctttcaaatcaaaataccgtgaagctgctgagcaacataaacaacagggtgtaagctttttggtgggagatgttgagtctagtcaaggtgctttccagtattttggactgaaagaagaacaagtacctctaattatcattcaacacaatgacgggaaaaagttttttaaacccaatttggaagctgatcacattccaacttggttgaaggcgtacaaggatggtaatgttgcaccatttgtgaagtctgaacccattcctgaagctaacgatgaacctgttaaagtggtagttgggaacagtcttgaggacattgttttcaaatctgggaagaatgttctgctggagttttatgctccctggtgtggtcattgcaaacagttggctccaatattggacgaagttgctatctcatatcaaagtgatgctgatgttgttattgcaaaactggatgcaactgccaacgatatcccaagtgaaacctttgatgtccaaggttatccaaccgtgtacttcaggtcagcaagtggaaagttatcacaatacgaagggggtaggaccaaggaagacatcatagaattcatcgaaaagaaccgggacaaacctgctcagcaagaacaaggacaagataaacctgctcagcaagaacaaggacaagatgagcaagaaaaaggaaaagatgagctt

SEQ ID NO: 242 - protein, 525 aa, Glycine max
MPKFFHSIFRGTMAGRVSTCFFFVFALSLLLPFQISAEESSEKEFVLTLDHSNFHDTVSKHDFIVVEFYAPWCGHCKKLAPEYEKAASILSSHDPPVVLAKIDANEEKNKDLASQYDVRGYPTIKILRNGGKNVQEYKGPREADGIVDYLKKQSGPASTEIKSADEATAFIGENKVAIVGVFPKFSGEEFDNFSALAEKLRSDYDFGHTLNAKHLPRGESSVSGPVVRLFKPFDELFVDFQDFNVEALEKFVEESSTPVVTVFNNDPSNHPFVAKFFNSPNAKAMLFINFTAEGAESFKSKYREAAEQHKQQGVSFLVGDVESSQGAFQYFGLKEEQVPLIIIQHNDGKKFFKPNLEADHIPTWLKAYKDGNVAPFVKSEPIPEANDEPVKVVVGNSLEDIVFKSGKNVLLEFYAPWCGHCKQLAPILDEVAISYQSDADVVIAKLDATANDIPSETFDVQGYPTVYFRSASGKLSQYEGGRTKEDIIEFIEKNRDKPAQQEQGQDKPAQQEQGQDEQEKGKDEL

SEQ ID NO: 243 - protein, 16 aa, Glycine max
AASILSSHDPPVVLAK

SEQ ID NO: 244 - protein, 9 aa, Glycine max
DGNVAPFVK

SEQ ID NO: 245 - protein, 15 aa, Glycine max
DLASQYDVRGYPTIK

SEQ ID NO: 246 - protein, 9 aa, Glycine max
EDIIEFIEK

SEQ ID NO: 247 - protein, 15 aa, Glycine max
EEQVPLIIIQHNDGK

SEQ ID NO: 248 - protein, 17 aa, Glycine max
EFVLTLDHSNFHDTVSK

SEQ ID NO: 249 - protein, 16 aa, Glycine max
FFKPNLEADHIPTWLK

SEQ ID NO: 250 - protein, 8 aa, Glycine max
FFNSPNAK

SEQ ID NO: 251 - protein, 15 aa, Glycine max
FSGEEFDNFSALAEK

SEQ ID NO: 252 - protein, 25 aa, Glycine max
FVEESSTPVVTVFNNDPSNHPFVAK

SEQ ID NO: 253 - DNA, 1509 bp, Glycine max
  source 1..1509: mol_type = other DNA
  CDS 1..1509
atggcgggtacggtttcgacttgtttcttcttcgtgtttcttctttctctccttcttccttttcagatctccgctgaggaatcctcggagaaggagttcgtactcacattggatcactctaactttcacgacactgtcagcaagcacgatttcatcgtcgtcgagttctacgccccatggtgtggtcactgtaagaagcttgctccggagtatgagaaagctgcttctatcttgagtagtcatgatcctcctattgttttggctaaagtagatgccaatgaggagaagaacaaagaccttgcatcacaatatgatgttaagggattcccaacaattaatattctgagaaatgggggaaagaatgttcaggaatacaaaggtccccgtgaagcagatggcatcgttgactatttgaagaaacaaagtggtcctgcttcaactgaaattaaatctgctgatgaagctactgcttttattggtgaaaataaagttgctattgttggagttttcccgaaattttctggagaggagtttgacaacttttctgccttagcagagaagttgcgttctgattatgactttggtcacactctgaatgccaaactccttccaagaggagagtcatcagtttctgggcccgtagttaggctgttcaagccattcgatgaactttttgttgacttccaggatttcaatgtggaagctctagagaagtttgttgaggaatctagtacccctgttgttactgtctttaacaatgaaccgagcaatcaccctttcgttgtcaaattcttcaacagtcccaacgcaaaggcaatgttgttcatcaacttcactgccgaaggtgctgaagctatcaaatcaaaataccgtgaagctgctgagcaatataaacaacagggtgtcagctttttggtgggagatgttgagtctagtcaaggtgctttccagtattttggactgaaagaagagcaagtacctctaattatcattcaacataatgatgggaaaaagttttttaaacccaatttggaagctgatcacattccaacctggttgaaggcatacaaggatggtcatgttgcaccatttgttaagtctgaacccattcccgagactaacgatgaacctgttaaagtggtagttggggccagtcttgaggacattgttttcaaatctgggaagaatgttctgctggagttttatgctccctggtgtggtcattgcaaacagttggctccaatattggatgaagttgctatctcatatcaaaatgaagctgatgttgttattgcaaaactggatgcaactgccaacgatatcccaagtgaaacctttgatgtccaaggttatcctaccgtgtacttcaggtcagcaagtggaaagttatcacaatacgacggcggtaggaccaaggaagacatcatagaattcatcgaaaagaaccgggataaacctgctcagcaagaacaaggaaaagatgagcaagaacaaggaaaagatgagctt

SEQ ID NO: 254 - protein, 503 aa, Glycine max
MAGTVSTCFFFVFLLSLLLPFQISAEESSEKEFVLTLDHSNFHDTVSKHDFIVVEFYAPWCGHCKKLAPEYEKAASILSSHDPPIVLAKVDANEEKNKDLASQYDVKGFPTINILRNGGKNVQEYKGPREADGIVDYLKKQSGPASTEIKSADEATAFIGENKVAIVGVFPKFSGEEFDNFSALAEKLRSDYDFGHTLNAKLLPRGESSVSGPVVRLFKPFDELFVDFQDFNVEALEKFVEESSTPVVTVFNNEPSNHPFVVKFFNSPNAKAMLFINFTAEGAEAIKSKYREAAEQYKQQGVSFLVGDVESSQGAFQYFGLKEEQVPLIIIQHNDGKKFFKPNLEADHIPTWLKAYKDGHVAPFVKSEPIPETNDEPVKVVVGASLEDIVFKSGKNVLLEFYAPWCGHCKQLAPILDEVAISYQNEADVVIAKLDATANDIPSETFDVQGYPTVYFRSASGKLSQYDGGRTKEDIIEFIEKNRDKPAQQEQGKDEQEQGKDEL

SEQ ID NO: 255 - protein, 16 aa, Glycine max
AASILSSHDPPIVLAK

SEQ ID NO: 256 - protein, 9 aa, Glycine max
DGHVAPFVK

SEQ ID NO: 257 - protein, 9 aa, Glycine max
DLASQYDVK

SEQ ID NO: 258 - protein, 9 aa, Glycine max
EDIIEFIEK

SEQ ID NO: 259 - protein, 15 aa, Glycine max
EEQVPLIIIQHNDGK

SEQ ID NO: 260 - protein, 17 aa, Glycine max
EFVLTLDHSNFHDTVSK

SEQ ID NO: 261 - protein, 16 aa, Glycine max
FFKPNLEADHIPTWLK

SEQ ID NO: 262 - protein, 8 aa, Glycine max
FFNSPNAK

SEQ ID NO: 263 - protein, 15 aa, Glycine max
FSGEEFDNFSALAEK

SEQ ID NO: 264 - protein, 25 aa, Glycine max
FVEESSTPVVTVFNNEPSNHPFVVK

SEQ ID NO: 265 - protein, 13 aa, Glycine max
GFPTINILRNGGK

SEQ ID NO: 266 - protein, 13 aa, Glycine max
GPREADGIVDYLK

SEQ ID NO: 267 - protein, 7 aa, Glycine max
LAPEYEK

SEQ ID NO: 268 - protein, 29 aa, Glycine max
LDATANDIPSETFDVQGYPTVYFRSASGK

SEQ ID NO: 269 - protein, 14 aa, Glycine max
LRSDYDFGHTLNAK

SEQ ID NO: 270 - protein, 10 aa, Glycine max
LSQYDGGRTK

SEQ ID NO: 271 - protein, 23 aa, Glycine max
QLAPILDEVAISYQNEADVVIAK

SEQ ID NO: 272 - protein, 24 aa, Glycine max
QQGVSFLVGDVESSQGAFQYFGLK

SEQ ID NO: 273 - protein, 10 aa, Glycine max
QSGPASTEIK

SEQ ID NO: 274 - protein, 13 aa, Glycine max
SADEATAFIGENK

SEQ ID NO: 275 - protein, 13 aa, Glycine max
SEPIPETNDEPVK

SEQ ID NO: 276 - protein, 9 aa, Glycine max
VAIVGVFPK

SEQ ID NO: 277 - protein, 13 aa, Glycine max
VVVGASLEDIVFK

SEQ ID NO: 278 - protein, 9 aa, Glycine max
YREAAEQYK

SEQ ID NO: 279 - DNA, 1467 bp, Glycine max
  source 1..1467: mol_type = other DNA
  CDS 1..1467
atgggtgtggctcaaaacattcccgatgcggaggagggaaccttggagattggaatggaatacagaactgtttctggagttgctgggccattggtcattcttgataaagttaagggacccaagtttcaagagattgttaacattcgcttaggagatggtactactcggcgtggacaagtgctagaagttgatggtgaaaaggctgttgttcaggtctttgagggtacatctgggattgacaataaatttacaactgtgcaatttactggagaggtgttaaaaactccagtatcattggacatgcttggccgcatattcaatggttctggaaaaccaattgataatggtccacccattttacctgaggcttacttggacatatcaggaagttccatcaaccctagtgagagaacctatccagaggagatgatacagacaggaatatctaccattgatgtcatgaattctattgctagaggtcaaaagatccctctattctcagctgctggtctcccccataatgaaattgctgcacagatatgccgtcaggctggcttagtcaagcgactagaaaagtctgataatcttctcgagggtggaggagaagaggacaattttgccattgtgtttgcagctatgggagttaacatggagactgcacagtttttcaaacgtgactttgaggaaaatggctcgatggagagagtgacccttttccttaatctggcaaatgatcctacgattgagcgtatcatcactcctcgtattgctcttactactgctgaatatttggcatatgaatgtggcaagcatgttcttgtgatactaactgatatgagttcttatgctgatgctcttcgtgaggtatctgctgcccgagaagaagtacctggtagacgtgggtatcccggctacatgtatacagatcttgcaacaatatacgaacgtgctggaagaattgaggggcgaaaaggctctattacacaaattccaattctaaccatgcctaatgatgatattacgcatccaactcctgatcttacaggatatattactgagggacagatatatatcgacagacagttgtataacaggcagatatatcctccaatcaatgtccttccatcattgtctcgtttgatgaagagtgccattggtgagggaatgacacgaaaggatcattctgatgtgtccaaccagctctatgcaaattatgccattggaaaggatgtccaggcaatgaaagccgtggtcggagaagaagcactttcatctgaggacctgctgtatttggaatttttggagaagtttgagaggaagtttgttgcccaaggagcatacgatacacgcaatatcttccagtcactagatcttgcatggactttgcttcggattttccctcgcgagcttctccaccgtattcctgcaaagactcttgatcagttctacagccgagatgccagtaac

SEQ ID NO: 280 - protein, 489 aa, Glycine max
MGVAQNIPDAEEGTLEIGMEYRTVSGVAGPLVILDKVKGPKFQEIVNIRLGDGTTRRGQVLEVDGEKAVVQVFEGTSGIDNKFTTVQFTGEVLKTPVSLDMLGRIFNGSGKPIDNGPPILPEAYLDISGSSINPSERTYPEEMIQTGISTIDVMNSIARGQKIPLFSAAGLPHNEIAAQICRQAGLVKRLEKSDNLLEGGGEEDNFAIVFAAMGVNMETAQFFKRDFEENGSMERVTLFLNLANDPTIERIITPRIALTTAEYLAYECGKHVLVILTDMSSYADALREVSAAREEVPGRRGYPGYMYTDLATIYERAGRIEGRKGSITQIPILTMPNDDITHPTPDLTGYITEGQIYIDRQLYNRQIYPPINVLPSLSRLMKSAIGEGMTRKDHSDVSNQLYANYAIGKDVQAMKAVVGEEALSSEDLLYLEFLEKFERKFVAQGAYDTRNIFQSLDLAWTLLRIFPRELLHRIPAKTLDQFYSRDASN

SEQ ID NO: 281 - protein, 17 aa, Glycine max
DHSDVSNQLYANYAIGK

SEQ ID NO: 282 - protein, 26 aa, Glycine max
FQEIVNIRLGDGTTRRGQVLEVDGEK

SEQ ID NO: 283 - protein, 12 aa, Glycine max
FTTVQFTGEVLK

SEQ ID NO: 284 - protein, 12 aa, Glycine max
TLDQFYSRDASN

SEQ ID NO: 285 - DNA, 1464 bp, Glycine max
  source 1..1464: mol_type = other DNA
  CDS 1..1464
atgggtgtggctgagaacattcccgatatggaggagggtaccttggagattggaatggaatacagaactgtatctggagttgctggaccattggtcattcttgacaaagttaagggacccaaatttcaagagattgttaatattcgtttaggagatggaactactcggcgtggacaagttctagaggttgatggcgaaaaggctgttgttcaggtatttgaaggcacatctgggatcgacaataagtttacaactgtgcaatttactggagaggtgttaaaaactcctgtctcactggacatgcttggacgcatatttaatggttctggaaaaccaattgataatggtccaccaattttacctgaggcttacctggatatttctggaagttccatcaatcctagtgagagaacctatcctgaggagatgatacagacaggaatatctacaattgatgtcatgaattctattgctagaggtcaaaagatccctctattctcagcagctggtctcccccataatgaaattgctgcacagatctgtcgtcaggctggcttagtcaagcgactagagaaatctgataatcttcttgagggtggagaagaggacaattttgctattgtgtttgcagctatgggagttaacatggagactgcacagtttttcaagcgtgactttgaggagaatggatcaatggagagagtgactcttttccttaatttggcaaatgatcctacaattgagcgtattatcactcctcgtattgctcttactactgctgaatatttggcttatgaatgtggcaagcacgttcttgtgatactcacagacatgagttcttatgctgatgctcttcgtgaggtatctgctgcccgagaagaagtgccgggtagacgtggttatcctggatatatgtatacagatcttgcaacaatttatgaacgagctgggagaattgaggggcgtaaaggctctattacacaaattccaattctaaccatgcccaatgatgatattacgcacccaactcctgatctaacaggatatatcactgaggggcagatatacatcgacaggcagctgtataatagacagatatacccaccaatcaacgtcctcccatcactatctcggttgatgaagagtgctattggtgagggaatgactagaagggatcattctgatgtgtccaaccagctctatgcaaattatgctattggaaaggatgtccaggcaatgaaagcagtggttggagaagaagcactttcatctgaggacctgctgtatctggaatttttggagaagtttgagaggaagtttgttgcccagggagcctatgacacccgcaatatattccagtcacttgatcttgcatggactttactccggattttccctcgtgaacttcttcatcgtataccagccaagactcttgaccagttttacagcagagatgctggtaat

SEQ ID NO: 286 - protein, 488 aa, Glycine max
MGVAENIPDMEEGTLEIGMEYRTVSGVAGPLVILDKVKGPKFQEIVNIRLGDGTTRRGQVLEVDGEKAVVQVFEGTSGIDNKFTTVQFTGEVLKTPVSLDMLGRIFNGSGKPIDNGPPILPEAYLDISGSSINPSERTYPEEMIQTGISTIDVMNSIARGQKIPLFSAAGLPHNEIAAQICRQAGLVKRLEKSDNLLEGGEEDNFAIVFAAMGVNMETAQFFKRDFEENGSMERVTLFLNLANDPTIERIITPRIALTTAEYLAYECGKHVLVILTDMSSYADALREVSAAREEVPGRRGYPGYMYTDLATIYERAGRIEGRKGSITQIPILTMPNDDITHPTPDLTGYITEGQIYIDRQLYNRQIYPPINVLPSLSRLMKSAIGEGMTRRDHSDVSNQLYANYAIGKDVQAMKAVVGEEALSSEDLLYLEFLEKFERKFVAQGAYDTRNIFQSLDLAWTLLRIFPRELLHRIPAKTLDQFYSRDAGN

SEQ ID NO: 287 - protein, 26 aa, Glycine max
FQEIVNIRLGDGTTRRGQVLEVDGEK

SEQ ID NO: 288 - protein, 12 aa, Glycine max
FTTVQFTGEVLK

SEQ ID NO: 289 - protein, 12 aa, Glycine max
TLDQFYSRDAGN

SEQ ID NO: 290 - DNA, 1407 bp, Glycine max
  source 1..1407: mol_type = other DNA
  CDS 1..1407
atggccaccgctgccgagaaactctccgctctcaaatccgccgtcgccggattgaacgaaatcagtgagaatgagaagaacggattcatcagcctcgtcggccgctatctcagtggcgaagcgcagcatgtggaatggagcaagatccagacgcctacggacgaagtggttgtgccttacgacactttggcgccaactcctgaaggttcttcggaggtgaagaatctattggacaagcttgtggtgttgaagctaaatggaggcttgggaacaactatgggttgcactggtcctaaatctgtaattgaagttcgtgatgggttgacatttctagatttaattgtcatccaaattgagaatctcaattccaaatatggaagcaatgttcctttgcttttgatgaattcattcaacactcatgatgacactcaaaagattgttgaaaaataccaaaactcaaatattgagattcatacttttaaccagagccagtatcctcgattggttgttgaggactttttgccattgccttccaaagggcatactgacaaggatggatggtaccctcctggccatggtgatgtcttcccatcattattgaacagtggcaaacttgatgcactattgtcacagggtaaagagtatgtgtttgttgccaattcggataacttgggagctatagttgacttgaaaatcttgaatcatttgatccagaacaagaatgaatactgtatggaggtgactcccaaaacattggctgatgtaaagggtggcactttgatttcttacgaaggaagggttcagctcctggaaattgcacaagtccccgatgaacatgtcaatgagttcaagtcaatagagaagttcaaaattttcaacacaaataatttgtgggtgaacttaaacgcagttaaaaggcttgttgaagctgatgctcttaagatggaaattattcccaatccaaaggaagttgacggaataaaagttcttcagctggaaactgcagctggtgctgcaataaggttctttgacaaggctattgggattaatgttcctcgatcacgattccttcctgtgaaggcaacttcagatttgcttcttgtccagtctgacctctacactttggaagacggatttgtcattcggaacaaagctagggaaaatcctgaaaacccttctattgaactgggaccagaatttaagaaggttagcaacttcttgggccgcttcaagtcaattcctagtatcgttgagcttgacagtctaaaagtggctggtgatgtatggtttggagctggtgttatcctcaaggggaaagtcagcattgtatcaaagcccggtgtaaagctggaagttcccgacggtgttgccattgtagacaaggaaattaatggcccagaggacctg

SEQ ID NO: 291 - protein, 469 aa, Glycine max
MATAAEKLSALKSAVAGLNEISENEKNGFISLVGRYLSGEAQHVEWSKIQTPTDEVVVPYDTLAPTPEGSSEVKNLLDKLVVLKLNGGLGTTMGCTGPKSVIEVRDGLTFLDLIVIQIENLNSKYGSNVPLLLMNSFNTHDDTQKIVEKYQNSNIEIHTFNQSQYPRLVVEDFLPLPSKGHTDKDGWYPPGHGDVFPSLLNSGKLDALLSQGKEYVFVANSDNLGAIVDLKILNHLIQNKNEYCMEVTPKTLADVKGGTLISYEGRVQLLEIAQVPDEHVNEFKSIEKFKIFNTNNLWVNLNAVKRLVEADALKMEIIPNPKEVDGIKVLQLETAAGAAIRFFDKAIGINVPRSRFLPVKATSDLLLVQSDLYTLEDGFVIRNKARENPENPSIELGPEFKKVSNFLGRFKSIPSIVELDSLKVAGDVWFGAGVILKGKVSIVSKPGVKLEVPDGVAIVDKEINGPEDL

SEQ ID NO: 292 - protein, 15 aa, Glycine max
AIGINVPRSRFLPVK

SEQ ID NO: 293 - protein, 17 aa, Glycine max
ARENPENPSIELGPEFK

SEQ ID NO: 294 - protein, 24 aa, Glycine max
ATSDLLLVQSDLYTLEDGFVIRNK

SEQ ID NO: 295 - protein, 8 aa, Glycine max
EINGPEDL

SEQ ID NO: 296 - protein, 18 aa, Glycine max
EYVFVANSDNLGAIVDLK

SEQ ID NO: 297 - protein, 9 aa, Glycine max
ILNHLIQNK

SEQ ID NO: 298 - protein, 26 aa, Glycine max
IQTPTDEVVVPYDTLAPTPEGSSEVK

SEQ ID NO: 299 - protein, 9 aa, Glycine max
LDALLSQGK

SEQ ID NO: 300 - protein, 9 aa, Glycine max
RLVEADALK

SEQ ID NO: 301 - protein, 14 aa, Glycine max
SAVAGLNEISENEK

SEQ ID NO: 302 - protein, 12 aa, Glycine max
SIPSIVELDSLK

SEQ ID NO: 303 - protein, 17 aa, Glycine max
VLQLETAAGAAIRFFDK

SEQ ID NO: 304 - protein, 9 aa, Glycine max
VSNFLGRFK

SEQ ID NO: 305 - DNA, 1353 bp, Glycine max
  source 1..1353: mol_type = other DNA
  CDS 1..1353
atgaccaccgccaccgagaagctctccgctctcaaatccgccgtcgccggattgaacgaaatcagtgagagtgagaagaacggattcatcagcctcgtcagccgctatctcagtggcgaagcgcagcatgtggaatggagcaagatccagacgcctacggacgaagtggttgtgccttacgacactttggcgccaactcctgatggttcttcggacgtgaagaatctattggacaagcttgtggtgttgaagctaaatggaggcttgggcacaactatgggttgcactggtcctaaatctgtaattgaagttcgtgatgggttgacatttctagatttaattgtgatccagattgagaatctcaattccaaatatggaagcaatgttcctttgcttttgatgaattcattcaacactcatgatgacactcaaaagattgttgaaaaataccaaaactccaatattgagattcatacttttaaccagggcatactgacaaggatggatggtaccctcctggcccatggagatgtctttccatcattattgaacagtggcaaacttgatgcactattgtcacagggtaaagagtatgtatttgttgccaattcagataacttgggagctatagttgacttgaaaatcttaaatcatttgatccagaacaagaatgaatactgtatggaggtgactcccaaaacattggctgatgtaaagggtggcactttgatttcttacgaaggaaggcttttggaaattgcacaagtcccagatgaacatgtcaatgagttcaagtcaatagagaagttcaaaattttcaacacaaataatttgtgggtgaacttaaatgcagttaaaaggcttgttgaagctgatgctcttaagatggaaattattcccaatccaaaggaagttgatggaataaaagttcttcagctggaaactgcagctggtgctgcaataaggttctttgacaaggctattgggattaatgttcctcgatcacgattccttcctgtgaaggcaacttcagatttgcttcttgtccagtctgacctctacactttggaagatggatttgtcattcggaacaaagctagggcaaatcctgaaaacccttctattgaacttgggccagaatttaagaaggttagcaacttcttgggccgcttcaagtcaattcccagtattgttgagcttgacagtctaaaagtggctggcaatgtatggtttggagctggtgttatcctcaagggaaaaatcagtatcgtggccaatcctggtgttaagctggaagttcccgatggtgctgtcatttcggataaggaaattaatggcccagaggacctcctg

SEQ ID NO: 306 - protein, 451 aa, Glycine max
MTTATEKLSALKSAVAGLNEISESEKNGFISLVSRYLSGEAQHVEWSKIQTPTDEVVVPYDTLAPTPDGSSDVKNLLDKLVVLKLNGGLGTTMGCTGPKSVIEVRDGLTFLDLIVIQIENLNSKYGSNVPLLLMNSFNTHDDTQKIVEKYQNSNIEIHTFNQGILTRMDGTLLAHGDVFPSLLNSGKLDALLSQGKEYVFVANSDNLGAIVDLKILNHLIQNKNEYCMEVTPKTLADVKGGTLISYEGRLLEIAQVPDEHVNEFKSIEKFKIFNTNNLWVNLNAVKRLVEADALKMEIIPNPKEVDGIKVLQLETAAGAAIRFFDKAIGINVPRSRFLPVKATSDLLLVQSDLYTLEDGFVIRNKARANPENPSIELGPEFKKVSNFLGRFKSIPSIVELDSLKVAGNVWFGAGVILKGKISIVANPGVKLEVPDGAVISDKEINGPEDLL

SEQ ID NO: 307 - protein, 15 aa, Glycine max
AIGINVPRSRFLPVK

SEQ ID NO: 308 - protein, 24 aa, Glycine max
ATSDLLLVQSDLYTLEDGFVIRNK

SEQ ID NO: 309 - protein, 18 aa, Glycine max
EYVFVANSDNLGAIVDLK

SEQ ID NO: 310 - protein, 9 aa, Glycine max
ILNHLIQNK

SEQ ID NO: 311 - protein, 26 aa, Glycine max
IQTPTDEVVVPYDTLAPTPDGSSDVK

SEQ ID NO: 312 - protein, 9 aa, Glycine max
LDALLSQGK

SEQ ID NO: 313 - protein, 12 aa, Glycine max
LEVPDGAVISDK

SEQ ID NO: 314 - protein, 9 aa, Glycine max
RLVEADALK

SEQ ID NO: 315 - protein, 14 aa, Glycine max
SAVAGLNEISESEK

SEQ ID NO: 316 - protein, 12 aa, Glycine max
SIPSIVELDSLK

SEQ ID NO: 317 - protein, 17 aa, Glycine max
VLQLETAAGAAIRFFDK

SEQ ID NO: 318 - protein, 9 aa, Glycine max
VSNFLGRFK

SEQ ID NO: 319 - DNA, 1332 bp, Glycine max
  source 1..1332: mol_type = other DNA
  CDS 1..1332
atggatgaggagtacgatgtgatcgtgttgggcacgggtctcaaggagtgcattctcagcggtcttctctccgtcgatggcctcaaggttctgcacatggacaggaatgactattacggaggagaatccacttcgctcaatcttattcagctttggaagagattcaggggagatgacaagcctcctccacatttgggagctagcagggactataacatcgatatgaaccctaagtttattatggctaatggcaatttggtgcgggttctcatacatactgatgttactaagtatctctctttcaaagctgtggatgggagctttgtgtttaataaaggaaaggttcataaggtgcccgcaaatgacatggaggccttgaagtctccacttatggggctatttgaaaaacgccgagcacgcaagtttttcatttatgtgcaaaattacaatgagagtgatcccaagacccatgaagggatggacttgacaagagtgactactaaagagttgatagcaaaatacggtcttgatgataacactatagacttcattggtcatgctttggcgcttcatagtgatgatcgctacttggctgagccagcactggacactgtgaagagaatgaagttatatgcagagtctcttgcacgctttcaaggagggtcaccttacatatatcctttgtatggtttaggagagcttccccaggcatttgcacggcttagtgctgtttatggtgggacatatatgttgaataagcctgagtgcaaggtagaatttgatgaggaaggaaaggttgttggtgtcacatctgaaggggaaactgcaaagtgcaaaaaagttgtttgtgatccatcatatttgcccggcaaggtgaggaaggttggtagggttgcaagagcaatagccataatgagtcacccaatccccagcactgatgattcccattcggtgcaaattattttaccacagaaacagttgggtcggaagtcagacatgtacctattttgttgttcatatactcacaatgttgctccaaagggaaaatttattgcatttgtatcaaccgaggcagagacagatcagccagcaattgaactaaaacctggaattgaccttctaggacctgttgatgagatattttttgatatctatgacagatatgaaccggtcaatgaacccactttggacaactgctttgtatcaacaagttatgatgctactactcactttgagtcgaccgtggatgatgttctcaacatgtatacattgataactggaaaggttattgacctgagtgtggatcttagtgctgctagtgctgcagaagag

SEQ ID NO: 320 - protein, 444 aa, Glycine max
MDEEYDVIVLGTGLKECILSGLLSVDGLKVLHMDRNDYYGGESTSLNLIQLWKRFRGDDKPPPHLGASRDYNIDMNPKFIMANGNLVRVLIHTDVTKYLSFKAVDGSFVFNKGKVHKVPANDMEALKSPLMGLFEKRRARKFFIYVQNYNESDPKTHEGMDLTRVTTKELIAKYGLDDNTIDFIGHALALHSDDRYLAEPALDTVKRMKLYAESLARFQGGSPYIYPLYGLGELPQAFARLSAVYGGTYMLNKPECKVEFDEEGKVVGVTSEGETAKCKKVVCDPSYLPGKVRKVGRVARAIAIMSHPIPSTDDSHSVQIILPQKQLGRKSDMYLFCCSYTHNVAPKGKFIAFVSTEAETDQPAIELKPGIDLLGPVDEIFFDIYDRYEPVNEPTLDNCFVSTSYDATTHFESTVDDVLNMYTLITGKVIDLSVDLSAASAAEE

SEQ ID NO: 321 - protein, 10 aa, Glycine max
AVDGSFVFNK

SEQ ID NO: 322 - protein, 14 aa, Glycine max
FFIYVQNYNESDPK

SEQ ID NO: 323 - protein, 8 aa, Glycine max
VEFDEEGK

SEQ ID NO: 324 - protein, 16 aa, Glycine max
VIDLSVDLSAASAAEE

SEQ ID NO: 325 - protein, 12 aa, Glycine max
VVGVTSEGETAK

SEQ ID NO: 326 - DNA, 1332 bp, Glycine max
  source 1..1332: mol_type = other DNA
  CDS 1..1332
atggatgaggagtacgatgtgatcgtgttgggcacgggtctcaaggagtgcattctcagcggtcttctctccgtcgatggcctcaaggttctgcatatggataggaacgactattacggaggagaatccacttcgctcaatcttattcagctttggaagagattcaggggagatgacaagcctcctccacatttgggcgctagcagggactacaacatcgatatgaaccccaagtttattatggctaatggcaatttggtgcgggttctcatacataccgatgttacaaagtatctctatttcaaagctgtggatgggagctatgtgtttaataaaggaaaggttcataaggtgcctgcaaacgacatggaggccttgaagtccccacttatggggctatttgaaaaacgccgggcacgcaagtttttcatttatgtgcaaaactacaacgagagcgatcccaagacccacgaggggatggacttgacaagagtgactactaaagagttgatagcaaaatacggccttgatgacaacactatagacttcattggtcatgctttggcgcttcatagcgatgatcgctacttggctgagccagcactggacaccgtgaagagaatgaagttatatgcagagtctcttgcacgctttcaaggagggtcaccttacatatatcctttgtatggtttaggagagcttccccaggcatttgcacggcttagtgctgtttatggtggaacatatatgttgaataagcctgagtgcaaggtagaatttgatgaggaaggaaaggttgttggtgttacatctgaaggggaaactgcaaagtgtaaaaaagttgtttgtgatccatcatatttgcctggcaaggtgaggaaggttggtagggttgcaagggcgatagccatcatgagccacccaatccccagcactgatgattcccattcagtgcaaattattttaccacagaaacagttgggtcggaagtcagacatgtacctattttgttgttcgtatactcacaatgttgctccaaagggaaaatttattgcatttgtatcaactgaggcagagacagatcagccggcaattggactgaaacctggaattgaccttctaggacctgttgatgagatattttttgatatctatgacagatatgaaccagtcaacgaacccactttggacaactgctttatatcaacaagttatgatgctactactcactttgagtcgactgtggatgacgttctcaacatgtatacattgataactggaaaggttattgacctcagtgtggatcttagtgctgctagtgctgcagaagag

SEQ ID NO: 327 - protein, 444 aa, Glycine max
MDEEYDVIVLGTGLKECILSGLLSVDGLKVLHMDRNDYYGGESTSLNLIQLWKRFRGDDKPPPHLGASRDYNIDMNPKFIMANGNLVRVLIHTDVTKYLYFKAVDGSYVFNKGKVHKVPANDMEALKSPLMGLFEKRRARKFFIYVQNYNESDPKTHEGMDLTRVTTKELIAKYGLDDNTIDFIGHALALHSDDRYLAEPALDTVKRMKLYAESLARFQGGSPYIYPLYGLGELPQAFARLSAVYGGTYMLNKPECKVEFDEEGKVVGVTSEGETAKCKKVVCDPSYLPGKVRKVGRVARAIAIMSHPIPSTDDSHSVQIILPQKQLGRKSDMYLFCCSYTHNVAPKGKFIAFVSTEAETDQPAIGLKPGIDLLGPVDEIFFDIYDRYEPVNEPTLDNCFISTSYDATTHFESTVDDVLNMYTLITGKVIDLSVDLSAASAAEE

SEQ ID NO: 328 - protein, 10 aa, Glycine max
AVDGSYVFNK

SEQ ID NO: 329 - protein, 14 aa, Glycine max
FFIYVQNYNESDPK

SEQ ID NO: 330 - protein, 8 aa, Glycine max
VEFDEEGK

SEQ ID NO: 331 - protein, 16 aa, Glycine max
VIDLSVDLSAASAAEE

SEQ ID NO: 332 - protein, 12 aa, Glycine max
VVGVTSEGETAK

SEQ ID NO: 333 - DNA, 1512 bp, Glycine max
  source 1..1512: mol_type = other DNA
  CDS 1..1512
atggcgatgagaaccaaactttctttagctatctttttcttttttcttttagccttattttcaaacctagcctttggcaaatgtaaagaaaccgaggtagaagaagaagaccctgagctcgtaacatgcaaacaccaatgccaacagccacagcaatacactgagggtgacaagcgggtatgcttgcaaagatgtgacaggtatcatcgtatgaagcaagagcgagagaaacaaatccaagaggaaactcgtgagaagaaagaggaggaaagccgtgaaagggaagaagaacaacaagagcagcatgaggaacaagatgaaaatccctacatttttgaggaagataaggattttgagaccagagtcgaaactgaaggtggcagaattcgggttctcaagaagttcactgaaaaatccaagcttcttcagggcattgagaacttccgtttggccattttggaagctagagcacacacgttcgtgtctcctcgccactttgattccgaggttgtttttttcaacatcaaggggcgagccgtacttgggttggtgagtgaaagtgaaacagaaaaaatcacccttgaacctggggacatgatacacataccagctggcaccccactgtacatcgttaacagagatgagaatgataagctcttccttgccatgctccatatacctgtctctgtctctactcctggaaaatttgaggaatttttcgggcctggaggacgagacccagaatcggtcctctcagcattcagctggaatgtgctgcaagctgcgctccaaaccccaaaaggaaagttagaaaagctttttgatcaacagaacgagggaagtattttcgcaataagcagagaacaggtgcgtgcgttggcccctaccaagaaaagctcttggtggccattcggtggcgaatccaagcctcaattcaatattttcagcaagcgtcccactatctccaacggatatggtcgtttaactgaagttggtcctgacgatgatgaaaagagttggcttcaaagactcaatctcatgcttacctttaccaacatcacccagagatctatgagtactattcactacaactcacatgcaacgaagatagcactggtgatcgatggcagagggcatcttcaaatatcatgtccacacatgtcatcaaggtcgtcacactcgaagcatgataagagtagcccttcataccatagaatcagttccgatttgaagcctggaatggtgtttgttgtccctcccggtcatcccttcgtcaccatagcttccaataaagagaatctcctcatgatttgcttcgaggttaacgctcgagacaacaagaagtttacatttgcagggaaggacaacattgtgagttctctggacaacgttgctaaggagcttgcctttaactatccttctgagatggtgaacggagtattcgaccgaaaggagagtttctttttcccttttgagttgccgagagaggagcgtggtcgtcgcgctgatgcg

SEQ ID NO: 334 - protein, 504 aa, Glycine max
MAMRTKLSLAIFFFFLLALFSNLAFGKCKETEVEEEDPELVTCKHQCQQPQQYTEGDKRVCLQRCDRYHRMKQEREKQIQEETREKKEEESREREEEQQEQHEEQDENPYIFEEDKDFETRVETEGGRIRVLKKFTEKSKLLQGIENFRLAILEARAHTFVSPRHFDSEVVFFNIKGRAVLGLVSESETEKITLEPGDMIHIPAGTPLYIVNRDENDKLFLAMLHIPVSVSTPGKFEEFFGPGGRDPESVLSAFSWNVLQAALQTPKGKLEKLFDQQNEGSIFAISREQVRALAPTKKSSWWPFGGESKPQFNIFSKRPTISNGYGRLTEVGPDDDEKSWLQRLNLMLTFTNITQRSMSTIHYNSHATKIALVIDGRGHLQISCPHMSSRSSHSKHDKSSPSYHRISSDLKPGMVFVVPPGHPFVTIASNKENLLMICFEVNARDNKKFTFAGKDNIVSSLDNVAKELAFNYPSEMVNGVFDRKESFFFPFELPREERGRRADA

SEQ ID NO: 335 - protein, 17 aa, Glycine max
DFETRVETEGGRIRVLK

SEQ ID NO: 336 - protein, 12 aa, Glycine max
DNIVSSLDNVAK

SEQ ID NO: 337 - protein, 15 aa, Glycine max
GRAVLGLVSESETEK

SEQ ID NO: 338 - protein, 25 aa, Glycine max
LFDQQNEGSIFAISREQVRALAPTK

SEQ ID NO: 339 - protein, 21 aa, Glycine max
RPTISNGYGRLTEVGPDDDEK

SEQ ID NO: 340 - DNA, 1467 bp, Glycine max
  source 1..1467: mol_type = other DNA
  CDS 1..1467
atggcgaccagagccaagctttctttagctatcttccttttctttcttttagccttgatttcaaacctagccttgggcaaacttaaagaaaccgaggtcgaagaagatcccgagctcgtaacatgcaaacaccagtgccaacagcaacggcaatacactgagagtgacaagcgaacatgcttgcaacaatgtgacagtatgaagcaagagcgagagaaacaagtcgaagaggaaactcgcgagaaggaagaagaacatcaagagcagcatgaggaggagcaagaccaaaatccctacgtttttgaagaagataaggatttttcgaccagagtcgaaacagaaggtggcagcattcgggttctcaagaagttcactgagaaatccaagcttcttcaaggcaatgagaatttccgtttggccatcttagaagctagagcacacacgttcgtgtccccacgccactttgattccgaggttgtcttgttcaacattaaggggagagccgtacttgggttggtgagggaaagtgaaacagaaaaaatcaccctagaacctggagacatgatacacataccagcaggcacaccactgtacatcgttaacagagatgagaatgagaagctcctccttgccatgctccatatacctgtctctactcctggaaaatttgaggaatttttcgggcctggaggacgagacccagaatcggtcctctcagcattcagctggaatgtgctgcaagctgcgctccaaaccccaaaaggaaagttagaaaggctttttaatcaacagaacgagggaagtattttcaaaataagcagagaacgggtgcgtgcgttggcccccaccaagaaaagctcttggtggccattcggcggcgaatccaaggctcaattcaatattttcagcaagcgtcccactttctccaacggatatggccgtttaactgaagttggtcctgatgatgaaaagagttggcttcaaagactcaacctcatgcttacctttaccaacatcacccagagatctatgagtactattcactacaactcacatgcaacgaagatagcactggtgatggatggtagagggcatcttcaaatatcatgtccacacatgtcatcaaggtcagactcaaagcatgataagagtagcccctcataccatagaatcagtgcggacttgaagcctggaatggtgtttgttgtccctcctggtcatcccttcgtcactatagcttccaataaagagaatctcctcataatttgcttcgaggttaacgttcgagacaacaagaagtttacgtttgcagggaaggacaacattgtgagctctctggacaacgtagctaaggagctggcctttaactatccttctgagatggtgaacggagtcttcgaaagaaaggagagtctctttttccccttcgagttgccgagcgaggagcgtggtcgtcgcgctgttgcg

SEQ ID NO: 341 - protein, 489 aa, Glycine max
MATRAKLSLAIFLFFLLALISNLALGKLKETEVEEDPELVTCKHQCQQQRQYTESDKRTCLQQCDSMKQEREKQVEEETREKEEEHQEQHEEEQDQNPYVFEEDKDFSTRVETEGGSIRVLKKFTEKSKLLQGNENFRLAILEARAHTFVSPRHFDSEVVLFNIKGRAVLGLVRESETEKITLEPGDMIHIPAGTPLYIVNRDENEKLLLAMLHIPVSTPGKFEEFFGPGGRDPESVLSAFSWNVLQAALQTPKGKLERLFNQQNEGSIFKISRERVRALAPTKKSSWWPFGGESKAQFNIFSKRPTFSNGYGRLTEVGPDDEKSWLQRLNLMLTFTNITQRSMSTIHYNSHATKIALVMDGRGHLQISCPHMSSRSDSKHDKSSPSYHRISADLKPGMVFVVPPGHPFVTIASNKENLLIICFEVNVRDNKKFTFAGKDNIVSSLDNVAKELAFNYPSEMVNGVFERKESLFFPFELPSEERGRRAVA

SEQ ID NO: 342 - protein, 8 aa, Glycine max
AQFNIFSK

SEQ ID NO: 343 - protein, 17 aa, Glycine max
DFSTRVETEGGSIRVLK

SEQ ID NO: 344 - protein, 12 aa, Glycine max
DNIVSSLDNVAK

SEQ ID NO: 345 - protein, 15 aa, Glycine max
GRAVLGLVRESETEK

SEQ ID NO: 346 - protein, 20 aa, Glycine max
RPTFSNGYGRLTEVGPDDEK

SEQ ID NO: 347 - DNA, 1266 bp, Glycine max
  source 1..1266: mol_type = other DNA
  CDS 1..1266
atggtgagaggattgctcaacaagctcgtctctcgctccctctcggtcgccggaaaatggcagcacaatcaactccgccgcctcaacatccacgagtaccagggtgctgaattgatgagcaaacacggagtcaacgttccgagaggcgtggctgtttcctctgttgaagaggccagaaaggtcatcaaggaccttttccctaatgaaaatgagttggtggttaagagtcaaattttagccggcggacgaggtttgggaactttcaaaagcggccttaagggaggagtgcacattgttaagactgaccaggttgaagacatagctgggaagatgcttggacagatactagttaccaaacagactggtcctcagggaaaaatagttagcaaggtttacttgtgtgaaaagctgtcacttgtaaatgagatgtactttgctataacactggatcgtacgagtgctggtccaattataattgcttgtagtaagggaggaaccagcattgaagaccttgcagagaaatttccggacatgattataaaggtacctgttgatgtttttgaaggtattactgatgaaggtgctgcaaaggttgttgatggtttggctctcaaagtggctgatagaaataagtcgattgaacaagtgaagaatttgtataaactttttgttgattgtgactgcactcttttggaaatcaatcccatagcggagacagctgataaccaattggtggctgctgatgctaagttgaattttgatgataatgctgcatatcgccagaaagagatattttctctccgtgatacaacacaagaggaccctcgagaggtgactgctgcaaaggcagatttaaattatattgggttagatggagaaattggctgcatggtgaatggagcagggttagcaatggccacaatggatataattaaattgcatgggggtactcctgccaattttctggatgtaggtggcaatgcctctgaaaaccaggttgttgaggcatttaaaatattgactgctgatgacaaagtgaaggcaattttggttaacatttttggtggtataatgaagtgtgatgttatagcaagtggaatagtgaatgctgcaaaagaggttgcactgaaagtaccagttgtagttcgtctggaaggcaccaatgttgatcaaggaaaaagaatattgaaggaaagtggtatggcattaataacggctgaagatttagatgatgcagctcagaaagcagtgaaagctgcttacaaa

SEQ ID NO: 348 - protein, 422 aa, Glycine max
MVRGLLNKLVSRSLSVAGKWQHNQLRRLNIHEYQGAELMSKHGVNVPRGVAVSSVEEARKVIKDLFPNENELVVKSQILAGGRGLGTFKSGLKGGVHIVKTDQVEDIAGKMLGQILVTKQTGPQGKIVSKVYLCEKLSLVNEMYFAITLDRTSAGPIIIACSKGGTSIEDLAEKFPDMIIKVPVDVFEGITDEGAAKVVDGLALKVADRNKSIEQVKNLYKLFVDCDCTLLEINPIAETADNQLVAADAKLNFDDNAAYRQKEIFSLRDTTQEDPREVTAAKADLNYIGLDGEIGCMVNGAGLAMATMDIIKLHGGTPANFLDVGGNASENQVVEAFKILTADDKVKAILVNIFGGIMKCDVIASGIVNAAKEVALKVPVVVRLEGTNVDQGKRILKESGMALITAEDLDDAAQKAVKAAYK

SEQ ID NO: 349 - protein, 12 aa, Glycine max
DLFPNENELVVK

SEQ ID NO: 350 - protein, 20 aa, Glycine max
EIFSLRDTTQEDPREVTAAK

SEQ ID NO: 351 - protein, 26 aa, Glycine max
LHGGTPANFLDVGGNASENQVVEAFK

SEQ ID NO: 352 - protein, 12 aa, Glycine max
LNFDDNAAYRQK

SEQ ID NO: 353 - protein, 14 aa, Glycine max
SQILAGGRGLGTFK

SEQ ID NO: 354 - protein, 10 aa, Glycine max
TDQVEDIAGK

SEQ ID NO: 355 - protein, 16 aa, Glycine max
VPVVVRLEGTNVDQGK

SEQ ID NO: 356 - protein, 8 aa, Glycine max
VVDGLALK

SEQ ID NO: 357 - DNA, 1266 bp, Glycine max
  source 1..1266: mol_type = other DNA
  CDS 1..1266
atggtgaggggattgctcaacaagctcgtctctcgctccctctccgtcgccggaaaatggcagcacaaccaactccgccgcctcaacatccacgagtaccagggcgctgaattgatgagcaaacacggagttaatgttccgagaggcgtggctgtttcctctgttgaagaggtcagaaaggccatcaaggaccttttccctaatgaaaatgagttggtggttaagagtcaaattttagccggtggaagaggtttgggaactttcaaaagcggccttaagggaggagtgcacattgttaagactgaccaggttgaagacatagctgggaagatgcttgggcagatactagttaccaaacagactggtcctcagggaaaaatagttagcaaggtttacttgtgtgaaaagctgtcactagtaaatgagatgtactttgctataacactggatcgtacgagtgctggtccaattataattgcttgtagtaagggaggaaccagcattgaagacctagcagagaaatttccggacatgattataaaggtacctgttgatgtttttgaaggaattactgatgaagatgctgcaaaggttgttgatggtttggctctcaaagtggctgatagaaataagtcgattgaacaagtgaagaatttgtataaactttttgttgattgtgactgcacccttttggaaatcaatcccattgcggagacagctgataaccaattggtggctgctgatgctaagttgaattttgacgataatgctgcatatcgccagaaagagatatttgctctccgtgatacaacacaagaggaccctcgagaggtgactgctgcaaaggcagacttaaattatattgggttagatggagaaataggctgcatggtgaatggagcagggttagcaatggccacaatggatataattaaattgcatgggggtactcctgccaattttttggatgtaggtggcaatgcctctgaaggccaggtggttgaggcatttaaaatattgactgctgatgacaaagtgaaggcaattttggttaacatttttgggggtataatgaagtgtgatgttatagcaagtggaatagtgaatgctgcaaaagaggttgcactgaaagtacccgtagtagttcgtctggaaggcaccaatgttgatcatggaaaaagaattttgaaggaaagtggtatggcgttgataacggctgaagatttagatgatgctgctcagaaagcagtgaaagctgcttacaaa

SEQ ID NO: 358 - protein, 422 aa, Glycine max
MVRGLLNKLVSRSLSVAGKWQHNQLRRLNIHEYQGAELMSKHGVNVPRGVAVSSVEEVRKAIKDLFPNENELVVKSQILAGGRGLGTFKSGLKGGVHIVKTDQVEDIAGKMLGQILVTKQTGPQGKIVSKVYLCEKLSLVNEMYFAITLDRTSAGPIIIACSKGGTSIEDLAEKFPDMIIKVPVDVFEGITDEDAAKVVDGLALKVADRNKSIEQVKNLYKLFVDCDCTLLEINPIAETADNQLVAADAKLNFDDNAAYRQKEIFALRDTTQEDPREVTAAKADLNYIGLDGEIGCMVNGAGLAMATMDIIKLHGGTPANFLDVGGNASEGQVVEAFKILTADDKVKAILVNIFGGIMKCDVIASGIVNAAKEVALKVPVVVRLEGTNVDHGKRILKESGMALITAEDLDDAAQKAVKAAYK

SEQ ID NO: 359 - protein, 12 aa, Glycine max
DLFPNENELVVK

SEQ ID NO: 360 - protein, 20 aa, Glycine max
EIFALRDTTQEDPREVTAAK

SEQ ID NO: 361 - protein, 26 aa, Glycine max
LHGGTPANFLDVGGNASEGQVVEAFK

SEQ ID NO: 362 - protein, 12 aa, Glycine max
LNFDDNAAYRQK

SEQ ID NO: 363 - protein, 14 aa, Glycine max
SQILAGGRGLGTFK

SEQ ID NO: 364 - protein, 10 aa, Glycine max
TDQVEDIAGK

SEQ ID NO: 365 - protein, 8 aa, Glycine max
VVDGLALK

SEQ ID NO: 366 - DNA, 1203 bp, Glycine max
  source 1..1203: mol_type = other DNA
  CDS 1..1203
atggcgactaagaggagcgtgggaacgttgaaggagggtgatttgaaggggaagagggtattcgtgagggtcgatctgaacgtgcctttggatgacaaccttaacatcaccgatgacactagagtccgtgctgctgttcccaccatcaagtacttgactggttatggtgctaaagtgatcctttctagccatttgggacgtccaaaaggtgtaacacctaaatacagtttgaagcctcttgtgccaaggctgtctcaacttcttggaattgaggttacgatggccaatgactctattggggaggaagttgaaaagttggttacacaacttccagaaggtggtgttttgcttctagagaatgtgaggttctacaaggaggaagagaagaatgatcctgaatttgcaaagaagttggcttctctagctgatctctatgtgaatgatgcatttggcactgcccacagagctcatgcttctacagaaggagttgccaaatacttgaagccctctgttgcaggattcctaatgcagaaggagcttgattatctagttggggctgtgtcaaaccccaagagaccatttgctgctattgtcggtggatcgaaggtgtcttccaagattggagttattgaatccttgttggagaaagttaatgttctcttgcttggtggaggaatgatctttaccttttacaaggctcagggttattcagttgggtcatctcttgtggaggaagacaagcttagccttgcaactacacttcttgaaaaggccaaggctaaaggggtttctttgttgcttccaactgatgttgtcatagcagacaagtttgctgctgatgctaacagcaagactgtgccggcatcaagcatcccagatgggtggatggggttggatattggtcctgattccatcaagacatttggtgaagcattggatacgactcagaccattatttggaatggaccaatgggtgtttttgagtttgataagtttgccacaggaacagaggctatagccaagaaactggcagagctgagtgggaagggagtgacaaccatcattggaggaggtgactccgttgccgctgtggagaaggttggacttgcagacaagatgagccacatctccactggtggtggtgccagcttagagcttcttgagggaaagcaactccctggtgtccttgctcttgatgatgct

SEQ ID NO: 367 - protein, 401 aa, Glycine max
MATKRSVGTLKEGDLKGKRVFVRVDLNVPLDDNLNITDDTRVRAAVPTIKYLTGYGAKVILSSHLGRPKGVTPKYSLKPLVPRLSQLLGIEVTMANDSIGEEVEKLVTQLPEGGVLLLENVRFYKEEEKNDPEFAKKLASLADLYVNDAFGTAHRAHASTEGVAKYLKPSVAGFLMQKELDYLVGAVSNPKRPFAAIVGGSKVSSKIGVIESLLEKVNVLLLGGGMIFTFYKAQGYSVGSSLVEEDKLSLATTLLEKAKAKGVSLLLPTDVVIADKFAADANSKTVPASSIPDGWMGLDIGPDSIKTFGEALDTTQTIIWNGPMGVFEFDKFATGTEAIAKKLAELSGKGVTTIIGGGDSVAAVEKVGLADKMSHISTGGGASLELLEGKQLPGVLALDDA

SEQ ID NO: 368 - protein, 15 aa, Glycine max
AQGYSVGSSLVEEDK

SEQ ID NO: 369 - protein, 13 aa, Glycine max
ELDYLVGAVSNPK

SEQ ID NO: 370 - protein, 8 aa, Glycine max
FAADANSK

SEQ ID NO: 371 - protein, 10 aa, Glycine max
FATGTEAIAK

SEQ ID NO: 372 - protein, 15 aa, Glycine max
GVSLLLPTDVVIADK

SEQ ID NO: 373 - protein, 17 aa, Glycine max
GVTTIIGGGDSVAAVEK

SEQ ID NO: 374 - protein, 10 aa, Glycine max
IGVIESLLEK

SEQ ID NO: 375 - protein, 28 aa, Glycine max
LASLADLYVNDAFGTAHRAHASTEGVAK

SEQ ID NO: 376 - protein, 10 aa, Glycine max
LSLATTLLEK

SEQ ID NO: 377 - protein, 20 aa, Glycine max
LVTQLPEGGVLLLENVRFYK

SEQ ID NO: 378 - protein, 7 aa, Glycine max
NDPEFAK

SEQ ID NO: 379 - protein, 11 aa, Glycine max
QLPGVLALDDA

SEQ ID NO: 380 - protein, 11 aa, Glycine max
RPFAAIVGGSK

SEQ ID NO: 381 - protein, 11 aa, Glycine max
VILSSHLGRPK

SEQ ID NO: 382 - protein, 8 aa, Glycine max
YLTGYGAK

SEQ ID NO: 383 - DNA, 1455 bp, Glycine max
  source 1..1455: mol_type = other DNA
  CDS 1..1455
atggcctcagccgcatcccccaccactttctctctcctccactccgccaccccttcctcctcctccaccgccacctccgcctccccctccccctcgcgcgtgcagctcacctcctccctccgcgcccgcccgctgcgccgcctgggcttcgccgcggcggacccgctgctcgcggcgagtgtagcggcgcgcgtggcggcggtgagaggaggaggaaagggcgcgaggggggtggtgtcgatggcgaagaagagcgtgggggacctgagcgcggcggacctgaaggggaagaaggtcttcgtcagggccgacctcaacgtcccgctcgacgacaaccagaacatcaccgacgacaccagaatccgcgccgccattcccaccatcaaacatctcatccaaaacggcgccaaagtcattctctccagccacttggggcggccgaagggtgtcactcccaaatacagcttggcgcctcttgtaccccggctttctgagctcattggtatccagattgtcaaggccgaggacagtattggtccagaagtagaaaagttggtggcttctcttccagatggaggtgttctccttctagaaaatgtgaggttttacaaggaggaagaaaagaatgaccccgagcatgcaaaaaagcttgcctctctggcagatctgtatgtgaatgatgcatttggtactgctcacagggcacatgcatcaacagagggtgttactaaatacctgaagccatctgttgctggttttcttttgcaaaaggaacttgattaccttgttggggcagtatcaaaccccaaaaggccatttgctgccattgttggtggttccaaagtctcatctaaaattggagtgattgagtcacttttagaaaaagttgacatccttcttcttggtggaggaatgatcttcacattttacaaggcacaaggtctttcagtgggttcatcccttgtagaagaagataagttggatcttgctacatcactacttgcaaaagccaaggaaaagggggtgtctctcttgttaccaagtgatgtggtgattgcagacaaatttgctcctgatgctaatagcaaggtcgtgccagcatctgccatccctgatggctggatgggattggatattggtccagattctgttaaatcattcagtgaagcattggatactacccaaaccatcatatggaatggaccaatgggagtgtttgagtttgacaagtttgctgttggtacagaggctattgcaaagaagctggctgacctcagtggaaagggggtgaccacaattattggaggaggagattctgttgcagctgtggagaaagtaggagttgctagtgtcatgagccacatatctactggtggtggtgccagtttggagttattggaaggcaaagagcttccaggagtccttgctcttgatgaagccgtcccagtcgctgtg

SEQ ID NO: 384 - protein, 485 aa, Glycine max
MASAASPTTFSLLHSATPSSSSTATSASPSPSRVQLTSSLRARPLRRLGFAAADPLLAASVAARVAAVRGGGKGARGVVSMAKKSVGDLSAADLKGKKVFVRADLNVPLDDNQNITDDTRIRAAIPTIKHLIQNGAKVILSSHLGRPKGVTPKYSLAPLVPRLSELIGIQIVKAEDSIGPEVEKLVASLPDGGVLLLENVRFYKEEEKNDPEHAKKLASLADLYVNDAFGTAHRAHASTEGVTKYLKPSVAGFLLQKELDYLVGAVSNPKRPFAAIVGGSKVSSKIGVIESLLEKVDILLLGGGMIFTFYKAQGLSVGSSLVEEDKLDLATSLLAKAKEKGVSLLLPSDVVIADKFAPDANSKVVPASAIPDGWMGLDIGPDSVKSFSEALDTTQTIIWNGPMGVFEFDKFAVGTEAIAKKLADLSGKGVTTIIGGGDSVAAVEKVGVASVMSHISTGGGASLELLEGKELPGVLALDEAVPVAV

SEQ ID NO: 385 - protein, 13 aa, Glycine max
ELDYLVGAVSNPK

SEQ ID NO: 386 - protein, 16 aa, Glycine max
ELPGVLALDEAVPVAV

SEQ ID NO: 387 - protein, 10 aa, Glycine max
FAVGTEAIAK

SEQ ID NO: 388 - protein, 15 aa, Glycine max
GVSLLLPSDVVIADK

SEQ ID NO: 389 - protein, 17 aa, Glycine max
GVTTIIGGGDSVAAVEK

SEQ ID NO: 390 - protein, 10 aa, Glycine max
IGVIESLLEK

SEQ ID NO: 391 - protein, 11 aa, Glycine max
RPFAAIVGGSK

SEQ ID NO: 392 - protein, 11 aa, Glycine max
VILSSHLGRPK

SEQ ID NO: 393 - DNA, 1203 bp, Glycine max
  source 1..1203: mol_type = other DNA
  CDS 1..1203
atggcgactaagaggagcgtgggaacgttgaaggagggtgatttgaaagggaagagggtgttcgtgagggtcgatctgaacgtgcctttggatgacaaccttaacatcaccgatgacactagagtccgtgctgctgttcccaccatcaagtacttaactggtcatggagccaaagtgatcctttctagccacttgggacgtcctaaaggtgtaacacctaaatacagtttgaagcctcttgtgccaaggctgtctcaacttctcggaattgaggttacgatggcaaatgactctattggggaggaagttgagaagttggttgcacaacttccagaaggtggtgttttgcttctagagaatgtgaggttctataaggaggaagagaagaatgaccctgagtttgcaaagaagttggcttctcttgctgatctctacgtgaatgatgctttcggcactgcccacagagctcatgcttctacagaaggagtggccaaatacttgaagccctctgttgcaggattccttatgcagaaggaacttgactatctagttggagctgtgtcaaaccccaagagaccatttgctgctatcgttggtgggtcaaaggtgtcttccaagattggagttattgagtccttgttggagaaagttaacgttctcttgcttggtggaggaatgatctttaccttttacaaggctcagggttattcagttggttcatctcttgtggaggaagacaagcttagccttgcaactacacttctcgaaaaggccaaggctaaaggggtttctttgttgcttccaactgatgtggtcatagcagacaagtttgctgctgatgctaacagcaagactgtgccagcatcaagcatcccagacgggtggatgggattagatattggtcccgattccatcaagacatttggtgaagcattggataagacccagaccgtcatttggaatggaccaatgggtgtttttgagtttgataagtttgccacaggaacagaggccatagccaagaaactggcagagctgagtgggaagggagtaacgaccatcattggaggaggtgactctgttgccgctgtggagaaggttggtcttgcagacaagatgagccacatctccactggtggtggtgccagcttagagcttcttgaggggaagcaactccctggtgtccttgcccttgatgatgct

SEQ ID NO: 394 - protein, 401 aa, Glycine max
MATKRSVGTLKEGDLKGKRVFVRVDLNVPLDDNLNITDDTRVRAAVPTIKYLTGHGAKVILSSHLGRPKGVTPKYSLKPLVPRLSQLLGIEVTMANDSIGEEVEKLVAQLPEGGVLLLENVRFYKEEEKNDPEFAKKLASLADLYVNDAFGTAHRAHASTEGVAKYLKPSVAGFLMQKELDYLVGAVSNPKRPFAAIVGGSKVSSKIGVIESLLEKVNVLLLGGGMIFTFYKAQGYSVGSSLVEEDKLSLATTLLEKAKAKGVSLLLPTDVVIADKFAADANSKTVPASSIPDGWMGLDIGPDSIKTFGEALDKTQTVIWNGPMGVFEFDKFATGTEAIAKKLAELSGKGVTTIIGGGDSVAAVEKVGLADKMSHISTGGGASLELLEGKQLPGVLALDDA

SEQ ID NO: 395 - protein, 15 aa, Glycine max
AQGYSVGSSLVEEDK

SEQ ID NO: 396 - protein, 13 aa, Glycine max
ELDYLVGAVSNPK

SEQ ID NO: 397 - protein, 8 aa, Glycine max
FAADANSK

SEQ ID NO: 398 - protein, 10 aa, Glycine max
FATGTEAIAK

SEQ ID NO: 399 - protein, 15 aa, Glycine max
GVSLLLPTDVVIADK

SEQ ID NO: 400 - protein, 17 aa, Glycine max
GVTTIIGGGDSVAAVEK

SEQ ID NO: 401 - protein, 10 aa, Glycine max
IGVIESLLEK

SEQ ID NO: 402 - protein, 28 aa, Glycine max
LASLADLYVNDAFGTAHRAHASTEGVAK

SEQ ID NO: 403 - protein, 10 aa, Glycine max
LSLATTLLEK

SEQ ID NO: 404 - protein, 20 aa, Glycine max
LVAQLPEGGVLLLENVRFYK

SEQ ID NO: 405 - protein, 7 aa, Glycine max
NDPEFAK

SEQ ID NO: 406 - protein, 11 aa, Glycine max
QLPGVLALDDA

SEQ ID NO: 407 - protein, 11 aa, Glycine max
RPFAAIVGGSK

SEQ ID NO: 408 - protein, 8 aa, Glycine max
TFGEALDK

SEQ ID NO: 409 - protein, 11 aa, Glycine max
VILSSHLGRPK

SEQ ID NO: 410 - DNA, 1143 bp, Glycine max
  source 1..1143: mol_type = other DNA
  CDS 1..1143
atgtcgtcgagcacagttggtcaagtcatcaaatgcaaagctgcggtttcatgggaggcagggaagccacttgtgattgaagaagtagaggtggcaccaccacaagctggtgaagtgcgtttgaagatcctctacacctctctttgccacactgatgtttacttctgggaagccaagggccaaactccattgtttcctcgtatatttggtcatgaagctggagggattgtggagagcgtaggtgagggtgtgactcatctcaaaccaggggaccatgccctcccagtgtttaccggggagtgtggagaatgccctcattgcaagtcagaggagagcaacatgtgtgatcttctcaggatcaacactgataggggtgtcatgatccatgacagccaaacaagattttctattaagggacaacctatttaccattttgttggtacctccacattcagtgaatacactgttgttcatgccggatgtgttgccaaagtcaaccctgctgccccacttgacaaaatttgtgttctcagttgtggaatttgcacaggtcttggggctactatcaatgttgcaaaaccaaaacctggttcttctgttgccatttttggacttggagctgttggccttgctgctgctgaaggggcaaggatttctggtgcatcaagaattattggggttgatttagtttctagcagatttgaagaagctaagaagtttggggtcaatgaatttgtgaacccaaaagaccatgacaagcctgtacaagaggtgattgctgcaatgacgaatgggggtgtggatcgtgctgttgaatgtactggcagcatccaagccatgatctcggcattcgaatgcgtccacgatggttggggtgttgctgtacttgttggagtgccaaacaaagatgatgcattcaaaactcatccagtgaatttcttgaacgagagaactctaaagggtaccttctacggaaactacaaaccccgcactgatcttccttctgttgtggagaagtacatgaatggggaattggaactggagaaattcatcactcacacagttccattctcagagattaacaaggcttttgattacatgctgaaaggggagtccatcagatgcatcattcgaatgggggag

SEQ ID NO: 411 - protein, 381 aa, Glycine max
MSSSTVGQVIKCKAAVSWEAGKPLVIEEVEVAPPQAGEVRLKILYTSLCHTDVYFWEAKGQTPLFPRIFGHEAGGIVESVGEGVTHLKPGDHALPVFTGECGECPHCKSEESNMCDLLRINTDRGVMIHDSQTRFSIKGQPIYHFVGTSTFSEYTVVHAGCVAKVNPAAPLDKICVLSCGICTGLGATINVAKPKPGSSVAIFGLGAVGLAAAEGARISGASRIIGVDLVSSRFEEAKKFGVNEFVNPKDHDKPVQEVIAAMTNGGVDRAVECTGSIQAMISAFECVHDGWGVAVLVGVPNKDDAFKTHPVNFLNERTLKGTFYGNYKPRTDLPSVVEKYMNGELELEKFITHTVPFSEINKAFDYMLKGESIRCIIRMGE

SEQ ID NO: 412 - protein, 10 aa, Glycine max
FGVNEFVNPK

SEQ ID NO: 413 - protein, 13 aa, Glycine max
FITHTVPFSEINK

SEQ ID NO: 414 - protein, 19 aa, Glycine max
GTFYGNYKPRTDLPSVVEK

SEQ ID NO: 415 - protein, 13 aa, Glycine max
THPVNFLNERTLK

SEQ ID NO: 416 - protein, 9 aa, Glycine max
VNPAAPLDK

SEQ ID NO: 417 - DNA, 1113 bp, Glycine max
  source 1..1113: mol_type = other DNA
  CDS 1..1113
atgcgtgcagctgcggtggcatgggaagcagggaagccactgtcaatagaaaccatagaagtggcaccaccccagaaaggtgaagtgcgtttgaggatcctcttcaactccctttgccgtagtgatgtttattggtgggatgctaaggaccagactcctctgtttcctcgtatcttaggccacgaagcttcagggattgtggagagcgtgggcgagggtgtgacacatctgaaaccaggtgaccatgcactcccaatattcactggggagtgtggtgaatgcacttattgtaagtctgaagagagcaacctgtgtgagctactcaggatcaacactgacaggggtgttatgctgagtgatggaaaaacaaggttctccaaaaatggccaacccatttaccacttcgttggaacctccactttcagtgaatacactgttctccatgaaggatgtgtagccaagattaaccctaatgccccacttgacaaagttgctattgtcagctgtggattctgcacaggttttggggctactgtgaatgttgcaaaaccaaagcctaataatactgttgctgtctttggcttaggagctgttggtcttgctgcctgtgaaggggcaagggtttctggtgcatctagaatcatcggggttgatttacttcccaatcgatttgaacaagccaaaaaatttggggtcactgattttgtaaacccgaaagatcacaacaaaccagtccaagaggtcattgctgaaatgactaatggaggagtagatcgtgctattgaatgcactggaagcatccaagcttcaatctcagcatttgaatgcactcatgatggttggggtactgctgtccttgttggtgtgccaaagaaggatgttgaattcaaaactaatcctatgaagttcatggaagggagaactcttaagggtaccttctatggtcactacagaccccgcactgacattcctggtgttgtggagaagtacctcaacaaggagttggaactagacaaatttatcactcactctgtgccattctcaaagatcaacactgcatttgatcttatgctgaaaggggagggcataaggtgcctcatctgcatggaagag

SEQ ID NO: 418 - protein, 371 aa, Glycine max
MRAAAVAWEAGKPLSIETIEVAPPQKGEVRLRILFNSLCRSDVYWWDAKDQTPLFPRILGHEASGIVESVGEGVTHLKPGDHALPIFTGECGECTYCKSEESNLCELLRINTDRGVMLSDGKTRFSKNGQPIYHFVGTSTFSEYTVLHEGCVAKINPNAPLDKVAIVSCGFCTGFGATVNVAKPKPNNTVAVFGLGAVGLAACEGARVSGASRIIGVDLLPNRFEQAKKFGVTDFVNPKDHNKPVQEVIAEMTNGGVDRAIECTGSIQASISAFECTHDGWGTAVLVGVPKKDVEFKTNPMKFMEGRTLKGTFYGHYRPRTDIPGVVEKYLNKELELDKFITHSVPFSKINTAFDLMLKGEGIRCLICMEE

SEQ ID NO: 419 - protein, 19 aa, Glycine max
GTFYGHYRPRTDIPGVVEK

SEQ ID NO: 420 - protein, 9 aa, Glycine max
INPNAPLDK

SEQ ID NO: 421 - DNA, 1140 bp, Glycine max
  source 1..1140: mol_type = other DNA
  CDS 1..1140
atgtctagcaccgttggccagaccatcaagtgcaaagctgcgattgcatgggaggctgggaagccgctggtgattgaagaagtggaggttgcgccaccgcaagccggtgaagtccgtttgaagatcctctacacctctctttgccacactgatgtttacttctgggatgcgaagggccagactccattgtttcctcgcatttttggccatgaagcttcggggattgtggagagcgtaggcgagggtgtgactcatctgaaaccaggtgaccatgccctccctgtgttcacaggagagtgtggagattgcgcccattgcaagtcagaggagagcaacatgtgtgagctactcaggatcaacaccgataggggtgtcatgatccatgatggccaatcaaggttctctaagaatggacaacccatacaccatttcttgggaacctctacattcagtgaatacactgttgtccatgctggatgtgttgcaaagatcaaccctgctgctccacttgacaaagtttgtgttctcagttgtggaatttgcacaggttttggtgctactgtaaatgtagcaaaaccaaaacctggttcctctgttgccatatttggacttggagctgttggccttgcggctgctgaaggtgcaagagtttcaggtgcttcaagaatcattggagttgatttagtttctgcccgatttgaagaagctaagaagtttggggttaatgagtttgtgaacccaaaggatcatgataaacctgtgcaacaggtaattgctgaaatgaccaatggaggtgtggatcgggctgttgaatgtactggcagcatccaagccatggtctcagcattcgaatgcgtccacgatggttggggtcttgctgtacttgttggtgtgcctagtaaagatgatgcattcaaaactgctcctattaatttcctgaacgagaggactcttaagggcaccttttatggcaactacaaaccacgcaccgatcttccatctgttgtcgagaagtacatgagtggggagctagaagtggacaaattcatcactcacacagttccattctcagagatcaacaaagcttttgatttaatgctgaagggacagtccattaggtgtatcatccgcatgcaagag

SEQ ID NO: 422 - protein, 380 aa, Glycine max
MSSTVGQTIKCKAAIAWEAGKPLVIEEVEVAPPQAGEVRLKILYTSLCHTDVYFWDAKGQTPLFPRIFGHEASGIVESVGEGVTHLKPGDHALPVFTGECGDCAHCKSEESNMCELLRINTDRGVMIHDGQSRFSKNGQPIHHFLGTSTFSEYTVVHAGCVAKINPAAPLDKVCVLSCGICTGFGATVNVAKPKPGSSVAIFGLGAVGLAAAEGARVSGASRIIGVDLVSARFEEAKKFGVNEFVNPKDHDKPVQQVIAEMTNGGVDRAVECTGSIQAMVSAFECVHDGWGLAVLVGVPSKDDAFKTAPINFLNERTLKGTFYGNYKPRTDLPSVVEKYMSGELEVDKFITHTVPFSEINKAFDLMLKGQSIRCIIRMQE

SEQ ID NO: 423 - protein, 10 aa, Glycine max
FGVNEFVNPK

SEQ ID NO: 424 - protein, 13 aa, Glycine max
FITHTVPFSEINK

SEQ ID NO: 425 - protein, 19 aa, Glycine max
GTFYGNYKPRTDLPSVVEK

SEQ ID NO: 426 - protein, 9 aa, Glycine max
INPAAPLDK

SEQ ID NO: 427 - protein, 13 aa, Glycine max
TAPINFLNERTLK

SEQ ID NO: 428 - DNA, 927 bp, Glycine max
  source 1..927: mol_type = other DNA
  CDS 1..927
atggcgtcaagacagcaattcaaggaagacagagccgaggcagctgcaaaactcgcagcgaaagacatcggcgacatcaacagaacaaacgagcgcgacgaggggtacgacttgcacgacgaagccaacttcaagcaagcacgagctgaagcagctgcaaagctggcggccaaggatctggaggacgctaacagattaagagacaacactttcaacacatggaaagagcaacatcagcatgatgagaacaaacccggtgtgatagggtccatgtttagagccgccaaggaagccgtggttgggaagcctcaccacgaggaagtttatgcaaaggaaacaaagatgggtgaatattcagattatgcgactcagaaggcgagggaaacgaagcagaaggctggggagtatacagactatgcggctcagaaagcaaaagaaaccaaagactatgctgctgagaaggctaagaatgctaaagataccactgtgcagaaagctggagagtacaaggattataccgctgagaaggctaagaatgctaaagataccactgtgcagaaagctggagaatacaaggattataccgctgagaaggcaattgaagggaaggacagtgcagtggggaagcttggggagctaaaggaatctgctgctgatgctgctaaaagggctatgggtttcttcaccggtggcaacaaagaccaaactagagaggaggaggaggaaacgaggcggagtatgcaggaggtgagggtgcaagataaggagtatggaacgggacgtggtactggagagaagttggtgataaaaatggaagagtcgcggccaggagcggtggcggatgcgctcaaagcagcgaatagggccatggacggcgatatggaggaggaaggcgtgcttcatgtggagcgtcgtagggagaaaatg

SEQ ID NO: 429 - protein, 309 aa, Glycine max
MASRQQFKEDRAEAAAKLAAKDIGDINRTNERDEGYDLHDEANFKQARAEAAAKLAAKDLEDANRLRDNTFNTWKEQHQHDENKPGVIGSMFRAAKEAVVGKPHHEEVYAKETKMGEYSDYATQKARETKQKAGEYTDYAAQKAKETKDYAAEKAKNAKDTTVQKAGEYKDYTAEKAKNAKDTTVQKAGEYKDYTAEKAIEGKDSAVGKLGELKESAADAAKRAMGFFTGGNKDQTREEEEETRRSMQEVRVQDKEYGTGRGTGEKLVIKMEESRPGAVADALKAANRAMDGDMEEEGVLHVERRREKM

SEQ ID NO: 430 - protein, 11 aa, Glycine max
AGEYTDYAAQK

SEQ ID NO: 431 - protein, 24 aa, Glycine max
DIGDINRTNERDEGYDLHDEANFK

SEQ ID NO: 432 - protein, 17 aa, Glycine max
DLEDANRLRDNTFNTWK

SEQ ID NO: 433 - protein, 15 aa, Glycine max
EAVVGKPHHEEVYAK

SEQ ID NO: 434 - DNA, 1104 bp, Glycine max
  source 1..1104: mol_type = other DNA
  CDS 1..1104
atgggcaaggtcaagatcggaatcaacggatttggaagaattggccgtttggtagccagagtggctctgcagagagacgatgttgaactcgttgccgttaacgaccctttcatcaccaccgattacatgacatacatgtttaaatacgacagtgttcatggacactggaagcatcacgatgtcaccgttaaggacgagaagacccttctcttcggtgacaagccagtcactatttttggacacaggtttttatttttcactttaatttttcttactgtttatgattgttgtgtcctgcatgtaaacgttgagtgttgttttctctgtgaactcagaaaccctgaagagatcccatgggggtcaactggagctgacatcattgttgagtccaccggagttttcaccgataaggacaaggccgccgcacatttgaagggtggtgcaaagaaggttattatttctgcccccagtaaggatgcccccatgtttgttgttggtgtcaacgagcacgagtacaagccagagcttgatattatttccaatgctagctgcacaaccaactgccttgccccacttgccaaggttatcaatgacaggtttggcattgttgagggtttgatgaccactgttcattccatcaccgctacccagaagactgttgatggaccatcagccaaggactggagaggtggaagagctgcttcatttaacatcattcctagcagcactggagctgccaaggctgttgggaaagtcctccctgctttgaatggaaaattgactggtatggcattccgtgttcccaccgtggatgtctctgttgttgacctcacagtgaggctggagaaagaagcttcctacgatgaaattaaaaatgctatcaaggaggaatcagagggcaagttgaagggaattcttggttacactgaagatgatgtggtctccactgactttatcggcgatagcagatcaagtatttttgatgcaaaggctggaattgcattgaataagaactttgtgaagcttgtttcttggtacgacaacgagtggggatacagctcacgtgtcattgatcttcttgtattcgttgccaagaagtctctt

SEQ ID NO: 435 - protein, 368 aa, Glycine max
MGKVKIGINGFGRIGRLVARVALQRDDVELVAVNDPFITTDYMTYMFKYDSVHGHWKHHDVTVKDEKTLLFGDKPVTIFGHRFLFFTLIFLTVYDCCVLHVNVECCFLCELRNPEEIPWGSTGADIIVESTGVFTDKDKAAAHLKGGAKKVIISAPSKDAPMFVVGVNEHEYKPELDIISNASCTTNCLAPLAKVINDRFGIVEGLMTTVHSITATQKTVDGPSAKDWRGGRAASFNIIPSSTGAAKAVGKVLPALNGKLTGMAFRVPTVDVSVVDLTVRLEKEASYDEIKNAIKEESEGKLKGILGYTEDDVVSTDFIGDSRSSIFDAKAGIALNKNFVKLVSWYDNEWGYSSRVIDLLVFVAKKSL

SEQ ID NO: 436 - protein, 8 aa, Glycine max
EASYDEIK

SEQ ID NO: 437 - protein, 27 aa, Glycine max
GILGYTEDDVVSTDFIGDSRSSIFDAK

SEQ ID NO: 438 - protein, 8 aa, Glycine max
VIISAPSK

SEQ ID NO: 439 - protein, 8 aa, Glycine max
VLPALNGK

SEQ ID NO: 440 - DNA, 1014 bp, Glycine max
  source 1..1014: mol_type = other DNA
  CDS 1..1014
atgggcaaggtcaagatcggaatcaacggatttggaagaattggccgtttggttgccagagtcgctctgcaaagagacgatgtggagctcgttgccgttaacgaccctttcattaccaccgattacatgacatacatgtttaaatacgacagtgttcacggacactggaagcatcacgatgtcaccgttaaggacgagaagacccttctcttcggtgacaagccagtcactgtttttggacacaggaaccctgaagagatcccatgggggtcaactggagctgatatcattgttgagtccaccggagttttcaccgataaggacaaggccgccgcacatttgaagggtggtgcgaagaaggttattatttctgcccccagtaaggatgcccccatgtttgttgttggtgttaacgagcacgagtacaagccagagcttgatattatttccaatgctagctgcacaaccaactgccttgctccacttgctaaggttattaatgacaggtttggcattgttgagggtttgatgaccactgttcattccatcactgccacccagaagactgttgatgggccatcagccaaagactggagaggtggaagagctgcttcatttaacatcattcctagcagcactggagctgccaaggctgttggaaaagtccttcctgctttgaacggaaaattgactggtatggcattccgtgttcccaccgtggatgtctctgttgttgacctcacagtgaggctggagaaggaagcctcctacgatgaaattaaaaatgctatcaaggaggaatcagagggcaagttgaagggaattcttggttacactgaagatgatgtggtctccaccgactttgtgggcgatagcagatcaagtatttttgatgcaaaggctggaattgcattgaataagaactttgtgaagcttgtctcttggtatgacaacgagtggggatacagctcacgtgtcattgacctgcttgtattcgttgccaagaagtctttt

SEQ ID NO: 441 - protein, 338 aa, Glycine max
MGKVKIGINGFGRIGRLVARVALQRDDVELVAVNDPFITTDYMTYMFKYDSVHGHWKHHDVTVKDEKTLLFGDKPVTVFGHRNPEEIPWGSTGADIIVESTGVFTDKDKAAAHLKGGAKKVIISAPSKDAPMFVVGVNEHEYKPELDIISNASCTTNCLAPLAKVINDRFGIVEGLMTTVHSITATQKTVDGPSAKDWRGGRAASFNIIPSSTGAAKAVGKVLPALNGKLTGMAFRVPTVDVSVVDLTVRLEKEASYDEIKNAIKEESEGKLKGILGYTEDDVVSTDFVGDSRSSIFDAKAGIALNKNFVKLVSWYDNEWGYSSRVIDLLVFVAKKSF

SEQ ID NO: 442 - protein, 8 aa, Glycine max
EASYDEIK

SEQ ID NO: 443 - protein, 27 aa, Glycine max
GILGYTEDDVVSTDFVGDSRSSIFDAK

SEQ ID NO: 444 - protein, 8 aa, Glycine max
VIISAPSK

SEQ ID NO: 445 - protein, 8 aa, Glycine max
VLPALNGK

SEQ ID NO: 446 - DNA, 1020 bp, Glycine max
  source 1..1020: mol_type = other DNA
  CDS 1..1020
atggcatcagataagaagattaggatcggcatcaacggatttggaaggattggccgtttggtggccagggttgcccttcaaaggaatgatgttgaacttgttgctgtcaacgatcctttcatcaccactgattacatgacatacatgttcaagtacgacaccgttcatggccagtggaagcattttgatgtgaaggtgaaggactctaagacccttctctttggtgagaagcccgtcaccgtttttggaatcaggaaccctgaggaaattccatggggtgaggttggtgctgattatgttgttgagtcaactggtgtattcactgacaaggacaaggctgctgctcacttgaagggtggtgccaagaaggttgtcatctctgccccaagcaaagatgcacccatgtttgttgttggtgttaacgagaaggagtacaagccagagcttgacattgtttccaatgctagctgcactaccaattgtcttgctcccctcgccaaggttatcaacgaccgatttggaattgtggagggtcttatgaccactgttcatgccatcacagctactcagaagactgttgatggtccatcaaacaaggactggaggggtggaagagctgcttccttcaatataattcccagcagcactggagctgctaaggctgtaggaaaagttcttccagcactgaatggtaaattgactggcatgtccttccgagtccccactgtagatgtttcagttgttgacctcactgtcaggctagagaagccagcaacctatgagcaaatcaaatctgctatcaaggaggaatcagagggcaaattgaagggtattttgggttacaccgaagatgatgttgtgtctactgattttgttggtgataacaggtctagcatatttgacgccaaggccggaatttctttgaacgacaactttgtaaaacttgtttcctggtatgacaacgaatggggttacagttcccgtgtgattgatttgattgtccacattgcatccgtggct

SEQ ID NO: 447 - protein, 340 aa, Glycine max
MASDKKIRIGINGFGRIGRLVARVALQRNDVELVAVNDPFITTDYMTYMFKYDTVHGQWKHFDVKVKDSKTLLFGEKPVTVFGIRNPEEIPWGEVGADYVVESTGVFTDKDKAAAHLKGGAKKVVISAPSKDAPMFVVGVNEKEYKPELDIVSNASCTTNCLAPLAKVINDRFGIVEGLMTTVHAITATQKTVDGPSNKDWRGGRAASFNIIPSSTGAAKAVGKVLPALNGKLTGMSFRVPTVDVSVVDLTVRLEKPATYEQIKSAIKEESEGKLKGILGYTEDDVVSTDFVGDNRSSIFDAKAGISLNDNFVKLVSWYDNEWGYSSRVIDLIVHIASVA

SEQ ID NO: 448 - protein, 11 aa, Glycine max
AGISLNDNFVK

SEQ ID NO: 449 - protein, 27 aa, Glycine max
GILGYTEDDVVSTDFVGDNRSSIFDAK

SEQ ID NO: 450 - protein, 8 aa, Glycine max
VLPALNGK

SEQ ID NO: 451 - protein, 8 aa, Glycine max
VVISAPSK

SEQ ID NO: 452 - DNA, 1050 bp, Glycine max
  source 1..1050: mol_type = other DNA
  CDS 1..1050
atggtgacctcgtcgcaaaagattctggttaccggcggtgccggtttcataggcacccacaccgtggtccagcttctcaaaggaggcttcagtgtttcaataatcgacaatttcgataactccgtcgtggaagcagtggaccgcgtccgccaagtggtgggccctcagctttctcagaacctggaattcacccagggcgatctcaggaatagggatgacttggagaaactcttctccagaacaacttttgatgccgtgatccactttgctggcctgaaagcggttgctgaaagcgtttccaagccccgtcgctattttgattttaatttggtcggcaccatcaacctctaccaagttatggcaaagtataattgcagaaagatggttttctcatcatctgcaaccgtttatggccaacctgaaaagataccgtgtgaggaggatttcaggttacaagctatgaatccctatggacggaccaagcttttcctggaagaaattgcccgagatattcagaaagctgaaccagaatggaagatcatattactgaggtacttcaatccggttggggctcatgaaagtggtaaactcggtgaagatcccaagggcatcccaaataacctcatgccttacattcagcaagtagccgttggaagattgccggaactcaatgtatatgggcatgattatcccacaagggatggctctgcgatccgggactatatccatgtgatggacttagcagatggtcatattgctgccttgcgaaagctcttcacaacagagaacataggttgtactgcttacaacctgggaactggtcgtggaacatccgtgcttgaaatggttacagcatttgaaaaggcgtctggcaagaaaattccagtaaaattatgtccaagaagaccgggagatgctactgaggtttatgcatctacagagagagctgagaaagaacttggatggaaagcaaaatatggtgtggatgagatgtgcagggaccaatggaattgggcaaagaacaatccccagggttacacggggaagcct

SEQ ID NO: 453 - protein, 350 aa, Glycine max
MVTSSQKILVTGGAGFIGTHTVVQLLKGGFSVSIIDNFDNSVVEAVDRVRQVVGPQLSQNLEFTQGDLRNRDDLEKLFSRTTFDAVIHFAGLKAVAESVSKPRRYFDFNLVGTINLYQVMAKYNCRKMVFSSSATVYGQPEKIPCEEDFRLQAMNPYGRTKLFLEEIARDIQKAEPEWKIILLRYFNPVGAHESGKLGEDPKGIPNNLMPYIQQVAVGRLPELNVYGHDYPTRDGSAIRDYIHVMDLADGHIAALRKLFTTENIGCTAYNLGTGRGTSVLEMVTAFEKASGKKIPVKLCPRRPGDATEVYASTERAEKELGWKAKYGVDEMCRDQWNWAKNNPQGYTGKP

SEQ ID NO: 454 - protein, 12 aa, Glycine max
LFLEEIARDIQK

SEQ ID NO: 455 - DNA, 1050 bp, Glycine max
  source 1..1050: mol_type = other DNA
  CDS 1..1050
atggtgtcttcctcccaacacattctggtcaccggtggtgccggtttcattggcacccacaccgtcgttcagcttctcaaagctggcttcagcgtttcaataatcgacaatttcgataactccgtcatggaagcagtggaccgcgtccgccaagtggttggccctctgctttctcagaacctccaattcacccagggcgatctccggaatagggatgacttggagaaactcttctccaaaacaacatttgatgccgtgatccactttgctggcttgaaagcggttgctgaaagcgttgcgaagccccgtcgctattttgattttaatttggttggcaccatcaacctctacgaatttatggcaaagtataattgcaaaaagatggttttctcatcatctgcaaccgtttatggccaacctgaaaagataccgtgtgaggaggatttcaagttacaagctatgaatccctatggacggaccaagcttttcctggaagaaattgcccgagatattcagaaagctgaaccagaatggaagatcatattactgagatacttcaatccagttggggctcatgaaagtggcaaactcggtgaagatcccaagggcatcccaaataacctcatgccttacattcagcaagtagctgttggaagattgactgaactcaatgtatacggtcatgattatccaacgagggatggctctgcgatccgggactatatccatgtgatggacttggcagatggccatattgctgccctgcgaaagctcttcacaacggagaacataggttgtactgcttacaacctgggaactggtcgtggaacatctgtgcttgaaatggttgcagcatttgaaaaggcttctggcaagaaaattccagtaaaattatgtccaagaagaccgggagatgcgactgaggtttatgcatctacagagagagctgagaaagaacttggttggaaggcaaactatggtgtggaggagatgtgcagggaccaatggaattgggcaaagaacaatccctggggttacgcggggaagcct

SEQ ID NO: 456 - protein, 350 aa, Glycine max
MVSSSQHILVTGGAGFIGTHTVVQLLKAGFSVSIIDNFDNSVMEAVDRVRQVVGPLLSQNLQFTQGDLRNRDDLEKLFSKTTFDAVIHFAGLKAVAESVAKPRRYFDFNLVGTINLYEFMAKYNCKKMVFSSSATVYGQPEKIPCEEDFKLQAMNPYGRTKLFLEEIARDIQKAEPEWKIILLRYFNPVGAHESGKLGEDPKGIPNNLMPYIQQVAVGRLTELNVYGHDYPTRDGSAIRDYIHVMDLADGHIAALRKLFTTENIGCTAYNLGTGRGTSVLEMVAAFEKASGKKIPVKLCPRRPGDATEVYASTERAEKELGWKANYGVEEMCRDQWNWAKNNPWGYAGKP

SEQ ID NO: 457 - protein, 12 aa, Glycine max
LFLEEIARDIQK

SEQ ID NO: 458 - protein, 13 aa, Glycine max
TTFDAVIHFAGLK

SEQ ID NO: 459 - DNA, 1341 bp, Glycine max
  source 1..1341: mol_type = other DNA
  CDS 1..1341
atgggtaaggaaaagactcacatcaacattgtcgtcattggacatgtcgactctgggaagtcaactaccactggtcatttgatctacaagcttggaggtattgacaagcgtgtgattgagaggttcgagaaggaggctgctgagatgaacaagaggtcattcaagtatgcctgggtgcttgacaagctcaaggctgagcgcgaaagaggaattaccattgatattgctttgtggaagtttgaaaccaccaagtactactgcacagtcatcgatgcccccggacatcgtgactttatcaagaacatgattactggtacctcccaggctgattgtgctgtccttatcattgactccaccactggtggttttgaagctggtatttctaaggatggacagactcgtgagcatgctcttcttgctttcacccttggtgtgaagcagatgatctgctgctgtaacaagatggatgccactaccccgaagtactctaaggctaggtatgatgaaattgtgaaggaagtctcttcctacttgaagaaggttggttacaacccagacaagattccatttgtgcccatctctggtttcgagggtgacaacatgattgagaggtccaccaaccttgactggtacaagggaccaactctccttgaggctcttgaccaaatcaatgagcccaagaggccctcagacaagccactcaggcttccattgcaggatgtctacaaaattggtggtattggtaccgtgccagtgggacgtgttgagactggtgtcgtgaagcccggtatggtggtgacttttggtcccactgggctgacaactgaggttaagtctgttgagatgcaccatgaggctctcacagaggctcttccaggtgacaatgttggctttaatgtgaagaatgttgcagtcaaggatctcaagcgtggttttgttgcatccaactccaaggatgaccctgccaaggaagctgccaacttcacatctcaagtcattatcatgaaccaccctggccagattggaaatggatacgcaccggtgcttgattgccacacttctcacattgctgtcaagttttctgaaatcttgaccaagattgacaggcgatctggtaaggagcttgagaaggagcccaaatttttgaagaatggtgatgcaggtatggttaagatgattccaaccaagcccatggtggttgaaactttctctgagtatcctccccttggtcgttttgctgtgagggacatgcgtcaaactgtggctgtcggagtcatcaagagtgttgagaagaaagaccccaccggagccaaggtcaccaaggccgctgccaagaagaag

SEQ ID NO: 460 - protein, 447 aa, Glycine max
MGKEKTHINIVVIGHVDSGKSTTTGHLIYKLGGIDKRVIERFEKEAAEMNKRSFKYAWVLDKLKAERERGITIDIALWKFETTKYYCTVIDAPGHRDFIKNMITGTSQADCAVLIIDSTTGGFEAGISKDGQTREHALLAFTLGVKQMICCCNKMDATTPKYSKARYDEIVKEVSSYLKKVGYNPDKIPFVPISGFEGDNMIERSTNLDWYKGPTLLEALDQINEPKRPSDKPLRLPLQDVYKIGGIGTVPVGRVETGVVKPGMVVTFGPTGLTTEVKSVEMHHEALTEALPGDNVGFNVKNVAVKDLKRGFVASNSKDDPAKEAANFTSQVIIMNHPGQIGNGYAPVLDCHTSHIAVKFSEILTKIDRRSGKELEKEPKFLKNGDAGMVKMIPTKPMVVETFSEYPPLGRFAVRDMRQTVAVGVIKSVEKKDPTGAKVTKAAAKKK

SEQ ID NO: 461 - protein, 8 aa, Glycine max
ARYDEIVK

SEQ ID NO: 462 - protein, 17 aa, Glycine max
DGQTREHALLAFTLGVK

SEQ ID NO: 463 - protein, 7 aa, Glycine max
EVSSYLK

SEQ ID NO: 464 - protein, 7 aa, Glycine max
FSEILTK

SEQ ID NO: 465 - protein, 10 aa, Glycine max
STTTGHLIYK

SEQ ID NO: 466 - protein, 15 aa, Glycine max
THINIVVIGHVDSGK

SEQ ID NO: 467 - DNA, 1341 bp, Glycine max
  source 1..1341: mol_type = other DNA
  CDS 1..1341
atgggtaaagagaaggttcacattaacattgtggtcattggccatgttgactctgggaagtcaaccaccactggccacttgatttacaagcttggaggtattgacaagcgtgttattgagaggtttgagaaagaagctgctgagatgaacaagaggtcattcaaatacgcctgggtgcttgacaagcttaaggctgagcgtgagagaggaattaccattgatattgcattatggaagtttgagaccaccaaatactactgcactgttattgatgctccaggacatcgtgatttcatcaagaacatgattactggaacttcccaggctgattgtgctgtccttatcattgattccacaactggtggttttgaagctggtatttccaaagatggacagactcgcgagcatgctctacttgccttcacccttggtgtcaagcaaatgatctgttgctgtaacaagatggatgccactaccccaaagtactctaaggccaggtatgaagaaattgtgaaggaagtttcttcctacttgaagaaggtaggttacaacccagacaagattccatttgttcccatctcaggttttgagggtgacaacatgattgagaggtccaccaatcttgattggtacaagggaccaacccttctcgaggctcttgaccagatcaatgagcccaagagaccctcagacaagcctttgaggcttcccttgcaggatgtctacaagattggtggtattggaacagtgccagtgggacgagttgaaactggtgtcttgaagcccggtatggtggtgacttttgctcccactggcctgacaactgaagttaagtctgtggagatgcaccatgaagctctccaggaggcccttccaggtgacaacgtaggatttaatgtgaagaatgttgcagtcaaggatctcaagcgtggttttgttgcctcaaactctaaggatgatcctgccaaggaggctgccaacttcacatcccaagtcattataatgaaccatcctggccaaattggaaatggttatgctcctgtgcttgactgccacacttctcacattgccgtcaagtttgccgaacttgtgaccaagattgacaggcgatctggtaaggagattgagaaggagcccaaattcttgaaaaatggagatgcaggttatgttaagatgattcccaccaagcctatggtggtcgaaactttctccgagtatcctcctcttggtcgttttgccgtgagggacatgcgacaaactgtggctgttggagtcatcaagagtgtggagaagaaggacccaactggagccaaggtcaccaaggctgcacagaagaagaag

SEQ ID NO: 468 - protein, 447 aa, Glycine max
MGKEKVHINIVVIGHVDSGKSTTTGHLIYKLGGIDKRVIERFEKEAAEMNKRSFKYAWVLDKLKAERERGITIDIALWKFETTKYYCTVIDAPGHRDFIKNMITGTSQADCAVLIIDSTTGGFEAGISKDGQTREHALLAFTLGVKQMICCCNKMDATTPKYSKARYEEIVKEVSSYLKKVGYNPDKIPFVPISGFEGDNMIERSTNLDWYKGPTLLEALDQINEPKRPSDKPLRLPLQDVYKIGGIGTVPVGRVETGVLKPGMVVTFAPTGLTTEVKSVEMHHEALQEALPGDNVGFNVKNVAVKDLKRGFVASNSKDDPAKEAANFTSQVIIMNHPGQIGNGYAPVLDCHTSHIAVKFAELVTKIDRRSGKEIEKEPKFLKNGDAGYVKMIPTKPMVVETFSEYPPLGRFAVRDMRQTVAVGVIKSVEKKDPTGAKVTKAAQKKK

SEQ ID NO: 469 - protein, 17 aa, Glycine max
DGQTREHALLAFTLGVK

SEQ ID NO: 470 - protein, 7 aa, Glycine max
EVSSYLK

SEQ ID NO: 471 - protein, 10 aa, Glycine max
STTTGHLIYK

SEQ ID NO: 472 - DNA, 1344 bp, Glycine max
  source 1..1344: mol_type = other DNA
  CDS 1..1344
atgggtaaggaaaaggttcacatcagtattgtggtcattggccatgtcgactctgggaaatccactaccactggtcacctgatttacaagcttggaggcattgacaagcgtgttattgagaggtttgagaaggaagctgctgagatgaacaagaggtctttcaagtatgcctgggtgctggacaaacttaaggctgagcgtgaaagaggaatcaccattgatattgctttgtggaagtttgaaacaacaaagtattattgcacagttattgatgcgcctggacatagggatttcattaagaatatgattactgggacatcccaagctgactgtgctgttcttatcattgattcgaccactggtggttttgaagctggtatttccaaggatggacagactcgtgaacatgctctgctttcattcacccttggtgtgaaacagatgatttgctgctgtaacaaaatggatgctactacacccaagtattccaaggccaggtatgatgaaattgtgaaggaagtctcttcctacttgaagaaagtaggatacaaccctgacaagattccttttgttcctatctctggttttgagggagacaacatgattgagaggtccacaaaccttgactggtacaagggtccaactctgcttgatgcacttgaccagattagtgagcccaagaggccctctgacaagcccctcaggcttccccttcaggatgtgtacaagattggaggtattggaactgtgccagtgggacgtgttgagaccggtgtcttgaagcctggaatggtggtgacttttgcaccaactggactgacaactgaagtcaagtctgtggagatgcaccatgaatctcttacagaggcacatcctggtgacaatgtgggattcaatgttaagaatgttgctgttaaggatttgaagcgtggttatgttgcctcaaactcaaaggatgaccctgcaaaggaggctgctaacttcacagcccaagtcatcatcatgaaccaccctggtcagattggaaatggctatgcccctgtcctcgactgccacacttctcacattgctgtcaagtttgctgaactcatgaccaagattgacaggcgatccggcaaagagcttgagaaggagcccaagtttttgaagaacggtgatgctggttttgttaagatgattccaaccaaacccatggttgttgaaactttctccgagtatcctccacttggtaggtttgctgttagggacatgcgtcaaactgttgctgtgggagtcatcaagaacgttgagaagaaggatcctaccggagccaaggtcaccaaggctgcccagaagaagaagtga

SEQ ID NO: 473 - protein, 447 aa, Glycine max
MGKEKVHISIVVIGHVDSGKSTTTGHLIYKLGGIDKRVIERFEKEAAEMNKRSFKYAWVLDKLKAERERGITIDIALWKFETTKYYCTVIDAPGHRDFIKNMITGTSQADCAVLIIDSTTGGFEAGISKDGQTREHALLSFTLGVKQMICCCNKMDATTPKYSKARYDEIVKEVSSYLKKVGYNPDKIPFVPISGFEGDNMIERSTNLDWYKGPTLLDALDQISEPKRPSDKPLRLPLQDVYKIGGIGTVPVGRVETGVLKPGMVVTFAPTGLTTEVKSVEMHHESLTEAHPGDNVGFNVKNVAVKDLKRGYVASNSKDDPAKEAANFTAQVIIMNHPGQIGNGYAPVLDCHTSHIAVKFAELMTKIDRRSGKELEKEPKFLKNGDAGFVKMIPTKPMVVETFSEYPPLGRFAVRDMRQTVAVGVIKNVEKKDPTGAKVTKAAQKKK

SEQ ID NO: 474 - protein, 8 aa, Glycine max
ARYDEIVK

SEQ ID NO: 475 - protein, 17 aa, Glycine max
DGQTREHALLSFTLGVK

SEQ ID NO: 476 - protein, 7 aa, Glycine max
EVSSYLK

SEQ ID NO: 477 - protein, 10 aa, Glycine max
STTTGHLIYK

SEQ ID NO: 478 - DNA, 882 bp, Glycine max
  source 1..882: mol_type = other DNA
  CDS 1..882
atggcttccggtgaacagaaattccctcctcaacaacaacaaacacagcctgggaaggagcatgctatgactccagtaccccaattcactagccctgactacaagccttcaaataaacttcaagggaagattgcattagtcactgggggtgattctgggattggacgagcggtgtgtaacttgtttgccttagaaggtgctaccgtggccttcacgtatgtgaaggggcatgaggacaaggacgcgagggacacattggaaatgatcaagagagcaaagacttcggatgccaaggatccaatggcaatagcatctgatttgggttacgatgagaactgcaagagggtggttgatgaggtcgtgagtgcttatggttgtattgacattctggtcaacaatgcagctgagcagtacgagtgtggaaccgtggaggacatagacgagcctaggcttgagagggtctttcgtacaaatatcttctcctatttcttcatggcgaggcatgccttgaagcacatgaaggaaggaagcagcattatcaacacgacatcagtgaatgcatacaagggacatgcgaaactattggactacacgtccaccaagggggcaattgtggcctatacaaggggtcttgcccttcagctggtgagtaagggaattcgggttaatggggtggctccagggcccatttggacccctttgataccagcctctttcaaggaggaagaaacggcccaatttggagcgcaggtcccaatgaagagagctggtcaacctattgaggttgctccttcctatgtttttcttgcttccaaccaatgctcctcttacataactggacaagtccttcaccccaatggtggaaccgttgtgaatggttaa

SEQ ID NO: 479 - protein, 293 aa, Glycine max
MASGEQKFPPQQQQTQPGKEHAMTPVPQFTSPDYKPSNKLQGKIALVTGGDSGIGRAVCNLFALEGATVAFTYVKGHEDKDARDTLEMIKRAKTSDAKDPMAIASDLGYDENCKRVVDEVVSAYGCIDILVNNAAEQYECGTVEDIDEPRLERVFRTNIFSYFFMARHALKHMKEGSSIINTTSVNAYKGHAKLLDYTSTKGAIVAYTRGLALQLVSKGIRVNGVAPGPIWTPLIPASFKEEETAQFGAQVPMKRAGQPIEVAPSYVFLASNQCSSYITGQVLHPNGGTVVNG

SEQ ID NO: 480 - protein, 15 aa, Glycine max
EGSSIINTTSVNAYK

SEQ ID NO: 481 - protein, 12 aa, Glycine max
FPPQQQQTQPGK

SEQ ID NO: 482 - protein, 17 aa, Glycine max
GAIVAYTRGLALQLVSK

SEQ ID NO: 483 - protein, 8 aa, Glycine max
LLDYTSTK

SEQ ID NO: 484 - DNA, 882 bp, Glycine max
  source 1..882: mol_type = other DNA
  CDS 1..882
atggcaagcaataaggagtctaagttcccagcacagagccagaaaactcagccaggaaaagaacatgtaatgaatccactcccacaagccacaaatcctgatcacaaggccgccaataaactccagggaaaggtggcgttggtgacaggaggtgactcaggaattggcagagcggtttgcctgtgtttcgcaaaagagggtgcaaccgtggcctttacatacgtaaagggccatgaggacagggataaagatgatactctgaagatgctgcttgaagctaagacaagtggtgcagacaatccattggcaatagcagcggatattggctttgatgagaactgcaaacaggtcattgaccttgttgtcaaagaatatggccgccttgatgttctggtcaacaatgcagctgagcagcatttgacaaactctgttgaggaaatcacacaacagcagcttgagagagtcttcggaaccaacatcttttctcagttctttttggtcaagcatgctctgaagcacatgaaagaagggagctgcatcataaactctacttcagttaatgcatacaatgggaatccagaagcgttggactacactgctaccaagggagcaattgtggccttcaccagaggtctttctcagcagctagcgagtaggggaattagggtgaatggtgtggcacctggcccagtttggacgccaatacaaccagcttcaaagcctgctgagatgattcagaacttggggtgtgaggtgccaatgaaccgcgtggctcagccttgtgagattgcaccatgttatttgttcttggcaacttgtcaggactcttcctactttactggccaagtcctccatccaaatggtgggatggtcgtcaacgct

SEQ ID NO: 485 - protein, 294 aa, Glycine max
MASNKESKFPAQSQKTQPGKEHVMNPLPQATNPDHKAANKLQGKVALVTGGDSGIGRAVCLCFAKEGATVAFTYVKGHEDRDKDDTLKMLLEAKTSGADNPLAIAADIGFDENCKQVIDLVVKEYGRLDVLVNNAAEQHLTNSVEEITQQQLERVFGTNIFSQFFLVKHALKHMKEGSCIINSTSVNAYNGNPEALDYTATKGAIVAFTRGLSQQLASRGIRVNGVAPGPVWTPIQPASKPAEMIQNLGCEVPMNRVAQPCEIAPCYLFLATCQDSSYFTGQVLHPNGGMVVNA

SEQ ID NO: 486 - protein, 11 aa, Glycine max
EGATVAFTYVK

SEQ ID NO: 487 - protein, 8 aa, Glycine max
QVIDLVVK

SEQ ID NO: 488 - DNA, 879 bp, Glycine max
  source 1..879: mol_type = other DNA
  CDS 1..879
atggcttcgggtgaaaagaaattcccacctcaacaacaacaaacacagcctgggaaggagcatgctatgaatccagtaccccaattcactagccctgactacaagccttcaaataaacttcaaggaaagatagcattagtgactgggggtgactctgggattggacgagcggtgtgtaacttgtttgccttagaaggtgctaccgtgggcttcacatatgtgaaggggcatgaggacaaggacgcgagggacacgttggaaatgatcaagagggcaaagacttcggatgctaaagatccaatggcggtaccagctgatttgggttacgacgagaattgcaagagagtggttgatgaggtcgtgaatgcttatggttgtattgacattctggtcaacaatgcagctgagcaatacgagtgtggaacagtggaggacattgatgagcctaggcttgagagggtctttcgtacaaatatcttctcctatttcttcatgaccaggcatgccttgaagcacatgaaggaaggaagcagcattatcaacacgacatcggtgaatgcatacaagggaaatgcgaaactattggactacacgtccacgaagggagcaattgtggcctatacaaggggacttgctcttcagttggtgagtaagggaattcgggttaatggggtggctcctgggcctatttggaccccattgataccctcctctttcaaggaggaagaaacggctcaatttggtgcccaggtgccaatgaagagagctggccagcctattgaggttgctccgtcttatgtttttcttgcttgcaaccaatgctcctcttacataactggacaagtccttcaccccaatggtggaaccgttgtcaatggt

SEQ ID NO: 489 - protein, 293 aa, Glycine max
MASGEKKFPPQQQQTQPGKEHAMNPVPQFTSPDYKPSNKLQGKIALVTGGDSGIGRAVCNLFALEGATVGFTYVKGHEDKDARDTLEMIKRAKTSDAKDPMAVPADLGYDENCKRVVDEVVNAYGCIDILVNNAAEQYECGTVEDIDEPRLERVFRTNIFSYFFMTRHALKHMKEGSSIINTTSVNAYKGNAKLLDYTSTKGAIVAYTRGLALQLVSKGIRVNGVAPGPIWTPLIPSSFKEEETAQFGAQVPMKRAGQPIEVAPSYVFLACNQCSSYITGQVLHPNGGTVVNG

SEQ ID NO: 490 - protein, 15 aa, Glycine max
EGSSIINTTSVNAYK

SEQ ID NO: 491 - protein, 12 aa, Glycine max
FPPQQQQTQPGK

SEQ ID NO: 492 - protein, 17 aa, Glycine max
GAIVAYTRGLALQLVSK

SEQ ID NO: 493 - protein, 8 aa, Glycine max
LLDYTSTK

SEQ ID NO: 494 - DNA, 3207 bp, Glycine max
  source 1..3207: mol_type = other DNA
  CDS 1..3207
atggcttctgagcaaatgcctcgcagagagaacaacagcaatagggtgatagagagagaggttcacgttgagacacacagaaaagagtccctccctgataagggaagggttgtgatagagagtgagaagaaggagaaagagagagagttggcttctgatagagccaggtctggaaatatagttaaggaggacaaagaggttgaagaaggaagaagaggaaaagaaagtggtggtggtggtggtgttcagcatgagaggaagtttgaaacgaaagttgaagagaagaagggttttggggaaaaggctgaactcgagggaataacaagagtggtggtgggaagagagtgcagcaaagaggagaaacaaggaagaacaagagagaaagtagaggcagagaaagcaagatcaatggagaaaactagagatgaagctaaaggaagagaacaagctgaaaccaaaggtcaaaccaagaagggtggtggtgaggaacagggaagaagaagagaagaaataggagaagaaaataaacaatccagcctcgaagagatttctaaatgcagagctgaagctgaagagaaagttaaggagagaaaggagagatcaaaacaagcagcaagtaatgaagctgccaaagagagaaacaagcaggcaaataaggaggcaacaaaggcaaaagatgtcacaagagagaagggtcaacaaggcaatcttaaaacaggaaatgagacacaagacattggccaaaagggttatggtggagcaaaggataccattggtagagtggacaacattactacaccacttgctaaaaaggctcaagttgctggtagcaccactgttcagtatgctggagagaaatcagcacaagccaaagatgctacggtaggatcaatggagaaaactacaaaggatgctaaaggaagagaaaaagcaggaaccaaaggtcaaacaaagaagggtggtggtgaggaacaaggaaaaagaagagaagaaacaggagaagaaaaacaatccagcctggaggagatttctaagcacagagctgaagctcaacaaaaagcaatgagttcaattgaaggtgctaaggagagatacgagagagcaaaacaagaagcaagtaaagcttccaaagagagaaacattcaagcaaataagagggctgaagaagaaagaagaaaagcagaaagtggtaggggtgttcatgtggataagtttgaagagaagaaggattcagtgctgaaagcagaactcgagagaagaacaagagaggtagagggaagagagtggaacaaagaggagaaacaaagaagttgtccaagagggaaagtagaggcagagaaagcaagaccaatggaagaaacaaaactggaagctaaaggaagagaaaaagctgaaaccaaagagggtggtggtgaggaaaaaggaatgagaagagaagaaataggagaagaaaataaacaatccagcctggaagagatttttaagtacagagctgaagctcaacagaaagttaaggagagaaacgagagagcaaaacaagcagcagaagctgccaaagagagaaacaagcaggcaaataaagggaatgaagaaggcagaagagaaagagaaagtggtgggggtgttcatcatgaggctaagtttgaaactaaaggtgaaaaaaagaaggatttagtgggaagagagcggaacaaagaggagaaacaaagaagcagtccaagaggaaaagtagaggcagagaaagcaagaccaatggaagaaacaaaggtggaagctaaaggaagagaaaaagatgtaaccaaagagggtggtgttgaggaacaaggaagaggaagaaaagaaaatgaacaatctagcttgaaagacatttccaagtacagagctgaagctcaacaaaattcattcaatgctactgaagctgctaaggaaagatacgagagggcaaaccaagcgacaagtgaagctgccaaagagagaaacaagcaggcaaataaggaggcaacaaaggcaaaagatgtcacaagagaaatggatcaacaaggcaatcttaaaacagaaaatgagacacaggacaatgaccagaagggttatggtggagcaaagtacaccattggtagtgtggaccacattactacaccatttattgtagaggccaatcttaaatcagaaaatcagatacaagacaatggccaaaagggttatggtggagcaaaggacacaattggtagtgtagaccacattactacaccacttgctgaaaaggccaaagctgctggtagcaccactgctcagtatgttggggagaaagcagcacaagccaaagatgctactgtgcaaactggcaagagtgctgcaaaagttgcctcagatttgagggacaaggctttggccacagggtggagtgctgcacattattctactgagataactgtggagggaaccaaggctgcaacaaatgttgttaagggagttgctgagtatgcaggacagaaggcttctgagcttgcagctatgtcagtggatactgcgaaaggtttagctgctgctgcagctggggagaatgccaaggagtacactgcaaggaagaaggtggaagctgagatggatttggaggccaaaagggaagctcaaaatcaggaatcgaaggagtggccatcagcaaaatccaccactgagagcattcaaagaggacaaggacaacaagaacagcatgagcaaggagaaaaatgggaacatggtgcacaaaggactaataagccacatgaaagcaacattgtggaaacctttgaggaaaacctaggtgggattggggaaaaaagggaacaaaaaccacttggaagcatcattggagagagctttcaaggagttcaagaaaaagggagtgaagtgatggcaagcattggtgaaaaactgggaagtgctgcacatacattagtgaagaagccattggataaggccactgagggagggagagaggtgttgggagctgtaggtgaaactgtggttgagattggtgaaaacatggtgaaaaaaccagcacagaaagtgcagcagcatcaaggtgaaggtggaggaggggtgctgaatgccattggtgaaaccatagcagagatagcagagacaactaaggtgattgttgctggagagggagaaaaggagataatagtaaaaacacatgaatttgaatatggagacttgaaacttgat

SEQ ID NO: 495 - protein, 1069 aa, Glycine max
MASEQMPRRENNSNRVIEREVHVETHRKESLPDKGRVVIESEKKEKERELASDRARSGNIVKEDKEVEEGRRGKESGGGGGVQHERKFETKVEEKKGFGEKAELEGITRVVVGRECSKEEKQGRTREKVEAEKARSMEKTRDEAKGREQAETKGQTKKGGGEEQGRRREEIGEENKQSSLEEISKCRAEAEEKVKERKERSKQAASNEAAKERNKQANKEATKAKDVTREKGQQGNLKTGNETQDIGQKGYGGAKDTIGRVDNITTPLAKKAQVAGSTTVQYAGEKSAQAKDATVGSMEKTTKDAKGREKAGTKGQTKKGGGEEQGKRREETGEEKQSSLEEISKHRAEAQQKAMSSIEGAKERYERAKQEASKASKERNIQANKRAEEERRKAESGRGVHVDKFEEKKDSVLKAELERRTREVEGREWNKEEKQRSCPRGKVEAEKARPMEETKLEAKGREKAETKEGGGEEKGMRREEIGEENKQSSLEEIFKYRAEAQQKVKERNERAKQAAEAAKERNKQANKGNEEGRRERESGGGVHHEAKFETKGEKKKDLVGRERNKEEKQRSSPRGKVEAEKARPMEETKVEAKGREKDVTKEGGVEEQGRGRKENEQSSLKDISKYRAEAQQNSFNATEAAKERYERANQATSEAAKERNKQANKEATKAKDVTREMDQQGNLKTENETQDNDQKGYGGAKYTIGSVDHITTPFIVEANLKSENQIQDNGQKGYGGAKDTIGSVDHITTPLAEKAKAAGSTTAQYVGEKAAQAKDATVQTGKSAAKVASDLRDKALATGWSAAHYSTEITVEGTKAATNVVKGVAEYAGQKASELAAMSVDTAKGLAAAAAGENAKEYTARKKVEAEMDLEAKREAQNQESKEWPSAKSTTESIQRGQGQQEQHEQGEKWEHGAQRTNKPHESNIVETFEENLGGIGEKREQKPLGSIIGESFQGVQEKGSEVMASIGEKLGSAAHTLVKKPLDKATEGGREVLGAVGETVVEIGENMVKKPAQKVQQHQGEGGGGVLNAIGETIAEIAETTKVIVAGEGEKEIIVKTHEFEYGDLKLD

SEQ ID NO: 496 - protein, 13 aa, Glycine max
AAGSTTAQYVGEK

SEQ ID NO: 497 - protein, 21 aa, Glycine max
ALATGWSAAHYSTEITVEGTK

SEQ ID NO: 498 - protein, 15 aa, Glycine max
AQVAGSTTVQYAGEK

SEQ ID NO: 499 - protein, 15 aa, Glycine max
DTIGRVDNITTPLAK

SEQ ID NO: 500 - protein, 16 aa, Glycine max
DTIGSVDHITTPLAEK

SEQ ID NO: 501 - protein, 16 aa, Glycine max
ERELASDRARSGNIVK

SEQ ID NO: 502 - protein, 15 aa, Glycine max
ERYERANQATSEAAK

SEQ ID NO: 503 - protein, 18 aa, Glycine max
GGGEEQGRRREEIGEENK

SEQ ID NO: 504 - protein, 12 aa, Glycine max
GLAAAAAGENAK

SEQ ID NO: 505 - protein, 9 aa, Glycine max
GRVVIESEK

SEQ ID NO: 506 - protein, 9 aa, Glycine max
GVAEYAGQK

SEQ ID NO: 507 - protein, 10 aa, Glycine max
LGSAAHTLVK

SEQ ID NO: 508 - protein, 9 aa, Glycine max
QSSLEEIFK

SEQ ID NO: 509 - protein, 9 aa, Glycine max
QSSLEEISK

SEQ ID NO: 510 - protein, 20 aa, Glycine max
REQKPLGSIIGESFQGVQEK

SEQ ID NO: 511 - protein, 11 aa, Glycine max
TGNETQDIGQK

SEQ ID NO: 512 - protein, 9 aa, Glycine max
VIVAGEGEK

SEQ ID NO: 513 - protein, 28 aa, Glycine max
VQQHQGEGGGGVLNAIGETIAEIAETTK

SEQ ID NO: 514 - DNA, 735 bp, Glycine max
  source 1..735: mol_type = other DNA
  CDS 1..735
atggcactgtgcaaccgcgtcagatcgggaatctctctcttcaccaaatccacgttcctcacttcccagcgatccactctccaacgatccctcgttgcccctttcatttccctcgcctccagaaattacgccaacgtgccggggcaaaaggaaaccaaagttaaggtccctctggctttgtttggaggttcaggaaactatgcctctgctttgtatattgcatctgtgaaagccaatgcagtggaaaaggttgagactgagcttctgcaatttgttgaggcagtaaagaatagtcccatattttcacagtttataagggacttgtctgtggctaaggatgttagagttaaagttatccaggatatttgtggggaagccaagttttcggatgttacaaagaacttccttgttattgttgctgagaatggaaggcttaaaaacatagacaccattgcaaagaggtttgcacagttgtcaatggcatataagggagaagtgcaagcgactgtgaccactgttgttcctcttccccctgaagaggaaaaggcattgaaggagactcttcaggaaataataggttctggagcgaaggttcatcttgaacagaagattgatccaagcatacttggtgggcttgtgcttgaatttagccagaaggtctttgacatgtcaattaagaccagggcacaacagatggagaggatcctccgagagcccgtgtccattgccaacatc

SEQ ID NO: 515 - protein, 245 aa, Glycine max
MALCNRVRSGISLFTKSTFLTSQRSTLQRSLVAPFISLASRNYANVPGQKETKVKVPLALFGGSGNYASALYIASVKANAVEKVETELLQFVEAVKNSPIFSQFIRDLSVAKDVRVKVIQDICGEAKFSDVTKNFLVIVAENGRLKNIDTIAKRFAQLSMAYKGEVQATVTTVVPLPPEEEKALKETLQEIIGSGAKVHLEQKIDPSILGGLVLEFSQKVFDMSIKTRAQQMERILREPVSIANI

SEQ ID NO: 516 - protein, 12 aa, Glycine max
ETLQEIIGSGAK

SEQ ID NO: 517 - DNA, 678 bp, Glycine max
  source 1..678: mol_type = other DNA
  CDS 1..678
atgaccacacaagtaccaccacacagtgtccaagtccacacaacaacacaccgctacgaagctggcgtcgtcccccccggagcccgtttcgaaccgccgcgttatgaagccggcgtcaaagcgccctccatttaccattccgagagaggtccaacgacttcccaggtcctcgccgtcctcgccggcctcccggtcggtggcatcctgctcctcctggcgggattgaccctggcaggaaccctcaccggactggcagttgccacgccgctcttcgtcctcttcagcccggtgctggttccagccacggtcgccatcggcctggccgtggccgggttcctgacgtcgggggccttcgggctcacggcgctgtcttccttctcctggatcctgaactacatccgggagacccagccagcctcggagaacctcgccgccgccgcgaagcatcacctcgccgaggcggcggagtacgtggggcagaagacgaaggaagtggggcagaagaccaaggaggttgggcaggatattcagagcaaggcacaggatacaagggaggcagctgcaagggatgcaagggaggcagctgcaagggatgcaagggaagctgctgcaagggatgcaaaggtggaggcaagggatgtaaagagaacaacagtgacagcaacaaccgcaaccgca

SEQ ID NO: 518 - protein, 226 aa, Glycine max
MTTQVPPHSVQVHTTTHRYEAGVVPPGARFEPPRYEAGVKAPSIYHSERGPTTSQVLAVLAGLPVGGILLLLAGLTLAGTLTGLAVATPLFVLFSPVLVPATVAIGLAVAGFLTSGAFGLTALSSFSWILNYIRETQPASENLAAAAKHHLAEAAEYVGQKTKEVGQKTKEVGQDIQSKAQDTREAAARDAREAAARDAREAAARDAKVEARDVKRTTVTATTATA

SEQ ID NO: 519 - protein, 9 aa, Glycine max
EVGQDIQSK

SEQ ID NO: 520 - protein, 11 aa, Glycine max
RTTVTATTATA

SEQ ID NO: 521 - DNA, 627 bp, Glycine max
  source 1..627: mol_type = other DNA
  CDS 1..627
atgacaaggaaacaagcaagttccacgagacgtggcggtctttcaggagtcttgcattccaaatccaaatcttctcctctactctccatatcccttgtcctatttgtagccatccttctcatcctttatgcttgtatcgggtcaggtatattgggaggaagaaaggatgtgattagcatggctgaagatgatttttcatgcacttttgaagttccaagtgcaattcctgttttgaagaatgcatatggtggcagcatgaaaaatgttttgcacgtaggccctgagagttgttcggtggtctccaaatttttaagagaaggggaaactgaagcatggggtgtggagccatatgacttagacgatgctgataggaactgcaaggctcttgtgcagaaaggcattatacgtgttgcagatataaaattccctttaccatacagggtgaagtctttttctcatgttatagtgtcagatgctttggattacctgtctccaaaatatataaacaaaactcttcctgagctagcaagggtatcttctgatggtattattattttcacaggttacccaggtcaaccgagagctaaaatagcaccattatccaaatttggccgtccacataggtgc

SEQ ID NO: 522 - protein, 209 aa, Glycine max
MTRKQASSTRRGGLSGVLHSKSKSSPLLSISLVLFVAILLILYACIGSGILGGRKDVISMAEDDFSCTFEVPSAIPVLKNAYGGSMKNVLHVGPESCSVVSKFLREGETEAWGVEPYDLDDADRNCKALVQKGIIRVADIKFPLPYRVKSFSHVIVSDALDYLSPKYINKTLPELARVSSDGIIIFTGYPGQPRAKIAPLSKFGRPHRC

SEQ ID NO: 523 - protein, 8 aa, Glycine max
NAYGGSMK

SEQ ID NO: 524 - DNA, 723 bp, Glycine max
  source 1..723: mol_type = other DNA
  CDS 1..723
atggccgcgcgagctctgttgaccagaaaaaccctagccaccgtgctccgcaacgacgcgaagcccataatcggagttggcataacagcagcggctactcattcacgcgggttgcacgtgtacacgctacccgatctggattacgactatggcgcactggagccagccatcagcggcgacatcatgcagctgcaccaccagaagcaccaccagacttacatcaccaactacaacaaggccctcgagcagctccaagacgccatcgccaagaaagattcctccgccgtcgttaagctccagggcgccatcaagttcaacggcggaggtcatgtcaaccattctattttctggaaaaatctagctcctgttcgtgaaggaggtggtgaaccacccaagggttcactgggatgggctattgacacacattttggttcttttgaagcattaatacaaaaagttaacgcagaaggtgctgcactacaggggtctggatgggtgtggcttggtctggacaaagagttgaagaggcttgtagttgaaaccactgccaaccaggacccactggttactaagggaccaaatttggttccattgattggtattgatgtttgggagcatgcgtactacttacagtacaagaatgttagaccagactatctgaagaacatttggaaagttattaattggaaatatgccagtgaagtgtatgagaaagagagctct

SEQ ID NO: 525 - protein, 241 aa, Glycine max
MAARALLTRKTLATVLRNDAKPIIGVGITAAATHSRGLHVYTLPDLDYDYGALEPAISGDIMQLHHQKHHQTYITNYNKALEQLQDAIAKKDSSAVVKLQGAIKFNGGGHVNHSIFWKNLAPVREGGGEPPKGSLGWAIDTHFGSFEALIQKVNAEGAALQGSGWVWLGLDKELKRLVVETTANQDPLVTKGPNLVPLIGIDVWEHAYYLQYKNVRPDYLKNIWKVINWKYASEVYEKESS

SEQ ID NO: 526 - protein, 11 aa, Glycine max
ALEQLQDAIAK

SEQ ID NO: 527 - protein, 11 aa, Glycine max
HHQTYITNYNK

SEQ ID NO: 528 - protein, 14 aa, Glycine max
NLAPVREGGGEPPK

SEQ ID NO: 529 - protein, 8 aa, Glycine max
NVRPDYLK

SEQ ID NO: 530 - protein, 16 aa, Glycine max
RLVVETTANQDPLVTK

SEQ ID NO: 531 - protein, 8 aa, Glycine max
YASEVYEK

SEQ ID NO: 532 - DNA, 720 bp, Glycine max
  source 1..720: mol_type = other DNA
  CDS 1..720
atggccgcgcgagccctgttgaccagaaaaaccctagccaccgttctccgcaacgacgcgaagcccataatcggagctggcatagctgcagctactcattctcgtgggttgcacgtgtacacgctccccgatctggattacgactatggcgctctggagccagccatcagcggcgaaatcatgcagctgcaccaccagaagcaccaccagacttacatcaccaacttcaacaaggccctcgagcagctccaagacgccgtcgccaagaaagattcctccgccgtcgttaagctccagggcgccatcaagttcaacggcggaggtcacgtcaaccattctattttctggaaaaatctagctcctgttcgtgaaggaggtggtgaaccacccaagggttcactgggatgggccattgacacacattttggctcttttgaagcattagtacaaaaagttaatgcagaaggtgctgcactacaggggtctggatgggtgtggcttggtctggacaaagagttgaagaggcttgtagttgaaaccactgccaaccaggacccactggttactaagggaccaaatttggttccattgcttggtattgatgtttgggagcatgcgtactacttacagtacaagaatgttagaccagactatctgaagaacatttggaaagttattaattggaaatatgccagtgaagtgtatgagaaagagagctct

SEQ ID NO: 533 - protein, 240 aa, Glycine max
MAARALLTRKTLATVLRNDAKPIIGAGIAAATHSRGLHVYTLPDLDYDYGALEPAISGEIMQLHHQKHHQTYITNFNKALEQLQDAVAKKDSSAVVKLQGAIKFNGGGHVNHSIFWKNLAPVREGGGEPPKGSLGWAIDTHFGSFEALVQKVNAEGAALQGSGWVWLGLDKELKRLVVETTANQDPLVTKGPNLVPLLGIDVWEHAYYLQYKNVRPDYLKNIWKVINWKYASEVYEKESS

SEQ ID NO: 534 - protein, 11 aa, Glycine max
ALEQLQDAVAK

SEQ ID NO: 535 - protein, 11 aa, Glycine max
HHQTYITNFNK

SEQ ID NO: 536 - protein, 14 aa, Glycine max
NLAPVREGGGEPPK

SEQ ID NO: 537 - protein, 8 aa, Glycine max
NVRPDYLK

SEQ ID NO: 538 - protein, 16 aa, Glycine max
RLVVETTANQDPLVTK

SEQ ID NO: 539 - protein, 8 aa, Glycine max
YASEVYEK

SEQ ID NO: 540 - DNA, 486 bp, Glycine max
  source 1..486: mol_type = other DNA
  CDS 1..486
atggctccaattgcagtgggagatgtcattccagacggcattttggcttacttggatgaagaaaacaaaccccaaactgtctctattcactctctcgccgccggcaagaaagtcatcatctttggggttcccggtgccttcactcctacatgcagcttgaagcacgtgcctggcttcattgagagagcagaagagctgaaaggaaagggtgtggacgaaataatctgtatcagcgtgaatgatccctttgtgatgaactcatgggccaaaacgttcccagagaacaagcatgttaagttccttgctgatggtgcagccaaatacaccaatgccctcggtcttgagcttgacctcaccgacaagggtctcggcgtccgctcaaagaggtttgctttgctggtggaagacctcaaggtgaaggttgcaaatgttgaaagtggaggagagttcaccatctccagtgctgaagagatcatcaaggccctc

SEQ ID NO: 541 - protein, 162 aa, Glycine max
MAPIAVGDVIPDGILAYLDEENKPQTVSIHSLAAGKKVIIFGVPGAFTPTCSLKHVPGFIERAEELKGKGVDEIICISVNDPFVMNSWAKTFPENKHVKFLADGAAKYTNALGLELDLTDKGLGVRSKRFALLVEDLKVKVANVESGGEFTISSAEEIIKAL

SEQ ID NO: 542 - protein, 13 aa, Glycine max
HVPGFIERAEELK

SEQ ID NO: 543 - protein, 10 aa, Glycine max
RFALLVEDLK

SEQ ID NO: 544 - protein, 20 aa, Glycine max
VANVESGGEFTISSAEEIIK

SEQ ID NO: 545 - protein, 14 aa, Glycine max
YTNALGLELDLTDK

SEQ ID NO: 546 - DNA, 486 bp, Glycine max
  source 1..486: mol_type = other DNA
  CDS 1..486
atggctccaattgcagtgggaaacgtgattccagacggcattttggcttacttggatgaagaaaacaaaccccaaactgtctccattcactctctcgccaccggcaagaaagtcatcatctttggggttcccggtgccttcactcctacttgcagcttgaagcacgttcctggcttcattgagagagcagaagagctgaaagggaagggtgtggacgaaataatctgtatcagtgtgaatgatccatttgtgatgaactcatgggccaaaacattcccagagaacaagcatgttaagttccttgctgatggtgcagccaaatacaccaatgcccttggtcttgagcttgacctcactgacaagggtctcggcgtccgctcaaagaggtttgctctgctggtggaagacctcaaggtgaaggttgcgaatgttgaaagtggaggagagttcaccatctccagtgctgaagagatcatcaaggccctt

SEQ ID NO: 547 - protein, 162 aa, Glycine max
MAPIAVGNVIPDGILAYLDEENKPQTVSIHSLATGKKVIIFGVPGAFTPTCSLKHVPGFIERAEELKGKGVDEIICISVNDPFVMNSWAKTFPENKHVKFLADGAAKYTNALGLELDLTDKGLGVRSKRFALLVEDLKVKVANVESGGEFTISSAEEIIKAL

SEQ ID NO: 548 - protein, 13 aa, Glycine max
HVPGFIERAEELK

SEQ ID NO: 549 - protein, 10 aa, Glycine max
RFALLVEDLK

SEQ ID NO: 550 - protein, 20 aa, Glycine max
VANVESGGEFTISSAEEIIK

SEQ ID NO: 551 - protein, 14 aa, Glycine max
YTNALGLELDLTDK

SEQ ID NO: 552 - DNA, 456 bp, Glycine max
  source 1..456: mol_type = other DNA
  CDS 1..456
atgtcgcagttgctggataaagccaagaacttcgtgtcggagaaggtaaatgatatggccaagccggaggcgagtgtcactgacgtggacttcaagcgcgtgagcaaagataatgtcgagtacttggccaaggtctctgttcgtaacccttattcaacttccatacccatttgtgagatcaactactctttcaaaagcgcatccagggagatagcatcagggaaaattccagacccaggatcgttgaaggcaaaggacacaacaatggtggatgtgccagttaaggttccatacagcatattgatgagcttggcaaaggacattggtgctgactgggacatagactatcaattggatcttggtctggttattgatgttcctgtcattggcatcttcaccattcctctctctcagaaaggagagatcaagctaccaaccctctctaccatgttcgcc

SEQ ID NO: 553 - protein, 152 aa, Glycine max
MSQLLDKAKNFVSEKVNDMAKPEASVTDVDFKRVSKDNVEYLAKVSVRNPYSTSIPICEINYSFKSASREIASGKIPDPGSLKAKDTTMVDVPVKVPYSILMSLAKDIGADWDIDYQLDLGLVIDVPVIGIFTIPLSQKGEIKLPTLSTMFA

SEQ ID NO: 554 - protein, 8 aa, Glycine max
DNVEYLAK

SEQ ID NO: 555 - DNA, 480 bp, Glycine max
  source 1..480: mol_type = other DNA
  CDS 1..480
atgtcggacgaagagcaccatttcgagtccaaggcagacgccggtgcctccaaaacctaccctcagcaagccggtaccattcgcaagaacggctacatcgtcatcaaaggacgcccctgcaaggttgtggaagtttcgacttccaaaactggaaagcacggtcacgccaagtgccactttgttggaattgatattttcactgccaagaaacttgaggatattgtgccctcttctcacaactgtgatgttcctcatgtgaatcgtactgattaccagctcattgatattgctgaggatggatttttgagtctgcttactgaaaatgggaacaccaaggatgacctcaagcttcccactgatgagagtctgctcactcagataaaggatggatttgctgagggcaaggatcttgttgtgtctgtcatgtctgctatgggtgaggaacagatttgcgccctgaaggatattgggccaaagaac

SEQ ID NO: 556 - protein, 160 aa, Glycine max
MSDEEHHFESKADAGASKTYPQQAGTIRKNGYIVIKGRPCKVVEVSTSKTGKHGHAKCHFVGIDIFTAKKLEDIVPSSHNCDVPHVNRTDYQLIDIAEDGFLSLLTENGNTKDDLKLPTDESLLTQIKDGFAEGKDLVVSVMSAMGEEQICALKDIGPKN

SEQ ID NO: 557 - protein, 12 aa, Glycine max
LPTDESLLTQIK

SEQ ID NO: 558 - protein, 7 aa, Glycine max
NGYIVIK

SEQ ID NO: 559 - DNA, 480 bp, Glycine max
  source 1..480: mol_type = other DNA
  CDS 1..480
atgtcggacgaagagcatcacttcgattctcaggcggatgcgggagcgtccaagacgtatcctcagcaggctggtaccattcgcaagaatggctacatcgtcatcaaatccaggccttgcaaggttgtagaggtctctacgtcaaagacaggcaaacatggacatgcaaagtgtcactttgctgcaattgatatttttaatgggaaaaagcttgaagatattgttccatcatcccacaactgtgatgttccacatgtgaatcgcactgactatcagcttattgatatctctgaagacggctttgtgagtctgttgactgataatggaaataccaaggatgatctgaagcttcctactgatgaggcattgcttgctcagattaaagatggattttctgaaggaaaagaccttgtggtttctgttatgtctgcaatgggagaagaacagatctgttcactgaaggacatcggtcccaaaaat

SEQ ID NO: 560 - protein, 160 aa, Glycine max
MSDEEHHFDSQADAGASKTYPQQAGTIRKNGYIVIKSRPCKVVEVSTSKTGKHGHAKCHFAAIDIFNGKKLEDIVPSSHNCDVPHVNRTDYQLIDISEDGFVSLLTDNGNTKDDLKLPTDEALLAQIKDGFSEGKDLVVSVMSAMGEEQICSLKDIGPKN

SEQ ID NO: 561 - protein, 7 aa, Glycine max
NGYIVIK

SEQ ID NO: 562 - protein, 11 aa, Glycine max
TYPQQAGTIRK

SEQ ID NO: 563 - protein, 8 aa, Glycine max
VVEVSTSK

SEQ ID NO: 564 - DNA, 501 bp, Glycine max
  source 1..501: mol_type = other DNA
  CDS 1..501
atggctagccaatcaaacactaaatcagttcacgatttcacagttaaagatgccaggggaaataatgttaatctcgccgattacaaaggaaaggtccttctcattgtcaatgttgcctcacaatgcggcttgactaattcaaactacacagagcttaaccaattgtatgagaaatacaaagggaaaggacttgaaattctggcattcccatgcaatcagtttggggctcaggagccaggaactaatgaagagattgtggagtttgcttgtactcgcttcaaagctgagtttcccattttcgacaaggtggatgtgaatggtgacaatgctgctccactgtacaagtttctgaaatcaagcaaaggtggactctttggggatagtatcaaatggaacttctccaaattccttgttgataaggatgggaatgtcgttgaccgttatgcacccacaacttctcctctcagcattgagaaagacataaagaagctgctagatgca

SEQ ID NO: 565 - protein, 167 aa, Glycine max
MASQSNTKSVHDFTVKDARGNNVNLADYKGKVLLIVNVASQCGLTNSNYTELNQLYEKYKGKGLEILAFPCNQFGAQEPGTNEEIVEFACTRFKAEFPIFDKVDVNGDNAAPLYKFLKSSKGGLFGDSIKWNFSKFLVDKDGNVVDRYAPTTSPLSIEKDIKKLLDA

SEQ ID NO: 566 - protein, 8 aa, Glycine max
AEFPIFDK

SEQ ID NO: 567 - protein, 13 aa, Glycine max
DARGNNVNLADYK

SEQ ID NO: 568 - protein, 19 aa, Glycine max
DGNVVDRYAPTTSPLSIEK

SEQ ID NO: 569 - protein, 9 aa, Glycine max
GGLFGDSIK

SEQ ID NO: 570 - protein, 13 aa, Glycine max
VDVNGDNAAPLYK

SEQ ID NO: 571 - DNA, 357 bp, Glycine max
  source 1..357: mol_type = other DNA
  CDS 1..357
atggcttccgaagaatcccagaacaagccccccagcgagcagtccactaccgaactcatcacgagcgcgaagctggtggcggaggcggcgcagtcggctctgaagagcgacagcgacaaagtggacaaggccaaggtggctgacgctgccggcgatcttctcgacgcggcggggaagtatggaaaacttgatgacaaacaaggcatagggcaatatgttgacaaggctgctgattatctgcataattaccagggtgataataccactgctccttcacagcctgcagagtctaaaggtgaagaaagtggtggtgggttaggtggtcttgcaaacttggctggaggcttcttcaaaaag

SEQ ID NO: 572 - protein, 119 aa, Glycine max
MASEESQNKPPSEQSTTELITSAKLVAEAAQSALKSDSDKVDKAKVADAAGDLLDAAGKYGKLDDKQGIGQYVDKAADYLHNYQGDNTTAPSQPAESKGEESGGGLGGLANLAGGFFKK

SEQ ID NO: 573 - protein, 23 aa, Glycine max
AADYLHNYQGDNTTAPSQPAESK

SEQ ID NO: 574 - protein, 11 aa, Glycine max
LVAEAAQSALK

SEQ ID NO: 575 - protein, 9 aa, Glycine max
QGIGQYVDK

SEQ ID NO: 576 - protein, 14 aa, Glycine max
VADAAGDLLDAAGK

SEQ ID NO: 577 - DNA, 777 bp, Glycine max
  source 1..777: mol_type = other DNA
  CDS 1..777
atggcggccgctccctcgcctcgcgaagagaacgtctacatggcgaagctcgccgagcaggccgagcgctacgaagagatggtggagttcatggagaaggtctccgccgccgccgacaacgaggaacttaccgtggaggagaggaacctcctctccgtcgcctacaagaacgtgatcggagcgcgccgcgcctcgtggcgcatcatctcctcgatcgagcagaaggaggagagccgcggcaacgaggatcacgtctccgtcatccgcgactaccgatccaagatcgagtccgagctctccaacatctgcgacggtatcctcaagctcctcgactctcgcctcattccatccgcttcctccggcgattccaaggtcttctacctcaagatgaagggagattaccacagataccttgccgagttcaagaccggcgccgagcgtaaggaggctgccgagagcactctctctgcatacaaagccgcacaggacattgcgaatgccgaactgcctcctactcatccaattaggcttggccttgctcttaacttttctgtattttactacgaaatccttaattctccggatcgtgcctgcaaccttgcaaaacaggcttttgatgaagctattgctgaattggatacactgggagaggagtcatacaaggatagcactttgatcatgcaactccttcgtgataacctcacactttggacctctgacatgcaggacgatggagcagatgaaattaaagaagcagcaccgaaaccggatgaccag

SEQ ID NO: 578 - protein, 259 aa, Glycine max
MAAAPSPREENVYMAKLAEQAERYEEMVEFMEKVSAAADNEELTVEERNLLSVAYKNVIGARRASWRIISSIEQKEESRGNEDHVSVIRDYRSKIESELSNICDGILKLLDSRLIPSASSGDSKVFYLKMKGDYHRYLAEFKTGAERKEAAESTLSAYKAAQDIANAELPPTHPIRLGLALNFSVFYYEILNSPDRACNLAKQAFDEAIAELDTLGEESYKDSTLIMQLLRDNLTLWTSDMQDDGADEIKEAAPKPDDQ

SEQ ID NO: 579 - protein, 11 aa, Glycine max
EAAESTLSAYK

SEQ ID NO: 580 - protein, 16 aa, Glycine max
LLDSRLIPSASSGDSK

SEQ ID NO: 581 - protein, 19 aa, Glycine max
QAFDEAIAELDTLGEESYK

SEQ ID NO: 582 - protein, 23 aa, Glycine max
VSAAADNEELTVEERNLLSVAYK

SEQ ID NO: 583 - DNA, 993 bp, Glycine max
  source 1..993: mol_type = other DNA
  CDS 1..993
atggcgttgaacaatggcgtccccgccaccggcaccggcctcatcgtcagcttcggtgagatgctcatcgacttcgtccccaccgtctctggcgtgtccctggccgaggcccctggcttcctcaaggcccccggcggcgcccccgctaacgtcgccatcgccgtgtcgcgcctcggcggcaaagccgccttcgtcggcaagctcggcgacgacgagttcggccacatgctcgccggaatcctcaaggaaaacggcgttcgcgccgacggcatcaactttgaccagggcgcacgcaccgccctggccttcgtgaccctacgcgccgacggggagcgtgagttcatgttctacagaaaccccagcgccgacatgctcctcaagcccgaagaactcaatctcgaactcatcagatctgcaaaagttttccattacggatcaatcagtttgatcgtggagccatgcagatcagcacacttgaaggcaatggaagtagccaaggaatctgggtgcttgctctcctatgaccccaaccttcgtctacctttgtggccttcggctgaggaagctcgtaagcaaatactgagcatttgggagaaggctgatttgatcaaggtcagtgatgcggagcttgagttcctcacaggaagtgacaagattgatgatgaatctgctttgtcattgtggcaccccaatttgaagttgctccttgtcactcttggggaacatggttccagatactacaccaagagtttcaaaggatcggtagatgctttccatgtcaatacagttgatacaactggtgccggtgattcctttgttggtgctttattggccaagattgtcgatgatcagtccatacttgaagatgaaccaaggttaagagaagtactaaagtttgcaaatgcatgtggagctattacaactacccaaaagggagcaattccggcccttcccaaagaggaggctgcactgaaactgatcaaaggggggtca

SEQ ID NO: 584 - protein, 331 aa, Glycine max
MALNNGVPATGTGLIVSFGEMLIDFVPTVSGVSLAEAPGFLKAPGGAPANVAIAVSRLGGKAAFVGKLGDDEFGHMLAGILKENGVRADGINFDQGARTALAFVTLRADGEREFMFYRNPSADMLLKPEELNLELIRSAKVFHYGSISLIVEPCRSAHLKAMEVAKESGCLLSYDPNLRLPLWPSAEEARKQILSIWEKADLIKVSDAELEFLTGSDKIDDESALSLWHPNLKLLLVTLGEHGSRYYTKSFKGSVDAFHVNTVDTTGAGDSFVGALLAKIVDDQSILEDEPRLREVLKFANACGAITTTQKGAIPALPKEEAALKLIKGGS

SEQ ID NO: 585 - protein, 19 aa, Glycine max
APGGAPANVAIAVSRLGGK

SEQ ID NO: 586 - protein, 27 aa, Glycine max
GSVDAFHVNTVDTTGAGDSFVGALLAK

SEQ ID NO: 587 - protein, 19 aa, Glycine max
IVDDQSILEDEPRLREVLK

SEQ ID NO: 588 - protein, 14 aa, Glycine max
VSDAELEFLTGSDK

SEQ ID NO: 589 - DNA, 759 bp, Glycine max
  source 1..759: mol_type = other DNA
  CDS 1..759
atgggcagaaagttcttcgttggtggcaactggaaatgcaatgggaccactgaggaggtgaagaagattgttactactttaaatgaagctaaagtccctggagaagatgttgtagaagttgttgtgagccctccttttgtgttccttccttttgtaaaaagtttgctgcgccctgatttccatgtctcggcccaaaattgttgggttcgcaaaggtggtgcttatactggagaggttagtgctgaaatgcttgttaatttgggaattccttgggttattattggtcactctgaacggaggcagcttttgaatgaatcaaatgagtttgtgggagataaagttgcctatgcacttcaacaaggtctgaaagttatagcatgcattggggaaactcttgaacagcgtgaagctggtacaacaacggctgttgttgctgagcaaacaaaagcaattgcagctaaaatatcaaattgggacaatgtcgttttggcctatgagccagtttgggccattggaacaggaaaggttgcaactcctgctcaggctcaagaggttcatgctgatttaaggaaatgggttcatgacaatgtgagtgctgaagttgctgcatctgtaagaattatctatggaggctctgtaaatggaggaaactgcaaagaattggcagcacagcccgatgttgatggatttttggttggtggtgcatccctcaaggcggaatttgtggacatcataaacgctgccactgtgaagaagaat

SEQ ID NO: 590 - protein, 253 aa, Glycine max
MGRKFFVGGNWKCNGTTEEVKKIVTTLNEAKVPGEDVVEVVVSPPFVFLPFVKSLLRPDFHVSAQNCWVRKGGAYTGEVSAEMLVNLGIPWVIIGHSERRQLLNESNEFVGDKVAYALQQGLKVIACIGETLEQREAGTTTAVVAEQTKAIAAKISNWDNVVLAYEPVWAIGTGKVATPAQAQEVHADLRKWVHDNVSAEVAASVRIIYGGSVNGGNCKELAAQPDVDGFLVGGASLKAEFVDIINAATVKKN

SEQ ID NO: 591 - protein, 13 aa, Glycine max
AEFVDIINAATVK

SEQ ID NO: 592 - protein, 19 aa, Glycine max
ELAAQPDVDGFLVGGASLK

SEQ ID NO: 593 - protein, 8 aa, Glycine max
FFVGGNWK

SEQ ID NO: 594 - protein, 9 aa, Glycine max
IVTTLNEAK

SEQ ID NO: 595 - protein, 16 aa, Glycine max
VATPAQAQEVHADLRK

SEQ ID NO: 596 - protein, 10 aa, Glycine max
VAYALQQGLK

SEQ ID NO: 597 - protein, 22 aa, Glycine max
VPGEDVVEVVVSPPFVFLPFVK

SEQ ID NO: 598 - DNA, 759 bp, Glycine max
  source 1..759: mol_type = other DNA
  CDS 1..759
atgggcagaaaattcttcgttggtggcaactggaaatgcaatgggaccactgaggaggtaaagaagattgttactactttgaatgaggctaaagtccctggagaagatgtcgtagaagttgttgtgagccctccttttgtgttccttcctgttgtaaaaagtttgctgcgccctgatttccatgtttcggcacaaaactgttgggttcgcaaaggtggtgcttataccggtgaggttagtgctgaaatgcttgttaatttgggaattccttgggttattattggtcactctgaacggaggcagcttttaaatgaatcaaatgagtttgtgggagataaagttgcctatgcacttcaacaaggtctaaaagttattgcatgcattggggagactctcgaacagcgtgaagctggtacaacaacggctgttgtttctgagcaaacaaaagcaattgcagctaaaatatcaaattgggacaatgtcgttttggcctacgagccagtttgggccattggaacaggaaaggttgctactcctgctcaggctcaagaggtccatgctgatttgaggaaatgggttcatgacaatgtgagtgctgaagttgctgcatctgtaagaattatctatggaggttctgtaaatggaggaaactgcaaagaattggccgcacagcccgatgttgatggatttttggttggtggtgcctccctgaagccggagttcgtggacatcataaatgctgccactgtgaagaagaat

SEQ ID NO: 599 - protein, 253 aa, Glycine max
MGRKFFVGGNWKCNGTTEEVKKIVTTLNEAKVPGEDVVEVVVSPPFVFLPVVKSLLRPDFHVSAQNCWVRKGGAYTGEVSAEMLVNLGIPWVIIGHSERRQLLNESNEFVGDKVAYALQQGLKVIACIGETLEQREAGTTTAVVSEQTKAIAAKISNWDNVVLAYEPVWAIGTGKVATPAQAQEVHADLRKWVHDNVSAEVAASVRIIYGGSVNGGNCKELAAQPDVDGFLVGGASLKPEFVDIINAATVKKN

SEQ ID NO: 600 - protein, 32 aa, Glycine max
ELAAQPDVDGFLVGGASLKPEFVDIINAATVK

SEQ ID NO: 601 - protein, 8 aa, Glycine max
FFVGGNWK

SEQ ID NO: 602 - protein, 9 aa, Glycine max
IVTTLNEAK

SEQ ID NO: 603 - protein, 16 aa, Glycine max
VATPAQAQEVHADLRK

SEQ ID NO: 604 - protein, 10 aa, Glycine max
VAYALQQGLK

SEQ ID NO: 605 - DNA, 765 bp, Glycine max
  source 1..765: mol_type = other DNA
  CDS 1..765
atggcctcattgggtgggttacaaaatgtgagcggcatcaattttcttatcaaagagaaaggatgttgtatgctgcagggtccaaaagtcaatgcaaagttcgagctgaagccgccaccatatccactgaatggtttggagccggtgatgagccagcagacacttgagtttcactgggggaagcaccacaagacttatgtggaaaatctgaaaaaacaagttgttgggacagagcttgatgggaagtcactagaagagattattgtcacatcatacaataagggtgacattcttccagctttcaacaatgcagcacaggtatggaaccatgacttcttctgggagtgcatgaaaccaggtggaggtggaaagccatccggggagcttctagaactgattgaaagagactttggttcatttgtaaaattccttgatgagttcaaggctgctgctgcaacacaatttggttcagggtgggcttggctagcatatagagcaagaaaatttgatggggaaaatgtagcaaatcctccttcacccgatgaggacaacaagctagtggtgctcaagagtcccaatgctgtgaacccccttgtttggggaggttactacccacttcttaccattgatgtttgggagcatgcttactaccttgattttcagaaccggcgtcctgattatatatcagtgttcatggataagcttgtttcctgggatgcagtgagctctagacttgaacaagctaaggctttaattaccagtgca

SEQ ID NO: 606 - protein, 255 aa, Glycine max
MASLGGLQNVSGINFLIKEKGCCMLQGPKVNAKFELKPPPYPLNGLEPVMSQQTLEFHWGKHHKTYVENLKKQVVGTELDGKSLEEIIVTSYNKGDILPAFNNAAQVWNHDFFWECMKPGGGGKPSGELLELIERDFGSFVKFLDEFKAAAATQFGSGWAWLAYRARKFDGENVANPPSPDEDNKLVVLKSPNAVNPLVWGGYYPLLTIDVWEHAYYLDFQNRRPDYISVFMDKLVSWDAVSSRLEQAKALITSA

SEQ ID NO: 607 - protein, 17 aa, Glycine max
FDGENVANPPSPDEDNK

SEQ ID NO: 608 - protein, 15 aa, Glycine max
LVSWDAVSSRLEQAK

SEQ ID NO: 609 - protein, 10 aa, Glycine max
QVVGTELDGK

SEQ ID NO: 610 - protein, 12 aa, Glycine max
SLEEIIVTSYNK

SEQ ID NO: 611 - protein, 7 aa, Glycine max
TYVENLK

SEQ ID NO: 612 - DNA, 732 bp, Glycine max
  source 1..732: mol_type = other DNA
  CDS 1..732
atggcctcattgggtgggttacaaaatctaagcggcatcaattttcttatcaaaaaagagggtccaaaagtcaatgcgaagttcgagctgaagccgccaccatatccactgaatggtttggagccggtgatgagccagcagacacttgagtttcattgggggaaacaccacaagacatatgtggaaaatctgaaaaaacaaattgttgggacagagcttgatgggaaatcactagaagagattatagtgacatcatataataagggtgacattcttccagctttcaacaatgcagcacaggtatggaaccatgacttcttctgggagtgcatgaaaccaggtggaggtggaaagccatccggggagcttctagaactgattgaaagagactttggctcatttgaaaaattccttgatgagtttaaggctgctgctgcaacacaatttggttcaggatgggcttggcttgcatatagagcaagcaaatttgatggggaaaatgcagcaaatcctccttcaccagatgaggacaacaagctagtggtgctcaagagtcccaatgctgtgaacccccttgtttggggaggctattatccacttcttaccattgatgtttgggagcatgcttacaaccttgattttcagaaccggcgccctgattatatatcagtgttcatggataagcttgtttcctgggatgcagtgagttctagacttgaacaagcaaggctt

SEQ ID NO: 613 - protein, 244 aa, Glycine max
MASLGGLQNLSGINFLIKKEGPKVNAKFELKPPPYPLNGLEPVMSQQTLEFHWGKHHKTYVENLKKQIVGTELDGKSLEEIIVTSYNKGDILPAFNNAAQVWNHDFFWECMKPGGGGKPSGELLELIERDFGSFEKFLDEFKAAAATQFGSGWAWLAYRASKFDGENAANPPSPDEDNKLVVLKSPNAVNPLVWGGYYPLLTIDVWEHAYNLDFQNRRPDYISVFMDKLVSWDAVSSRLEQARL

SEQ ID NO: 614 - protein, 10 aa, Glycine max
QIVGTELDGK

SEQ ID NO: 615 - protein, 12 aa, Glycine max
SLEEIIVTSYNK

SEQ ID NO: 616 - protein, 7 aa, Glycine max
TYVENLK

SEQ ID NO: 617 - DNA, 1419 bp, Glycine max
  source 1..1419: mol_type = other DNA
  CDS 1..1419
atggcttctaaggtggtttcggttttggttatagcgatgatgttgtttgccatgaactgtaactgcactagtgttggccacatgccttcaaccaaagaggaaggtcacgattttcaggagtctaaagccaagaccacacaaactgcgaacaaggccatggaaactggtaaagagggacaagaagccgcagagtcatggacagaatgggctaaagagaaactcagtgaaggtcttggcttcaaacatgatcaggaatccaaagaatccaccactaacaaagtctctgattatgccactgacactgcacagaaatccaaggattatgcaactgacactgctcagaaatcaaaggactatgcaggtgatgctgctcagaaatcaaaggactacgcaggtgatgctgctcagaaaaccaaagactatgccagtgacactgcacagacatcaaaggactatgctggtgatgctgctcagaaatcaaagggctatgttggtgatgctgcacaaaagaccaaagaatatgttggtgatgcagcacagaagacaaaggattatgccactgacgcagcacagaagaccaaagattatgctactcagaagaccaaggactatgcaagtgatgcaactgatgctgcaaagaagactaaagattatgctgctcagaagactaaggactatgcaagtgaagctagtgatgttgcacagaacacaaaagattatgctgctcagaagaccaaggactatgccagcggtggtgcgcagaagaccaaggattatgccagcggtggtgcgcagaagaccaaggactatgcaagtgatgctgcgcagaagaccaaggactatgcaagtgatggtgctcaaaagagcaaagaatatgcgggtgatgtagcactgaatgccaaagattatgctcaaaaaagcaaagattacgcaggtgatgctgcacagaatgtcaaagattatgcaagtgatgcagtgcagaagagaaaagagtattctggtgatgcttctcacaagagcaaagaggcttctgattatgccagtgagacggctaagaagaccaaagattatgtcggtgatgctgctcagagaagcaaaggggctgctgaatatgctagtgatgcggctcagaggaccaaagagtatgctggtgatgctaccaagagaagcaaagaggcttctaatgatcatgccaatgacatggctcagaagaccaaagactacgccagtgacactgctcagagaaccaaggaaaaactccaagacattgcatctgaagctggtcaatacagtgcagagaaagctagagaaatgaaggatgcagcagcagagaaggcaagtgacatagcaaaggctgcaaagcaaaaatcacaagaggtgaaagagaaattaggtggacaacaccgggatgccgagctt

SEQ ID NO: 618 - protein, 473 aa, Glycine max
MASKVVSVLVIAMMLFAMNCNCTSVGHMPSTKEEGHDFQESKAKTTQTANKAMETGKEGQEAAESWTEWAKEKLSEGLGFKHDQESKESTTNKVSDYATDTAQKSKDYATDTAQKSKDYAGDAAQKSKDYAGDAAQKTKDYASDTAQTSKDYAGDAAQKSKGYVGDAAQKTKEYVGDAAQKTKDYATDAAQKTKDYATQKTKDYASDATDAAKKTKDYAAQKTKDYASEASDVAQNTKDYAAQKTKDYASGGAQKTKDYASGGAQKTKDYASDAAQKTKDYASDGAQKSKEYAGDVALNAKDYAQKSKDYAGDAAQNVKDYASDAVQKRKEYSGDASHKSKEASDYASETAKKTKDYVGDAAQRSKGAAEYASDAAQRTKEYAGDATKRSKEASNDHANDMAQKTKDYASDTAQRTKEKLQDIASEAGQYSAEKAREMKDAAAEKASDIAKAAKQKSQEVKEKLGGQHRDAEL

SEQ ID NO: 619 - protein, 11 aa, Glycine max
DYAGDAAQNVK

SEQ ID NO: 620 - protein, 9 aa, Glycine max
DYASDAAQK

SEQ ID NO: 621 - protein, 11 aa, Glycine max
DYASDATDAAK

SEQ ID NO: 622 - protein, 9 aa, Glycine max
DYASDAVQK

SEQ ID NO: 623 - protein, 11 aa, Glycine max
DYASDTAQRTK

SEQ ID NO: 624 - protein, 11 aa, Glycine max
DYASDTAQTSK

SEQ ID NO: 625 - protein, 14 aa, Glycine max
DYASEASDVAQNTK

SEQ ID NO: 626 - protein, 9 aa, Glycine max
DYATDAAQK

SEQ ID NO: 627 - protein, 11 aa, Glycine max
DYVGDAAQRSK

SEQ ID NO: 628 - protein, 11 aa, Glycine max
EASDYASETAK

SEQ ID NO: 629 - protein, 11 aa, Glycine max
EYAGDVALNAK

SEQ ID NO: 630 - protein, 9 aa, Glycine max
EYVGDAAQK

SEQ ID NO: 631 - protein, 14 aa, Glycine max
GAAEYASDAAQRTK

SEQ ID NO: 632 - protein, 9 aa, Glycine max
GYVGDAAQK

SEQ ID NO: 633 - protein, 15 aa, Glycine max
LQDIASEAGQYSAEK

SEQ ID NO: 634 - protein, 8 aa, Glycine max
LSEGLGFK

SEQ ID NO: 635 - protein, 11 aa, Glycine max
VSDYATDTAQK

SEQ ID NO: 636 - DNA, 1374 bp, Glycine max
  source 1..1374: mol_type = other DNA
  CDS 1..1374
atggcttcgagggtggtttcggttttggtgatagcgatgatgttgtttgccatgaactgtaactgcactagtgttggccacatgccttcaaccaaagaggaaggtcacgattttcaggaggctaaagccaagacaacacaaactgcgaacaaggccatggaaactggtaaagatgggaaagaagccgcagagtcatggacagaatgggctaaagagaaactcactgaaggtcttggcttcaaacatgatcaagaatccaccactaagaaagtctctgactatgccggtgatgctgctcagaaatcaaaggactatgccggtgacactgctcagaaaaccaaggactatgccggtgacactgctcagaaaaccaaggactatgccggtgacactgcacagacatcaaaggactatgccggtgatgctgctcagaaatcaaagggctatgtcggtgatgctgcacaaaagaccaaagaatatgttggtgatgctgctcagaagaccatggattatgccactgatgcagcacagaagaccaaagattatgctactcagaagactaaggactatgcaagtgatgcaactgatgctgcaaaaaagactaaagattatgctgctcagaagaccaaggattatgcaagtgatgcaactgatgctgcaaaaaagactaaggattatgctgcgcagaagaccaaggactatgccagtggtggtgcgcagaagaccaaggactatgccagtggtgctgcgcagaagaccaaggactatgcaagtgatgctgcgcagaggaccaaggaccatgcaagtgatggtgctcaaaagagcaaagaatatgcgggtgatgtagcacagaatgccaaagattatgctcaaaagagcaaagattatgcgggtgatgctgtacagaatatcaaagattatgcaaatgatgcagcgcacaagagcaaagattatgctggtgatgcttctcacaagagcaaagaagcttctgattatgccagtgagacggctcagaagaccaaagactatgttggtgatgctgctcagaaaagcaaagaggcttctgaatatgctagtgatgcggctcagaggaccaaagagtatgctggtgatgccaccaagagaagcaaagaggcttctgatcatgccaatgacatggctcggaagaccaaagactacgccagtgacactgctcagagaaccaaggaaaaactccaagacattgcatctgaggctggtcaatacagtacagagaaggccagagaaatgaaggatgcagcagcagagaaggctagtgacattgcaaaggctgcaaagcaaaaatcacaagagctgaaagacaaattaggtggacaacaccgggatgctgagctt

SEQ ID NO: 637 - protein, 458 aa, Glycine max
MASRVVSVLVIAMMLFAMNCNCTSVGHMPSTKEEGHDFQEAKAKTTQTANKAMETGKDGKEAAESWTEWAKEKLTEGLGFKHDQESTTKKVSDYAGDAAQKSKDYAGDTAQKTKDYAGDTAQKTKDYAGDTAQTSKDYAGDAAQKSKGYVGDAAQKTKEYVGDAAQKTMDYATDAAQKTKDYATQKTKDYASDATDAAKKTKDYAAQKTKDYASDATDAAKKTKDYAAQKTKDYASGGAQKTKDYASGAAQKTKDYASDAAQRTKDHASDGAQKSKEYAGDVAQNAKDYAQKSKDYAGDAVQNIKDYANDAAHKSKDYAGDASHKSKEASDYASETAQKTKDYVGDAAQKSKEASEYASDAAQRTKEYAGDATKRSKEASDHANDMARKTKDYASDTAQRTKEKLQDIASEAGQYSTEKAREMKDAAAEKASDIAKAAKQKSQELKDKLGGQHRDAEL

SEQ ID NO: 638 - protein, 11 aa, Glycine max
DYAGDAVQNIK

SEQ ID NO: 639 - protein, 11 aa, Glycine max
DYASDAAQRTK

SEQ ID NO: 640 - protein, 11 aa, Glycine max
DYASDATDAAK

SEQ ID NO: 641 - protein, 11 aa, Glycine max
DYASDTAQRTK

SEQ ID NO: 642 - protein, 9 aa, Glycine max
DYVGDAAQK

SEQ ID NO: 643 - protein, 12 aa, Glycine max
EASDYASETAQK

SEQ ID NO: 644 - protein, 14 aa, Glycine max
EASEYASDAAQRTK

SEQ ID NO: 645 - protein, 11 aa, Glycine max
EYAGDVAQNAK

SEQ ID NO: 646 - protein, 9 aa, Glycine max
GYVGDAAQK

SEQ ID NO: 647 - protein, 15 aa, Glycine max
LQDIASEAGQYSTEK

SEQ ID NO: 648 - protein, 8 aa, Glycine max
LTEGLGFK

SEQ ID NO: 649 - protein, 11 aa, Glycine max
VSDYAGDAAQK